(19) United States
(12) Patent Application Publication
(10) Pub. No.: US 2026/0196349 A1
(43) Pub. Date: Jul. 9, 2026

(54) PAN-CANCER AND TISSUE-SPECIFIC CANCER PROTEINS AND METHODS OF USE THEREOF

(71) Applicant: Novelna Inc., Palo Alto, CA (US)

(72) Inventor: Afshin Ashkan, Santa Clara, CA (US)

(21) Appl. No.: 19/134,728

(22) PCT Filed: Dec. 4, 2023
(86) PCT No.: PCT/US2023/082330

§ 371 Date: May 31, 2025

Related U.S. Application Data
(60) Provisional application No. 63/429,803, filed on Dec. 2, 2022.
(60) Provisional application No. 63/446,007, filed on Feb. 15, 2023.

(51) Int. Cl. G16H 50/20; G01N 33/68; G16B 25/10; G16H 10/40; G16H 20/10

(52) U.S. Cl. CPC G16H 50/20; G01N 33/6893; G16B 25/10; G16H 10/40; G16H 20/10

(57) ABSTRACT

Methods of evaluating a subject for cancer, comprising: determining in a biological sample from the subject a concentration of one or more pan-cancer detection proteins, and determining in a biological sample from the subject a concentration of one or more tissue-specific cancer localization proteins. In addition, methods of treating cancer in a subject comprising determining in a biological sample from the subject a concentration of one or more pan-cancer detection proteins, and determining in a biological sample from the subject a concentration of one or more tissue-specific cancer localization proteins and applying a classifier to the concentration to identify that the subject has cancer; determining in a biological sample from the subject a concentration of one or more tissue-specific cancer localization proteins and applying a classifier to the concentration to identify with which tissue the cancer is associated; and administering a treatment to the subject.

### CROSS-REFERENCE TO RELATED APPLICATIONS

This application claims the benefit of U.S. Provisional Application No. 63/429,803, filed on Dec. 2, 2022, and 63/446,007, filed on Feb. 15, 2023, each of which is incorporated herein by reference in its entirety.

### BACKGROUND

Cancer continues to be one of the deadliest threats to human health. In the U.S., cancer is the second leading cause of death after heart disease, accounting for approximately one in four deaths, and within the next five years cancer is expected to surpass cardiovascular diseases as the number one cause of death. Despite the significant medical advances in the treatment of certain cancers, the overall five-year survival rate for all cancers has improved only by about 10% in the past 20 years.

Many cancers are particularly dangerous because of how rapidly cancer cells can metastasize and grow, which makes its early detection essential. For example, the five-year survival rate for lung cancer is over 50% when the disease is still localized within the lungs, and only 5% when the disease has spread to other organs. However, most lung cancer cases are diagnosed when already metastatic (57%) while only 16% are diagnosed at an early stage. Similarly, the five-year survival rate for breast cancer is around 85% when the disease is detected at stage I, and only about 10% when stage IV metastatic disease is detected. Moreover, the 5-year survival rate for colorectal cancer (CRC) is >90% when detected at stage I, but only about 10% for those in whom stage IV metastatic disease is detected.

As a result, screening programs have been developed to identify individuals with cancerous or precancerous conditions. Examples include mammography scanning to detect tumors associated with breast cancer, cervical smear sample testing for HPV and/or inspection for abnormal cervical cells to identify risk of cervical cancer, Fecal Immunochemical Testing (FIT) and/or colonoscopy to monitor for colorectal cancer (CRC), and low dose computed tomography (LDCT) scanning as an early detection measure for lung cancer.

Yet, the majority of commonly occurring cancers are not screened for including, for instance, lymphoma, kidney, bladder, pancreatic, uterus, myeloma, thyroid, ovarian or liver cancers. And even for the cancers that are screened for, the techniques may not be able to discern whether tissue growths are cancerous or benign. There remains a need for both pan- and tissue-specific detection of cancer.

### SUMMARY OF THE INVENTION

Some of the main aspects of the present invention are summarized below. Additional aspects are described in the Detailed Description of the Invention, Examples. Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

One aspect of the invention relates to a method of evaluating a subject for cancer, comprising: (I) determining in a biological sample from the subject a concentration of one or more proteins selected from Table 101 or Table 1001; and (II) determining in a biological sample from the subject a concentration of one or more proteins selected from each of Tables 201-218 or each of Tables 2001-2018; or determining in a biological sample from the subject a concentration of each protein from Table 251, 252, 253, or 254; thereby evaluating the subject for cancer. In some embodiments, the method may further comprise administering a treatment to the subject.

Another aspect relates to a method of treating cancer in a subject, comprising (a) acquiring results from an evaluation of the subject that determined to which tissue the cancer is associated, and (b) administering a treatment to the subject; in which the evaluation comprises (i) determining in a biological sample from the subject a concentration of one or more proteins selected from Table 101 or Table 1001, and applying a classifier to the concentration of the one or more proteins to identify that the subject has cancer, and (ii) determining in a biological sample from the subject a concentration of one or more proteins selected from each of Tables 201-218 or each of Tables 2001-2018, and applying a classifier to the concentration of the one or more proteins to identify with which tissue the cancer is associated; or determining in a biological sample from the subject a concentration of each of the proteins of Tables 251, 252, 253, or 254, and applying a classifier to the concentration of each protein to identify with which tissue the cancer is associated.

A further aspect of the invention relates to a method of evaluating a subject for a cancer associated with a tissue selected from bladder, bone, brain, breast, cervix, colon (colorectal), esophagus, kidney, liver, lung—small cell (SC), lung—non-small cell (NSC), ovary, pancreas, prostate, stomach, testis, thyroid, and uterus, the method comprising: determining in a biological sample from the subject a concentration of one or more proteins selected from Tables 301-318 that respectively correspond to said tissues; thereby evaluating the subject for cancer. In some embodiments, the method further comprises administering a treatment to the subject.

Yet another aspect of the invention relates to a method of treating cancer in a subject, wherein the cancer is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising: (a) acquiring results from an evaluation of the subject that determined the subject has cancer associated with the tissue, and (b) administering a treatment to the subject; in which the evaluation comprises (i) determining in a biological sample from the subject a concentration of one or more proteins selected from Tables 301-318 that respectively correspond to said tissues, and (ii) applying a classifier to the concentration of the one or more proteins to identify with which tissue the cancer is associated.

Another aspect of the invention relates to a method of evaluating a subject who is suspected of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising: determining in a biological sample from the subject a concentration of one or more proteins selected from Tables 201-218 or from Tables 2001-2018 that respectively correspond to said tissues; thereby evaluating the subject for cancer. In some embodiments, the method further comprises administering a treatment to the subject.

Yet another aspect of the invention relates to a method of treating cancer in a subject who is suspected of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising: (a) acquiring results from an evaluation of the subject that determined the subject has cancer associated with the tissue, and (b) administering a treatment to the subject; in which the evaluation comprises (i) determining in a biological sample from the subject a concentration of one or more proteins selected from Tables 201-218 or from Tables 2001-2018 that respectively correspond to said tissues, and (ii) applying a classifier to the concentration of the one or more proteins to identify that the subject has cancer associated with said tissue.

In some embodiments, the cancer associated with bone is an osteosarcoma. In some embodiments, the cancer associated with the brain is an astrocytoma. In some embodiments, the cancer associated with the kidney is a hepatocellular carcinoma.

Another aspect of the invention relates to a method of treating cancer in a subject, comprising (a) acquiring results from a method of evaluating a subject of the present invention, and (b) administering a treatment to the subject.

Yet, a further aspect of the invention relates to a method of measuring amounts of proteins in a subject, the method comprising determining individual amounts of one or more proteins selected from Table 101 or from Table 1001, or selected from any of Tables 201-218 or any of Tables 2001-2018, or selected from any of Tables 301-318; or the method comprising determining individual amounts of each protein of Table 251, 252, 253, or 254.

In addition, an aspect of the invention relates to a method of evaluating a treatment for cancer in a subject, the method comprising administering a treatment for cancer, and determining in a biological sample from the subject a concentration of one or more proteins, in which the one or more proteins are selected from any of Tables 101, 1001, 201-218, 2001-2018, or 301-318; or determining in a biological sample from the subject a concentration of each of proteins are selected from any of Table 251, 252, 253, or 254. Further aspects relate to methods of evaluating the efficacy of a treatment in a subject, adjusting a treatment for cancer in a subject, and monitoring for cancer recurrence in a subject, the methods comprising administering a treatment for cancer, and determining in a biological sample from the subject a concentration of one or more proteins, in which the one or more proteins are selected from any of Tables 101, 1001, 201-218, 2001-2018, or 301-318; or determining in a biological sample from the subject a concentration of each of proteins are selected from any of Table 251, 252, 253, or 254.

### BRIEF DESCRIPTION OF THE FIGURES

FIG. 1 shows an overview of the analysis, described in Example 1.

FIG. 2 shows accuracy, measured as AUC of ROC, of detecting cancer in a subject associated with the bladder, using random combinations of two to 15 proteins selected from Table 201.A.1 (Panel A), Table 201.B.1 (Panel B), and Table 201.C.1 (Panel C), as described in Example 1. The process of selecting the random combinations of each number of proteins (two proteins, three proteins, etc.) was performed for 1000 iterations.

FIG. 3 shows accuracy, measured as AUC of ROC, of detecting cancer in a subject associated with bone, using random combinations of two to 15 proteins selected from Table 202.A.1 (Panel A), Table 202.B.1 (Panel B), and Table 202.C.1 (Panel C), as described in Example 1. The process of selecting the random combinations of each number of proteins (two proteins, three proteins, etc.) was performed for 1000 iterations.

FIG. 4 shows accuracy, measured as AUC of ROC, of detecting cancer in a subject associated with the brain, using random combinations of two to 15 proteins selected from Table 203.A.1 (Panel A), Table 203.B.1 (Panel B), and Table 203.C.1 (Panel C), as described in Example 1. The process of selecting the random combinations of each number of proteins (two proteins, three proteins, etc.) was performed for 1000 iterations.

FIG. 5 shows accuracy, measured as AUC of ROC, of detecting cancer in a subject associated with the breast, using random combinations of two to 15 proteins selected from Table 204.A.1 (Panel A) and Table 204.B.1 (Panel B), as described in Example 1. The process of selecting the random combinations of each number of proteins (two proteins, three proteins, etc.) was performed for 1000 iterations.

FIG. 6 shows accuracy, measured as AUC of ROC, of detecting cancer in a subject associated with the cervix, using random combinations of two to 15 proteins selected from Table 205.A.1 (Panel A) and Table 205.B.1 (Panel B), as described in Example 1. The process of selecting the random combinations of each number of proteins (two proteins, three proteins, etc.) was performed for 1000 iterations.

FIG. 7 shows accuracy, measured as AUC of ROC, of detecting cancer in a subject associated with the colon, using random combinations of two to 15 proteins selected from Table 206.A.1 (Panel A), Table 206.B.1 (Panel B), and Table 206.C.1 (Panel C), as described in Example 1. The process of selecting the random combinations of each number of proteins (two proteins, three proteins, etc.) was performed for 1000 iterations.

FIG. 8 shows accuracy, measured as AUC of ROC, of detecting cancer in a subject associated with the esophagus, using random combinations of two to 15 proteins selected from Table 207.A.1 (Panel A), Table 207.B.1 (Panel B), and Table 207.C.1 (Panel C), as described in Example 1. The process of selecting the random combinations of each number of proteins (two proteins, three proteins, etc.) was performed for 1000 iterations.

FIG. 9 shows accuracy, measured as AUC of ROC, of detecting cancer in a subject associated with the kidney, using random combinations of two to 15 proteins selected from Table 208.A.1 (Panel A), Table 208.B.1 (Panel B), and Table 208.C.1 (Panel C), as described in Example 1. The process of selecting the random combinations of each number of proteins (two proteins, three proteins, etc.) was performed for 1000 iterations.

FIG. 10 shows accuracy, measured as AUC of ROC, of detecting cancer in a subject associated with the liver, using random combinations of two to 15 proteins selected from Table 209.A.1 (Panel A), Table 209.B.1 (Panel B), and Table 209.C.1 (Panel C), as described in Example 1. The process of selecting the random combinations of each number of proteins (two proteins, three proteins, etc.) was performed for 1000 iterations.

FIG. 11 shows accuracy, measured as AUC of ROC, of detecting cancer in a subject associated with the lung—small cell, using random combinations of two to 15 proteins selected from Table 210.A.1 (Panel A), Table 210.B.1 (Panel B), and Table 210.C.1 (Panel C), as described in Example 1. The process of selecting the random combinations of each number of proteins (two proteins, three proteins, etc.) was performed for 1000 iterations.

FIG. 12 shows accuracy, measured as AUC of ROC, of detecting cancer in a subject associated with the lung—non-small cell, using random combinations of two to 15 proteins selected from Table 211.A.1 (Panel A), Table 211.B.1 (Panel B), and Table 211.C.1 (Panel C), as described in Example 1. The process of selecting the random combinations of each number of proteins (two proteins, three proteins, etc.) was performed for 1000 iterations.

FIG. 13 shows accuracy, measured as AUC of ROC, of detecting cancer in a subject associated with the ovary, using random combinations of two to 15 proteins selected from Table 212.A.1 (Panel A) and Table 212.B.1 (Panel B), as described in Example 1. The process of selecting the random combinations of each number of proteins (two proteins, three proteins, etc.) was performed for 1000 iterations.

FIG. 14 shows accuracy, measured as AUC of ROC, of detecting cancer in a subject associated with the pancreas, using random combinations of two to 15 proteins selected from Table 213.A.1 (Panel A), Table 213.B.1 (Panel B), and Table 213.C.1 (Panel C), as described in Example 1. The process of selecting the random combinations of each number of proteins (two proteins, three proteins, etc.) was performed for 1000 iterations.

FIG. 15 shows accuracy, measured as AUC of ROC, of detecting cancer in a subject associated with the prostate, using random combinations of two to 15 proteins selected from Table 214.A.1 (Panel A) and Table 214.B.1 (Panel B), as described in Example 1. The process of selecting the random combinations of each number of proteins (two proteins, three proteins, etc.) was performed for 1000 iterations.

FIG. 16 shows accuracy, measured as AUC of ROC, of detecting cancer in a subject associated with the stomach, using random combinations of two to 15 proteins selected from Table 215.A.1 (Panel A). Table 215.B.1 (Panel B), and Table 215.C.1 (Panel C), as described in Example 1. The process of selecting the random combinations of each number of proteins (two proteins, three proteins, etc.) was performed for 1000 iterations.

FIG. 17 shows accuracy, measured as AUC of ROC, of detecting cancer in a subject associated with the testis, using random combinations of two to 15 proteins selected from Table 216.A.1 (Panel A) and Table 216.B.1 (Panel B), as described in Example 1. The process of selecting the random combinations of each number of proteins (two proteins, three proteins, etc.) was performed for 1000 iterations.

FIG. 18 shows accuracy, measured as AUC of ROC, of detecting cancer in a subject associated with the thyroid, using random combinations of two to 15 proteins selected from Table 217.A.1 (Panel A), Table 217.B.1 (Panel B), and Table 217.C.1 (Panel C), as described in Example 1. The process of selecting the random combinations of each number of proteins (two proteins, three proteins, etc.) was performed for 1000 iterations.

FIG. 19 shows accuracy, measured as AUC of ROC, of detecting cancer in a subject associated with the uterus, using random combinations of two to 15 proteins selected from Table 218.A.1 (Panel A) and Table 218.B.1 (Panel B), as described in Example 1. The process of selecting the random combinations of each number of proteins (two proteins, three proteins, etc.) was performed for 1000 iterations.

FIG. 20 shows accuracy of detecting cancer in a subject, measured as area-under-the-curve (AUC) of receiver operating curves (ROC), using random combinations of two to 20 proteins selected from Table 101.A.1 (Panel A), Table 101.B.1 (Panel B), and Table 101.C.1 (Panel C), as described in Example 1. The process of selecting the random combinations of each number of proteins (two proteins, three proteins, etc.) was performed for 1000 iterations.

FIG. 21 shows accuracy, measured as AUC of ROC, of detecting cancer in a subject associated with the bladder, using random combinations of two to 15 proteins selected from Table 301.A.1 (Panel A), Table 301.B.1 (Panel B), and Table 301.C.1 (Panel C), as described in Example 1. The process of selecting the random combinations of each number of proteins (two proteins, three proteins, etc.) was performed for 1000 iterations.

FIG. 22 shows accuracy, measured as AUC of ROC, of detecting cancer in a subject associated with bone, using random combinations of two to 15 proteins selected from Table 302.A.1 (Panel A), Table 302.B.1 (Panel B), and Table 302.C.1 (Panel C), as described in Example 1. The process of selecting the random combinations of each number of proteins (two proteins, three proteins, etc.) was performed for 1000 iterations.

FIG. 23 shows accuracy, measured as AUC of ROC, of detecting cancer in a subject associated with the brain, using random combinations of two to 15 proteins selected from Table 303.A.1 (Panel A), Table 303.B.1 (Panel B), and Table 303.C.1 (Panel C), as described in Example 1. The process of selecting the random combinations of each number of proteins (two proteins, three proteins, etc.) was performed for 1000 iterations.

FIG. 24 shows accuracy, measured as AUC of ROC, of detecting cancer in a subject associated with the breast, using random combinations of two to 15 proteins selected from Table 304.A.1 (Panel A) and Table 304.B.1 (Panel B), as described in Example 1. The process of selecting the random combinations of each number of proteins (two proteins, three proteins, etc.) was performed for 1000 iterations.

FIG. 25 shows accuracy, measured as AUC of ROC, of detecting cancer in a subject associated with the cervix, using random combinations of two to 15 proteins selected from Table 305.A.1 (Panel A) and Table 305.B.1 (Panel B), as described in Example 1. The process of selecting the random combinations of each number of proteins (two proteins, three proteins, etc.) was performed for 1000 iterations.

FIG. 26 shows accuracy, measured as AUC of ROC, of detecting cancer in a subject associated with the colon, using random combinations of two to 15 proteins selected from Table 306.A.1 (Panel A), Table 306.B.1 (Panel B), and Table 306.C.1 (Panel C), as described in Example 1. The process of selecting the random combinations of each number of proteins (two proteins, three proteins, etc.) was performed for 1000 iterations.

FIG. 27 shows accuracy, measured as AUC of ROC, of detecting cancer in a subject associated with the esophagus, using random combinations of two to 15 proteins selected from Table 307.A.1 (Panel A). Table 307.B.1 (Panel B), and Table 307.C.1 (Panel C), as described in Example 1. The process of selecting the random combinations of each number of proteins (two proteins, three proteins, etc.) was performed for 1000 iterations.

FIG. 28 shows accuracy, measured as AUC of ROC, of detecting cancer in a subject associated with the kidney, using random combinations of two to 15 proteins selected from Table 308.A.1 (Panel A), Table 308.B.1 (Panel B), and Table 308.C.1 (Panel C), as described in Example 1. The process of selecting the random combinations of each number of proteins (two proteins, three proteins, etc.) was performed for 1000 iterations.

FIG. 29 shows accuracy, measured as AUC of ROC, of detecting cancer in a subject associated with the liver, using random combinations of two to 15 proteins selected from Table 309.A.1 (Panel A), Table 309.B.1 (Panel B), and Table 309.C.1 (Panel C), as described in Example 1. The process of selecting the random combinations of each number of proteins (two proteins, three proteins, etc.) was performed for 1000 iterations.

FIG. 30 shows accuracy, measured as AUC of ROC, of detecting cancer in a subject associated with the lung—small cell, using random combinations of two to 15 proteins selected from Table 310.A.1 (Panel A), Table 310.B.1 (Panel B), and Table 310.C.1 (Panel C), as described in Example 1. The process of selecting the random combinations of each number of proteins (two proteins, three proteins, etc.) was performed for 1000 iterations.

FIG. 31 shows accuracy, measured as AUC of ROC, of detecting cancer in a subject associated with the lung—non-small cell, using random combinations of two to 15 proteins selected from Table 311.A.1 (Panel A), Table 311.B.1 (Panel B), and Table 311.C.1 (Panel C), as described in Example 1. The process of selecting the random combinations of each number of proteins (two proteins, three proteins, etc.) was performed for 1000 iterations.

FIG. 32 shows accuracy, measured as AUC of ROC, of detecting cancer in a subject associated with the ovary, using random combinations of two to 15 proteins selected from Table 312.A.1 (Panel A) and Table 312.B.1 (Panel B), as described in Example 1. The process of selecting the random combinations of each number of proteins (two proteins, three proteins, etc.) was performed for 1000 iterations.

FIG. 33 shows accuracy, measured as AUC of ROC, of detecting cancer in a subject associated with the pancreas, using random combinations of two to 15 proteins selected from Table 313.A.1 (Panel A), Table 313.B.1 (Panel B), and Table 313.C.1 (Panel C), as described in Example 1. The process of selecting the random combinations of each number of proteins (two proteins, three proteins, etc.) was performed for 1000 iterations.

FIG. 34 shows accuracy, measured as AUC of ROC, of detecting cancer in a subject associated with the prostate, using random combinations of two to 15 proteins selected from Table 314.A.1 (Panel A) and Table 314.B.1 (Panel B), as described in Example 1. The process of selecting the random combinations of each number of proteins (two proteins, three proteins, etc.) was performed for 1000 iterations.

FIG. 35 shows accuracy, measured as AUC of ROC, of detecting cancer in a subject associated with the stomach, using random combinations of two to 15 proteins selected from Table 315.A.1 (Panel A), Table 315.B.1 (Panel B), and Table 315.C.1 (Panel C), as described in Example 1. The process of selecting the random combinations of each number of proteins (two proteins, three proteins, etc.) was performed for 1000 iterations.

FIG. 36 shows accuracy, measured as AUC of ROC, of detecting cancer in a subject associated with the testis, using random combinations of two to 15 proteins selected from Table 316.A.1 (Panel A) and Table 316.B.1 (Panel B), as described in Example 1. The process of selecting the random combinations of each number of proteins (two proteins, three proteins, etc.) was performed for 1000 iterations.

FIG. 37 shows accuracy, measured as AUC of ROC, of detecting cancer in a subject associated with the thyroid, using random combinations of two to 15 proteins selected from Table 317.A.1 (Panel A), Table 317.B.1 (Panel B), and Table 317.C.1 (Panel C), as described in Example 1. The process of selecting the random combinations of each number of proteins (two proteins, three proteins, etc.) was performed for 1000 iterations.

FIG. 38 shows accuracy, measured as AUC of ROC, of detecting cancer in a subject associated with the uterus, using random combinations of two to 15 proteins selected from Table 318.A.1 (Panel A) and Table 318.B.1 (Panel B), as described in Example 1. The process of selecting the random combinations of each number of proteins (two proteins, three proteins, etc.) was performed for 1000 iterations.

FIG. 39 shows volcano plots of the pan-cancer detection proteins based on p-value among both sexes (Panel A), females (Panel B), and males (Panel C), as described in Example 2. The plots illustrate differential abundances of proteins in cancer versus healthy plasma samples among these groups. The top 100 proteins based on their p-value are highlighted in red.

FIG. 40 shows correlation matrices of the top 100 pan-cancer detection proteins based on p-value among both sexes (Panel A), females (Panel B), and males (Panel C), as described in Example 2.

FIG. 41 shows plots ranking the top ten pan-cancer detection proteins for females (Panel A) and for males (Panel B), and for ten randomly selected proteins (Panel C), in both females and males, as described in Example 2. These results demonstrate the differential expression of the proteins between the genders.

FIG. 42 shows a scatter plot of p-values for each of the top 100 pan-cancer detection proteins among females and males, as described in Example 2.

FIG. 43 shows Venn diagram of the top 100 pan-cancer detection proteins based on the Volcano plots in females and males, as described in Example 2. The Venn diagram demonstrates that there is very little overlap between the top 100 proteins for each gender.

FIG. 44 shows relationship between the number of pan-cancer detection proteins and performance of the proteins for females (Panel A) and males (Panel B), as described in Example 2.

FIG. 45 illustrates differential expression of 10-protein detection panel in tumor and normal plasma samples in females, as described in Example 2. The results are shown for the proteins listed in Table 101.B.3, i.e., IGSF21 (p<0.001) (Panel A), DSG4 (p<0.000) (Panel B). EDAR (p<0.001) (Panel C), CST6 (p<0.001) (Panel D), FLT (p<0.001) (Panel E). FGA (p<0.00l) (Panel F), TRIM58 (p<0.001) (Panel G), MTPN (p<0.001) (Panel H), DDT (p<0.001) (Panel I), and SH3BGRL2 (p<0.001) (Panel J).

FIG. 46 illustrates differential expression of 10-protein detection panel in tumor and normal plasma samples in males, as described in Example 2. The results are shown for the proteins listed in Table 101.B.4, i.e., CCL7 (p<0.020) (Panel A), CCL11 (p<0.006) (Panel B). ENPP5 (p<0.001) (Panel C), COL15A1 (p<0.013) (Panel D), MMP1 (p<0.001) (Panel E). NPPC (p<0.010) (Panel F), GLP1R (p<0.007) (Panel G), ARF6 (p<0.007) (Panel H), WNT9A (p<0.001) (Panel I), and SHBG (p<0.001) (Panel J).

FIG. 47 shows receiver operator characteristic (ROC) curves of 10-protein pan-cancer detection panels for females (Panel A) and males (Panel B), highlighting 99% specificity point, as described in Example 2.

FIG. 48 shows matrices depicting the correlation between each of the top 10 pan-cancer detection proteins for females (Panel A) and males (Panel B), as described in Example 2.

FIG. 49 shows sensitivity at 0.99 specificity (Panel A) and AUC (Panel B) of 10-protein pan-cancer detection for females at cancer stages I, II, and III, as described in Example 2.

FIG. 50 shows sensitivity at 0.99 specificity (Panel A) and AUC (Panel B) of 10-protein pan-cancer detection for males at cancer stages I, II, and III, as described in Example 2.

FIG. 51 shows accuracy, measured as AUC of ROC (Panel A), and sensitivity when specificity is at 0.99 (Panel B) of 10-protein pan-cancer detection for unseen cancers for females, as described in Example 2.

FIG. 52 shows accuracy, as measured by AUC of ROC (Panel A), and sensitivity at 0.99 specificity (Panel B) of 10-protein pan-cancer detection for unseen cancers for males, as described in Example 2.

FIG. 53 shows accuracy, as measured by AUC of ROC, of localization panels for each cancer among females, as described in Example 2. Panel A shows a summary of the AUC values, and Panels B-Q shows the ROCs for the localization panels for breast, cervical, ovarian, uterine, brain, bladder, colon, esophageal, liver, kidney, lung—non-small cell (NSC), bone, pancreas, lung—small cell (SC), stomach, and thyroid cancer, respectively.

FIG. 54 shows accuracy, as measured by AUC of ROC, of localization panels for each cancer among males, as described in Example 2. Panel A shows a summary of the AUC values, and Panels B-O shows the ROCs for the localization panels for prostate, testis, brain, bladder, colon, esophageal, liver, kidney, lung—non-small cell (NSC), bone, pancreas, lung—small cell (SC), stomach, and thyroid cancer, respectively.

FIG. 55 shows overall performance of the 150-protein cancer localization panel across cancers in females, as described in Example 2. Panels A and B show confusion matrices that illustrate the performance of the panel in correctly identifying the source of the cancer in the first guess (Panel A) and second guess (Panel B). Panel C shows a bar graph depicting the overall performance of the localization panel.

FIG. 56 shows overall performance of the 150-protein cancer localization panel across cancers in males, as described in Example 2. Panels A and B show confusion matrices that illustrate the performance of the panel in correctly identifying the source of the cancer in the first guess (Panel A) and second guess (Panel B). Panel C shows a bar graph depicting the overall performance of the localization panel.

FIG. 57 shows accuracy of detecting cancer in a subject, measured as AUC or ROC, using random combinations of two to 20 proteins selected from Table 1001.A.1 (Panel A), Table 1001.B.1 (Panel B), and Table 1001.C.1 (Panel C) using a first classifier, as described in Example 3. The process of selecting the random combinations of each number of proteins (two proteins, three proteins, etc.) was performed for 1000 iterations.

FIG. 58 shows accuracy of detecting cancer in a subject, measured as AUC or ROC, using random combinations of two to 20 proteins selected from Table 1001.A.1 (Panel A), Table 1001.B.1 (Panel B), and Table 1001.C.1 (Panel C) using a second classifier, as described in Example 3. The process of selecting the random combinations of each number of proteins (two proteins, three proteins, etc.) was performed for 1000 iterations.

FIG. 59 shows accuracy, as measured by AUC of ROC and presented as ROC, of pan-cancer panels shown in Tables 1001.A.1 (Panel A), 1001.B.1 (Panel B), and 1001.C.1 (Panel C) using a first classifier for detecting cancer among mixed-gender subjects, female subjects, and male subjects, respectively, as described in Example 3.

FIG. 60 shows accuracy, as measured by AUC of ROC and presented as ROC, of pan-cancer panels shown in Tables 1001.A.1 (Panel A), 1001.B.1 (Panel B), and 1001.C.1 (Panel C) using a second classifier for detecting cancer among mixed-gender subjects, female subjects, and male subjects, respectively, as described in Example 3.

FIG. 61 shows accuracy, measured as AUC of ROC, of detecting bladder cancer in a subject using random combinations of two to 15 proteins as described in Example 3. The process of selecting the random combinations of each number of proteins (two proteins, three proteins, etc.) was performed for 1000 iterations. Panels A-C shows results of detecting bladder cancer using proteins selected from Tables 2001.A.1, 2001.B.1, and 2001.C.1, respectively.

FIG. 62 shows accuracy, measured as AUC of ROC, of detecting bone cancer (osteosarcoma) in a subject using random combinations of two to 15 proteins as described in Example 3. The process of selecting the random combinations of each number of proteins (two proteins, three proteins, etc.) was performed for 1000 iterations. Panels A-C shows results of detecting bone cancer using proteins selected from Tables 2002.A.1, 2002.B.1, and 2002.C.1, respectively.

FIG. 63 shows accuracy, measured as AUC of ROC, of detecting brain cancer (astrocytoma) in a subject using random combinations of two to 15 proteins as described in Example 3. The process of selecting the random combinations of each number of proteins (two proteins, three proteins, etc.) was performed for 1000 iterations. Panels A-C shows results of detecting brain cancer using proteins selected from Tables 2003.A.1, 2003.B.1, and 2003.C.1, respectively.

FIG. 64 shows accuracy, measured as AUC of ROC, of detecting breast cancer in a subject using random combinations of two to 15 proteins selected from Table 2004.A.1 as described in Example 3. The process of selecting the random combinations of each number of proteins (two proteins, three proteins, etc.) was performed for 1000 iterations.

FIG. 65 shows accuracy, measured as AUC of ROC, of detecting cervical cancer in a subject using random combinations of two to 15 proteins selected from Table 2005.A.1 as described in Example 3. The process of selecting the random combinations of each number of proteins (two proteins, three proteins, etc.) was performed for 1000 iterations.

FIG. 66 shows accuracy, measured as AUC of ROC, of detecting colorectal cancer in a subject using random combinations of two to 15 proteins as described in Example 3. The process of selecting the random combinations of each number of proteins (two proteins, three proteins, etc.) was performed for 1000 iterations. Panels A-C shows results of detecting colorectal cancer using proteins selected from Tables 2006.A.1, 2006.B.1, and 2006.C.1, respectively.

FIG. 67 shows accuracy, measured as AUC of ROC, of detecting esophogeal cancer in a subject using random combinations of two to 15 proteins as described in Example 3. The process of selecting the random combinations of each number of proteins (two proteins, three proteins, etc.) was performed for 1000 iterations. Panels A-C shows results of detecting esophogeal cancer using proteins selected from Tables 2007.A.1, 2007.B.1, and 2007.C.1, respectively.

FIG. 68 shows accuracy, measured as AUC of ROC, of detecting kidney cancer (hepatocellular carcinoma) in a subject using random combinations of two to 15 proteins as described in Example 3. The process of selecting the random combinations of each number of proteins (two proteins, three proteins, etc.) was performed for 1000 iterations. Panels A-C shows results of detecting kidney cancer using proteins selected from Tables 2008.A.1, 2008.B.1, and 2008.C.1, respectively.

FIG. 69 shows accuracy, measured as AUC of ROC, of detecting liver cancer in a subject using random combinations of two to 15 proteins as described in Example 3. The process of selecting the random combinations of each number of proteins (two proteins, three proteins, etc.) was performed for 1000 iterations. Panels A-C shows results of detecting liver cancer using proteins selected from Tables 2009.A.1, 2009.B.1, and 2009.C.1, respectively.

FIG. 70 shows accuracy, measured as AUC of ROC, of detecting SC lung cancer in a subject using random combinations of two to 15 proteins as described in Example 3. The process of selecting the random combinations of each number of proteins (two proteins, three proteins, etc.) was performed for 1000 iterations. Panels A-C shows results of detecting SC lung cancer using proteins selected from Tables 2010.A.1, 2010.B.1, and 2010.C.1, respectively.

FIG. 71 shows accuracy, measured as AUC of ROC, of detecting NSC lung cancer in a subject using random combinations of two to 15 proteins as described in Example 3. The process of selecting the random combinations of each number of proteins (two proteins, three proteins, etc.) was performed for 1000 iterations. Panels A-C shows results of detecting NSC cancer using proteins selected from Tables 2011.A.1, 2011.B.1, and 2011.C.1, respectively.

FIG. 72 shows accuracy, measured as AUC of ROC, of detecting ovarian cancer in a subject using random combinations of two to 15 proteins selected from Table 2012.A.1 as described in Example 3. The process of selecting the random combinations of each number of proteins (two proteins, three proteins, etc.) was performed for 1000 iterations.

FIG. 73 shows accuracy, measured as AUC of ROC, of detecting pancreatic cancer in a subject using random combinations of two to 15 proteins as described in Example 3. The process of selecting the random combinations of each number of proteins (two proteins, three proteins, etc.) was performed for 1000 iterations. Panels A-C shows results of detecting cancer using proteins selected from Tables 2013.A.1, 2013.B.1, and 2013.C.1, respectively.

FIG. 74 shows accuracy, measured as AUC of ROC, of detecting prostate cancer in a subject using random combinations of two to 15 proteins selected from Table 2014.A.1 as described in Example 3. The process of selecting the random combinations of each number of proteins (two proteins, three proteins, etc.) was performed for 1000 iterations.

FIG. 75 shows accuracy, measured as AUC of ROC, of detecting stomach cancer in a subject using random combinations of two to 15 proteins as described in Example 3. The process of selecting the random combinations of each number of proteins (two proteins, three proteins, etc.) was performed for 1000 iterations. Panels A-C shows results of detecting stomach cancer using proteins selected from Tables 2015.A.1, 2015.B.1, and 2015.C.1, respectively.

FIG. 76 shows accuracy, measured as AUC of ROC, of detecting testicular cancer in a subject using random combinations of two to 15 proteins selected from Table 2016.A.1 as described in Example 3. The process of selecting the random combinations of each number of proteins (two proteins, three proteins, etc.) was performed for 1000 iterations.

FIG. 77 shows accuracy, measured as AUC of ROC, of detecting thyroid cancer in a subject using random combinations of two to 15 proteins as described in Example 3. The process of selecting the random combinations of each number of proteins (two proteins, three proteins, etc.) was performed for 1000 iterations. Panels A-C shows results of detecting thyroid cancer using proteins selected from Tables 2017.A.1, 2017.B.1, and 2017.C.1, respectively.

FIG. 78 shows accuracy, measured as AUC of ROC, of detecting uterine cancer in a subject using random combinations of two to 15 proteins selected from Table 2018.A.1 as described in Example 3. The process of selecting the random combinations of each number of proteins (two proteins, three proteins, etc.) was performed for 1000 iterations.

FIG. 79 shows accuracy, as measured by AUC of ROC and presented as ROC, of localization panels shown in Tables 2001.A.1 (Panel A), 2001.B.1 (Panel B), and 2001.C.1 (Panel C) for bladder cancer among mixed-gender subjects, female subjects, and male subjects, respectively, as described in Example 3.

FIG. 80 shows accuracy, as measured by AUC of ROC and presented as ROC, of localization panels shown in Tables 2002.A.1 (Panel A), 2002.B.1 (Panel B), and 2002.C.1 (Panel C) for bone cancer (osteosarcoma) among mixed-gender subjects, female subjects, and male subjects, respectively, as described in Example 3.

FIG. 81 shows accuracy, as measured by AUC of ROC and presented as ROC, of localization panels shown in Tables 2003.A.1 (Panel A), 2003.B.1 (Panel B), and 2003.C.1 (Panel C) for brain cancer (astrocytoma) among mixed-gender subjects, female subjects, and male subjects, respectively, as described in Example 3.

FIG. 82 shows accuracy, as measured by AUC of ROC and presented as ROC, of localization panel shown in Table 2004.A.1 for breast cancer among females subjects as described in Example 3.

FIG. 83 shows accuracy, as measured by AUC of ROC and presented as ROC, of localization panel shown in Table 2005.A.1 for cervical cancer among females subjects as described in Example 3.

FIG. 84 shows accuracy, as measured by AUC of ROC and presented as ROC, of localization panels shown in Tables 2006.A.1 (Panel A), 2006.B.1 (Panel B), and 2006.C.1 (Panel C) for colorectal cancer among mixed-gender subjects, female subjects, and male subjects, respectively, as described in Example 3.

FIG. 85 shows accuracy, as measured by AUC of ROC and presented as ROC, of localization panels shown in Tables 2007.A.1 (Panel A), 2007.B.1 (Panel B), and 2007.C.1 (Panel C) for esophogeal cancer among mixed-gender subjects, female subjects, and male subjects, respectively, as described in Example 3.

FIG. 86 shows accuracy, as measured by AUC of ROC and presented as ROC, of localization panels shown in Tables 2008.A.1 (Panel A), 2008.B.1 (Panel B), and 2008.C.1 (Panel C) for kidney cancer (hepatocellular carcinoma) among mixed-gender subjects, female subjects, and male subjects, respectively, as described in Example 3.

FIG. 87 shows accuracy, as measured by AUC of ROC and presented as ROC, of localization panels shown in Tables 2009.A.1 (Panel A), 2009.B.1 (Panel B), and 2009.C.1 (Panel C) for liver cancer among mixed-gender subjects, female subjects, and male subjects, respectively, as described in Example 3.

FIG. 88 shows accuracy, as measured by AUC of ROC and presented as ROC, of localization panels shown in Tables 2010.A.1 (Panel A), 2010.B.1 (Panel B), and 2010.C.1 (Panel C) for SC lung cancer among mixed-gender subjects, female subjects, and male subjects, respectively, as described in Example 3.

FIG. 89 shows accuracy, as measured by AUC of ROC and presented as ROC, of localization panels shown in Tables 2011.A.1 (Panel A), 2011.B.1 (Panel B), and 2011.C.1 (Panel C) for NSC lung cancer among mixed-gender subjects, female subjects, and male subjects, respectively, as described in Example 3.

FIG. 90 shows accuracy, as measured by AUC of ROC and presented as ROC, of localization panel shown in Table 2012.A.1 for ovarian cancer among females subjects as described in Example 3.

FIG. 91 shows accuracy, as measured by AUC of ROC and presented as ROC, of localization panels shown in Tables 2013.A.1 (Panel A), 2013.B.1 (Panel B), and 2013.C.1 (Panel C) for pancreatic cancer among mixed-gender subjects, female subjects, and male subjects, respectively, as described in Example 3.

FIG. 92 shows accuracy, as measured by AUC of ROC and presented as ROC, of localization panel shown in Table 2014.A.1 for prostate cancer among females subjects as described in Example 3.

FIG. 93 shows accuracy, as measured by AUC of ROC and presented as ROC, of localization panels shown in Tables 2015.A.1 (Panel A), 2015.B.1 (Panel B), and 2015.C.1 (Panel C) for stomach cancer among mixed-gender subjects, female subjects, and male subjects, respectively, as described in Example 3.

FIG. 94 shows accuracy, as measured by AUC of ROC and presented as ROC, of localization panel shown in Table 2016.A.1 for testicular cancer among females subjects as described in Example 3.

FIG. 95 shows accuracy, as measured by AUC of ROC and presented as ROC, of localization panels shown in Tables 2017.A.1 (Panel A), 2017.B.1 (Panel B), and 2017.C.1 (Panel C) for thyroid cancer among mixed-gender subjects, female subjects, and male subjects, respectively, as described in Example 3.

FIG. 96 shows accuracy, as measured by AUC of ROC and presented as ROC, of localization panel shown in Table 2018.A.1 for uterine cancer among females subjects as described in Example 3.

### DETAILED DESCRIPTION OF THE INVENTION

The present invention involves the use of proteins in pan-cancer and tissue-specific cancer detection methods. Such use can be applied in methods of evaluating a subject for cancer, methods of treating subjects for cancer, among others.

The practice of the present invention can employ, unless otherwise indicated, conventional techniques of proteomics, bioinformatics, oncology, and pharmacology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values ±10% of the stated number. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

An “effective amount” of a composition as disclosed herein is an amount sufficient to carry out a specifically stated purpose. An “effective amount” can be determined empirically and in a routine manner, in relation to the stated purpose, route of administration, and dosage form.

The term “pan-cancer” is used to indicate a relation or association with a non-specific proliferative disease. Thus, for example, a “pan-cancer” analysis refers to an analysis for a non-specific proliferative disease in a subject, and does not detect or is used to identify a specific proliferative disease.

The term “tissue-specific cancer” is used to indicate a relation or association with a proliferative disease of a specific tissue. Thus, for example, a “tissue-specific” analysis refers to an analysis for a proliferative disease of a specific organ or tissue in a subject.

The term “detection analysis” or “detection protein” refers to an analysis or protein that is used to determine whether any cancer (e.g., “pan-cancer detection analysis” or “pan-cancer detection protein”) or a specific type of cancer (e.g., “tissue-specific cancer detection analysis” or “tissue-specific cancer detection protein”) is present in a subject.

The term “localization analysis” or “localization protein” refers to an analysis or protein that is used to determine the location of cancer in a subject.

The term “subject” or “individual” or “patient” means any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

The term “early-stage” in the context of cancer (e.g., “early-stage cancer” or cancer that “is early-stage”) refers generally to a level of advancement of the cancer prior to the cancer spreading to lymph nodes or tissues that are distant from the tissue of origin. In some embodiments, an early-stage cancer can refer to a cancer that is a Stage 0, Stage I, or Stage II cancer, based on the stage classification known in the art that grades cancer from Stage 0 (e.g., carcinoma in situ, where the cancer is still only in the layer of cells where it started and has not advanced farther), through Stages I-III (e.g., cancer is present—the higher the number, the larger the tumor and the more it has spread into nearby tissues), and to Stage IV (e.g., the cancer has spread to distant parts of the body). In some embodiments, this stage classification incorporates the TNM System, which evaluates the cancer based on the size and extent of the main tumor (“T”), the number of nearby lymph nodes that have cancer (“N”), and the extent to which the cancer has metastasized (“M”).

The term “symptomatic” means to exhibit one or more signs or features that are regarded as indicative, or are known to be associated with, a disease or condition. A subject may be considered as “symptomatic” of cancer based on symptoms that are known in the art to be associated with cancer in general or for specific types of cancer. Examples include, but are not limited to, fatigue: lump or area of thickening that can be felt under the skin; weight changes, including unintended loss or gain; skin changes, such as yellowing, darkening, or redness of the skin, sores that will not heal, or changes to existing moles; changes in bowel or bladder habits; persistent cough or trouble breathing: difficulty swallowing; hoarseness; persistent indigestion or discomfort after eating; persistent, unexplained muscle or joint pain; persistent, unexplained fevers or night sweats; and unexplained bleeding or bruising.

A subject may be considered as “suspected of having a cancer” due to the presence of symptoms, i.e., the subject is symptomatic: genetic markers (e.g., mutations in BRCA1, BRCA2, RAS, BRAF, etc.); patient's habits or medical history; patient's family medical history; examination or tests known in the art for which the outcome is associated with cancer or risk of cancer, etc.

The term “asymptomatic” means to not exhibit any signs or features that are regarded as indicative, or are known to be associated with, a disease or condition.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder if the patient shows, e.g., total, partial, or transient alleviation or elimination of symptoms associated with the disease or disorder.

The term “ROC” or “ROC curve” is used to refer to a receiver operator characteristic curve. A ROC curve can be a graphical representation of the performance of a classifier system. For any given method, a ROC can be generated by plotting the sensitivity against the specificity. The sensitivity and specificity of a method for detecting the presence of a cancer or a specific type of cancer can be determined at various concentrations of proteins in a sample from the subject. The AUC of a ROC curve is a metric that can provide a measure of diagnostic utility of a method, taking into account both the sensitivity and specificity of the method. The AUC can range from 0.5 to 1.0, where a value closer to 0.5 can indicate that the method has limited diagnostic utility (e.g., lower sensitivity and/or specificity) and a value closer to 1.0 indicates the method has greater diagnostic utility (e.g., higher sensitivity and/or specificity).

The term “third party” means a person or group different from the two persons or groups primarily involved. For example, in a multi-step method involving a subject, a third party can be a person/group other than the subject and the person/group primarily responsible for the performance of the steps. In such an example, a third party may perform one of the steps in the method. As another example, in a treatment method involving administration of a treatment to a subject, a third party may be a person/group other than the subject and the person/group administering the treatment.

The term “cancer recurrence” refers to a return of cancer after a period of remission. The cancer can reappear in the same, or close to, the place that it was previously found (local recurrence); in the lymph nodes and tissue located in the vicinity of the original cancer (regional recurrence); or in areas farther away from the original cancer (distant recurrence).

### Methods of the Invention

An aspect of the present invention relates to a method of evaluating a subject for cancer. The method comprises a pan-cancer detection analysis, which comprises determining in a biological sample from the subject a concentration of one or more proteins, also referred to herein as “pan-cancer detection proteins,” selected from Table 101 or selected from Table 1001. In some embodiments, the pan-cancer detection analysis is included in a phase (I) of the method, and the method further comprises a phase (II) involving a tissue-specific cancer localization analysis. Phase (II) comprises determining in a biological sample from the subject a concentration of one or more proteins, also referred to herein as “tissue-specific cancer localization proteins,” selected from each of Tables 201-218 or each of Tables 2001-2018. Tables 201-218 and Tables 2001-2018 provide tissue-specific cancer localization proteins associated with cancer of the following tissues/associated with the following cancers: bladder/bladder cancer, bone/bone cancer such as an osteosarcoma, brain/brain cancer such as an astrocytoma, breast/breast cancer, cervix/cervical cancer, colon/colon cancer (colorectal cancer), esophagus/esophageal cancer, kidney/kidney cancer such as a hepatocellular carcinoma, liver/liver cancer, lung—small cell/lung—small cell cancer (SC lung cancer), lung—non-small cell/lung—non-small cell cancer (NSC lung cancer), ovary/ovarian cancer, pancreas/pancreatic cancer, prostate/prostate cancer, stomach/stomach cancer, testis/testicular cancer, thyroid/thyroid cancer, or uterus/uterine cancer, respectively. Alternatively, Phase (II) comprises determining in a biological sample from the subject a concentration of each of the tissue-specific cancer localization proteins of Table 251, 252, 253, or 254.

In some embodiments, if Phase (I) involves analysis of pan-cancer detection proteins selected from Table 101, then Phase (II) involves analysis of tissue-specific cancer localization proteins selected from each of Tables 201-218. In some embodiments, if Phase (I) involves analysis of pan-cancer detection proteins selected from Table 1001, then Phase (II) involves analysis of tissue-specific cancer localization proteins selected from each of Tables 2001-2018.

In some embodiments, the cancer is early-stage, and phase (II) comprises determining in a biological sample from the subject a concentration of each of the tissue-specific cancer localization proteins of Table 251, 252, 253, or 254.

In preferred embodiments, the sample is already separated/obtained from the subject at the time of the pan-cancer detection analysis. In preferred embodiments, the sample is already separated/obtained from the subject at the time of the tissue-specific cancer localization analysis.

In some embodiments, the subject is suspected of having cancer, such as symptomatic of having cancer. For example, the subject may be suspected of having a cancer that is associated with a tissue selected from bladder, bone, brain, breast, cervix, colon, esophagus, kidney, liver, lung—SC, lung—NSC, ovary, pancreas, prostate, stomach, testis, thyroid, and uterus. As another example, the subject may be suspected of having a cancer comprising bladder cancer, bone cancer, brain cancer, breast cancer, cervical cancer, colon/colorectal cancer, esophageal cancer, kidney cancer, liver cancer, SC lung cancer, NSC lung cancer, ovarian cancer, pancreatic cancer, prostate cancer, stomach cancer, testicular cancer, thyroid cancer, or uterine cancer.

In preferred embodiments, the subject is asymptomatic for cancer. For example, the subject may be asymptomatic of having a cancer that is associated with a tissue selected from bladder, bone, brain, breast, cervix, colon, esophagus, kidney, liver, lung—SC, lung—NSC, ovary, pancreas, prostate, stomach, testis, thyroid, and uterus. As another example, the subject may be asymptomatic of having a cancer comprising bladder cancer, bone cancer, brain cancer, breast cancer, cervical cancer, colon cancer, esophageal cancer, kidney cancer, liver cancer, SC lung cancer, NSC lung cancer, ovarian cancer, pancreatic cancer, prostate cancer, stomach cancer, testicular cancer, thyroid cancer, or uterine cancer.

In some embodiments, phase (I) is performed prior to phase (II). In other embodiments, the performance of phase (I) overlaps with the performance of phase (II).

In some embodiments, phase (I) may be performed by a third party, who provides the determination of phase (I) for the performance of phase (II). In other embodiments, phase (II) is performed by a third party, in which the method further comprises providing the determination of phase (I) to the third party.

In embodiments of the invention, the pan-cancer detection analysis of phase (I) identifies whether the subject has a cancer, in which phase (I) further comprises applying a classifier to the concentration of the one or more pan-cancer detection proteins. The classifier identifies whether the concentration of the one or more pan-cancer detection proteins is indicative of whether the subject has cancer.

In embodiments of the invention, the tissue-specific cancer localization analysis of phase (II) identifies which cancer the subject has, in which phase (II) further comprises applying a classifier to the concentration of the one or more tissue-specific cancer localization proteins, or applying a classifier to the concentration of each of the tissue-specific cancer localization proteins. The classifier identifies whether the concentration of the one or more tissue-specific cancer localization proteins or each of the tissue-specific cancer localization proteins is indicative of which cancer the subject has.

In some embodiments, phase (II) is performed only if the pan-cancer detection analysis of phase (I) determines that the subject has a cancer.

In embodiments of the invention, the methods of evaluating a subject further comprise administering a treatment. In some embodiments, the treatment is administered when it is determined in phase (I) that the subject has cancer, and it is determined in phase (II) the type of cancer.

To this end, an aspect of the present invention relates to a method of treating cancer in a subject, comprising (a) acquiring results from methods of evaluating a subject for cancer as described herein, and (b) administering a treatment to the subject. In some embodiments, the results from methods of evaluating a subject for cancer are provided by a third party. In some embodiments, the treatment is responsive to the results, e.g., responsive to the type of cancer identified by the results.

Another aspect of the present invention relates to a method of treating cancer in a subject, comprising (a) acquiring results from an evaluation of the subject that determined to which tissue the cancer is associated, in which the evaluation comprises (i) determining in a biological sample from the subject a concentration of one or more proteins selected from Table 101 or selected from Table 1001, and applying a classifier to the concentration of the one or more proteins to identity that the subject has cancer, and (ii) determining in a biological sample from the subject a concentration of one or more proteins selected from each of Tables 201-218 or selected from each of Tables 2001-2018, and applying a classifier to the concentration of the one or more proteins to identify with which tissue the cancer is associated; and (b) administering a treatment to the subject, e.g., a treatment for the cancer identified in the results of (a). Alternatively, the method comprises (a) acquiring results from an evaluation of the subject that determined to which tissue the cancer is associated, in which the evaluation comprises (i) determining in a biological sample from the subject a concentration of one or more proteins selected from Table 101 or selected from Table 1001, and applying a classifier to the concentration of the one or more proteins to identify that the subject has cancer, and (ii) determining in a biological sample from the subject a concentration of each of the proteins of Table 251, 252, 253, or 254, and applying a classifier to the concentration of each protein to identify with which tissue the cancer is associated; and (b) administering a treatment to the subject, e.g., a treatment for the cancer identified in the results of (a). In some embodiments, the results in (a) are acquired from a third party.

In some embodiments, if the concentration of one or more proteins from Table 101 is determined in (i), then (ii) comprises determining in a biological sample from the subject a concentration of one or more proteins selected from each of Tables 201-218. In some embodiments, if the concentration of one or more proteins from Table 1001 is determined in (i), then (ii) comprises determining in a biological sample from the subject a concentration of one or more proteins selected from each of Tables 2001-2018.

In some embodiments, the cancer is early-stage, and the method comprises (a) acquiring results from an evaluation of the subject that determined to which tissue the cancer is associated, in which the evaluation comprises (i) determining in a biological sample from the subject a concentration of one or more proteins selected from Table 101 or selected from Table 1001, and applying a classifier to the concentration of the one or more proteins to identify that the subject has cancer, and (ii) determining in a biological sample from the subject a concentration of each of the proteins of Table 251, 252, 253, or 254, and applying a classifier to the concentration of each protein to identify with which tissue the cancer is associated; and (b) administering a treatment to the subject, e.g., a treatment for the cancer identified in the results of (a).

Yet, another aspect of the present invention relates to a method of treating cancer in a subject, comprising (a) determining in a biological sample from the subject a concentration of one or more proteins selected from Table 101 or selected from Table 1001, and applying a classifier to the concentration of the one or more proteins to identify that the subject has cancer; (b) determining in a biological sample from the subject a concentration of one or more proteins selected from each of Tables 201-218 or selected from each of Tables 2001-2018, and applying a classifier to the concentration of the one or more proteins to identify with which tissue the cancer is associated; and (c) administering to the subject a treatment, e.g., a treatment for the cancer identified in (b). Alternatively, the method comprises (a) determining in a biological sample from the subject a concentration of one or more proteins selected from Table 101 or selected from Table 1001, and applying a classifier to the concentration of the one or more proteins to identify that the subject has cancer; (b) determining in a biological sample from the subject a concentration of each of the proteins of Table 251, 252, 253, or 254, and applying a classifier to the concentration of each protein to identify with which tissue the cancer is associated; and (c) administering to the subject a treatment, e.g., a treatment for the cancer identified in (b).

In some embodiments, if the concentration of one or more proteins from Table 101 is determined in (a), then (b) comprises determining in a biological sample from the subject a concentration of one or more proteins selected from each of Tables 201-218. In some embodiments, if the concentration of one or more proteins from Table 1001 is determined in (a), then (b) comprises determining in a biological sample from the subject a concentration of one or more proteins selected from each of Tables 2001-2018.

In some embodiments, the cancer is early-stage, and the method comprises (a) determining in a biological sample from the subject a concentration of one or more proteins selected from Table 101 or selected from Table 1001, and applying a classifier to the concentration of the one or more proteins to identify that the subject has cancer; (b) determining in a biological sample from the subject a concentration of each of the proteins of Table 251, 252, 253, or 254, and applying a classifier to the concentration of each protein to identify with which tissue the cancer is associated; and (c) administering to the subject a treatment, e.g., a treatment for the cancer identified in (b).

Another aspect of the present invention relates to a method of evaluating a subject for a cancer that is associated with any one of the following tissues: bladder, bone, brain, breast, cervix, colon, esophagus, kidney, liver, lung—small cell, lung—non-small cell, ovary, pancreas, prostate, stomach, testis, thyroid, and uterus; or a method of evaluating a subject for a cancer comprising bladder cancer, bone cancer, brain cancer, breast cancer, cervical cancer, colon cancer, esophageal cancer, kidney cancer, liver cancer, lung—small cell cancer, lung—non-small cell cancer, ovarian cancer, pancreatic cancer, prostate cancer, stomach cancer, testicular cancer, thyroid cancer, or uterine cancer. The method comprises determining in a biological sample from the subject a concentration of one or more proteins, also referred to herein as “tissue-specific cancer detection proteins,” selected from any one of Tables 301-318. Tables 301-318 provide tissue-specific cancer detection proteins associated with cancer of the following tissues/cancers: bladder/bladder cancer, bone/bone cancer, brain/brain cancer, breast/breast cancer, cervix/cervical cancer, colon/colon cancer, esophagus/esophageal cancer, kidney/kidney cancer, liver/liver cancer, lung—small cell/lung—small cell cancer, lung—non-small cell/lung—non-small cell cancer, ovary/ovarian cancer, pancreas/pancreatic cancer, prostate/prostate cancer, stomach/stomach cancer, testis/testicular cancer, thyroid/thyroid cancer, and uterus/uterine cancer, respectively.

In preferred embodiments, the sample is already separated/obtained from the subject at the time of the evaluation.

In preferred embodiments, the subject is suspected of having cancer, such as symptomatic of having cancer. For example, the subject may be suspected of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus. As another example, the subject may be suspected of having a cancer comprising bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

In some embodiments, the subject is asymptomatic for cancer. For example, the subject may be asymptomatic of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus. As another example, the subject may be asymptomatic of having a cancer comprising bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

In some embodiments, the subject is evaluated for a cancer that is associated with the lung, colon, breast, ovary, or pancreas.

In embodiments of the invention, the method identifies whether the subject has the cancer for which the subject is being evaluated. The method may further comprise applying a classifier to the concentration of the one or more tissue-specific cancer detection proteins. The classifier identifies whether the concentration of the one or more tissue-specific cancer detection proteins is indicative that the subject has the cancer for which the subject is being evaluated.

In embodiments of the invention, the methods of evaluating a subject further comprise administering a treatment. In some embodiments, the treatment is administered when it is determined that the subject has the cancer for which the subject is being evaluated.

To this end, an aspect of the present invention relates to a method of treating cancer in a subject, comprising (a) acquiring results from methods of evaluating a subject for cancer as described herein, and (b) administering a treatment to the subject. In some embodiments, the results from methods of evaluating a subject for cancer are provided by a third party. In some embodiments, the treatment is responsive to the results, e.g., responsive to the type of cancer that the subject has.

Another aspect of the present invention relates to a method of treating cancer in a subject, in which the cancer is associated with a tissue selected from bladder, bone, brain, breast, cervix, colon, esophagus, kidney, liver, lung—small cell, lung—non-small cell, ovary, pancreas, prostate, stomach, testis, thyroid, and uterus; or a method of treating cancer in a subject, in which the cancer comprises bladder cancer, bone cancer, brain cancer, breast cancer, cervical cancer, colon cancer, esophageal cancer, kidney cancer, liver cancer, lung—small cell cancer, lung—non-small cell cancer, ovarian cancer, pancreatic cancer, prostate cancer, stomach cancer, testicular cancer, thyroid cancer, or uterine cancer; in which the method comprises (a) acquiring results from an evaluation of the subject that determined the subject has cancer associated with the tissue; (b) administering a treatment to the 30ubjectt. e.g., a treatment for the cancer identified in (a), in which the evaluation comprises: (I) determining in a biological sample from the subject a concentration of one or more proteins selected from Tables 301-318 that respectively correspond to said tissues, and (II) applying a classifier to the concentration of the one or more proteins to identify with which tissue the cancer is associated. In some embodiments, the results in (a) are acquired from a third party.

Yet another aspect of the present invention relates to a method of treating cancer in a subject, in which the cancer is associated with a tissue selected from bladder, bone, brain, breast, cervix, colon, esophagus, kidney, liver, lung—small cell, lung—non-small cell, ovary, pancreas, prostate, stomach, testis, thyroid, and uterus; or a method of treating cancer in a subject, in which the cancer comprises bladder cancer, bone cancer, brain cancer, breast cancer, cervical cancer, colon cancer, esophageal cancer, kidney cancer, liver cancer, lung—small cell cancer, lung—non-small cell cancer, ovarian cancer, pancreatic cancer, prostate cancer, stomach cancer, testicular cancer, thyroid cancer, or uterine cancer; in which the method comprises (a) determining in a biological sample from the subject a concentration of one or more proteins selected from Tables 301-318 that respectively correspond to said tissues, acquiring results from an evaluation of the subject that determined the subject has cancer associated with the tissue: (b) applying a classifier to the concentration of the one or more proteins to identify with which tissue the cancer is associated; and (c) administering a treatment to the subject, e.g., a treatment for the cancer identified in (b).

Another aspect of the present invention relates to a method of evaluating a subject who is suspected of having a cancer that is associated with any one of the following tissues: bladder, bone, brain, breast, cervix, colon, esophagus, kidney, liver, lung—small cell, lung—non-small cell, ovary, pancreas, prostate, stomach, testis, thyroid, and uterus; or a method of evaluating a subject who is suspected of having cancer comprising bladder cancer, bone cancer, brain cancer, breast cancer, cervical cancer, colon cancer, esophageal cancer, kidney cancer, liver cancer, lung—small cell cancer, lung—non-small cell cancer, ovarian cancer, pancreatic cancer, prostate cancer, stomach cancer, testicular cancer, thyroid cancer, or uterine cancer; in which the method comprises determining in a biological sample from the subject a concentration of one or more tissue-specific cancer localization proteins selected from corresponding Tables 201-218 or selected from corresponding Tables 2001-2018. Alternatively, the method comprises determining in a biological sample from the subject a concentration of each of the proteins of Table 251, 252, 253, or 254.

In some embodiments, the cancer is early-stage, and the method comprises determining in a biological sample from the subject a concentration of each of the proteins of Table 251, 252, 253, or 254.

In embodiments, the subject is suspected of having cancer, such as symptomatic of having cancer. For example, the subject may be suspected of having a cancer that is associated with a tissue selected from bladder, bone, brain, breast, cervix, colon, esophagus, kidney, liver, lung—SC, lung—NSC, ovary, pancreas, prostate, stomach, testis, thyroid, and uterus. As another example, the subject may be suspected of having a cancer comprising bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

In some embodiments, the subject is suspected of having a cancer that is associated with the lung, colon, breast, ovary, or pancreas.

In embodiments of the invention, the method identifies whether the subject has the cancer for which the subject is being evaluated. The method may further comprise applying a classifier to the concentration of the one or more tissue-specific cancer localization proteins. The classifier identifies whether the concentration of the one or more tissue-specific cancer localization proteins is indicative that the subject has the cancer for which the subject is being evaluated.

In embodiments of the invention, the methods of evaluating a subject further comprise administering a treatment. In some embodiments, the treatment is administered when it is determined that the subject has the cancer for which the subject is being evaluated.

To this end, an aspect of the present invention relates to a method of treating cancer in a subject, comprising (a) acquiring results from methods of evaluating a subject for cancer as described herein, and (b) administering a treatment to the subject. In some embodiments, the results from methods of evaluating a subject for cancer are provided by a third party. In some embodiments, the treatment is responsive to the results, e.g., responsive to the type of cancer that the subject has.

Another aspect of the present invention relates to a method of treating cancer in a subject, who is suspected of having a cancer that is associated with any one of the following tissues: bladder, bone, brain, breast, cervix, colon, esophagus, kidney, liver, lung—small cell, lung—non-small cell, ovary, pancreas, prostate, stomach, testis, thyroid, and uterus; or a method of treating cancer in a subject how is suspected of having cancer comprising bladder cancer, bone cancer such as an osteosarcoma, brain cancer such as an astrocytoma, breast cancer, cervical cancer, colon cancer, esophageal cancer, kidney cancer such as a hepatocellular carcinoma, liver cancer, SC lung cancer, NSC lung cancer, ovarian cancer, pancreatic cancer, prostate cancer, stomach cancer, testicular cancer, thyroid cancer, or uterine cancer; in which the method comprises (a) acquiring results from an evaluation of the subject that determined the subject has cancer associated with the tissue; (b) administering a treatment to the subject, in which the evaluation comprises: (i) determining in a biological sample from the subject a concentration of one or more proteins selected from Tables 201-218 or selected from Tables 2001-2018 that respectively correspond to said tissues, and (ii) applying a classifier to the concentration of the one or more proteins to identify with which tissue the cancer is associated. Alternatively, the method comprises (a) acquiring results from an evaluation of the subject that determined the subject has cancer associated with the tissue; (b) administering a treatment to the subject, in which the evaluation comprises: (i) determining in a biological sample from the subject a concentration of each of the proteins of Table 251, 252, 253, or 254, and (ii) applying a classifier to the concentration of each protein to identify with which tissue the cancer is associated. In some embodiments, the results in (a) are acquired from a third party.

In some embodiments, the cancer is early-stage, and the method comprises (a) acquiring results from an evaluation of the subject that determined the subject has cancer associated with the tissue; (b) administering a treatment to the subject, in which the evaluation comprises: (i) determining in a biological sample from the subject a concentration of each of the proteins of Table 251, 252, 253, or 254, and (ii) applying a classifier to the concentration of each protein to identify with which tissue the cancer is associated.

Yet another aspect of the present invention relates to a method of treating cancer in a subject, who is suspected of having a cancer that is associated with any one of the following tissues: bladder, bone, brain, breast, cervix, colon, esophagus, kidney, liver, lung—small cell, lung—non-small cell, ovary, pancreas, prostate, stomach, testis, thyroid, and uterus; or a method of treating cancer in a subject how is suspected of having cancer comprising bladder cancer, bone cancer, brain cancer, breast cancer, cervical cancer, colon cancer, esophageal cancer, kidney cancer, liver cancer, lung—small cell cancer, lung—non-small cell cancer, ovarian cancer, pancreatic cancer, prostate cancer, stomach cancer, testicular cancer, thyroid cancer, or uterine cancer; in which the method comprises (a) determining in a biological sample from the subject a concentration of one or more proteins selected from Tables 201-218 or selected from Tables 2001-2018 that respectively correspond to said tissues; (b) applying a classifier to the concentration to identify with which tissue the cancer is associated; and (c) administering a treatment to the subject, e.g., a treatment for the cancer identified in (b). Alternatively, the method comprises (a) determining in a biological sample from the subject a concentration of each of the proteins of Table 251, 252, 253, or 254; (b) applying a classifier to the concentration of each protein to identify with which tissue the cancer is associated; and (c) administering a treatment to the subject, e.g., a treatment for the cancer identified in (b).

In some embodiments, the cancer is early-stage, and the method comprises (a) determining in a biological sample from the subject a concentration of each of the proteins of Table 251, 252, 253, or 254; (b) applying a classifier to the concentration of each protein to identify with which tissue the cancer is associated; and (c) administering a treatment to the subject, e.g., a treatment for the cancer identified in (b).

In addition, an aspect of the present invention relates to a method of evaluating a treatment for cancer in a subject. The method comprises (a) administering a treatment for cancer, and (b) determining in a biological sample from the subject a concentration of one or more proteins. The one or more proteins may be one or more pan-cancer detection proteins (i.e., one or more proteins selected from Table 101 or selected from Table 1001), one or more tissue-specific cancer localization proteins (i.e., one or more proteins selected from Tables 201-218 or selected from Tables 2001-2018), one or more tissue-specific detection proteins (i.e., one or more proteins selected from Tables 301-318), or a combination thereof.

In some embodiments, the cancer is associated with any one of the following tissues: bladder, bone, brain, breast, cervix, colon, esophagus, kidney, liver, lung—small cell, lung—non-small cell, ovary, pancreas, prostate, stomach, testis, thyroid, or uterus. In some embodiments, the cancer comprises bladder cancer, bone cancer such as an osteosarcoma, brain cancer such as an astrocytoma, breast cancer, cervical cancer, colon cancer, esophageal cancer, kidney cancer such as a hepatocellular carcinoma, liver cancer, SC lung cancer, NSC lung cancer, ovarian cancer, pancreatic cancer, prostate cancer, stomach cancer, testicular cancer, thyroid cancer, or uterine cancer

In preferred embodiments, the sample is already separated/obtained from the subject at the time of performing (b).

In some embodiments, administration of the treatment in (a) may be performed by a third party. In other embodiments, determining the concentration of one or more proteins in (b) may be performed by a third party.

In embodiments of the invention, the one or more proteins (e.g., the pan-cancer detection proteins) identifies whether the subject has any cancer after treatment, or the one or more proteins (e.g., the tissue-specific cancer localization proteins or tissue-specific cancer detection proteins) identifies whether the subject has a specific cancer after treatment. Thus, the method may further comprise applying a classifier to the concentration of the one or more proteins that identifies whether the concentration of the one or more proteins is indicative of the subject having any cancer or having a specific cancer.

The treatment may be any known treatment for cancer as known in the art and as described herein. The administration of the treatment in (a) may comprise a single administration or occurrence of a therapy, or may comprise multiple administrations or occurrences of a therapy.

The determination in a biological sample from the subject a concentration of one or more proteins in (b) may be performed more than once. The determination may overlap with the administration of the treatment in (a) or may occur after the administration of the treatment in (a).

In embodiments in which determination in a biological sample from the subject a concentration of one or more proteins in (b) is occurring after the administration of the treatment in (a), the determination may occur immediately after the administration of the treatment or a period of time after the administration of the treatment. The period of time may be one day or more, or one week or more, or one month or more, or one year or more; including one day, or two days, or three days, or four days, or five days, or six days, or about one week, or about two weeks, or about three weeks, or about four weeks, or about five weeks, or about six weeks, or about seven weeks, or about eight weeks, or about nine weeks, or about ten weeks, or about 11 weeks, or about 12 weeks, or about one month, or about two months, or about three months, or about four months, or about five months, or about six months, or about seven months, or about eight months, or about nine months, or about ten months, or about 11 months, or about 12 months, or about one year, or about two years, or about three years, or about four years, or about five years, or about six years, or about seven years, or about eight years, or about nine years, or about ten years, or about 11 years, or about 12 years, or about 13 years, or about 14 years, or about 15 years, or about 16 years, or about 17 years, or about 18 years, or about 19 years, or about 20 years, or about 21 years, or about 22 years, or about 23 years, or about 24 years, or about 25 years, or about 26 years, or about 27 years, or about 28 years, or about 29 years, or about 30 years, or more; including any ranges formed with these time periods as endpoints, for examples about 4 weeks to about 13 years, about 7 months to about 3 years, etc.

In some embodiments, the presence of any cancer or of a specific type of cancer after treatment may be indicative that the treatment was not effective. Thus, another aspect of the invention is a method of evaluating the efficacy of a treatment, comprising (a) administering a treatment for cancer, and (b) determining in a biological sample from the subject a concentration of one or more proteins, as described herein. Yet another aspect is a method of treatment, comprising (a) administering a treatment for cancer, and (b) determining in a biological sample from the subject a concentration of one or more proteins, as described herein, to evaluate whether the treatment was effective.

In some embodiments, the presence of any cancer or of a specific type of cancer after treatment may be indicative that the treatment requires adjustment. Thus, another aspect of the invention is a method of adjusting a treatment for cancer, comprising (a) administering a treatment for cancer, and (b) determining in a biological sample from the subject a concentration of one or more proteins, as described herein. Yet another aspect is a method of treatment, comprising (a) administering a treatment for cancer, and (b) determining in a biological sample from the subject a concentration of one or more proteins, as described herein, to evaluate whether the treatment requires adjustment; such method may further comprise administering an adjustment treatment.

In some embodiments, the presence of any cancer or of a specific type of cancer after treatment may be indicative of cancer recurrence. Thus, another aspect of the invention is a method of monitoring for cancer recurrence, comprising (a) administering a treatment for cancer, and (b) determining in a biological sample from the subject a concentration of one or more proteins, as described herein. Yet another aspect is a method of treatment, comprising (a) administering a treatment for cancer, and (b) determining in a biological sample from the subject a concentration of one or more proteins, as described herein, to evaluate cancer recurrence. In some embodiments, the method may further comprise administering a second treatment when it is determined that the cancer is recurring. The second treatment may be different from the original treatment, for example, a different therapy or different dosage of the same therapy.

An aspect of the present invention relates to a method of measuring amounts of proteins in a subject, the method comprising determining individual amounts of one or more pan-cancer detection proteins selected from Table 101.

An aspect of the present invention relates to a method of measuring amounts of proteins in a subject, the method comprising determining individual amounts of one or more pan-cancer detection proteins selected from Table 1001.

Another aspect of the present invention relates to a method of measuring amounts of proteins in a subject, the method comprising determining individual amounts of one or more tissue-specific cancer localization proteins selected any of Tables 201-218.

Another aspect of the present invention relates to a method of measuring amounts of proteins in a subject, the method comprising determining individual amounts of one or more tissue-specific cancer localization proteins selected any of Tables 2001-2018.

Another aspect of the present invention relates to a method of measuring amounts of proteins in a subject, the method comprising determining individual amounts of each of the tissue-specific cancer localization proteins of Table 251, 252, 253, or 254.

Another aspect of the present invention relates to a method of measuring amounts of proteins in a subject, the method comprising determining individual amounts of one or more tissue-specific cancer localization proteins selected any of Tables 301-318.

In some embodiments, the individual amounts of the one or more proteins is determined in a biological sample from the subject.

In some embodiments of the present invention, for any of the pan-cancer detection proteins, the tissue-specific cancer localization protein, or the tissue-specific cancer detection proteins, the methods may comprise determining the concentration of two or more, or three or more, or four or more, or five or more, or six or more, or seven or more, or eight or more, or nine or more, or ten or more, or about 15 or more, or about 20 or more, or about 25 or more, or about 30 or more, or about 35 or more, or about 40 or more, or about 45 or more, or about 50 or more, proteins; and/or no more than three, or no more than four, or no more than five, or no more than six, or no more than seven, or no more than eight, or no more than nine, or no more than ten, or no more than about 15, or no more than about 20, or no more than about 25, or no more than about 30, or no more than about 35, or no more than about 40, or no more than about 45, or no more than about 50, or no more than about 60, or no more than about 70, or no more than about 80, or no more than about 90, or no more than about 100, or no more than about 125, or no more than about 150, or no more than about 175, or no more than about 200, or no more than about 225, or no more than about 250, or no more than about 275, or no more than about 300, proteins; including any number of proteins chosen from two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100, and including any ranges thereof, for example, about two to 300 proteins, or about two to 250 proteins, or about two to 200 proteins, or about two to 150 proteins, or about two to 100 proteins, or about two to 90 proteins, or about two to 80 proteins, or about two to 75 proteins, or about two to 70 proteins, or about two to 60 proteins, or about two to 50 proteins, or about two to 40 proteins, or about two to 30 proteins, or about two to 25 proteins, or about two to 20 proteins, or about two to 15 proteins, or about two to ten proteins, or about two to five proteins, or about three to 300 proteins, or about three to 250 proteins, or about three to 200 proteins, or about three to 150 proteins, or about three to 100 proteins, or about three to 90 proteins, or about three to 80 proteins, or about three to 75 proteins, or about three to 70 proteins, or about three to 60 proteins, or about three to 50 proteins, or about three to 40 proteins, or about three to 30 proteins, or about three to 25 proteins, or about three to 20 proteins, or about three to 15 proteins, or about three to ten proteins, or about three to five proteins, or about five to 300 proteins, or about five to 250 proteins, or about five to 200 proteins, or about five to 150 proteins, or about five to 100 proteins, or about five to 90 proteins, or about five to 80 proteins, or about five to 75 proteins, or about five to 70 proteins, or about five to 60 proteins, or about five to 50 proteins, or about five to 40 proteins, or about five to 30 proteins, or about five to 25 proteins, or about five to 20 proteins, or about five to 15 proteins, or about five to ten proteins, or about ten to 300 proteins, or about ten to 250 proteins, or about ten to 200 proteins, or about ten to 150 proteins, or about ten to 100 proteins, or about ten to 90 proteins, or about ten to 80 proteins, or about ten to 75 proteins, or about ten to 70 proteins, or about ten to 60 proteins, or about ten to 50 proteins, or about ten to 40 proteins, or about ten to 30 proteins, or about ten to 25 proteins, or about ten to 20 proteins, or about ten to 15 proteins, or about 15 to 300 proteins, or about 15 to 250 proteins, or about 15 to 200 proteins, or about 15 to 150 proteins, or about 15 to 100 proteins, or about 15 to 90 proteins, or about 15 to 80 proteins, or about 15 to 75 proteins, or about 15 to 70 proteins, or about 15 to 60 proteins, or about 15 to 50 proteins, or about 15 to 40 proteins, or about 15 to 30 proteins, or about 15 to 25 proteins, or about 15 to 20 proteins, or about 20 to 300 proteins, or about 20 to 250 proteins, or about 20 to 200 proteins, or about 20 to 150 proteins, or about 20 to 100 proteins, or about 20 to 90 proteins, or about 20 to 80 proteins, or about 20 to 75 proteins, or about 20 to 70 proteins, or about 20 to 60 proteins, or about 20 to 50 proteins, or about 20 to 40 proteins, or about 20 to 30 proteins, or about 20 to 25 proteins, or about 25 to 300 proteins, or about 25 to 250 proteins, or about 25 to 200 proteins, or about 25 to 150 proteins, or about 25 to 100 proteins, or about 25 to 90 proteins, or about 25 to 80 proteins, or about 25 to 75 proteins, or about 25 to 70 proteins, or about 25 to 60 proteins, or about 25 to 50 proteins, or about 25 to 40 proteins, or about 25 to 30 proteins, or about 30 to 300 proteins, or about 30 to 250 proteins, or about 30 to 200 proteins, or about 30 to 150 proteins, or about 30 to 100 proteins, or about 30 to 90 proteins, or about 30 to 80 proteins, or about 30 to 75 proteins, or about 30 to 70 proteins, or about 30 to 60 proteins, or about 30 to 50 proteins, or about 30 to 40 proteins, or about 40 to 300 proteins, or about 40 to 250 proteins, or about 40 to 200 proteins, or about 40 to 150 proteins, or about 40 to 100 proteins, or about 40 to 90 proteins, or about 40 to 80 proteins, or about 40 to 75 proteins, or about 40 to 70 proteins, or about 40 to 60 proteins, or about 40 to 50 proteins, or about 50 to 300 proteins, or about 50 to 250 proteins, or about 50 to 200 proteins, or about 50 to 150 proteins, or about 50 to 100 proteins, or about 50 to 90 proteins, or about 50 to 80 proteins, or about 50 to 75 proteins, or about 50 to 70 proteins, or about 50 to 60 proteins, or about 75 to 300 proteins, or about 75 to 250 proteins, or about 75 to 200 proteins, or about 75 to 150 proteins, or about 75 to 100 proteins, or about 100 to 300 proteins, or about 100 to 250 proteins, or about 100 to 200 proteins, or about 100 to 150 proteins.

In some embodiments, the methods may comprise determining the concentration of each of the pan-cancer detection proteins, the tissue-specific cancer localization protein, or the tissue-specific cancer detection proteins. In certain embodiments, the methods may comprise determining the concentration of each protein of Table 251, or 252, or 253, or 254.

In some embodiments, the number of proteins for which the concentration is determined may be sufficient to achieve an AUC of a ROC curve of at least about 0.6. In certain embodiments, the number of proteins for which the concentration is determined may be sufficient to achieve an AUC of a ROC curve of at least about 0.7, or at least about 0.8, or at least about 0.9.

The sample may be any biological sample capable of being obtained from the subject, and encompass fluids, solids, tissues, and gases. In some embodiments, the sample may be a blood product, such as plasma, serum and the like. In some embodiments, the sample may be a urine sample, saliva sample, cerebrospinal fluid (CSF) sample, sweat sample, or tear sample.

The treatment administered to the subjects according to the methods described herein may be treatments known in the art. Examples of such treatments include, but are not limited to, surgery, such as tumor resection surgery; radiation therapy; chemotherapy; immunotherapy; hormone therapy: CAR-T cell therapy; and stem cell therapy. In preferred embodiments, the treatment is specific to the type of cancer that the subject is determined to have.

In certain embodiments, a cancer patient subjected to a method of the invention is successfully treated if the patient's survival is longer than the median survival of patients having the same type of cancer as the cancer patient. For example, treatment of lung cancer would be successful in a subject treated by the methods of the invention if the subject survives longer than the median survival of lung cancer patients who have not been treated by the methods of the invention. Survival can be overall survival, i.e., length of time a patient lives, or progression-free survival. i.e., length of time a patient is treated without progression of the disease. Survival can be measured from the date of diagnosis or from the date that treatment commences. Overall survival, median overall survival, progression-free survival, and median progression-free survival can be determined by methods known in the art and/or by those described herein.

In certain embodiments a cancer patient subjected to a method of the invention is successfully treated if the patient has an improved response to the anti-cancer therapy compared with a patient having the same type of cancer who has not been subjected to a method of the invention. For example, treatment of bladder cancer would be successful in a subject treated by the methods of the invention if the subject has an improved response compared to the median response of patients who have not been treated by the methods of the invention. Response to anti-cancer treatment can be measured by known methods appropriate to the cancer type, for instance, using Response Evaluation Criteria in Solid Tumors (RECIST). Patients evaluated using RECIST can have a complete response (CR), a partial response (PR), stable disease (SD), or progressive disease (PD). An improved response can also be assessed by other criteria, for example, duration of response, reduction in tumor volume, minimum residual disease (MRD), and the like.

### Detection and Localization Proteins

Tables 101, 1001, 201-218, 2001-2018, and 301-318 may consist of sub-tables marked by “.A.1”, “.A.2”, “.A.3”, “.A.4”, “.B.1”, “.B.2”, “.B.3”, “.B.4”, “.C.1”, “.C.2” “.C.3”, and/or “.C.4” (for example, Tables 101.A.1, 101.A.2, 101.A.3, 101.A.4, 101.B.1, 101.B.2, 101.B.3, 101.B.4, 101.C.1, 101.C.2, 101.C.3, and 101.C.4 together form Table 101; Tables 201.A.1, 201.A.2, 201.B.1, 201.B.2, 201.B.3, 201.B.4, 201.C.1, 201.C.2, 201.C.3, and 201.C.4 together form Table 201; etc.).

### For Methods Involving Pan-Cancer Detection Proteins

In some embodiments, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.A.1 or Table 1001.A.1. In certain embodiments, two or more pan-cancer direction proteins are selected from Table 101.A.1 or Table 1001.A.1. In preferred embodiments, the one or more pan-cancer detection proteins are selected from Table 101.A.2, or 1001.A.2, 1001.A.3, or 1001.A.4.

In some embodiments in which the subject is female, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.B.1 or Table 1001.B.1. In certain embodiments, two or more pan-cancer direction proteins are selected from Table 101.B.1 or Table 1001.B.1. In preferred embodiments, the one or more pan-cancer detection proteins are selected from Table 101.B.2, 1001.B.2, 1001.B.3, or 1001.B.4.

In some embodiments in which the subject is male, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.C.1 or Table 1001.C.1. In certain embodiments, two or more pan-cancer direction proteins are selected from Table 101.C.1 or Table 1001.C.1. In preferred embodiments, the one or more pan-cancer detection proteins are selected from Table 101.C.2, 1001.C.2, 1001.C.3, or 1001.C.4

In some embodiments in which the subject is being evaluated for early-stage cancer, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.A.3 or Table 1001.A.1. In certain embodiments, two or more pan-cancer direction proteins are selected from Table 101.A.3 or Table 1001.A.1. In preferred embodiments, the one or more pan-cancer detection proteins are selected from Table 101.A.4, 1001.A.2, or 1001.A.3.

In some embodiments in which the subject is female and is being evaluated for early-stage cancer, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.B.3 or 1001.B.1. In certain embodiments, two or more pan-cancer direction proteins are selected from Table 101.B.3 or 1001.B.1. In preferred embodiments, the one or more pan-cancer detection proteins are selected from Table 101.B.4, 1001.B.2, or 1001.B.3.

In some embodiments in which the subject is male and is being evaluated for early-stage cancer, the one or more pan-cancer detection proteins may be selected from any of Tables 101.A.1, 101.A.2, 101.A.3, 101.A.4, 101.B.1, 101.B.2, 101.B.3, 101.B.4, 101.C.1, 101.C.2, 101.C.3, 101.C.4, 1001.A.1, 1001.A.2, 1001.A.3, 1001.A.4, 1001.B.1, 1001.B.2, 1001.B.3, 1001.B.4, 1001.C.1, 1001.C.2, 1001.C.3, and 1001.C.4. In some embodiments, the one or more pan-cancer detection proteins are selected from Table 101.C.3 or 1001.C.1. In certain embodiments, two or more pan-cancer direction proteins are selected from Table 101.C.3 or 1001.C.1. In preferred embodiments, the one or more pan-cancer detection proteins are selected from Table 101.C.4, 1001.C.2, or 1001.C.3.

### For Methods Involving Tissue-Specific Cancer Localization Proteins

In embodiments in which one or more tissue-specific localization proteins are selected from Table 201 or Table 2001, the one or more tissue-specific localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific localization proteins are selected from Table 201.A.1 or Table 2001.A.1. In certain embodiments, two or more tissue-specific localization proteins are selected from Table 201.A.1 or Table 2001.A.1. In preferred embodiments, the one or more tissue-specific localization proteins are selected from Table 201.A.2, 2001.A.2, or 2001.A.3.

In embodiments in which one or more tissue-specific cancer localization proteins are selected from Table 201 or Table 2001 and the subject is female, the one or more tissue-specific cancer localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific cancer localization proteins are selected from Table 201.B.1 or Table 2001.B.1. In certain embodiments, two or more tissue-specific cancer localization proteins are selected from Table 201.B.1 or Table 2001.B.1. In preferred embodiments, the one or more tissue-specific cancer localization proteins are selected from Table 201.B.2, 2001.B.2, or 2001.B.3.

In embodiments in which one or more tissue-specific cancer localization proteins are selected from Table 201 or Table 2001 and the subject is male, the one or more tissue-specific cancer localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific cancer localization proteins are selected from Table 201.C.1 or Table 2001.C.1. In certain embodiments, two or more tissue-specific cancer localization proteins are selected from Table 201.C.1 or Table 2001.C.1. In preferred embodiments, the one or more tissue-specific cancer localization proteins are selected from Table 201.C.2, 2001.C.2, or 2001.C.3.

In embodiments in which one or more tissue-specific cancer localization proteins are selected from Table 201 or Table 2001 and the subject is being evaluated for early-stage cancer, the one or more tissue-specific cancer localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific cancer localization proteins are selected from Table 201.A.1 or Table 2001.A.1. In certain embodiments, two or more tissue-specific cancer localization proteins are selected from Table 201.A.1 or Table 2001.A.1. In preferred embodiments, the one or more tissue-specific cancer localization proteins are selected from Table 201.A.2, 2001.A.2, or 2001.A.3.

In embodiments in which one or more tissue-specific cancer localization proteins are selected from Table 201 or Table 2001, the subject is being evaluated for early-stage cancer, and the subject is female, the one or more tissue-specific cancer localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific cancer localization proteins are selected from Table 201.B.3 or Table 2001.B.1. In certain embodiments, two or more tissue-specific cancer localization proteins are selected from Table 201.B.3 or Table 2001.B.1. In preferred embodiments, the one or more tissue-specific cancer localization proteins are selected from Table 201.B.4, 2001.B.2, or 2001.B.3.

In embodiments in which one or more tissue-specific cancer localization proteins are selected from Table 201 or Table 2001, the subject is being evaluated for early-stage cancer, and the subject is male, the one or more tissue-specific cancer localization proteins may be selected from any of Tables 201.A.1, 201.A.2, 201.B.1, 201.B.2, 201.B.3, 201.B.4, 201.C.1, 201.C.2, 201.C.3, 201.C.4, 2001.A.1, 2001.A.2, 2001.A.3, 2001.B.1, 2001.B.2, 2001.B.3, 2001.C.1, 2001.C.2, and 2001.C.3. In some embodiments, the one or more tissue-specific cancer localization proteins are selected from Table 201.C.3 or Table 2001.C.1. In certain embodiments, two or more tissue-specific cancer localization proteins are selected from Table 201.C.3 or Table 2001.C.1. In preferred embodiments, the one or more tissue-specific cancer localization proteins are selected from Table 201.C.4, 2001.C.2, or 2001.C.3.

In embodiments in which one or more tissue-specific localization proteins are selected from Table 202 or Table 2002, the one or more tissue-specific localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific localization proteins are selected from Table 202.A.1 or Table 2002.A.1. In certain embodiments, two or more tissue-specific localization proteins are selected from Table 202.A.1 or Table 2002.A.1. In preferred embodiments, the one or more tissue-specific localization proteins are selected from Table 202.A.2, 2002.A.2, or 2002.A.3.

In embodiments in which one or more tissue-specific cancer localization proteins are selected from Table 202 or Table 2002 and the subject is female, the one or more tissue-specific cancer localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific cancer localization proteins are selected from Table 202.B.1 or Table 2002.B.1. In certain embodiments, two or more tissue-specific cancer localization proteins are selected from Table 202.B.1 or Table 2002.B.1. In preferred embodiments, the one or more tissue-specific cancer localization proteins are selected from Table 202.B.2, 2002.B.2, or 2002.B.3.

In embodiments in which one or more tissue-specific cancer localization proteins are selected from Table 202 or Table 2002 and the subject is male, the one or more tissue-specific cancer localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific cancer localization proteins are selected from Table 202.C.1 or Table 2002.C.1. In certain embodiments, two or more tissue-specific cancer localization proteins are selected from Table 202.C.1 or Table 2002.C.1. In preferred embodiments, the one or more tissue-specific cancer localization proteins are selected from Table 202.C.2, 2002.C.2, or 2002.C.3.

In embodiments in which one or more tissue-specific cancer localization proteins are selected from Table 202 or Table 2002 and the subject is being evaluated for early-stage cancer, the one or more tissue-specific cancer localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific cancer localization proteins are selected from Table 202.A.1 or Table 2002.A.1. In certain embodiments, two or more tissue-specific cancer localization proteins are selected from Table 202.A.1 or Table 2002.A.1. In preferred embodiments, the one or more tissue-specific cancer localization proteins are selected from Table 202.A.2, 2002.A.2, or 2002.A.3.

In embodiments in which one or more tissue-specific cancer localization proteins are selected from Table 202 or Table 2002, the subject is being evaluated for early-stage cancer, and the subject is female, the one or more tissue-specific cancer localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific cancer localization proteins are selected from Table 202.B.3 or Table 2002.B.1. In certain embodiments, two or more tissue-specific cancer localization proteins are selected from Table 202.B.3 or Table 2002.B.1. In preferred embodiments, the one or more tissue-specific cancer localization proteins are selected from Table 202.B.4, 2002.B.2, or 2002.B.3.

In embodiments in which one or more tissue-specific cancer localization proteins are selected from Table 202 or Table 2002, the subject is being evaluated for early-stage cancer, and the subject is male, the one or more tissue-specific cancer localization proteins may be selected from any of Tables 202.A.1, 202.A.2, 202.B.1, 202.B.2, 202.B.3, 202.B.4, 202.C.1, 202.C.2, 202.C.3, 2002.A.1, 2002.A.2, 2002.A.3, 2002.B.1, 2002.B.2, 2002.B.3, 2002.C.1, 2002.C.2, and 2002.C.3. In some embodiments, the one or more tissue-specific cancer localization proteins are selected from Table 202.C.3 or Table 2002.C.1. In certain embodiments, two or more tissue-specific cancer localization proteins are selected from Table 202.C.3 or Table 2002.C.1. In preferred embodiments, the one or more tissue-specific cancer localization proteins are selected from Table 2002.C.2 or Table 2002.C.3.

In embodiments in which one or more tissue-specific localization proteins are selected from Table 203 or Table 2003, the one or more tissue-specific localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific localization proteins are selected from Table 203.A.1 or Table 2003.A.1. In certain embodiments, two or more tissue-specific localization proteins are selected from Table 203.A.1 or Table 2003.A.1. In preferred embodiments, the one or more tissue-specific localization proteins are selected from Table 203.A.2, 2003.A.2, or 2003.A.3.

In embodiments in which two or more tissue-specific cancer localization proteins are selected from Table 203 or Table 2003 and the subject is female, the one or more tissue-specific cancer localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific cancer localization proteins are selected from Table 203.B.1 or Table 2003.B.1. In certain embodiments, two or more tissue-specific cancer localization proteins are selected from Table 203.B.1 or Table 2003.B.1. In preferred embodiments, the one or more tissue-specific cancer localization proteins are selected from Table 203.B.2, 2003.B.2, or 2003.B.3.

In embodiments in which one or more tissue-specific cancer localization proteins are selected from Table 203 or Table 2003 and the subject is male, the one or more tissue-specific cancer localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific cancer localization proteins are selected from Table 203.C.1 or Table 2003.C.1. In certain embodiments, two or more tissue-specific cancer localization proteins are selected from Table 203.C.1 or Table 2003.C.1. In preferred embodiments, the one or more tissue-specific cancer localization proteins are selected from Table 203.C.2, 2003.C.2, or 2003.C.3.

In embodiments in which one or more tissue-specific cancer localization proteins are selected from Table 203 or Table 2003 and the subject is being evaluated for early-stage cancer, the one or more tissue-specific cancer localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific cancer localization proteins are selected from Table 203.A.1 or Table 2003.A.1. In certain embodiments, two or more tissue-specific cancer localization proteins are selected from Table 203.A.1 or Table 2003.A.1. In preferred embodiments, the one or more tissue-specific cancer localization proteins are selected from Table 203.A.2, 2003.A.2, or 2003.A.3.

In embodiments in which one or more tissue-specific cancer localization proteins are selected from Table 203 or Table 2003, the subject is being evaluated for early-stage cancer, and the subject is female, the one or more tissue-specific cancer localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific cancer localization proteins are selected from Table 203.B.3 or Table 2003.B.1. In certain embodiments, two or more tissue-specific cancer localization proteins are selected from Table 203.B.3 or Table 2003.B.1. In preferred embodiments, the one or more tissue-specific cancer localization proteins are selected from Table 203.B.4, 2003.B.2, or 2003.B.3.

In embodiments in which one or more tissue-specific cancer localization proteins are selected from Table 203 or Table 2003, the subject is being evaluated for early-stage cancer, and the subject is male, the one or more tissue-specific cancer localization proteins may be selected from any of Tables 203.A.1, 203.A.2, 203.B.1, 203.B.2, 203.B.3, 203.B.4, 203.C.1, 203.C.2, 203.C.3, 203.C.4, 2003.A.1, 2003.A.2, 2003.A.3, 2003.B.1, 2003.B.2, 2003.B.3, 2003.C.1, 2003.C.2, and 2003.C.3. In some embodiments, the one or more tissue-specific cancer localization proteins are selected from Table 203.C.3 or Table 2003.C.1. In certain embodiments, two or more tissue-specific cancer localization proteins are selected from Table 203.C.3 or Table 2003.C.1. In preferred embodiments, the one or more tissue-specific cancer localization proteins are selected from Table 203.C.4, 2003.C.2, or 2003.C.3.

In embodiments in which one or more tissue-specific localization proteins are selected from Table 204 or Table 2004, the one or more tissue-specific localization proteins may be selected from any of Tables 204.A.1, 204.A.2, 204.B.1, 204.B.2, 204.B.3, 204.B.4, 2004.A.1, 2004.A.2, and 2004.A.3. In some embodiments, the one or more tissue-specific localization proteins are selected from Table 204.A.1 or Table 2004.A.1. In certain embodiments, two or more tissue-specific localization proteins are selected from Table 204.A.1 or Table 2004.A.1. In preferred embodiments, the one or more tissue-specific localization proteins are selected from Table 204.A.2, 2004.A.2, or 2004.A.3.

In embodiments in which one or more tissue-specific cancer localization proteins are selected from Table 204 or Table 2004 and the subject is female, the one or more tissue-specific cancer localization proteins may be selected from any of Tables 204.A.1, 204.A.2, 204.B.1, 204.B.2, 204.B.3, 204.B.4, 2004.A.1, 2004.A.2, and 2004.A.3. In some embodiments, the one or more tissue-specific cancer localization proteins are selected from Table 204.B.1 or Table 2004.A.1. In certain embodiments, two or more tissue-specific cancer localization proteins are selected from Table 204.B.1 or Table 2004.A.1. In preferred embodiments, the one or more tissue-specific cancer localization proteins are selected from Table 204.B.2, 2004.A.2, or 2004.A.3.

In embodiments in which one or more tissue-specific cancer localization proteins are selected from Table 204 or Table 2004 and the subject is being evaluated for early-stage cancer, the one or more tissue-specific cancer localization proteins may be selected from any of Tables 204.A.1, 204.A.2, 204.B.1, 204.B.2, 204.B.3, 204.B.4, 2004.A.1, 2004.A.2, and 2004.A.3. In some embodiments, the one or more tissue-specific cancer localization proteins are selected from Table 204.A.1 or Table 2004.A.1. In certain embodiments, two or more tissue-specific cancer localization proteins are selected from Table 204.A.1 or Table 2004.A.1. In preferred embodiments, the one or more tissue-specific cancer localization proteins are selected from Table 204.A.2, 2004.A.2, or 2004.A.3.

In embodiments in which one or more tissue-specific cancer localization proteins are selected from Table 204 or Table 2004, the subject is being evaluated for early-stage cancer, and the subject is female, the one or more tissue-specific cancer localization proteins may be selected from any of Tables 204.A.1, 204.A.2, 204.B.1, 204.B.2, 204.B.3, 204.B.4, 2004.A.1, 2004.A.2, and 2004.A.3. In some embodiments, the one or more tissue-specific cancer localization proteins are selected from Table 204.B.3 or Table 2004.A.1. In certain embodiments, two or more tissue-specific cancer localization proteins are selected from Table 204.B.3 or Table 2004.A.1. In preferred embodiments, the one or more tissue-specific cancer localization proteins are selected from Table 204.B.4, 2004.A.2, or 2004.A.3.

In embodiments in which one or more tissue-specific localization proteins are selected from Table 205 or Table 2005, the one or more tissue-specific localization proteins may be selected from any of Tables 205.A.1, 205.A.2, 205.B.1, 205.B.2, 205.B.3, 205.B.4, 2005.A.1, 2005.A.2, and 2005.A.3. In some embodiments, the one or more tissue-specific localization proteins are selected from Table 205.A.1 or Table 2005.A.1. In certain embodiments, two or more tissue-specific localization proteins are selected from Table 205.A.1 or Table 2005.A.1. In preferred embodiments, the one or more tissue-specific localization proteins are selected from Table 205.A.2, 2005.A.2, or 2005.A.3.

In embodiments in which two or more tissue-specific cancer localization proteins are selected from Table 205 or Table 2005 and the subject is female, the one or more tissue-specific cancer localization proteins may be selected from any of Tables 205.A.1, 205.A.2, 205.B.1, 205.B.2, 205.B.3, 205.B.4, 2005.A.1, 2005.A.2, and 2005.A.3. In some embodiments, the one or more tissue-specific cancer localization proteins are selected from Table 205.B.1 or Table 2005.A.1. In certain embodiments, two or more tissue-specific cancer localization proteins are selected from Table 205.B.1 or Table 2005.A.1. In preferred embodiments, the one or more tissue-specific cancer localization proteins are selected from Table 205.B.2, 2005.A.2, or 2005.A.3.

In embodiments in which one or more tissue-specific cancer localization proteins are selected from Table 205 or Table 2005 and the subject is being evaluated for early-stage cancer, the one or more tissue-specific cancer localization proteins may be selected from any of Tables 205.A.1, 205.A.2, 205.B.1, 205.B.2, 205.B.3, 205.B.4, 2005.A.1, 2005.A.2, and 2005.A.3. In some embodiments, the one or more tissue-specific cancer localization proteins are selected from Table 205.A.1 or Table 2005.A.1. In certain embodiments, two or more tissue-specific cancer localization proteins are selected from Table 205.A.1 or Table 2005.A.1. In preferred embodiments, the one or more tissue-specific cancer localization proteins are selected from Table 205.A.2, 2005.A.2, or 2005.A.3.

In embodiments in which one or more tissue-specific cancer localization proteins are selected from Table 205 or Table 2005, the subject is being evaluated for early-stage cancer, and the subject is female, the one or more tissue-specific cancer localization proteins may be selected from any of Tables 205.A.1, 205.A.2, 205.B.1, 205.B.2, 205.B.3, 205.B.4, 2005.A.1, 2005.A.2, and 2005.A.3. In some embodiments, the one or more tissue-specific cancer localization proteins are selected from Table 205.B.3 or Table 2005.A.1. In certain embodiments, two or more tissue-specific cancer localization proteins are selected from Table 205.B.3 or Table 2005.A.1. In preferred embodiments, the one or more tissue-specific cancer localization proteins are selected from Table 205.B.4, 2005.A.2, or 2005.A.3.

In embodiments in which one or more tissue-specific localization proteins are selected from Table 206 or Table 2006, the one or more tissue-specific localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific localization proteins are selected from Table 206.A.1 or Table 2006.A.1. In certain embodiments, two or more tissue-specific localization proteins are selected from Table 206.A.1 or Table 2006.A.1. In preferred embodiments, the one or more tissue-specific localization proteins are selected from Table 206.A.2, 2006.A.2, or 2006.A.3.

In embodiments in which one or more tissue-specific cancer localization proteins are selected from Table 206 or Table 2006 and the subject is female, the one or more tissue-specific cancer localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific cancer localization proteins are selected from Table 206.B.1 or Table 2006.B.1. In certain embodiments, two or more tissue-specific cancer localization proteins are selected from Table 206.B.1 or Table 2006.B.1. In preferred embodiments, the one or more tissue-specific cancer localization proteins are selected from Table 206.B.2, 2006.B.2, or 2006.B.3.

In embodiments in which one or more tissue-specific cancer localization proteins are selected from Table 206 or Table 2006 and the subject is male, the one or more tissue-specific cancer localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific cancer localization proteins are selected from Table 206.C.1 or Table 2006.C.1. In certain embodiments, two or more tissue-specific cancer localization proteins are selected from Table 206.C.1 or Table 2006.C.1. In preferred embodiments, the one or more tissue-specific cancer localization proteins are selected from Table 206.C.2, 2006.C.2, or 2006.C.3.

In embodiments in which one or more tissue-specific cancer localization proteins are selected from Table 206 or Table 2006 and the subject is being evaluated for early-stage cancer, the one or more tissue-specific cancer localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific cancer localization proteins are selected from Table 206.A.1 or Table 2006.A.1. In certain embodiments, two or more tissue-specific cancer localization proteins are selected from Table 206.A.1 or Table 2006.A.1. In preferred embodiments, the one or more tissue-specific cancer localization proteins are selected from Table 206.A.2, 2006.A.2, or 2006.A.3.

In embodiments in which one or more tissue-specific cancer localization proteins are selected from Table 206 or Table 2006, the subject is being evaluated for early-stage cancer, and the subject is female, the one or more tissue-specific cancer localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific cancer localization proteins are selected from Table 206.B.3 or Table 2006.B.1. In certain embodiments, two or more tissue-specific cancer localization proteins are selected from Table 206.B.3 or Table 2006.B.1. In preferred embodiments, the one or more tissue-specific cancer localization proteins are selected from Table 206.B.4, 2006.B.2, or 2006.B.3.

In embodiments in which one or more tissue-specific cancer localization proteins are selected from Table 206 or Table 2006, the subject is being evaluated for early-stage cancer, and the subject is male, the one or more tissue-specific cancer localization proteins may be selected from any of Tables 206.A.1, 206.A.2, 206.B.1, 206.B.2, 206.B.3, 206.B.4, 206.C.1, 206.C.2, 206.C.3, 2006.A.1, 2006.A.2, 2006.A.3, 2006.B.1, 2006.B.2, 2006.B.3, 2006.C.1, 2006.C.2, and 2006.C.3. In some embodiments, the one or more tissue-specific cancer localization proteins are selected from Table 206.C.3 or Table 2006.C.1. In certain embodiments, two or more tissue-specific cancer localization proteins are selected from Table 206.C.3 or Table 2006.C.1. In preferred embodiments, the one or more tissue-specific cancer localization proteins are selected from Table 2006.C.2 or Table 2006.C.3.

In embodiments in which one or more tissue-specific localization proteins are selected from Table 207 or Table 2007, the one or more tissue-specific localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific localization proteins are selected from Table 207.A.1 or Table 2007.A.1. In certain embodiments, two or more tissue-specific localization proteins are selected from Table 207.A.1 or Table 2007.A.1. In preferred embodiments, the one or more tissue-specific localization proteins are selected from Table 207.A.2, 2007.A.2, or 2007.A.3.

In embodiments in which one or more tissue-specific cancer localization proteins are selected from Table 207 or Table 2007 and the subject is female, the one or more tissue-specific cancer localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific cancer localization proteins are selected from Table 207.B.1 or Table 2007.B.1. In certain embodiments, two or more tissue-specific cancer localization proteins are selected from Table 207.B.1 or Table 2007.B.1. In preferred embodiments, the one or more tissue-specific cancer localization proteins are selected from Table 207.B.2, 2007.B.2, or 2007.B.3.

In embodiments in which one or more tissue-specific cancer localization proteins are selected from Table 207 or Table 2007 and the subject is male, the one or more tissue-specific cancer localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific cancer localization proteins are selected from Table 207.C.1 or Table 2007.C.1. In certain embodiments, two or more tissue-specific cancer localization proteins are selected from Table 207.C.1 or Table 2007.C.1. In preferred embodiments, the one or more tissue-specific cancer localization proteins are selected from Table 207.C.2, 2007.C.2, or 2007.C.3.

In embodiments in which one or more tissue-specific cancer localization proteins are selected from Table 207 or Table 2007 and the subject is being evaluated for early-stage cancer, the one or more tissue-specific cancer localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific cancer localization proteins are selected from Table 207.A.1 or Table 2007.A.1. In certain embodiments, two or more tissue-specific cancer localization proteins are selected from Table 207.A.1 or Table 2007.A.1. In preferred embodiments, the one or more tissue-specific cancer localization proteins are selected from Table 207.A.2, 2007.A.2, or 2007.A.3.

In embodiments in which one or more tissue-specific cancer localization proteins are selected from Table 207 or Table 2007, the subject is being evaluated for early-stage cancer, and the subject is female, the one or more tissue-specific cancer localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific cancer localization proteins are selected from Table 207.B.3 or Table 2007.B.1. In certain embodiments, two or more tissue-specific cancer localization proteins are selected from Table 207.B.3 or Table 2007.B.1. In preferred embodiments, the one or more tissue-specific cancer localization proteins are selected from Table 207.B.4, 2007.B.2, or 2007.B.3.

In embodiments in which one or more tissue-specific cancer localization proteins are selected from Table 207 or Table 2007, the subject is being evaluated for early-stage cancer, and the subject is male, the one or more tissue-specific cancer localization proteins may be selected from any of Tables 207.A.1, 207.A.2, 207.B.1, 207.B.2, 207.B.3, 207.B.4, 207.C.1, 207.C.2, 207.C.3, 2007.A.1, 2007.A.2, 2007.A.3, 2007.B.1, 2007.B.2, 2007.B.3, 2007.C.1, 2007.C.2, and 2007.C.3. In some embodiments, the one or more tissue-specific cancer localization proteins are selected from Table 207.C.3 or Table 2007.C.1. In some embodiments, two or more tissue-specific cancer localization proteins are selected from Table 207.C.3 or Table 2007.C.1. In certain embodiments, two or more tissue-specific cancer localization proteins are selected from Table 207.C.3 or Table 2007.C.1. In preferred embodiments, the one or more tissue-specific cancer localization proteins are selected from Table 2007.C.2 or Table 2007.C.3.

In embodiments in which one or more tissue-specific localization proteins are selected from Table 208 or Table 2008, the one or more tissue-specific localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific localization proteins are selected from Table 208.A.1 or Table 2008.A.1. In certain embodiments, two or more tissue-specific localization proteins are selected from Table 208.A.1 or Table 2008.A.1. In preferred embodiments, the one or more tissue-specific localization proteins are selected from Table 208.A.2, 2008.A.2, or 2008.A.3.

In embodiments in which one or more tissue-specific cancer localization proteins are selected from Table 208 or Table 2008 and the subject is female, the one or more tissue-specific cancer localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific cancer localization proteins are selected from Table 208.B.1 or Table 2008.B.1. In certain embodiments, two or more tissue-specific cancer localization proteins are selected from Table 208.B.1 or Table 2008.B.1. In preferred embodiments, the one or more tissue-specific cancer localization proteins are selected from Table 208.B.2, 2008.B.2, or 2008.B.3.

In embodiments in which one or more tissue-specific cancer localization proteins are selected from Table 208 or Table 2008 and the subject is male, the one or more tissue-specific cancer localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific cancer localization proteins are selected from Table 208.C.1 or Table 2008.C.1. In certain embodiments, two or more tissue-specific cancer localization proteins are selected from Table 208.C.1 or Table 2008.C.1. In preferred embodiments, the one or more tissue-specific cancer localization proteins are selected from Table 208.C.2, 2008.C.2, or 2008.C.3.

In embodiments in which one or more tissue-specific cancer localization proteins are selected from Table 208 or Table 2008 and the subject is being evaluated for early-stage cancer, the one or more tissue-specific cancer localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific cancer localization proteins are selected from Table 208.A.1 or Table 2008.A.1. In certain embodiments, two or more tissue-specific cancer localization proteins are selected from Table 208.A.1 or Table 2008.A.1. In preferred embodiments, the one or more tissue-specific cancer localization proteins are selected from Table 208.A.2, 2008.A.2, or 2008.A.3.

In embodiments in which one or more tissue-specific cancer localization proteins are selected from Table 208 or Table 2008, the subject is being evaluated for early-stage cancer, and the subject is female, the one or more tissue-specific cancer localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific cancer localization proteins are selected from Table 208.B.3 or Table 2008.B.1. In certain embodiments, two or more tissue-specific cancer localization proteins are selected from Table 208.B.3 or Table 2008.B.1. In preferred embodiments, the one or more tissue-specific cancer localization proteins are selected from Table 208.B.4, 2008.B.2, or 2008.B.3.

In embodiments in which one or more tissue-specific cancer localization proteins are selected from Table 208 or Table 2008, the subject is being evaluated for early-stage cancer, and the subject is male, the one or more tissue-specific cancer localization proteins may be selected from any of Tables 208.A.1, 208.A.2, 208.B.1, 208.B.2, 208.B.3, 208.B.4, 208.C.1, 208.C.2, 208.C.3, 208.C.4, 2008.A.1, 2008.A.2, 2008.A.3, 2008.B.1, 2008.B.2, 2008.B.3, 2008.C.1, 2008.C.2, and 2008.C.3. In some embodiments, the one or more tissue-specific cancer localization proteins are selected from Table 208.C.3 or Table 2008.C.1. In certain embodiments, two or more tissue-specific cancer localization proteins are selected from Table 208.C.3 or Table 2008.C.1. In preferred embodiments, the one or more tissue-specific cancer localization proteins are selected from Table 208.C.4, 2008.C.2, or 2008.C.3.

In embodiments in which one or more tissue-specific localization proteins are selected from Table 209 or Table 2009, the one or more tissue-specific localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific localization proteins are selected from Table 209.A.1 or Table 2009.A.1. In certain embodiments, two or more tissue-specific localization proteins are selected from Table 209.A.1 or Table 2009.A.1. In preferred embodiments, the one or more tissue-specific localization proteins are selected from Table 209.A.2, 2009.A.2, or 2009.A.3.

In embodiments in which one or more tissue-specific cancer localization proteins are selected from Table 209 or Table 2009 and the subject is female, the one or more tissue-specific cancer localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific cancer localization proteins are selected from Table 209.B.1 or Table 2009.B.1. In certain embodiments, two or more tissue-specific cancer localization proteins are selected from Table 209.B.1 or Table 2009.B.1. In preferred embodiments, the one or more tissue-specific cancer localization proteins are selected from Table 209.B.2, 2009.B.2, or 2009.B.3.

In embodiments in which one or more tissue-specific cancer localization proteins are selected from Table 209 or Table 2009 and the subject is male, the one or more tissue-specific cancer localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific cancer localization proteins are selected from Table 209.C.1 or Table 2009.C.1. In certain embodiments, two or more tissue-specific cancer localization proteins are selected from Table 209.C.1 or Table 2009.C.1. In preferred embodiments, the one or more tissue-specific cancer localization proteins are selected from Table 209.C.2, 2009.C.2, or 2009.C.3.

In embodiments in which one or more tissue-specific cancer localization proteins are selected from Table 209 or Table 2009 and the subject is being evaluated for early-stage cancer, the one or more tissue-specific cancer localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific cancer localization proteins are selected from Table 209.A.1 or Table 2009.A.1. In certain embodiments, two or more tissue-specific cancer localization proteins are selected from Table 209.A.1 or Table 2009.A.1. In preferred embodiments, the one or more tissue-specific cancer localization proteins are selected from Table 209.A.2, 2009.A.2, or 2009.A.3.

In embodiments in which one or more tissue-specific cancer localization proteins are selected from Table 209 or Table 2009, the subject is being evaluated for early-stage cancer, and the subject is female, the one or more tissue-specific cancer localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific cancer localization proteins are selected from Table 209.B.3 or Table 2009.B.1. In certain embodiments, two or more tissue-specific cancer localization proteins are selected from Table 209.B.3 or Table 2009.B.1. In preferred embodiments, the one or more tissue-specific cancer localization proteins are selected from Table 209.B.4, 2009.B.2, or 2009.B.3.

In embodiments in which one or more tissue-specific cancer localization proteins are selected from Table 209 or Table 2009, the subject is being evaluated for early-stage cancer, and the subject is male, the one or more tissue-specific cancer localization proteins may be selected from any of Tables 209.A.1, 209.A.2, 209.B.1, 209.B.2, 209.B.3, 209.B.4, 209.C.1, 209.C.2, 209.C.3, 209.C.4, 2009.A.1, 2009.A.2, 2009.A.3, 2009.B.1, 2009.B.2, 2009.B.3, 2009.C.1, 2009.C.2, and 2009.C.3. In some embodiments, the one or more tissue-specific cancer localization proteins are selected from Table 209.C.3 or Table 2009.C.1. In certain embodiments, two or more tissue-specific cancer localization proteins are selected from Table 209.C.3 or Table 2009.C.1. In preferred embodiments, the one or more tissue-specific cancer localization proteins are selected from Table 209.C.4, 2009.C.2, or 2009.C.3.

In embodiments in which one or more tissue-specific localization proteins are selected from Table 210 or Table 2010, the one or more tissue-specific localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific localization proteins are selected from Table 210.A.1 or Table 2010.A.1. In certain embodiments, two or more tissue-specific localization proteins are selected from Table 210.A.1 or Table 2010.A.1. In preferred embodiments, the one or more tissue-specific localization proteins are selected from Table 210.A.2, 2010.A.2, or 2010.A.3.

In embodiments in which one or more tissue-specific cancer localization proteins are selected from Table 210 or Table 2010 and the subject is female, the one or more tissue-specific cancer localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific cancer localization proteins are selected from Table 210.B.1 or Table 2010.B.1. In certain embodiments, two or more tissue-specific cancer localization proteins are selected from Table 210.B.1 or Table 2010.B.1. In preferred embodiments, the one or more tissue-specific cancer localization proteins are selected from Table 210.B.2, 2010.B.2, or 2010.B.3.

In embodiments in which one or more tissue-specific cancer localization proteins are selected from Table 210 or Table 2010 and the subject is male, the one or more tissue-specific cancer localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific cancer localization proteins are selected from Table 210.C.1 or Table 2010.C.1. In certain embodiments, two or more tissue-specific cancer localization proteins are selected from Table 210.C.1 or Table 2010.C.1. In preferred embodiments, the one or more tissue-specific cancer localization proteins are selected from Table 210.C.2, 2010.C.2, or 2010.C.3.

In embodiments in which one or more tissue-specific cancer localization proteins are selected from Table 210 or Table 2010 and the subject is being evaluated for early-stage cancer, the one or more tissue-specific cancer localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific cancer localization proteins are selected from Table 210.A.1 or Table 2010.A.1. In certain embodiments, two or more tissue-specific cancer localization proteins are selected from Table 210.A.1 or Table 2010.A.1. In preferred embodiments, the one or more tissue-specific cancer localization proteins are selected from Table 210.A.2, 2010.A.2, or 2010.A.3.

In embodiments in which one or more tissue-specific cancer localization proteins are selected from Table 210 or Table 2010, the subject is being evaluated for early-stage cancer, and the subject is female, the one or more tissue-specific cancer localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific cancer localization proteins are selected from Table 210.B.3 or Table 2010.B.1. In certain embodiments, two or more tissue-specific cancer localization proteins are selected from Table 210.B.3 or Table 2010.B.1. In preferred embodiments, the one or more tissue-specific cancer localization proteins are selected from Table 210.B.4, 2010.B.2, or 2010.B.3.

In embodiments in which one or more tissue-specific cancer localization proteins are selected from Table 210 or Table 2010, the subject is being evaluated for early-stage cancer, and the subject is male, the one or more tissue-specific cancer localization proteins may be selected from any of Tables 210.A.1, 210.A.2, 210.B.1, 210.B.2, 210.B.3, 210.B.4, 210.C.1, 210.C.2, 210.C.3, 210.C.4, 2010.A.1, 2010.A.2, 2010.A.3, 2010.B.1, 2010.B.2, 2010.B.3, 2010.C.1, 2010.C.2, and 2010.C.3. In some embodiments, the one or more tissue-specific cancer localization proteins are selected from Table 210.C.3 or Table 2010.C.1. In certain embodiments, two or more tissue-specific cancer localization proteins are selected from Table 210.C.3 or Table 2010.C.1. In preferred embodiments, the one or more tissue-specific cancer localization proteins are selected from Table 210.C.4, 2010.C.2, or 2010.C.3.

In embodiments in which one or more tissue-specific localization proteins are selected from Table 211 or Table 2011, the one or more tissue-specific localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific localization proteins are selected from Table 211.A.1 or Table 2011.A.1. In certain embodiments, two or more tissue-specific localization proteins are selected from Table 211.A.1 or Table 2011.A.1. In preferred embodiments, the one or more tissue-specific localization proteins are selected from Table 211.A.2 or 2011.A.2.

In embodiments in which one or more tissue-specific cancer localization proteins are selected from Table 211 or Table 2011 and the subject is female, the one or more tissue-specific cancer localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific cancer localization proteins are selected from Table 211.B.1 or Table 2011.B.1. In certain embodiments, two or more tissue-specific cancer localization proteins are selected from Table 211.B.1 or Table 2011.B.1. In preferred embodiments, the one or more tissue-specific cancer localization proteins are selected from Table 211.B.2, 2011.B.2, or 2011.B.3.

In embodiments in which one or more tissue-specific cancer localization proteins are selected from Table 211 or Table 2011 and the subject is male, the one or more tissue-specific cancer localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific cancer localization proteins are selected from Table 211.C.1 or Table 2011.C.1. In certain embodiments, two or more tissue-specific cancer localization proteins are selected from Table 211.C.1 or Table 2011.C.1. In preferred embodiments, the one or more tissue-specific cancer localization proteins are selected from Table 211.C.2, 2011.C.2, or 2011.C.3.

In embodiments in which one or more tissue-specific cancer localization proteins are selected from Table 211 or Table 2011 and the subject is being evaluated for early-stage cancer, the one or more tissue-specific cancer localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific cancer localization proteins are selected from Table 211.A.1 or Table 2011.A.1. In certain embodiments, two or more tissue-specific cancer localization proteins are selected from Table 211.A.1 or Table 2011.A.1. In preferred embodiments, the one or more tissue-specific cancer localization proteins are selected from Table 211.A.2 or 2011.A.2.

In embodiments in which one or more tissue-specific cancer localization proteins are selected from Table 211 or Table 2011, the subject is being evaluated for early-stage cancer, and the subject is female, the one or more tissue-specific cancer localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific cancer localization proteins are selected from Table 211.B.3 or Table 2011.B.1. In certain embodiments, two or more tissue-specific cancer localization proteins are selected from Table 211.B.3 or Table 2011.B.1. In preferred embodiments, the one or more tissue-specific cancer localization proteins are selected from Table 211.B.4, 2011.B.2, or 2011.B.3.

In embodiments in which one or more tissue-specific cancer localization proteins are selected from Table 211 or Table 2011, the subject is being evaluated for early-stage cancer, and the subject is male, the one or more tissue-specific cancer localization proteins may be selected from any of Tables 211.A.1, 211.A.2, 211.B.1, 211.B.2, 211.B.3, 211.B.4, 211.C.1, 211.C.2, 211.C.3, 2011.A.1, 2011.A.2, 2011.B.1, 2011.B.2, 2011.B.3, 2011.C.1, 2011.C.2, and 2011.C.3. In some embodiments, the one or more tissue-specific cancer localization proteins are selected from Table 211.C.3 or Table 2011.C.1. In some embodiments, two or more tissue-specific cancer localization proteins are selected from Table 211.C.3 or Table 2011.C.1. In preferred embodiments, the one or more tissue-specific cancer localization proteins are selected from Table 2011.C.2 or Table 2011.C.3.

In embodiments in which one or more tissue-specific localization proteins are selected from Table 212 or Table 2012, the one or more tissue-specific localization proteins may be selected from any of Tables 212.A.1, 212.A.2, 212.B.1, 212.B.2, 212.B.3, 212.B.4, 2012.A.1, 2012.A.2, and 2012.A.3. In some embodiments, the one or more tissue-specific localization proteins are selected from Table 212.A.1 or Table 2012.A.1. In certain embodiments, two or more tissue-specific localization proteins are selected from Table 212.A.1 or Table 2012.A.1. In preferred embodiments, the one or more tissue-specific localization proteins are selected from Table 212.A.2, 2012.A.2, or 2012.A.3.

In embodiments in which two or more tissue-specific cancer localization proteins are selected from Table 212 or Table 2012 and the subject is female, the one or more tissue-specific cancer localization proteins may be selected from any of Tables 212.A.1, 212.A.2, 212.B.1, 212.B.2, 212.B.3, 212.B.4, 2012.A.1, 2012.A.2, and 2012.A.3. In some embodiments, the one or more tissue-specific cancer localization proteins are selected from Table 212.B.1 or Table 2012.A.1. In certain embodiments, two or more tissue-specific cancer localization proteins are selected from Table 212.B.1 or Table 2012.A.1. In preferred embodiments, the one or more tissue-specific cancer localization proteins are selected from Table 212.B.2, 2012.A.2, or 2012.A.3.

In embodiments in which one or more tissue-specific cancer localization proteins are selected from Table 212 or Table 2012 and the subject is being evaluated for early-stage cancer, the one or more tissue-specific cancer localization proteins may be selected from any of Tables 212.A.1, 212.A.2, 212.B.1, 212.B.2, 212.B.3, 212.B.4, 2012.A.1, 2012.A.2, and 2012.A.3. In some embodiments, the one or more tissue-specific cancer localization proteins are selected from Table 212.A.1 or Table 2012.A.1. In certain embodiments, two or more tissue-specific cancer localization proteins are selected from Table 212.A.1 or Table 2012.A.1. In preferred embodiments, the one or more tissue-specific cancer localization proteins are selected from Table 212.A.2, 2012.A.2, or 2012.A.3.

In embodiments in which one or more tissue-specific cancer localization proteins are selected from Table 212 or Table 2012, the subject is being evaluated for early-stage cancer, and the subject is female, the one or more tissue-specific cancer localization proteins may be selected from any of Tables 212.A.1, 212.A.2, 212.B.1, 212.B.2, 212.B.3, 212.B.4, 2012.A.1, 2012.A.2, and 2012.A.3. In some embodiments, the one or more tissue-specific cancer localization proteins are selected from Table 212.B.3 or Table 2012.A.1. In certain embodiments, two or more tissue-specific cancer localization proteins are selected from Table 212.B.3 or Table 2012.A.1. In preferred embodiments, the one or more tissue-specific cancer localization proteins are selected from Table 212.B.4, 2012.A.2, or 2012.A.3.

In embodiments in which one or more tissue-specific localization proteins are selected from Table 213 or Table 2013, the one or more tissue-specific localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific localization proteins are selected from Table 213.A.1 or Table 2013.A.1. In certain embodiments, two or more tissue-specific localization proteins are selected from Table 213.A.1 or Table 2013.A.1. In preferred embodiments, the one or more tissue-specific localization proteins are selected from Table 213.A.2, 2013.A.2, or 2013.A.3.

In embodiments in which one or more tissue-specific cancer localization proteins are selected from Table 213 or Table 2013 and the subject is female, the one or more tissue-specific cancer localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific cancer localization proteins are selected from Table 213.B.1 or Table 2013.B.1. In certain embodiments, two or more tissue-specific cancer localization proteins are selected from Table 213.B.1 or Table 2013.B.1. In preferred embodiments, the one or more tissue-specific cancer localization proteins are selected from Table 213.B.2, 2013.B.2, or 2013.B.3.

In embodiments in which one or more tissue-specific cancer localization proteins are selected from Table 213 or Table 2013 and the subject is male, the one or more tissue-specific cancer localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific cancer localization proteins are selected from Table 213.C.1 or Table 2013.C.1. In certain embodiments, two or more tissue-specific cancer localization proteins are selected from Table 213.C.1 or Table 2013.C.1. In preferred embodiments, the one or more tissue-specific cancer localization proteins are selected from Table 213.C.2, 2013.C.2, or 2013.C.3.

In embodiments in which one or more tissue-specific cancer localization proteins are selected from Table 213 or Table 2013 and the subject is being evaluated for early-stage cancer, the one or more tissue-specific cancer localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific cancer localization proteins are selected from Table 213.A.1 or Table 2013.A.1. In certain embodiments, two or more tissue-specific cancer localization proteins are selected from Table 213.A.1 or Table 2013.A.1. In preferred embodiments, the one or more tissue-specific cancer localization proteins are selected from Table 213.A.2, 2013.A.2, or 2013.A.3.

In embodiments in which one or more tissue-specific cancer localization proteins are selected from Table 213 or Table 2013, the subject is being evaluated for early-stage cancer, and the subject is female, the one or more tissue-specific cancer localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific cancer localization proteins are selected from Table 213.B.3 or Table 2013.B.1. In some embodiments, two or more tissue-specific cancer localization proteins are selected from Table 213.B.3 or Table 2013.B.1. In preferred embodiments, the one or more tissue-specific cancer localization proteins are selected from Table 2013.B.2 or Table 2013.B.3.

In embodiments in which one or more tissue-specific cancer localization proteins are selected from Table 213 or Table 2013, the subject is being evaluated for early-stage cancer, and the subject is male, the one or more tissue-specific cancer localization proteins may be selected from any of Tables 213.A.1, 213.A.2, 213.B.1, 213.B.2, 213.B.3, 213.C.1, 213.C.2, 213.C.3, 2013.A.1, 2013.A.2, 2013.A.3, 2013.B.1, 2013.B.2, 2013.B.3, 2013.C.1, 2013.C.2, and 2013.C.3. In some embodiments, the one or more tissue-specific cancer localization proteins are selected from Table 213.C.3 or Table 2013.C.1. In some embodiments, two or more tissue-specific cancer localization proteins are selected from Table 213.C.3 or Table 2013.C.1. In preferred embodiments, the one or more tissue-specific cancer localization proteins are selected from Table 2013.C.2 or Table 2013.C.3.

In embodiments in which one or more tissue-specific localization proteins are selected from Table 214 or Table 2014, the one or more tissue-specific localization proteins may be selected from any of Tables 214.A.1, 214.A.2, 214.B.1, 214.1B.2, 214.B.3, 214.B.4, 2014.A.1, 2014.A.2, and 2014.A.3. In some embodiments, the one or more tissue-specific localization proteins are selected from Table 214.A.1 or Table 2014.A.1. In certain embodiments, two or more tissue-specific localization proteins are selected from Table 214.A.1 or Table 2014.A.1. In preferred embodiments, the one or more tissue-specific localization proteins are selected from Table 214.A.2, 2014.A.2, or 2014.A.3.

In embodiments in which two or more tissue-specific cancer localization proteins are selected from Table 214 or Table 2014 and the subject is male, the one or more tissue-specific cancer localization proteins may be selected from any of Tables 214.A.1, 214.A.2, 214.B.1, 214.B.2, 214.B.3, 214.B.4, 2014.A.1, 2014.A.2, and 2014.A.3. In some embodiments, the one or more tissue-specific cancer localization proteins are selected from Table 214.B.1 or Table 2014.A.1. In certain embodiments, two or more tissue-specific cancer localization proteins are selected from Table 214.1B.1 or Table 2014.A.1. In preferred embodiments, the one or more tissue-specific cancer localization proteins are selected from Table 214.B.2, 2014.A.2, or 2014.A.3.

In embodiments in which one or more tissue-specific cancer localization proteins are selected from Table 214 or Table 2014 and the subject is being evaluated for early-stage cancer, the one or more tissue-specific cancer localization proteins may be selected from any of Tables 214.A.1, 214.A.2, 214.B.1, 214.1B.2, 214.B.3, 214.B.4, 2014.A.1, 2014.A.2, and 2014.A.3. In some embodiments, the one or more tissue-specific cancer localization proteins are selected from Table 214.A.1 or Table 2014.A.1. In certain embodiments, two or more tissue-specific cancer localization proteins are selected from Table 214.A.1 or Table 2014.A.1. In preferred embodiments, the one or more tissue-specific cancer localization proteins are selected from Table 214.A.2, 2014.A.2, or 2014.A.3.

In embodiments in which one or more tissue-specific cancer localization proteins are selected from Table 214 or Table 2014, the subject is being evaluated for early-stage cancer, and the subject is male, the one or more tissue-specific cancer localization proteins may be selected from any of Tables 214.A.1, 214.A.2, 214.B.1, 214.B.2, 214.B.3, 214.B.4, 2014.A.1, 2014.A.2, and 2014.A.3. In some embodiments, the one or more tissue-specific cancer localization proteins are selected from Table 214.B.3 or Table 2014.A.1. In certain embodiments, two or more tissue-specific cancer localization proteins are selected from Table 214.B.3 or Table 2014.A.1. In preferred embodiments, the one or more tissue-specific cancer localization proteins are selected from Table 214.B.4, 2014.A.2, or 2014.A.3.

In embodiments in which one or more tissue-specific localization proteins are selected from Table 215 or Table 2015, the one or more tissue-specific localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific localization proteins are selected from Table 215.A.1 or Table 2015.A.1. In certain embodiments, two or more tissue-specific localization proteins are selected from Table 215.A.1 or Table 2015.A.1. In preferred embodiments, the one or more tissue-specific localization proteins are selected from Table 215.A.2, 2015.A.2, or 2015.A.3.

In embodiments in which one or more tissue-specific cancer localization proteins are selected from Table 215 or Table 2015 and the subject is female, the one or more tissue-specific cancer localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific cancer localization proteins are selected from Table 215.B.1 or Table 2015.B.1. In certain embodiments, two or more tissue-specific cancer localization proteins are selected from Table 215.B.1 or Table 2015.B.1. In preferred embodiments, the one or more tissue-specific cancer localization proteins are selected from Table 215.B.2, 2015.B.2, or 2015.B.3.

In embodiments in which one or more tissue-specific cancer localization proteins are selected from Table 215 or Table 2015 and the subject is male, the one or more tissue-specific cancer localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific cancer localization proteins are selected from Table 215.C.1 or Table 2015.C.1. In certain embodiments, two or more tissue-specific cancer localization proteins are selected from Table 215.C.1 or Table 2015.C.1. In preferred embodiments, the one or more tissue-specific cancer localization proteins are selected from Table 215.C.2, 2015.C.2, or 2015.C.3.

In embodiments in which one or more tissue-specific cancer localization proteins are selected from Table 215 or Table 2015 and the subject is being evaluated for early-stage cancer, the one or more tissue-specific cancer localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific cancer localization proteins are selected from Table 215.A.1 or Table 2015.A.1. In certain embodiments, two or more tissue-specific cancer localization proteins are selected from Table 215.A.1 or Table 2015.A.1. In preferred embodiments, the one or more tissue-specific cancer localization proteins are selected from Table 215.A.2, 2015.A.2, or 2015.A.3.

In embodiments in which one or more tissue-specific cancer localization proteins are selected from Table 215 or Table 2015, the subject is being evaluated for early-stage cancer, and the subject is female, the one or more tissue-specific cancer localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific cancer localization proteins are selected from Table 215.B.3 or Table 2015.B.1. In certain embodiments, two or more tissue-specific cancer localization proteins are selected from Table 215.B.3 or Table 2015.B.1. In preferred embodiments, the one or more tissue-specific cancer localization proteins are selected from Table 215.B.4, 2015.B.2, or 2015.B.3.

In embodiments in which one or more tissue-specific cancer localization proteins are selected from Table 215 or Table 2015, the subject is being evaluated for early-stage cancer, and the subject is male, the one or more tissue-specific cancer localization proteins may be selected from any of Tables 215.A.1, 215.A.2, 215.B.1, 215.B.2, 215.B.3, 215.B.4, 215.C.1, 215.C.2, 215.C.3, 215.C.4, 2015.A.1, 2015.A.2, 2015.A.3, 2015.B.1, 2015.B.2, 2015.B.3, 2015.C.1, 2015.C.2, and 2015.C.3. In some embodiments, the one or more tissue-specific cancer localization proteins are selected from Table 215.C.3 or Table 2015.C.1. In certain embodiments, two or more tissue-specific cancer localization proteins are selected from Table 215.C.3 or Table 2015.C.1. In preferred embodiments, the one or more tissue-specific cancer localization proteins are selected from Table 215.C.4, 2015.C.2, or 2015.C.3.

In embodiments in which one or more tissue-specific localization proteins are selected from Table 216 or Table 2016, the one or more tissue-specific localization proteins may be selected from any of Tables 216.A.1, 216.A.2, 216.B.1, 216.B.2, 216.B.3, 216.B.4, 2016.A.1, 2016.A.2, and 2016.A.3. In some embodiments, the one or more tissue-specific localization proteins are selected from Table 216.A.1 or Table 2016.A.1. In certain embodiments, two or more tissue-specific localization proteins are selected from Table 216.A.1 or Table 2016.A.1. In preferred embodiments, the one or more tissue-specific localization proteins are selected from Table 216.A.2, 2016.A.2, or 2016.A.3.

In embodiments in which one or more tissue-specific cancer localization proteins are selected from Table 216 or Table 2016 and the subject is male, the one or more tissue-specific cancer localization proteins may be selected from any of Tables 216.A.1, 216.A.2, 216.B.1, 216.B.2, 216.B.3, 216.B.4, 2016.A.1, 2016.A.2, and 2016.A.3. In some embodiments, the one or more tissue-specific cancer localization proteins are selected from Table 216.B.1 or Table 2016.A.1. In certain embodiments, two or more tissue-specific cancer localization proteins are selected from Table 216.B.1 or Table 2016.A.1. In preferred embodiments, the one or more tissue-specific cancer localization proteins are selected from Table 216.B.2, 2016.A.2, or 2016.A.3.

In embodiments in which one or more tissue-specific cancer localization proteins are selected from Table 216 or Table 2016 and the subject is being evaluated for early-stage cancer, the one or more tissue-specific cancer localization proteins may be selected from any of Tables 216.A.1, 216.A.2, 216.B.1, 216.B.2, 216.B.3, 216.B.4, 2016.A.1, 2016.A.2, and 2016.A.3. In some embodiments, the one or more tissue-specific cancer localization proteins are selected from Table 216.A.1 or Table 2016.A.1. In certain embodiments, two or more tissue-specific cancer localization proteins are selected from Table 216.A.1 or Table 2016.A.1. In preferred embodiments, the one or more tissue-specific cancer localization proteins are selected from Table 216.A.2, 2016.A.2, or 2016.A.3.

In embodiments in which one or more tissue-specific cancer localization proteins are selected from Table 216 or Table 2016, the subject is being evaluated for early-stage cancer, and the subject is male, the one or more tissue-specific cancer localization proteins may be selected from any of Tables 216.A.1, 216.A.2, 216.B.1, 216.B.2, 216.B.3, 216.B.4, 2016.A.1, 2016.A.2, and 2016.A.3. In some embodiments, the one or more tissue-specific cancer localization proteins are selected from Table 216.B.3 or Table 2016.A.1. In certain embodiments, two or more tissue-specific cancer localization proteins are selected from Table 216.B.3 or Table 2016.A.1. In preferred embodiments, the one or more tissue-specific cancer localization proteins are selected from Table 216.B.4, 2016.A.2, or 2016.A.3.

In embodiments in which one or more tissue-specific localization proteins are selected from Table 217 or Table 2017, the one or more tissue-specific localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific localization proteins are selected from Table 217.A.1 or Table 2017.A.1. In certain embodiments, two or more tissue-specific localization proteins are selected from Table 217.A.1 or Table 2017.A.1. In preferred embodiments, the one or more tissue-specific localization proteins are selected from Table 217.A.2, 2017.A.2, or 2017.A.3.

In embodiments in which one or more tissue-specific cancer localization proteins are selected from Table 217 or Table 2017 and the subject is female, the one or more tissue-specific cancer localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific cancer localization proteins are selected from Table 217.B.1 or Table 2017.B.1. In certain embodiments, two or more tissue-specific cancer localization proteins are selected from Table 217.B.1 or Table 2017.B.1. In preferred embodiments, the one or more tissue-specific cancer localization proteins are selected from Table 217.B.2, 2017.B.2, or 2017.B.3.

In embodiments in which one or more tissue-specific cancer localization proteins are selected from Table 217 or Table 2017 and the subject is male, the one or more tissue-specific cancer localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific cancer localization proteins are selected from Table 217.C.1 or Table 2017.C.1. In certain embodiments, two or more tissue-specific cancer localization proteins are selected from Table 217.C.1 or Table 2017.C.1. In preferred embodiments, the one or more tissue-specific cancer localization proteins are selected from Table 217.C.2, 2017.C.2, or 2017.C.3.

In embodiments in which one or more tissue-specific cancer localization proteins are selected from Table 217 or Table 2017 and the subject is being evaluated for early-stage cancer, the one or more tissue-specific cancer localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific cancer localization proteins are selected from Table 217.A.1 or Table 2017.A.1. In certain embodiments, two or more tissue-specific cancer localization proteins are selected from Table 217.A.1 or Table 2017.A.1. In preferred embodiments, the one or more tissue-specific cancer localization proteins are selected from Table 217.A.2, 2017.A.2, or 2017.A.3.

In embodiments in which one or more tissue-specific cancer localization proteins are selected from Table 217 or Table 2017, the subject is being evaluated for early-stage cancer, and the subject is female, the one or more tissue-specific cancer localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific cancer localization proteins are selected from Table 217.B.3 or Table 2017.B.1. In certain embodiments, two or more tissue-specific cancer localization proteins are selected from Table 217.B.3 or Table 2017.B.1. In preferred embodiments, the one or more tissue-specific cancer localization proteins are selected from Table 217.B.4, 2017.B.2, or 2017.B.3.

In embodiments in which one or more tissue-specific cancer localization proteins are selected from Table 217 or Table 2017, the subject is being evaluated for early-stage cancer, and the subject is male, the one or more tissue-specific cancer localization proteins may be selected from any of Tables 217.A.1, 217.A.2, 217.B.1, 217.B.2, 217.B.3, 217.B.4, 217.C.1, 217.C.2, 217.C.3, 217.C.4, 2017.A.1, 2017.A.2, 2017.A.3, 2017.B.1, 2017.B.2, 2017.B.3, 2017.C.1, 2017.C.2, and 2017.C.3. In some embodiments, the one or more tissue-specific cancer localization proteins are selected from Table 217.C.3 or Table 2017.C.1. In certain embodiments, two or more tissue-specific cancer localization proteins are selected from Table 217.C.3 or Table 2017.C.1. In preferred embodiments, the one or more tissue-specific cancer localization proteins are selected from Table 217.C.4, 2017.C.2, or 2017.C.3.

In embodiments in which one or more tissue-specific localization proteins are selected from Table 218 or Table 2018, the one or more tissue-specific localization proteins may be selected from any of Tables 218.A.1, 218.A.2, 218.B.1, 218.B.2, 218.B.3, 218.B.4, 2018.A.1, 2018.A.2, and 2018.A.3. In some embodiments, the one or more tissue-specific localization proteins are selected from Table 218.A.1 or Table 2018.A.1. In certain embodiments, two or more tissue-specific localization proteins are selected from Table 218.A.1 or Table 2018.A.1. In preferred embodiments, the one or more tissue-specific localization proteins are selected from Table 218.A.2, 2018.A.2, or 2018.A.3.

In embodiments in which one or more tissue-specific cancer localization proteins are selected from Table 218 or Table 2018 and the subject is female, the one or more tissue-specific cancer localization proteins may be selected from any of Tables 218.A.1, 218.A.2, 218.B.1, 218.B.2, 218.B.3, 218.B.4, 2018.A.1, 2018.A.2, and 2018.A.3. In some embodiments, the one or more tissue-specific cancer localization proteins are selected from Table 218.B.1 or Table 2018.A.1. In certain embodiments, two or more tissue-specific cancer localization proteins are selected from Table 218.B.1 or Table 2018.A.1. In preferred embodiments, the one or more tissue-specific cancer localization proteins are selected from Table 218.B.2, 2018.A.2, or 2018.A.3.

In embodiments in which one or more tissue-specific cancer localization proteins are selected from Table 218 or Table 2018 and the subject is being evaluated for early-stage cancer, the one or more tissue-specific cancer localization proteins may be selected from any of Tables 218.A.1, 218.A.2, 218.B.1, 218.B.2, 218.B.3, 218.B.4, 2018.A.1, 2018.A.2, and 2018.A.3, 2018.A.1, 2018.A.2, and 2018.A.3. In some embodiments, the one or more tissue-specific cancer localization proteins are selected from Table 218.A.1 or Table 2018.A.1. In certain embodiments, two or more tissue-specific cancer localization proteins are selected from Table 218.A.1 or Table 2018.A.1. In preferred embodiments, the one or more tissue-specific cancer localization proteins are selected from Table 218.A.2, 2018.A.2, or 2018.A.3.

In embodiments in which one or more tissue-specific cancer localization proteins are selected from Table 218 or Table 2018, the subject is being evaluated for early-stage cancer, and the subject is male, the one or more tissue-specific cancer localization proteins may be selected from any of Tables 218.A.1, 218.A.2, 218.B.1, 218.B.2, 218.B.3, 218.B.4, 2018.A.1, 2018.A.2, and 2018.A.3. In some embodiments, the one or more tissue-specific cancer localization proteins are selected from Table 218.B.3 or Table 2018.A.1. In certain embodiments, two or more tissue-specific cancer localization proteins are selected from Table 218.B.3 or Table 2018.A.1. In preferred embodiments, the one or more tissue-specific cancer localization proteins are selected from Table 218.B.4, 2018.A.2, or 2018.A.3.

In some embodiments, including embodiments in which the subject is being evaluated for early-stage cancer, the tissue-specific localization proteins are from Table 251, 252, 253, or 254. In some embodiments, the tissue-specific localization proteins are from Table 251 or Table 253. In preferred embodiments, the tissue-specific localization proteins are from Table 252 or Table 254.

In some embodiments, including embodiments in which the subject is being evaluated for early-stage cancer and the subject is female, the tissue-specific localization proteins are from Table 251, 252, 253, or 254. In some embodiments, the tissue-specific localization proteins are from Table 251 or Table 252. In preferred embodiments, the tissue-specific localization proteins are from Table 252.

In some embodiments, including embodiments in which the subject is being evaluated for early-stage cancer and the subject is male, the tissue-specific localization proteins are from Table 251, 252, 253, or 254. In some embodiments, the tissue-specific localization proteins are from Table 253 or Table 254. In preferred embodiments, the tissue-specific localization proteins are from Table 254.

### For Methods Involving Tissue-Specific Cancer Detection Proteins

In embodiments in which a subject is evaluated for a cancer that is associated with the bladder or is evaluated for bladder cancer, the one or more tissue-specific detection proteins may be selected from any of Tables 301.A.1, 301.A.2, 301.B.1, 301.B.2, 301.C.1, and 301.C.2. In some embodiments, the one or more tissue-specific detection proteins are selected from Table 301.A.1. In certain embodiments, two or more tissue-specific detection proteins are selected from Table 301.A.1. In preferred embodiments, the one or more tissue-specific detection proteins are selected from Table 301.A.2.

In embodiments in which a subject is evaluated for a cancer that is associated with the bladder or is evaluated for bladder cancer, and the subject is female, the one or more tissue-specific cancer detection proteins may be selected from any of Tables 301.A.1, 301.A.2, 301.B.1, 301.B.2, 301.C.1, and 301.C.2. In some embodiments, the one or more tissue-specific cancer detection proteins are selected from Table 301.B.1. In certain embodiments, two or more tissue-specific cancer detection proteins are selected from Table 301.B.1. In preferred embodiments, the one or more tissue-specific cancer detection proteins are selected from Table 301.B.2.

In embodiments in which a subject is evaluated for a cancer that is associated with the bladder or is evaluated for bladder cancer, and the subject is male, the one or more tissue-specific cancer detection proteins may be selected from any of Tables 301.A.1, 301.A.2, 301.B.1, 301.B.2, 301.C.1, and 301.C.2. In some embodiments, the one or more tissue-specific cancer detection proteins are selected from Table 301.C.1. In certain embodiments, two or more tissue-specific cancer detection proteins are selected from Table 301.C.1. In preferred embodiments, the one or more tissue-specific cancer detection proteins are selected from Table 301.C.2.

In embodiments in which a subject is evaluated for a cancer that is associated with bone or is evaluated for bone cancer, the one or more tissue-specific detection proteins may be selected from any of Tables 302.A.1, 302.A.2, 302.B.1, 302.B.2, 302.C.1, and 302.C.2. In some embodiments, the one or more tissue-specific detection proteins are selected from Table 302.A.1. In certain embodiments, two or more tissue-specific detection proteins are selected from Table 302.A.1. In preferred embodiments, the one or more tissue-specific detection proteins are selected from Table 302.A.2.

In embodiments in which a subject is evaluated for a cancer that is associated with bone or is evaluated for bone cancer, and the subject is female, the one or more tissue-specific cancer detection proteins may be selected from any of Tables 302.A.1, 302.A.2, 302.B.1, 302.B.2, 302.C.1, and 302.C.2. In some embodiments, the one or more tissue-specific cancer detection proteins are selected from Table 302.B.1. In certain embodiments, two or more tissue-specific cancer detection proteins are selected from Table 302.B.1. In preferred embodiments, the one or more tissue-specific cancer detection proteins are selected from Table 302.B.2.

In embodiments in which a subject is evaluated for a cancer that is associated with bone or is evaluated for bone cancer, and the subject is male, the one or more tissue-specific cancer detection proteins may be selected from any of Tables 302.A.1, 302.A.2, 302.B.1, 302.B.2, 302.C.1, and 302.C.2. In some embodiments, the one or more tissue-specific cancer detection proteins are selected from Table 302.C.1. In certain embodiments, two or more tissue-specific cancer detection proteins are selected from Table 302.C.1. In preferred embodiments, the one or more tissue-specific cancer detection proteins are selected from Table 302.C.2.

In embodiments in which a subject is evaluated for a cancer that is associated with the brain or is evaluated for brain cancer, the one or more tissue-specific detection proteins may be selected from any of Tables 303.A.1, 303.A.2, 303.B.1, 303.B.2, 303.C.1, and 303.C.2. In some embodiments, the one or more tissue-specific detection proteins are selected from Table 303.A.1. In certain embodiments, two or more tissue-specific detection proteins are selected from Table 303.A.1. In preferred embodiments, the one or more tissue-specific detection proteins are selected from Table 303.A.2.

In embodiments in which a subject is evaluated for a cancer that is associated with the brain or is evaluated for brain cancer, and the subject is female, the one or more tissue-specific cancer detection proteins may be selected from any of Tables 303.A.1, 303.A.2, 303.B.1, 303.B.2, 303.C.1, and 303.C.2. In some embodiments, the one or more tissue-specific cancer detection proteins are selected from Table 303.B.1. In certain embodiments, two or more tissue-specific cancer detection proteins are selected from Table 303.B.1. In preferred embodiments, the one or more tissue-specific cancer detection proteins are selected from Table 303.B.2.

In embodiments in which a subject is evaluated for a cancer that is associated with the brain or is evaluated for brain cancer, and the subject is male, the one or more tissue-specific cancer detection may be selected from any of Tables 303.A.1, 303.A.2, 303.B.1, 303.B.2, 303.C.1, and 303.C.2. In some embodiments, the one or more tissue-specific cancer detection proteins are selected from Table 303.C.1. In certain embodiments, two or more tissue-specific cancer detection proteins are selected from Table 303.C.1. In preferred embodiments, the one or more tissue-specific cancer detection proteins are selected from Table 303.C.2.

In embodiments in which a subject is evaluated for a cancer that is associated with the breast or is evaluated for breast cancer, the one or more tissue-specific detection proteins may be selected from any of Tables 304.A.1, 304.A.2, 304.B.1, and 304.B.2. In some embodiments, the one or more tissue-specific detection proteins are selected from Table 304.A.1. In certain embodiments, two or more tissue-specific detection proteins are selected from Table 304.A.1. In preferred embodiments, the one or more tissue-specific detection proteins are selected from Table 304.A.2.

In embodiments in which a subject is evaluated for a cancer that is associated with the breast or is evaluated for breast cancer, and the subject is female, the one or more tissue-specific cancer detection proteins may be selected from any of Tables 304.A.1, 304.A.2, 304.B.1, and 304.B.2. In some embodiments, the one or more tissue-specific cancer detection proteins are selected from Table 304.B.1. In certain embodiments, two or more tissue-specific cancer detection proteins are selected from Table 304.B.1. In preferred embodiments, the one or more tissue-specific cancer detection proteins are selected from Table 304.B.2.

In embodiments in which a subject is evaluated for a cancer that is associated with the cervix or is evaluated for cervical cancer, the one or more tissue-specific detection proteins may be selected from any of Tables 305.A.1, 305.A.2, 305.B.1, and 305.B.2. In some embodiments, the one or more tissue-specific detection proteins are selected from Table 305.A.1. In certain embodiments, two or more tissue-specific detection proteins are selected from Table 305.A.1. In preferred embodiments, the one or more tissue-specific detection proteins are selected from Table 305.A.2.

In embodiments in which a subject is evaluated for a cancer that is associated with the cervix or is evaluated for cervical cancer, and the subject is female, the one or more tissue-specific cancer detection proteins may be selected from any of Tables 305.A.1, 305.A.2, 305.B.1, and 305.1.2. In some embodiments, the one or more tissue-specific cancer detection proteins are selected from Table 305.B.1. In certain embodiments, two or more tissue-specific cancer detection proteins are selected from Table 305.B.1. In preferred embodiments, the one or more tissue-specific cancer detection proteins are selected from Table 305.B.2.

In embodiments in which a subject is evaluated for a cancer that is associated with the colon or is evaluated for colon cancer, the one or more tissue-specific detection proteins may be selected from any of Tables 306.A.1, 306.A.2, 306.B.1, 306.B.2, 306.C.1, and 306.C.2. In some embodiments, the one or more tissue-specific detection proteins are selected from Table 306.A.1. In certain embodiments, two or more tissue-specific detection proteins are selected from Table 306.A.1. In preferred embodiments, the one or more tissue-specific detection proteins are selected from Table 306.A.2.

In embodiments in which a subject is evaluated for a cancer that is associated with the colon or is evaluated for colon cancer, and the subject is female, the one or more tissue-specific cancer detection proteins may be selected from any of Tables 306.A.1, 306.A.2, 306.B.1, 306.B.2, 306.C.1, and 306.C.2. In some embodiments, the one or more tissue-specific cancer detection proteins are selected from Table 306.B.1. In certain embodiments, two or more tissue-specific cancer detection proteins are selected from Table 306.B.1. In preferred embodiments, the one or more tissue-specific cancer detection proteins are selected from Table 306.B.2.

In embodiments in which a subject is evaluated for a cancer that is associated with the colon or is evaluated for colon cancer, and the subject is male, the one or more tissue-specific cancer detection proteins may be selected from any of Tables 306.A.1, 306.A.2, 306.B.1, 306.B.2, 306.C.1, and 306.C.2. In some embodiments, the one or more tissue-specific cancer detection proteins are selected from Table 306.C.1. In certain embodiments, two or more tissue-specific cancer detection proteins are selected from Table 306.C.1. In preferred embodiments, the one or more tissue-specific cancer detection proteins are selected from Table 306.C.2.

In embodiments in which a subject is evaluated for a cancer that is associated with the esophagus or is evaluated for esophageal cancer, the one or more tissue-specific detection proteins may be selected from any of Tables 307.A.1, 307.A.2, 307.B.1, 307.B.2, 307.C.1, and 307.C.2. In some embodiments, the one or more tissue-specific detection proteins are selected from Table 307.A.1. In certain embodiments, two or more tissue-specific detection proteins are selected from Table 307.A.1. In preferred embodiments, the one or more tissue-specific detection proteins are selected from Table 307.A.2.

In embodiments in which a subject is evaluated for a cancer that is associated with the esophagus or is evaluated for esophageal cancer, and the subject is female, the one or more tissue-specific cancer detection proteins may be selected from any of Tables 307.A.1, 307.A.2, 307.B.1, 307.B.2, 307.C.1, and 307.C.2. In some embodiments, the one or more tissue-specific cancer detection proteins are selected from Table 307.B.1. In certain embodiments, two or more tissue-specific cancer detection proteins are selected from Table 307.B.1. In preferred embodiments, the one or more tissue-specific cancer detection proteins are selected from Table 307.B.2.

In embodiments in which a subject is evaluated for a cancer that is associated with the esophagus or is evaluated for esophageal cancer, and the subject is male, the one or more tissue-specific cancer detection proteins may be selected from any of Tables 307.A.1, 307.A.2, 307.B.1, 307.B.2, 307.C.1, and 307.C.2. In some embodiments, the one or more tissue-specific cancer detection proteins are selected from Table 307.C.1. In certain embodiments, two or more tissue-specific cancer detection proteins are selected from Table 307.C.1. In preferred embodiments, the one or more tissue-specific cancer detection proteins are selected from Table 307.C.2.

In embodiments in which a subject is evaluated for a cancer that is associated with the kidney or is evaluated for kidney cancer, the one or more tissue-specific detection proteins may be selected from any of Tables 308.A.1, 308.A.2, 308.B.1, 308.B.2, 308.C.1, and 308.C.2. In some embodiments, the one or more tissue-specific detection proteins are selected from Table 308.A.1. In certain embodiments, two or more tissue-specific detection proteins are selected from Table 308.A.1. In preferred embodiments, the one or more tissue-specific detection proteins are selected from Table 308.A.2.

In embodiments in which a subject is evaluated for a cancer that is associated with the kidney or is evaluated for kidney cancer, and the subject is female, the one or more tissue-specific cancer detection proteins may be selected from any of Tables 308.A.1, 308.A.2, 308.B.1, 308.B.2, 308.C.1, and 308.C.2. In some embodiments, the one or more tissue-specific cancer detection proteins are selected from Table 308.B.1. In certain embodiments, two or more tissue-specific cancer detection proteins are selected from Table 308.B.1. In preferred embodiments, the one or more tissue-specific cancer detection proteins are selected from Table 308.B.2.

In embodiments in which a subject is evaluated for a cancer that is associated with the kidney or is evaluated for kidney cancer, and the subject is male, the one or more tissue-specific cancer detection proteins may be selected from any of Tables 308.A.1, 308.A.2, 308.B.1, 308.B.2, 308.C.1, and 308.C.2. In some embodiments, the one or more tissue-specific cancer detection proteins are selected from Table 308.C.1. In certain embodiments, two or more tissue-specific cancer detection proteins are selected from Table 308.C.1. In preferred embodiments, the one or more tissue-specific cancer detection proteins are selected from Table 308.C.2.

In embodiments in which a subject is evaluated for a cancer that is associated with the liver or is evaluated for liver cancer, the one or more tissue-specific detection proteins may be selected from any of Tables 309.A.1, 309.A.2, 309.B.1, 309.B.2, 309.C.1, and 309.C.2. In some embodiments, the one or more tissue-specific detection proteins are selected from Table 309.A.1. In certain embodiments, two or more tissue-specific detection proteins are selected from Table 309.A.1. In preferred embodiments, the one or more tissue-specific detection proteins are selected from Table 309.A.2.

In embodiments in which a subject is evaluated for a cancer that is associated with the liver or is evaluated for liver cancer, and the subject is female, the one or more tissue-specific cancer detection proteins may be selected from any of Tables 309.A.1, 309.A.2, 309.B.1, 309.B.2, 309.C.1, and 309.C.2. In some embodiments, the one or more tissue-specific cancer detection proteins are selected from Table 309.B.1. In certain embodiments, two or more tissue-specific cancer detection proteins are selected from Table 309.B.1. In preferred embodiments, the one or more tissue-specific cancer detection proteins are selected from Table 309.B.2.

In embodiments in which a subject is evaluated for a cancer that is associated with the liver or is evaluated for liver cancer, and the subject is male, the one or more tissue-specific cancer detection proteins may be selected from any of Tables 309.A.1, 309.A.2, 309.B.1, 309.B.2, 309.C.1, and 309.C.2. In some embodiments, the one or more tissue-specific cancer detection proteins are selected from Table 309.C.1. In certain embodiments, two or more tissue-specific cancer detection proteins are selected from Table 309.C.1. In preferred embodiments, the one or more tissue-specific cancer detection proteins are selected from Table 309.C.2.

In embodiments in which a subject is evaluated for a cancer that is associated with the lung—small cell or is evaluated for lung—small cell cancer, the one or more tissue-specific detection proteins may be selected from any of Tables 310.A.1, 310.A.2, 310.B.1, 310.B.2, 310.C.1, and 310.C.2. In some embodiments, the one or more tissue-specific detection proteins are selected from Table 310.A.1. In certain embodiments, two or more tissue-specific detection proteins are selected from Table 310.A.1. In preferred embodiments, the one or more tissue-specific detection proteins are selected from Table 310.A.2.

In embodiments in which a subject is evaluated for a cancer that is associated with the lung—small cell or is evaluated for lung—small cell cancer, and the subject is female, the one or more tissue-specific cancer detection proteins may be selected from any of Tables 310.A.1, 310.A.2, 310.B.1, 310.B.2, 310.C.1, and 310.C.2. In some embodiments, the one or more tissue-specific cancer detection proteins are selected from Table 310.B.1. In certain embodiments, two or more tissue-specific cancer detection proteins are selected from Table 310.B.1. In preferred embodiments, the one or more tissue-specific cancer detection proteins are selected from Table 310.B.2.

In embodiments in which a subject is evaluated for a cancer that is associated with the lung—small cell or is evaluated for lung—small cell cancer, and the subject is male, the one or more tissue-specific cancer detection proteins may be selected from any of Tables 310.A.1, 310.A.2, 310.B.1, 310.B.2, 310.C.1, and 310.C.2. In some embodiments, the one or more tissue-specific cancer detection proteins are selected from Table 310.C.1. In certain embodiments, two or more tissue-specific cancer detection proteins are selected from Table 310.C.1. In preferred embodiments, the one or more tissue-specific cancer detection proteins are selected from Table 310.C.2.

In embodiments in which a subject is evaluated for a cancer that is associated with the lung—non-small cell or is evaluated for lung—non-small cell cancer, the one or more tissue-specific detection proteins may be selected from any of Tables 311.A.1, 311.A.2, 311.B.1, 311.B.2, 311.C.1, and 311.C.2. In some embodiments, the one or more tissue-specific detection proteins are selected from Table 311.A.1. In certain embodiments, two or more tissue-specific detection proteins are selected from Table 311.A.1. In preferred embodiments, the one or more tissue-specific detection proteins are selected from Table 311.A.2.

In embodiments in which a subject is evaluated for a cancer that is associated with the lung—non-small cell or is evaluated for lung—non-small cell cancer, and the subject is female, the one or more tissue-specific cancer detection proteins may be selected from any of Tables 311.A.1, 311.A.2, 311.B.1, 311.B.2, 311.C.1, and 311.C.2. In some embodiments, the one or more tissue-specific cancer detection proteins are selected from Table 311.B.1. In certain embodiments, two or more tissue-specific cancer detection proteins are selected from Table 311.B.1. In preferred embodiments, the one or more tissue-specific cancer detection proteins are selected from Table 311.B.2.

In embodiments in which a subject is evaluated for a cancer that is associated with the lung—non-small cell or is evaluated for lung—non-small cell cancer, and the subject is male, the one or more tissue-specific cancer detection proteins may be selected from any of Tables 311.A.1, 311.A.2, 311.B.1, 311.B.2, 311.C.1, and 311.C.2. In some embodiments, the one or more tissue-specific cancer detection proteins are selected from Table 311.C.1. In certain embodiments, two or more tissue-specific cancer detection proteins are selected from Table 311.C.1. In preferred embodiments, the one or more tissue-specific cancer detection proteins are selected from Table 311.C.2.

In embodiments in which a subject is evaluated for a cancer that is associated with the ovary or is evaluated for ovarian cancer, the one or more tissue-specific detection proteins may be selected from any of Tables 312.A.1, 312.A.2, 312.B.1, and 312.B.2. In some embodiments, the one or more tissue-specific detection proteins are selected from Table 312.A.1. In certain embodiments, two or more tissue-specific detection proteins are selected from Table 312.A.1. In preferred embodiments, the one or more tissue-specific detection proteins are selected from Table 312.A.2.

In embodiments in which a subject is evaluated for a cancer that is associated with the ovary or is evaluated for ovarian cancer, and the subject is female, the one or more tissue-specific cancer detection proteins may be selected from any of Tables 312.A.1, 312.A.2, 312.B.1, and 312.B.2. In some embodiments, the one or more tissue-specific cancer detection proteins are selected from Table 312.B.1. In certain embodiments, two or more tissue-specific cancer detection proteins are selected from Table 312.B.1. In preferred embodiments, the one or more tissue-specific cancer detection proteins are selected from Table 312.B.2.

In embodiments in which a subject is evaluated for a cancer that is associated with the pancreas or is evaluated for pancreatic cancer, the one or more tissue-specific detection proteins may be selected from any of Tables 313.A.1, 313.A.2, 313.B.1, 313.B.2, 313.C.1, and 313.C.2. In some embodiments, the one or more tissue-specific detection proteins are selected from Table 313.A.1. In certain embodiments, two or more tissue-specific detection proteins are selected from Table 313.A.1. In preferred embodiments, the one or more tissue-specific detection proteins are selected from Table 313.A.2.

In embodiments in which a subject is evaluated for a cancer that is associated with the pancreas or is evaluated for pancreatic cancer, and the subject is female, the one or more tissue-specific cancer detection proteins may be selected from any of Tables 313.A.1, 313.A.2, 313.B.1, 313.1.2, 313.C.1, and 313.C.2. In some embodiments, the one or more tissue-specific cancer detection proteins are selected from Table 313.B.1. In certain embodiments, two or more tissue-specific cancer detection proteins are selected from Table 313.B.1. In preferred embodiments, the one or more tissue-specific cancer detection proteins are selected from Table 313.B.2.

In embodiments in which a subject is evaluated for a cancer that is associated with the pancreas or is evaluated for pancreatic cancer, and the subject is male, the one or more tissue-specific cancer detection proteins may be selected from any of Tables 313.A.1, 313.A.2, 313.B.1, 313.B.2, 313.C.1, and 313.C.2. In some embodiments, the one or more tissue-specific cancer detection proteins are selected from Table 313.C.1. In certain embodiments, two or more tissue-specific cancer detection proteins are selected from Table 313.C.1. In preferred embodiments, the one or more tissue-specific cancer detection proteins are selected from Table 313.C.2.

In embodiments in which a subject is evaluated for a cancer that is associated with the prostate or is evaluated for prostate cancer, the one or more tissue-specific detection proteins may be selected from any of Tables 314.A.1, 314.A.2, 314.B.1, and 314.B.2. In some embodiments, the one or more tissue-specific detection proteins are selected from Table 314.A.1. In certain embodiments, two or more tissue-specific detection proteins are selected from Table 314.A.1. In preferred embodiments, the one or more tissue-specific detection proteins are selected from Table 314.A.2.

In embodiments in which a subject is evaluated for a cancer that is associated with the prostate or is evaluated for prostate cancer, and the subject is male, the one or more tissue-specific cancer detection proteins may be selected from any of Tables 314.A.1, 314.A.2, 314.B.1, and 314.B.2. In some embodiments, the one or more tissue-specific cancer detection proteins are selected from Table 314.B.1. In certain embodiments, two or more tissue-specific cancer detection proteins are selected from Table 314.B.1. In preferred embodiments, the one or more tissue-specific cancer detection proteins are selected from Table 314.B.2.

In embodiments in which a subject is evaluated for a cancer that is associated with the stomach or is evaluated for stomach cancer, the one or more tissue-specific detection proteins may be selected from any of Tables 315.A.1, 315.A.2, 315.B.1, 315.B.2, 315.C.1, and 315.C.2. In some embodiments, the one or more tissue-specific detection proteins are selected from Table 315.A.1. In certain embodiments, two or more tissue-specific detection proteins are selected from Table 315.A.1. In preferred embodiments, the one or more tissue-specific detection proteins are selected from Table 315.A.2.

In embodiments in which a subject is evaluated for a cancer that is associated with the stomach or is evaluated for stomach cancer, and the subject is female, the one or more tissue-specific cancer detection proteins may be selected from any of Tables 315.A.1, 315.A.2, 315.B.1, 315.B.2, 315.C.1, and 315.C.2. In some embodiments, the one or more tissue-specific cancer detection proteins are selected from Table 315.B.1. In certain embodiments, two or more tissue-specific cancer detection proteins are selected from Table 315.B.1. In preferred embodiments, the one or more tissue-specific cancer detection proteins are selected from Table 315.B.2.

In embodiments in which a subject is evaluated for a cancer that is associated with the stomach or is evaluated for stomach cancer, and the subject is male, the one or more tissue-specific cancer detection proteins may be selected from any of Tables 315.A.1, 315.A.2, 315.B.1, 315.B.2, 315.C.1, and 315.C.2. In some embodiments, the one or more tissue-specific cancer detection proteins are selected from Table 315.C.1. In certain embodiments, two or more tissue-specific cancer detection proteins are selected from Table 315.C.1. In preferred embodiments, the one or more tissue-specific cancer detection proteins are selected from Table 315.C.2.

In embodiments in which a subject is evaluated for a cancer that is associated with the testes or is evaluated for testicular cancer, the one or more tissue-specific detection proteins may be selected from any of Tables 316.A.1, 316.A.2, 316.B.1, and 316.B.2. In some embodiments, the one or more tissue-specific detection proteins are selected from Table 316.A.1. In certain embodiments, two or more tissue-specific detection proteins are selected from Table 316.A.1. In preferred embodiments, the one or more tissue-specific detection proteins are selected from Table 316.A.2.

In embodiments in which a subject is evaluated for a cancer that is associated with the testes or is evaluated for testicular cancer, and the subject is male, the one or more tissue-specific cancer detection proteins may be selected from any of Tables 316.A.1, 316.A.2, 316.B.1, and 316.B.2. In some embodiments, the one or more tissue-specific cancer detection proteins are selected from Table 316.B.1. In certain embodiments, two or more tissue-specific cancer detection proteins are selected from Table 316.B.1. In preferred embodiments, the one or more tissue-specific cancer detection proteins are selected from Table 316.B.2.

In embodiments in which a subject is evaluated for a cancer that is associated with the thyroid or is evaluated for thyroid cancer, the one or more tissue-specific detection proteins may be selected from any of Tables 317.A.1, 317.A.2, 317.B.1, 317.B.2, 317.C.1, and 317.C.2. In some embodiments, the one or more tissue-specific detection proteins are selected from Table 317.A.1. In certain embodiments, two or more tissue-specific detection proteins are selected from Table 317.A.1. In preferred embodiments, the one or more tissue-specific detection proteins are selected from Table 317.A.2.

In embodiments in which a subject is evaluated for a cancer that is associated with the thyroid or is evaluated for thyroid cancer, and the subject is female, the one or more tissue-specific cancer detection proteins may be selected from any of Tables 317.A.1, 317.A.2, 317.B.1, 317.B.2, 317.C.1, and 317.C.2. In some embodiments, the one or more tissue-specific cancer detection proteins are selected from Table 317.B.1. In certain embodiments, two or more tissue-specific cancer detection proteins are selected from Table 317.B.1. In preferred embodiments, the one or more tissue-specific cancer detection proteins are selected from Table 317.B.2.

In embodiments in which a subject is evaluated for a cancer that is associated with the thyroid or is evaluated for thyroid cancer, and the subject is male, the one or more tissue-specific cancer detection proteins may be selected from any of Tables 317.A.1, 317.A.2, 317.B.1, 317.B.2, 317.C.1, and 317.C.2. In some embodiments, the one or more tissue-specific cancer detection proteins are selected from Table 317.C.1. In certain embodiments, two or more tissue-specific cancer detection proteins are selected from Table 317.C.1. In preferred embodiments, the one or more tissue-specific cancer detection proteins are selected from Table 317.C.2.

In embodiments in which a subject is evaluated for a cancer that is associated with the uterus or is evaluated for uterine cancer, the one or more tissue-specific detection proteins may be selected from any of Tables 318.A.1, 318.A.2, 318.B.1, and 318.B.2. In some embodiments, the one or more tissue-specific detection proteins are selected from Table 318.A.1. In certain embodiments, two or more tissue-specific detection proteins are selected from Table 318.A.1. In preferred embodiments, the one or more tissue-specific detection proteins are selected from Table 318.A.2.

In embodiments in which a subject is evaluated for a cancer that is associated with the uterus or is evaluated for uterine cancer, and the subject is female, the one or more tissue-specific cancer detection proteins may be selected from any of Tables 318.A.1, 318.A.2, 318.B.1, and 318.B.2. In some embodiments, the one or more tissue-specific cancer detection proteins are selected from Table 318.B.1. In certain embodiments, two or more tissue-specific cancer detection proteins are selected from Table 318.B.1. In preferred embodiments, the one or more tissue-specific cancer detection proteins are selected from Table 318.B.2.

### Protein Concentration Measurement and Application of Classifiers

The concentration of proteins in the sample may be measured using protein quantitation techniques known in the art. Such techniques include, but are not limited to, enzyme-linked immunosorbent assays, chemiluminescence immunoassays, immunohistochemistry, liquid-bead immunoassays, mass spectrometry, aptamer-based assays, reverse phase protein arrays, proximity extension assay (PEA), and a combination thereof.

In the methods described herein, the concentration of the two or more proteins are used and combined with mathematical, statistical, and machine-learning methods to create secondary features. One or more proteins with and without secondary features and baseline features, including age, sex, race and ethnicity, past medical history, family history, patient's lab values, comorbidities, and concomitant medications, are used in one or more predictive models to calculate a score.

Machine learning and statistical analyses techniques used to generate features and the final score for each cancer are included but not limited to the following concepts and methods: Supervised learning concepts may include AODE; Artificial neural network, such as Backpropagation, Auto encoders, Hopfield networks, Boltzmann machines, Restricted Boltzmann Machines, and Spiking neural networks: Bayesian statistics, such as Bayesian network and Bayesian knowledge base; Case-based reasoning; Gaussian process regression; Gene expression programming: Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning: Ripple down rules, a knowledge acquisition methodology; Symbolic machine learning algorithms; Support vector machines; Random Forests; Ensembles of classifiers, such as Bootstrap aggregating (bagging) and Boosting (meta-algorithm): Ordinal classification; Information fuzzy networks (IFN); Conditional Random Field: ANOVA; Linear classifiers, such as Fisher's linear discriminant, Linear regression, Logistic regression, Multinomial logistic regression, I Bayes classifier, Perceptron, Support vector machines; Quadratic classifiers; k-nearest neighbor; Boosting; Decision trees, such as C4.5. Random forests, ID3, CART, SLIQ SPRINT; Bayesian networks, sucINaive Bayes; and Hidden Markov models. Unsupervised learning concepts may include; Expectation-maximization algorithm: Vector Quantization: Generative topographic map; Information bottleneck method; Artificial neural network, such as Self-organizing map; Association rule learning, such as Apriori algorithm, Eclat algorithm, and FP growth algorithm; Hierarchical clusterings such as Single linkage clustering and Conceptual clustering; Cluster analysis, such as K-means algorithm, Fuzzy clustering. DBSCAN, and OPTICS algorithm; and Outlier Detection, such as Local Outlier Factor. Semi-supervised learning concepts may include; Generative models; Low-density separation; Graph-based methods, and Co-training. Reinforcement learning concepts may include Temporal difference learning; Q-learning. Learning Automata, and SARSA. Deep learning concepts may include Deep belief networks: Deep Boltzmann machines; Deep Convolutional neural networks; Deep Recurrent neural networks; and Hierarchical temporal memory.

For concentrations obtained from detection proteins, one or more features are fed into one or more computation models. The classifiers are used to calculate a score for the patient. The scores of different classifiers may be combined to identify the patient as having cancer or not, or having a specific cancer or not. The computational model may use one or more proteins or secondary features with and without baseline features that could generate a (ROC curve greater than or equal to 0.6. This step determines if the sample indicates the presence of any cancer (as determined using pan-cancer detection proteins), or the presence of a specific cancer (as determined using tissue-specific cancer detection proteins).

For concentrations obtained from localization proteins, protein concentrations and/or secondary features are fed into one or more predictive models. The features could be similar or different from what was used in determining any cancer status. The classifiers are used to calculate a score for the patient for the possible cancer types, i.e., cancers of the bladder, bone, brain, breast, cervix, colon, esophagus, kidney, liver, lung—small cell, lung—non-small cell, ovary, pancreas, prostate, stomach, testis, thyroid, and uterine. In some embodiments, the classifier determines an estimate of the probability that the patient has the particular cancer, and the estimated probability can be compared among each cancer. The predictive models use the proteins or derived secondary features that could generate a ROC curve greater than or equal to 0.6. The scores are combined to discriminate different cancer types.

In the methods comprising phase (I) and phase (II), the scores from the two phases are combined in an ensemble model weighted by the performance and uncertainty of the estimate to identify one or more cancer types.

Generally, machine learning algorithms are used to construct models that accurately assign class labels to examples based on the input features that describe the example.

The classifier may be used to distinguish cancer from non-cancer, and distinguish specific cancer, including bladder, bone, brain, breast, cervix, colon, esophagus, kidney, liver, lung—small cell, lung—non-small cell, ovary, pancreas, prostate, stomach, testis, thyroid, and uterine, and distinguish cancer subtype by tissue, biomarkers, mutation, standard treatment or combination of them including but not limited to following cancer and subtypes: Lung cancer: non-small cell lung cancer, squamous cell lung cancer, adenocarcinoma: breast cancer: HR+, HR−, TNBC. The classifier may separate the tumor by biomarker, including HER2, High MSI; mutations: BRCA1, BRCA2, RAS, and BRAF.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1

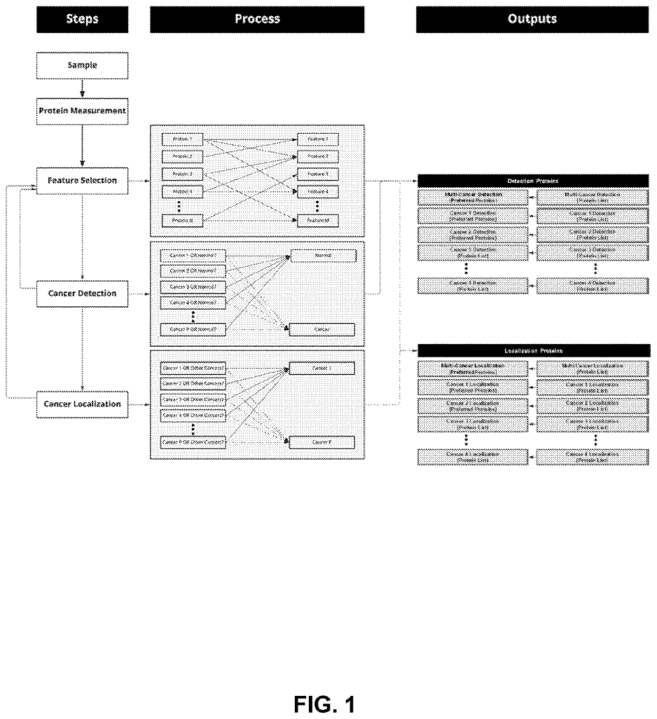

Analyses were performed to identify the pan-cancer proteins and tissue-specific cancer proteins of the present invention. An overview of the analysis is presented in FIG. 1.

### Sample Collection

Plasma samples from 18 early-stage solid tumors, including cancers of the bladder, bone, brain, breast, cervix, colon, esophagus, kidney, liver, lung—small cell, lung—non-small cell, ovary, pancreas, prostate, stomach, testis, thyroid, and uterine. Plasma samples were also conducted from healthy individuals without underlying diseases. Samples from cancer patients were included only if the diagnosis was confirmed by a pathologist and the patient was treatment naïve.

### Protein Measurement

Liquid biopsy offers the possibility to assess the state of health of specific tissues in the body by detecting their released molecules in easily accessible body fluids such as blood. The approach offers a means to screen for disease processes anywhere in the body, and it represents a simple and practical alternative to taking biopsies of specific organs for molecular analysis.

While any protein measurement technique could have been used, including enzyme-linked immunosorbent assays (ELISA), chemiluminescence immunoassays (CLIA), immunohistochemistry (IHC), liquid-bead immunoassays, mass spectrometry, aptamer-based assays, reverse phase protein arrays (RPPA), etc., a proximity extension assay (PEA) was employed to evaluate proteins in plasma. In PEA, each protein was recognized by two antibodies for proper detection. In proximity assays, each of the two antibodies was conjugated to one of two different DNA oligonucleotides, and the reagents were incubated with the samples in solution. The proximity reactions underwent a dilution step. Oligonucleotides on pairs of antibodies that remain in proximity by virtue of having bound the same protein molecule then underwent DNA ligation (proximity ligation assay) or DNA polymerization (proximity extension assay). The effect of the ligation or polymerization reactions was to create amplifiable reporter DNA strands for sensitive readout via, for example, real-time PCR or next-generation sequencing, and the assays could be performed in high multiplex. By constructing the assays so that only proper pairs of antibodies can yield detection signals, but no other combination of antibodies, the detection of many different proteins in parallel was possible without eroding detection specificity by reactions of noncognate pairs.

The analytical performance of the panels was validated for sensitivity, dynamic range, specificity, precision, and scalability. The analytical measuring range was defined by the lower limit of quantification (LLOQ) and upper limit of quantification (ULOQ) and reported in pg/mL. The high dose hook effect (a state of antigen excess relative to the reagent antibodies resulting in falsely lower values) was also determined for each analyte.

All assays were thoroughly validated for precision (repeatability and reproducibility). Intra-assay variation (within-run) was calculated as the mean CV for 6 individual samples, within each of 7 separate runs during the validation studies. Inter-assay variation (between-runs) was calculated as the mean CV, for the same 6 individual samples, among 7 separate runs during the validation studies. Across all 3072 assays, the mean intra-assay and inter-assay variations observed were 8% and 11%, respectively.

Each protein analyte was addressed by a matched pair of antibodies, coupled to unique, partially complementary oligonucleotides and measured by quantitative real-time PCR. Validation of the readout specificity for all of the panels was carried out using a simple, sequential approach in which pools of protein analytes were tested with all 92 antibody probe pairs in the panel.

### Feature Selection

Proteins were used to create features that could be used for the classification of samples. The proteins were categorized based on their concentration or their patterns of change detected by different statistical or machine-learning techniques to create new features.

Machine learning and statistical analyses techniques used to generate features and the final score for each cancer were included but not limited to the following concepts and methods: supervised learning concepts that may include AODE; artificial neural network, such as Backpropagation, Auto encoders, Hopfield networks, Boltzmann machines, Restricted Boltzmann Machines, and Spiking neural networks; Bayesian statistics, such as Bayesian network and Bayesian knowledge base; case-based reasoning; Gaussian process regression; gene expression programming; group method of data handling (GMDH); inductive logic programming; instance-based learning; lazy learning; learning Automata; learning vector quantization: logistic model tree; minimum message length (decision trees, decision graphs, etc.), such as nearest neighbor algorithm and analogical modeling; probability approximately correct learning (PAC) learning; ripple down rules, a knowledge acquisition methodology; symbolic machine learning algorithms; support vector machines; random forests; ensembles of classifiers, such as bootstrap aggregating (bagging) and boosting (meta-algorithm): ordinal classification; information fuzzy networks (IFN); conditional random field; ANOVA; linear classifiers, such as Fisher's linear discriminant, linear regression, logistic regression, multinomial logistic regression, naive Bayes classifier, Perceptron, support vector machines; quadratic classifiers; k-nearest neighbor; boosting; decision trees, such as C4.5, random forests, ID3, CART. SLIQ SPRINT; Bayesian networks, such as Naive Bayes; and Hidden Markov models, cUnsupervised learning concepts may include; expectation-maximization algorithm; vector quantization; generative topographic map; information bottleneck method; artificial neural network, such as self-organizing map; association rule learning, such as Apriori algorithm. Eclat algorithm, and FP growth algorithm; hierarchical clusterings such as single linkage clustering and conceptual clustering; cluster analysis, such as K-means algorithm, fuzzy clustering, DBSCAN, and OPTICS algorithm; and outlier detection, such as local outlier factor. Semi-supervised learning concepts may include: generative models; low-density separation; graph-based methods, and co-training. Reinforcement learning concepts may include temporal difference learning; Q-learning, learning automata, and SARSA. Deep learning concepts may include deep belief networks; deep Boltzmann machines; deep convolutional neural networks; deep recurrent neural networks; and hierarchical temporal memory.

### Cancer Detection Proteins

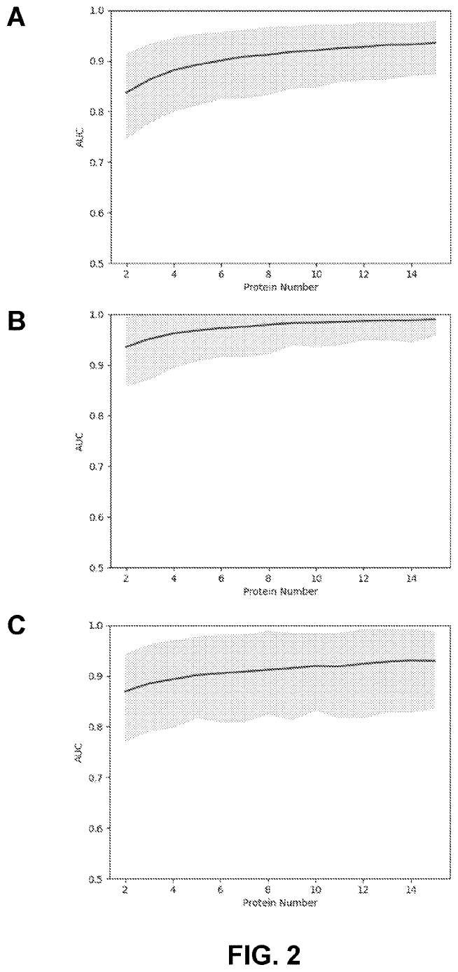

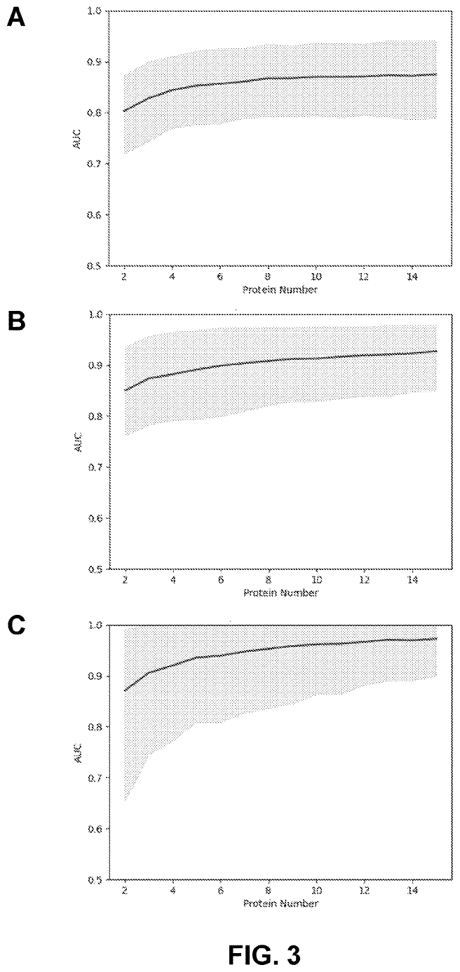

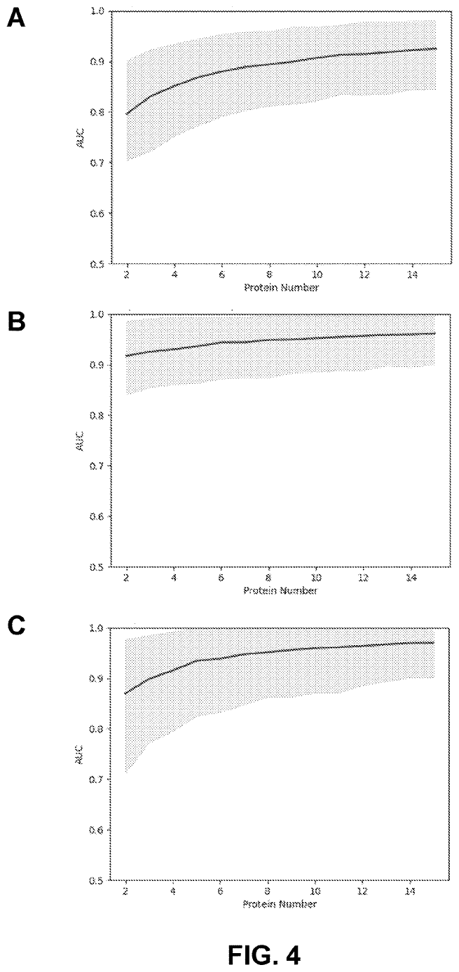

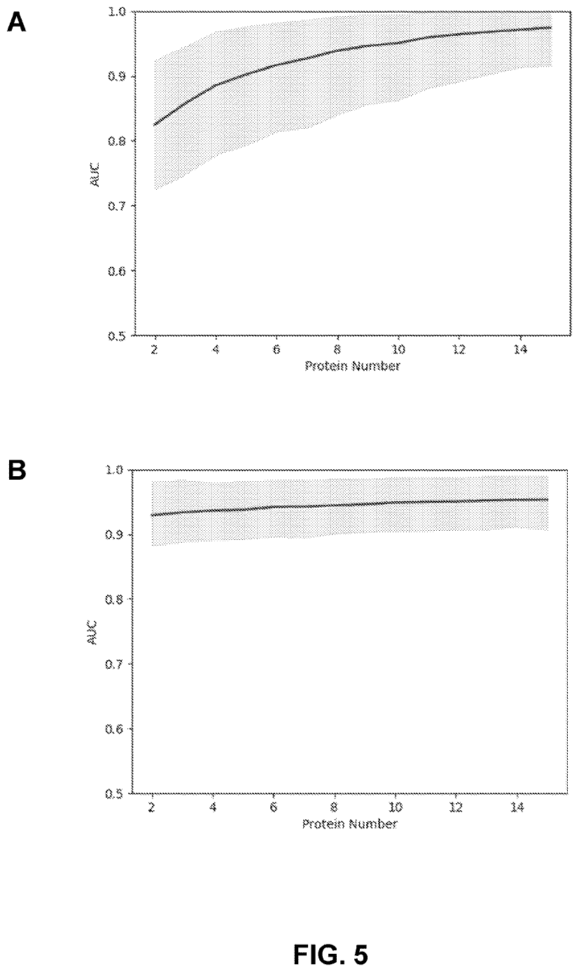

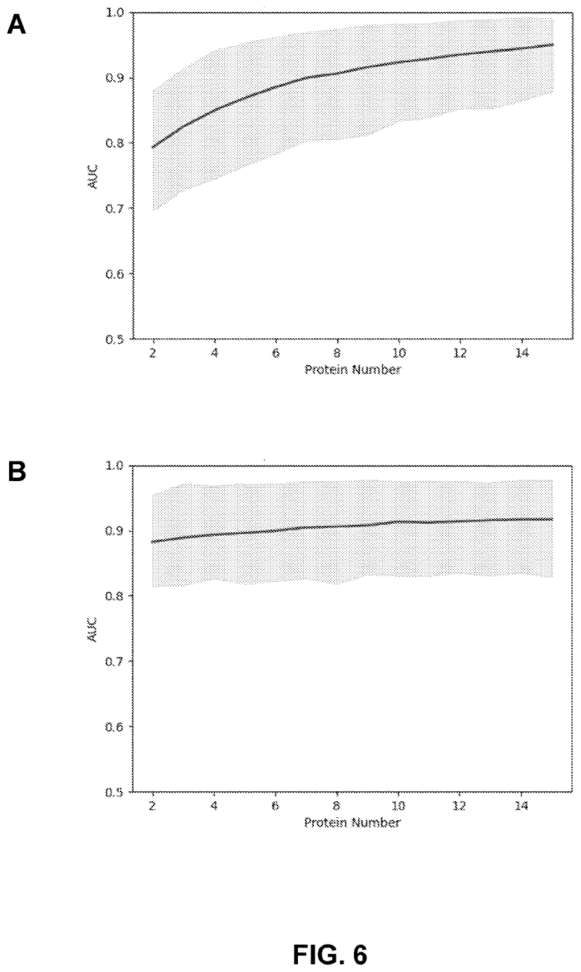

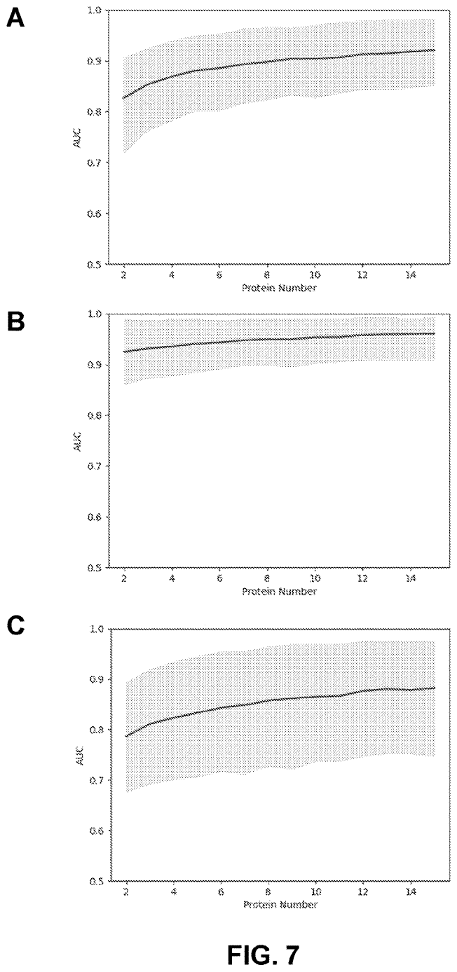

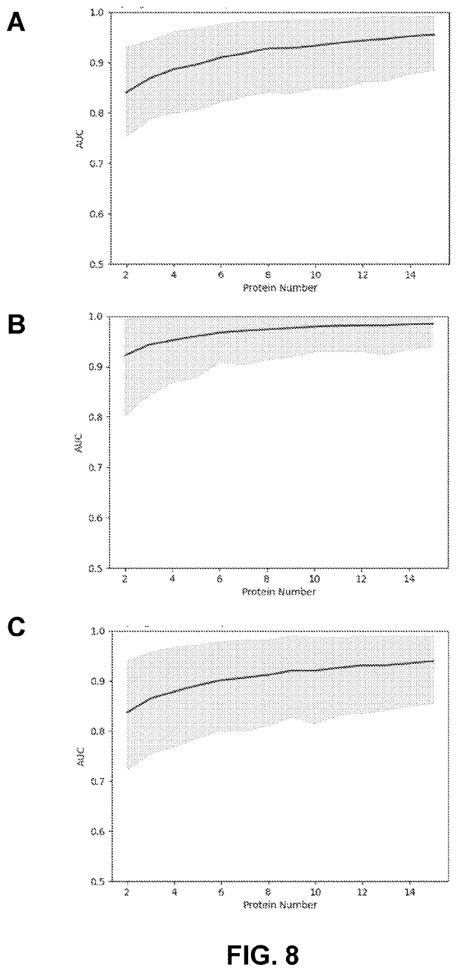

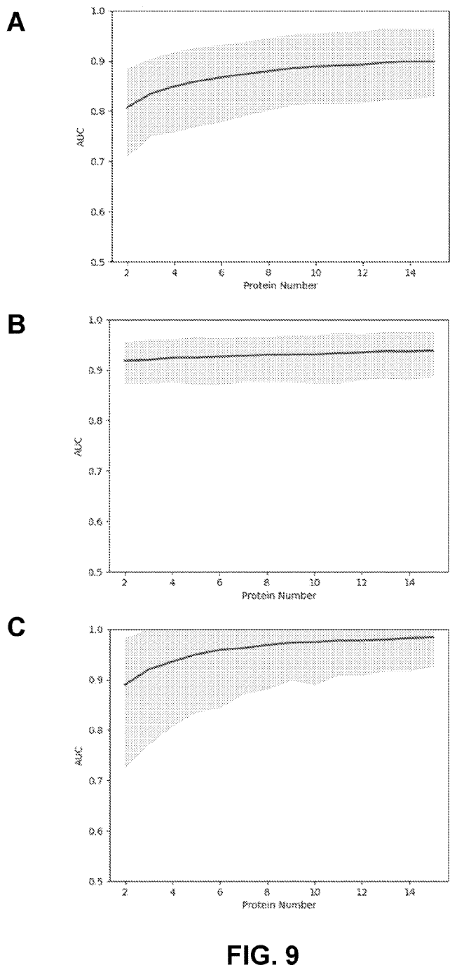

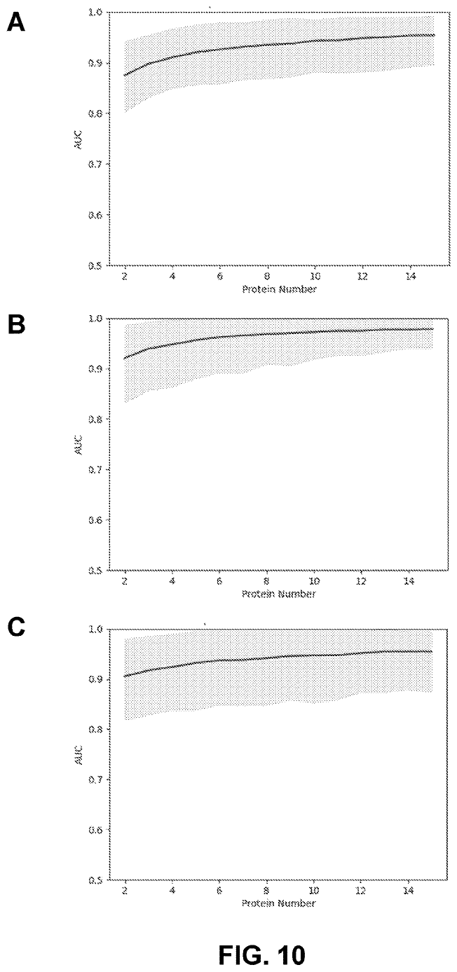

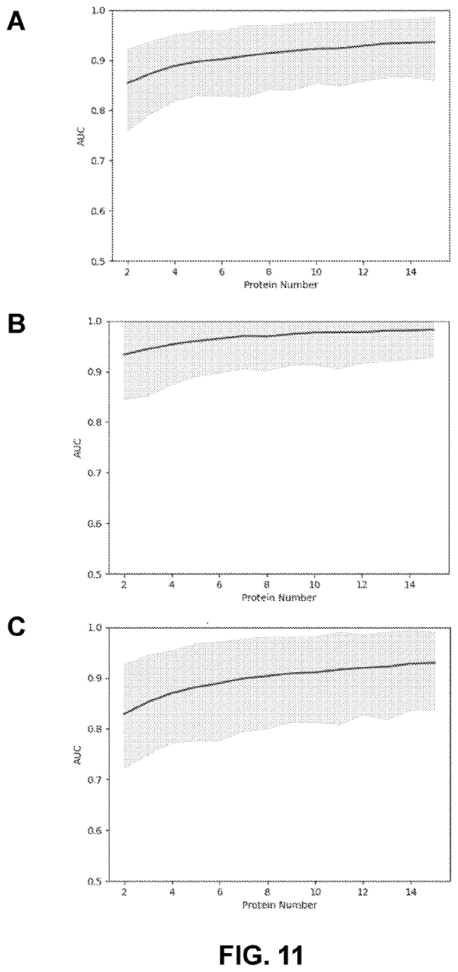

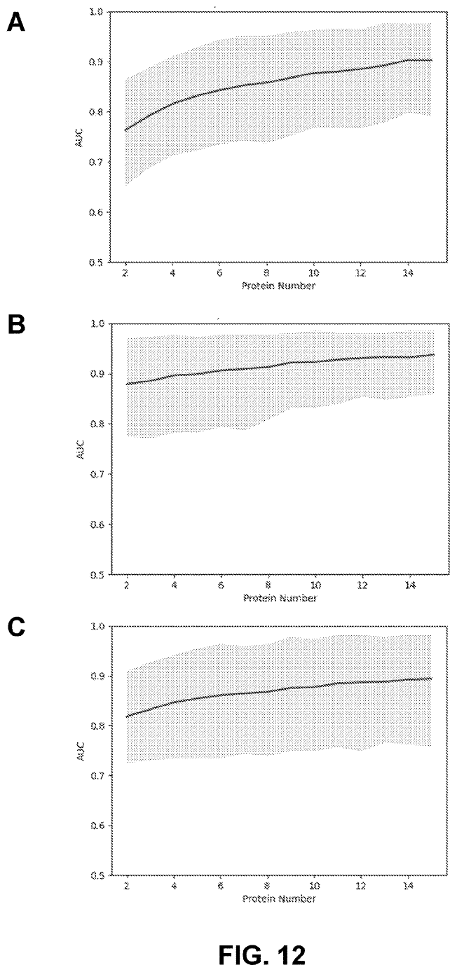

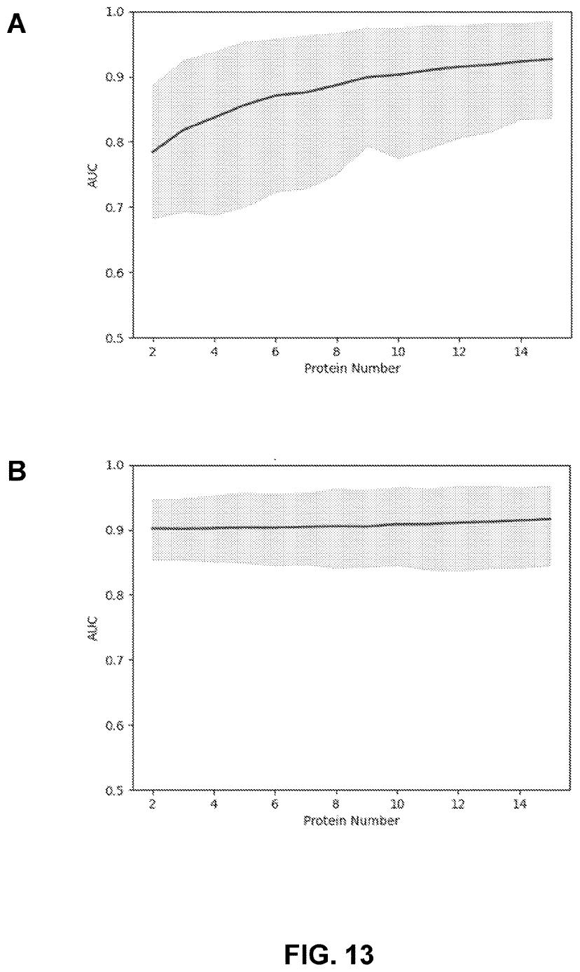

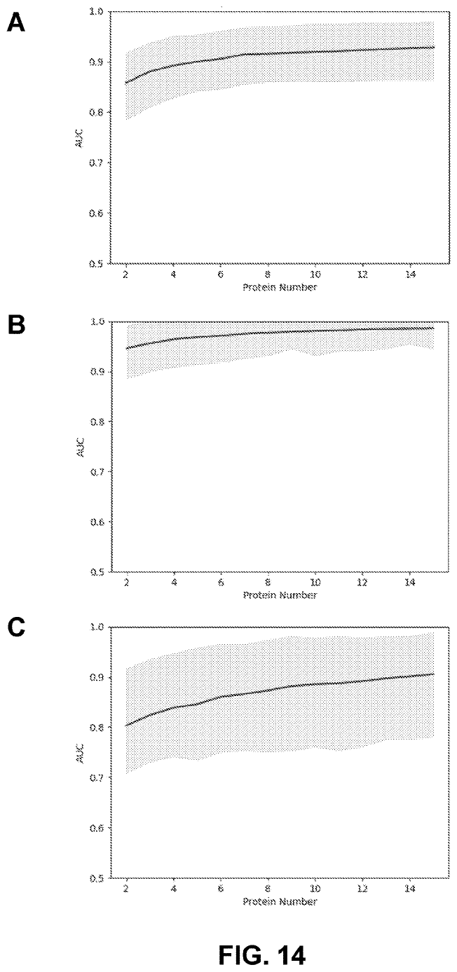

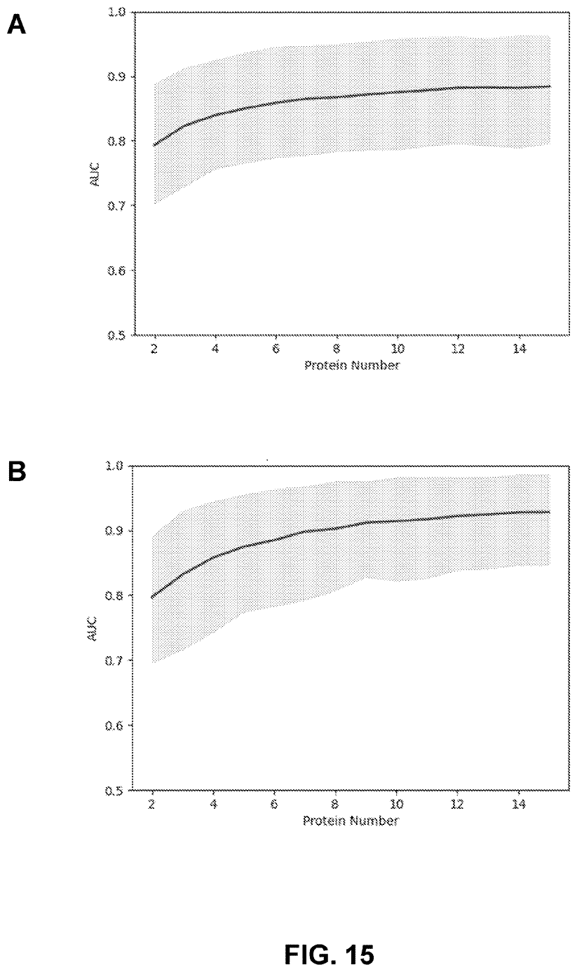

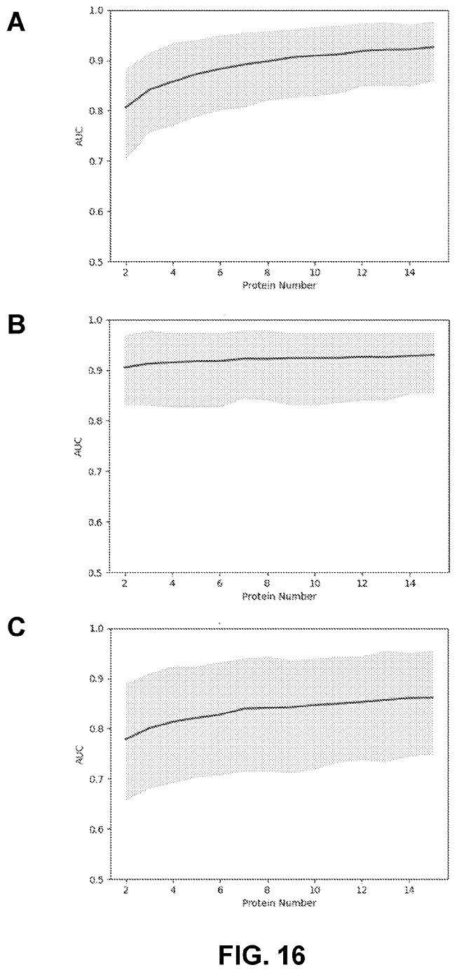

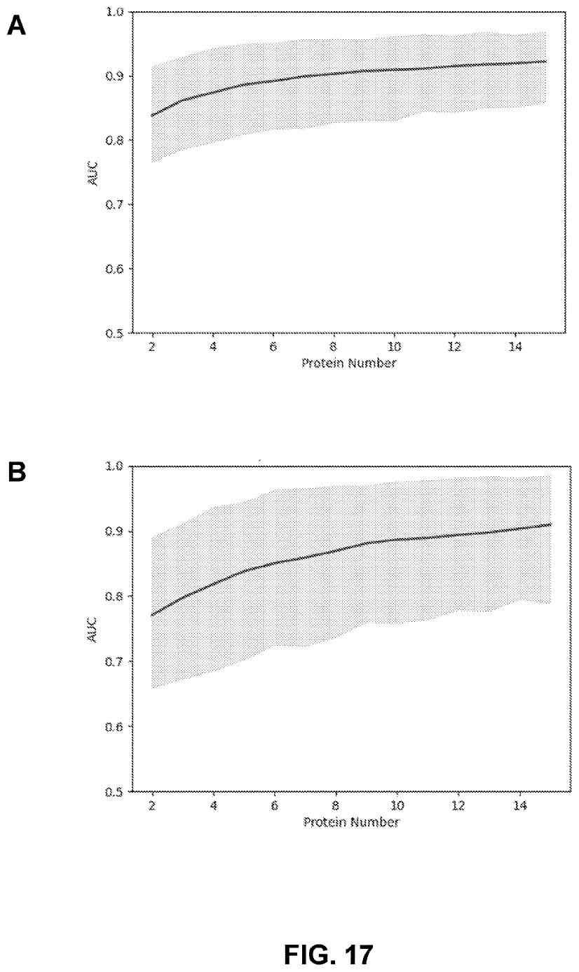

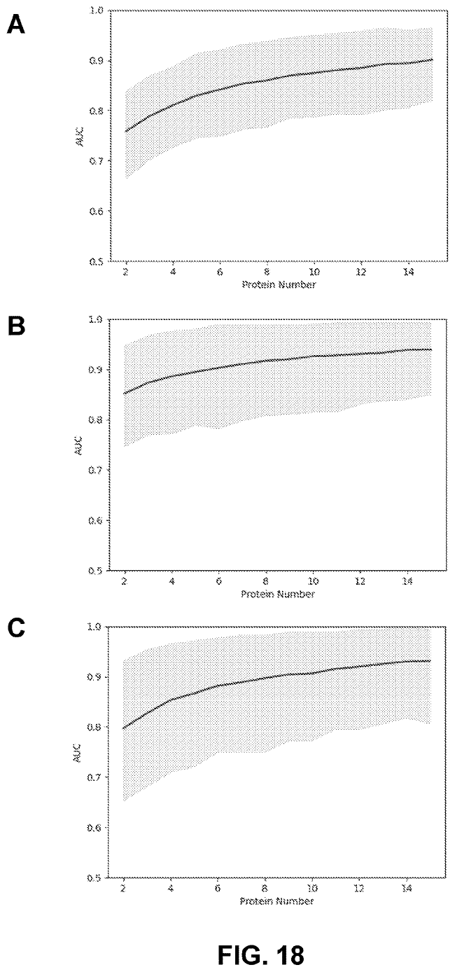

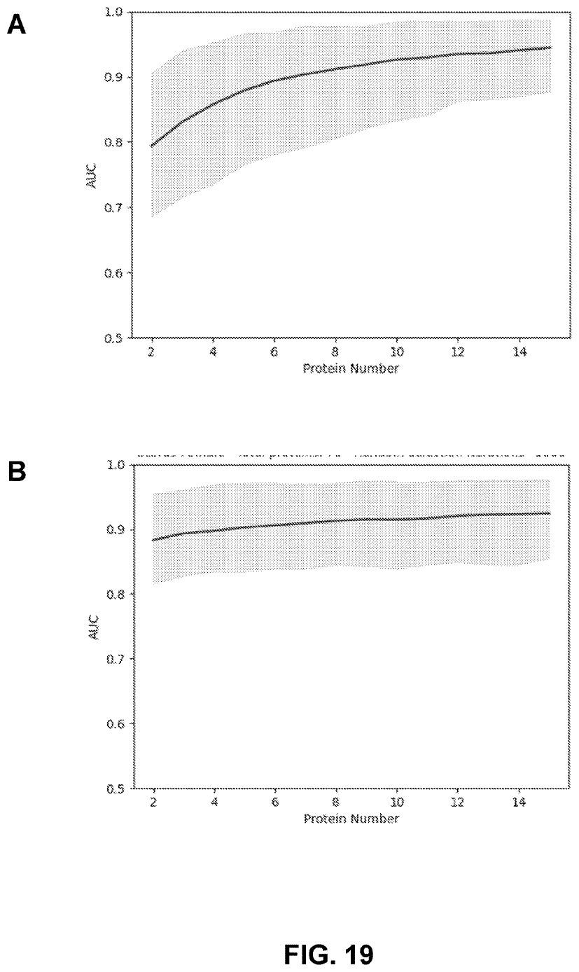

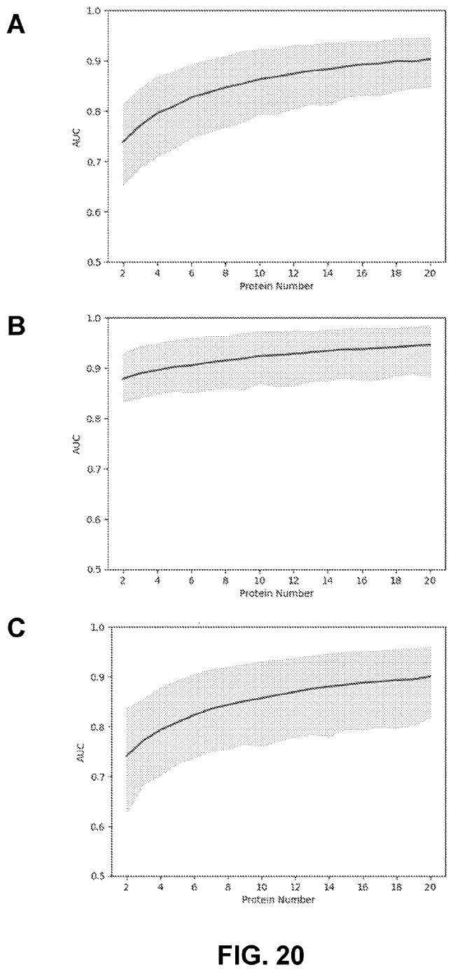

One or more features were fed into one or more computation models. The classifiers were used to calculate a score for the patient. The scores of different classifiers were combined to identify the patient as having specific cancer or not. The computational model only selected protein or protein combinations that could generate a receiver operating characteristic (ROC) curve of greater than or equal to 0.6. The resulting tissue-specific cancer detection proteins are shown in Tables 301-318, corresponding to detection proteins of cancer associated with the bladder, bone, brain, breast, cervix, colon, esophagus, kidney, liver, lung—small cell, lung—non-small cell, ovary, pancreas, prostate, stomach, testis, thyroid, and uterine, respectively. Tables 301-318 also provide individual AUC values for each of the proteins, indicating the accuracy at which each protein can detect the corresponding cancer that is associated with a tissue. FIGS. 2-19 shows that the accuracy is over 0.6 when any two proteins through any 20 proteins are randomly selected.

The tissue-specific cancer detection proteins were aggregated to create the pan-cancer detection proteins of Table 101 that can be used in a pan-cancer detection analysis to detect whether a subject has cancer. Table 101 also provides individual AUC values for each of the proteins, indicating the accuracy at which each protein can detect whether cancer is present in the subject. That any combination of two or more proteins from these tables can detect whether the subject has cancer is demonstrated in FIG. 20, which shows that the accuracy is over 0.6 when any two proteins through any 20 proteins are randomly selected.

### Cancer Localization

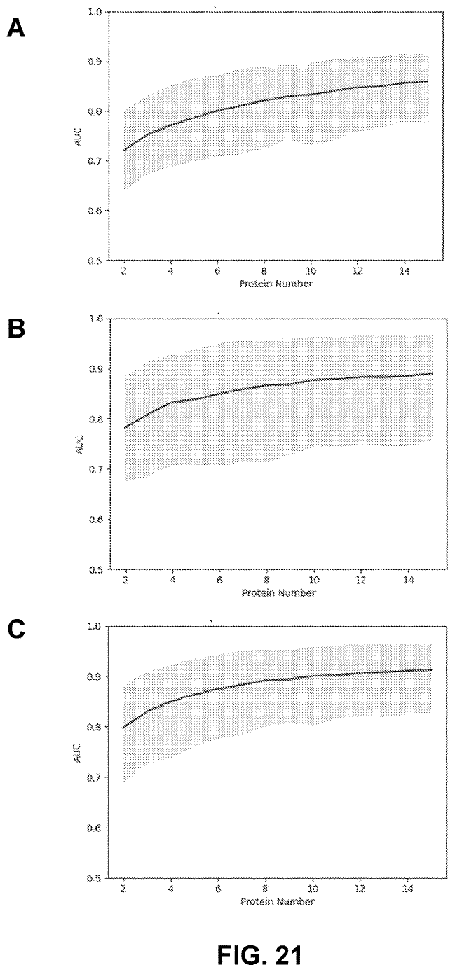

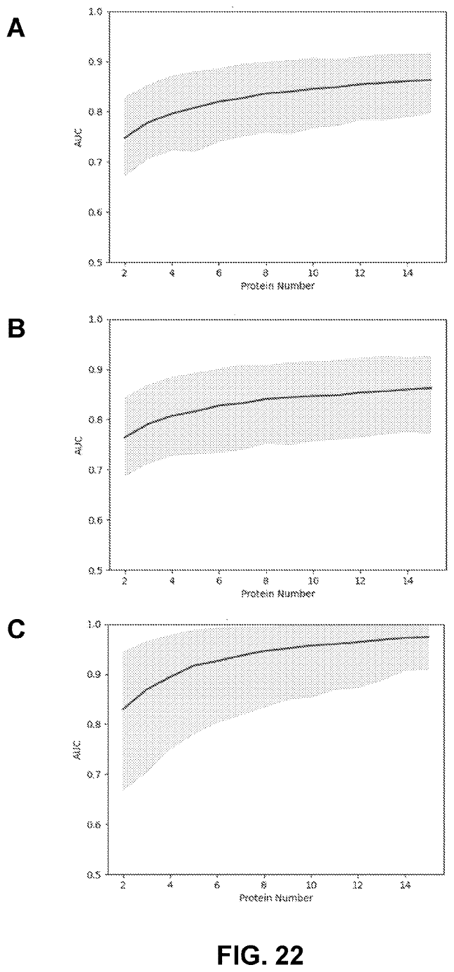

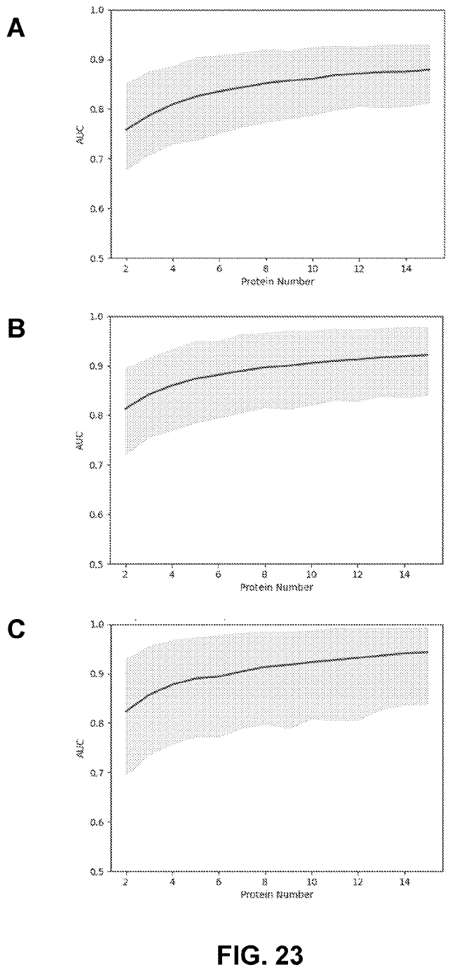

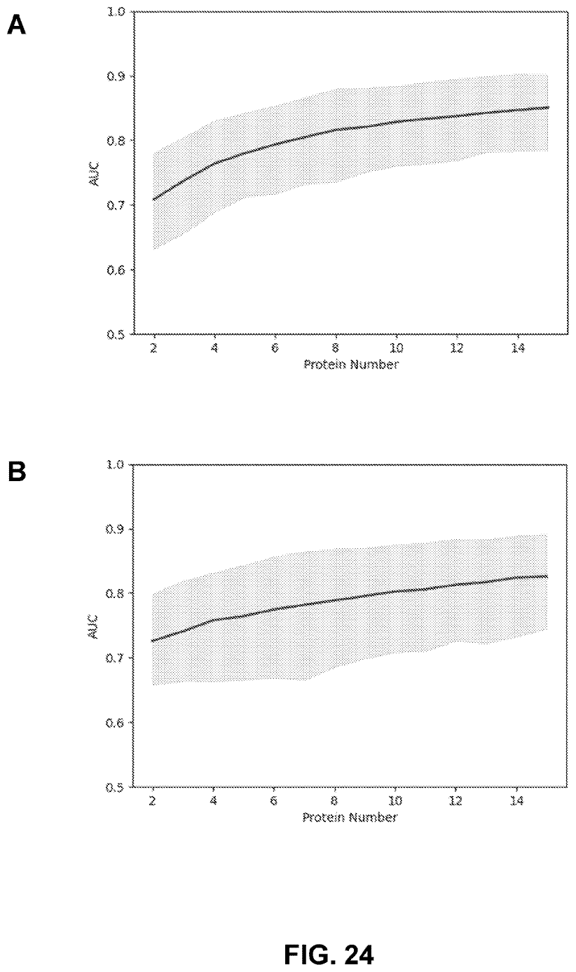

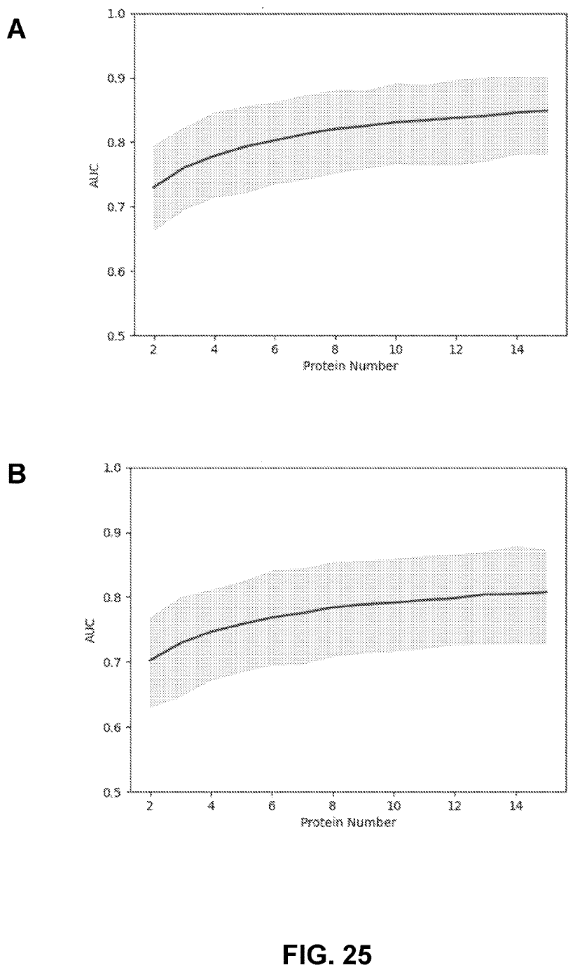

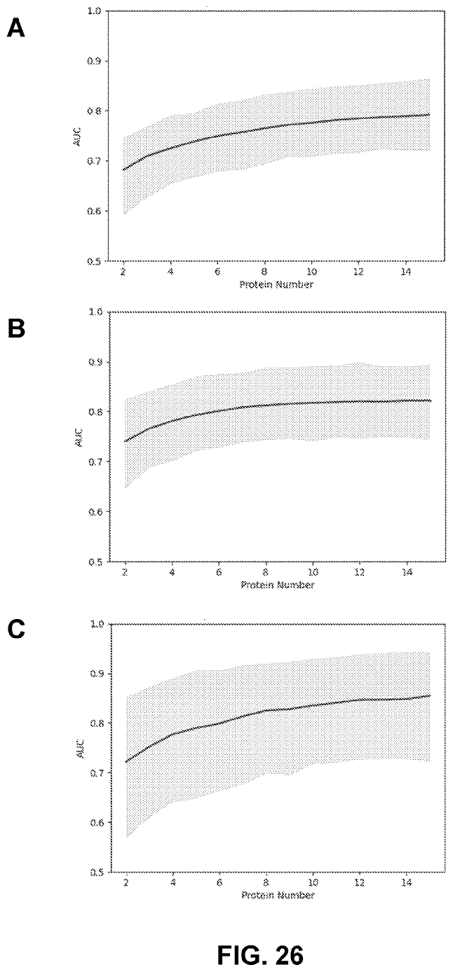

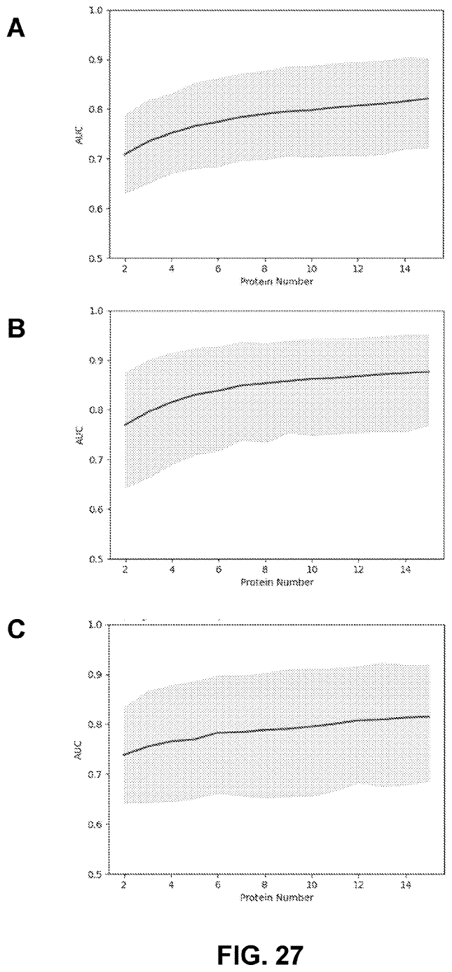

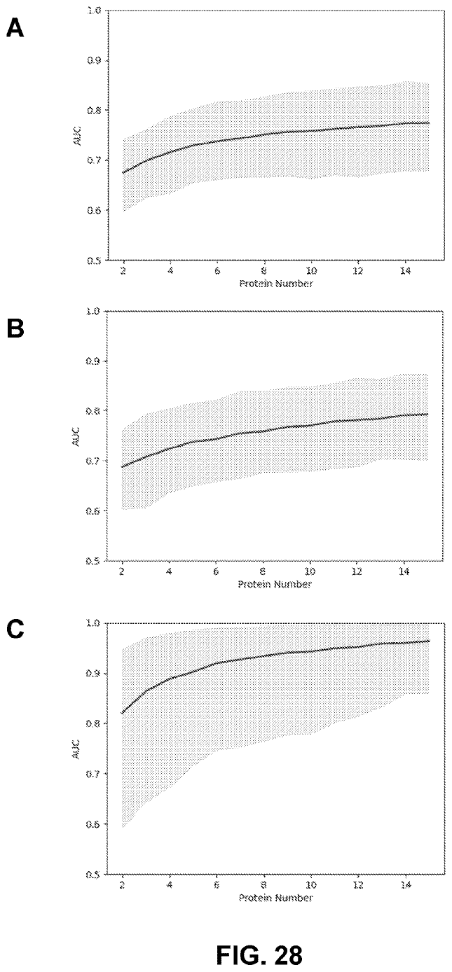

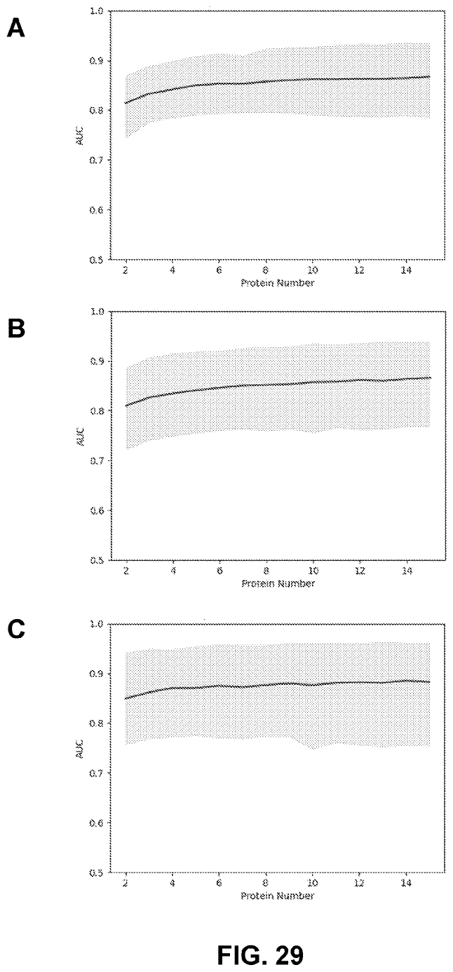

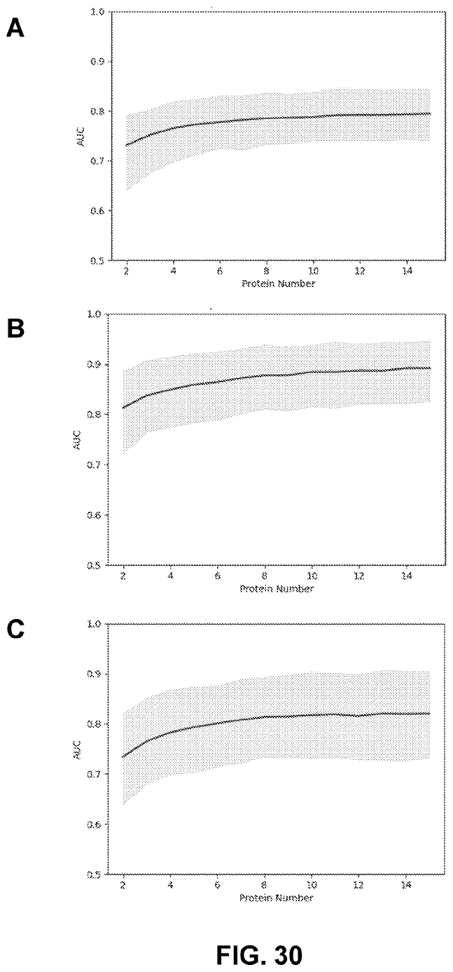

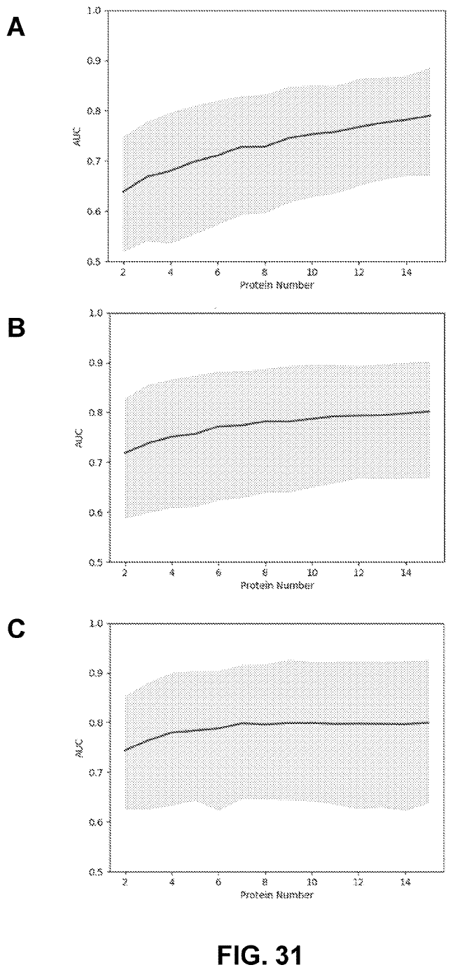

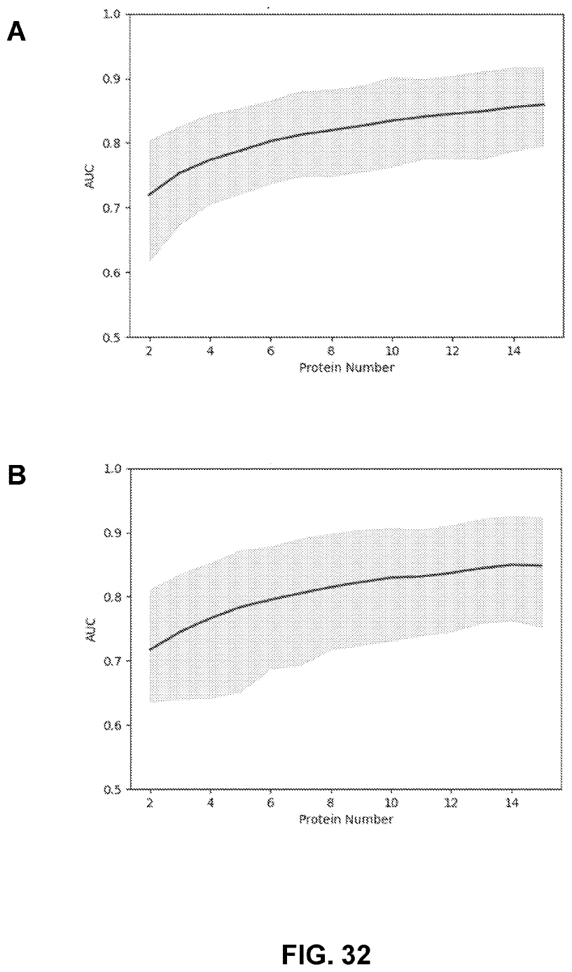

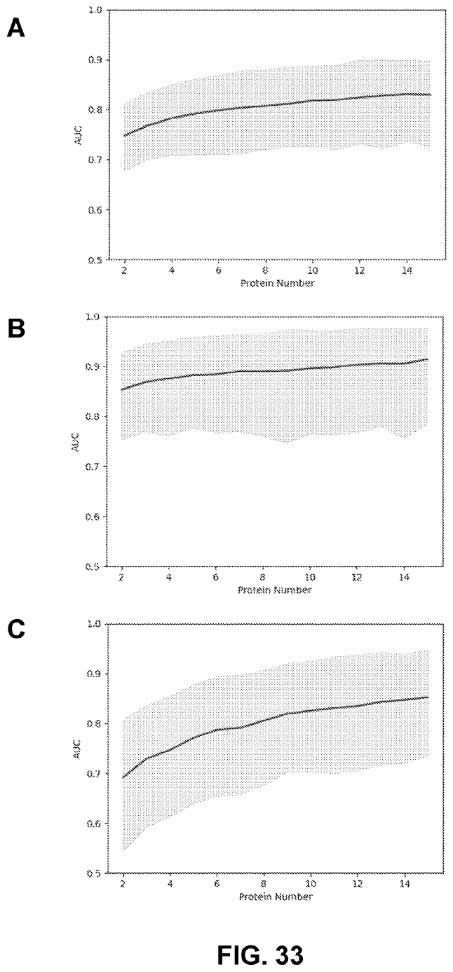

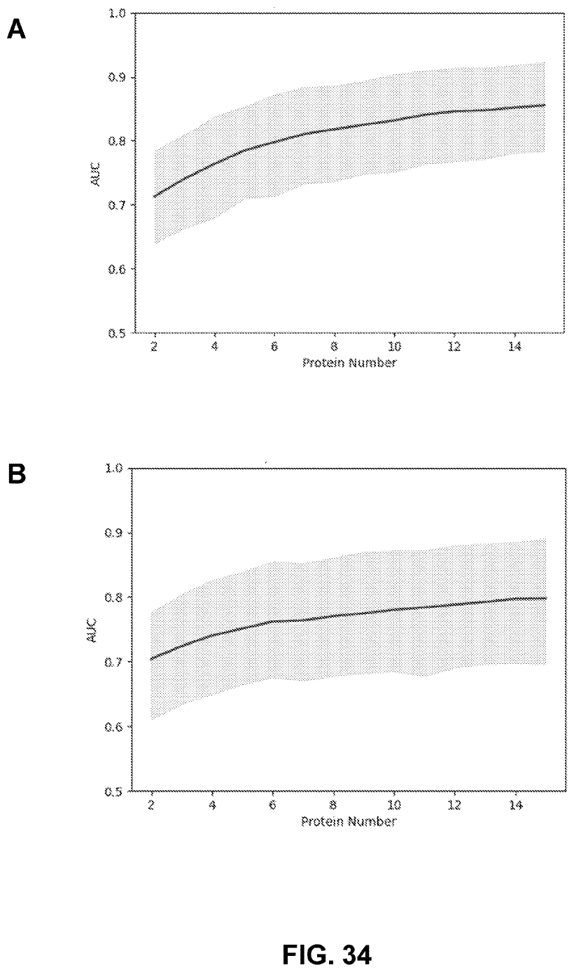

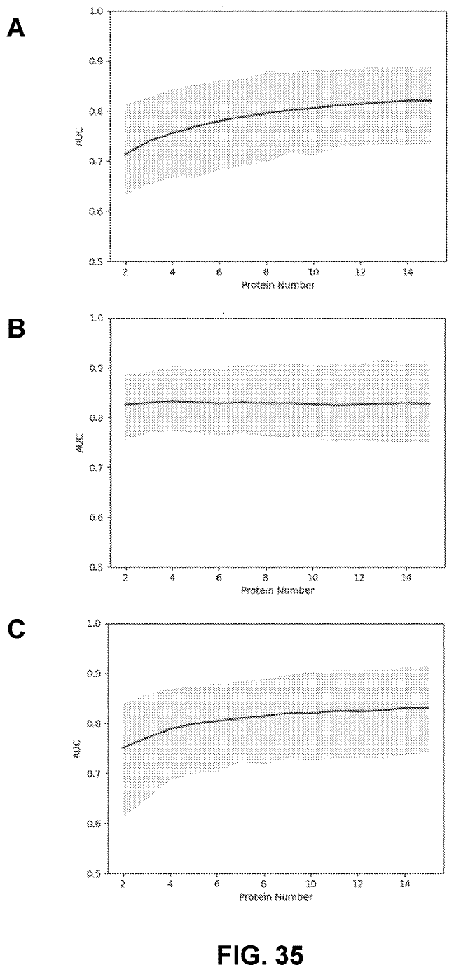

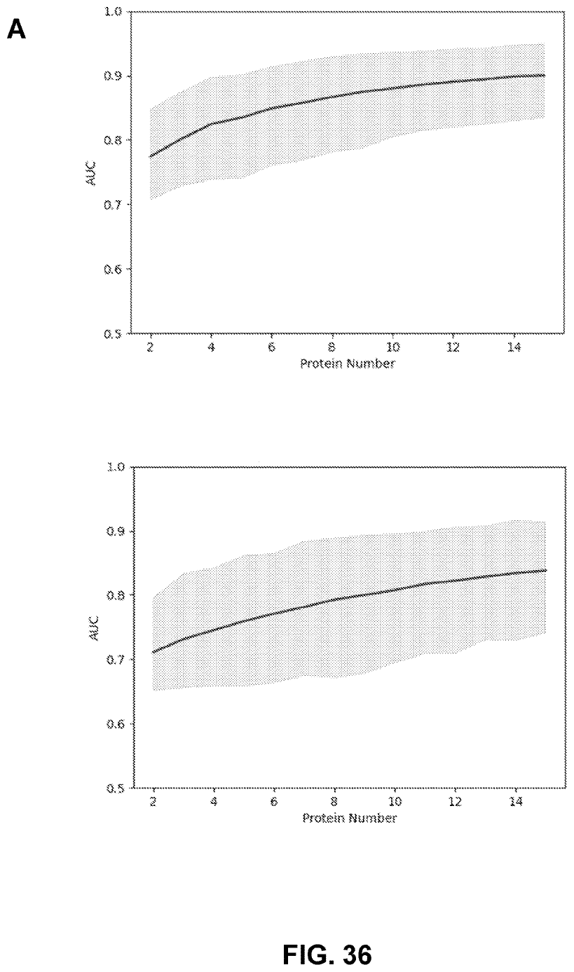

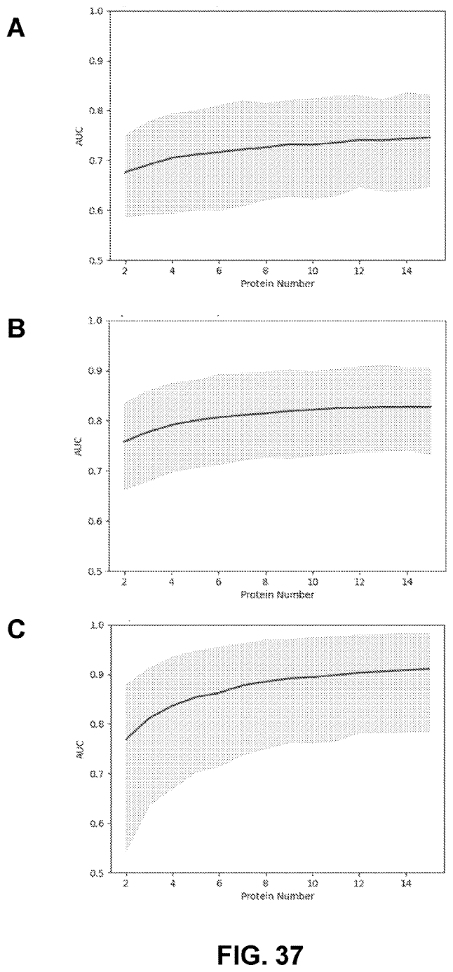

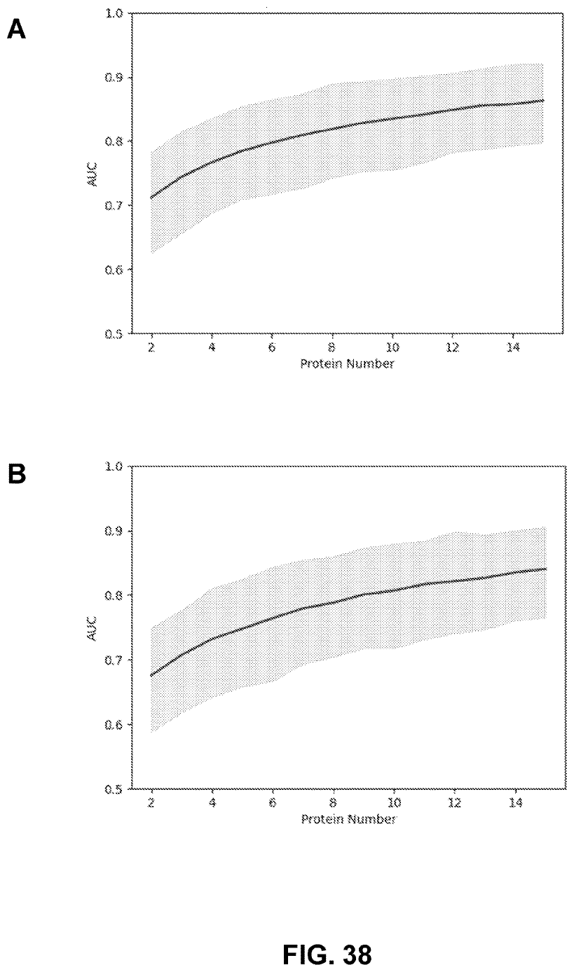

One or more features were fed into one or more computation models. The features could be similar or different from what was used in determining cancer versus non-cancer status. The classifiers were used to calculate a score for the patient for every possible cancer type, including cancers of the bladder, bone, brain, breast, cervix, colon, esophagus, kidney, liver, lung—small cell, lung—non-small cell, ovary, pancreas, prostate, stomach, testis, thyroid, and uterine. The computational model only selected protein or protein combinations that could generate a receiver operating characteristic (ROC) curve of greater than or equal to 0.6. The scores are combined to discriminate the cancer type. The resulting tissue-specific cancer localization proteins are shown in Tables 201-218, corresponding to detection proteins of cancer associated with bladder, bone, brain, breast, cervix, colon, esophagus, kidney, liver, lung—small cell, lung—non-small cell, ovary, pancreas, prostate, stomach, testis, thyroid, and uterine, respectively. Tables 201-218 also provide individual AUC values for each of the proteins, indicating the accuracy at which each protein can detect the corresponding cancer that is associated with a tissue. That any combination of two or more proteins from these tables can detect whether the subject has the specific cancer is demonstrated in FIGS. 21-38, which shows that the accuracy is over 0.6 when any two proteins through any 20 proteins are randomly selected.

### Example 2

Analyses were performed to identify pan-cancer proteins and tissue-specific cancer proteins for detection of early stages of cancer growth.

### Methods

Plasma samples from a total of 440 individuals, either healthy or diagnosed with one of 18 types of early-stage solid tumors, i.e., cancers of the bladder, bone, brain, breast, cervix, colon, esophagus, kidney, liver, lung—small cell, lung—non-small cell, ovary, pancreas, prostate, stomach, testis, thyroid, and uterine. Deep proteomics profiling of plasma samples was conducted using Proximity Extension Assay technology and measured nearly 3,000 proteins in each sample. Then, cancer protein signatures for cancer detection were identified using two main steps: (1) searching for a limited number of proteins that could identify any cancer in its early stages (stages I and II), and (2) classifying each type of cancer against the others to find a cancer-specific signature for localization (i.e., tissue of origin).

All proteins were evaluated to test the cancer localization protein signature for each cancer versus other cancers in the dataset. Localization analysis was done for male and female samples separately. A bootstrap method combined with LASSO regression was performed over the whole data set to identify the proteins with stronger cancer association and best statistical significance. Then, a k-fold train/test validation was performed to evaluate the performance of trained selected features on the test dataset. The top 20% of cancer specific protein pairs with the strongest associations were selected, which resulted in different best sets for the cancers with the highest overall average performance.

A probability score was calculated at each step to classify a person's protein array sample. A multi-step approach was used to define a minimal set of proteins (also known as the “best set”) that could classify samples from a stable model and be generalizable. The dataset was split into a training set and a test set to evaluate the performance of intermediate models and guide protein selection, optimizing the model parameters and final fit. Machine learning penalized regression and resampling methods were used to manage sample uncertainty and calculate different scoring methods to choose proteins that were not only associated with cancer detection but also contributed to the model performance in the presence of other proteins. The predicted probability for each sample was calculated using the best-set proteins at each step and this probability was applied to each cancer to classify each sample.

### Results

Table 1 shows the characteristics of the samples included in the study. All patients diagnosed with cancer were treatment naive.

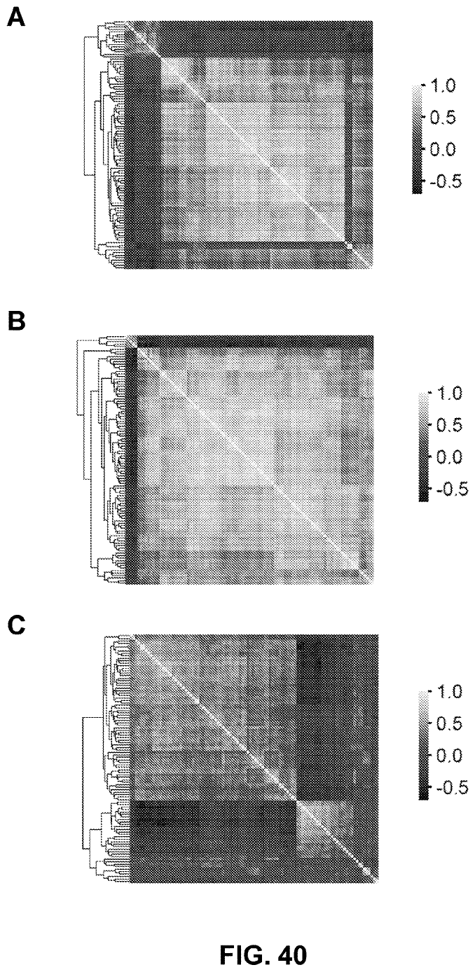

Of the total number of 3072 proteins analyzed, 280 were not included for further analysis due to the quality measurements warnings. Protein abundances showed different correlation levels. The correlation matrix shows more protein pairs with a higher positive correlation (see FIG. 40). The correlation is a limiting factor in the analysis.

In further analysis, proteins were selected out of internally correlated sets to be able to classify more patients. The comparison of the protein levels between normal and any cancer samples identified many good candidates to be included in the multi-protein prediction model. The level of significance of difference between cancer and normal samples were significantly different between male and females. The proteins in females provided a greater differentiation and the levels were more different than male proteins.

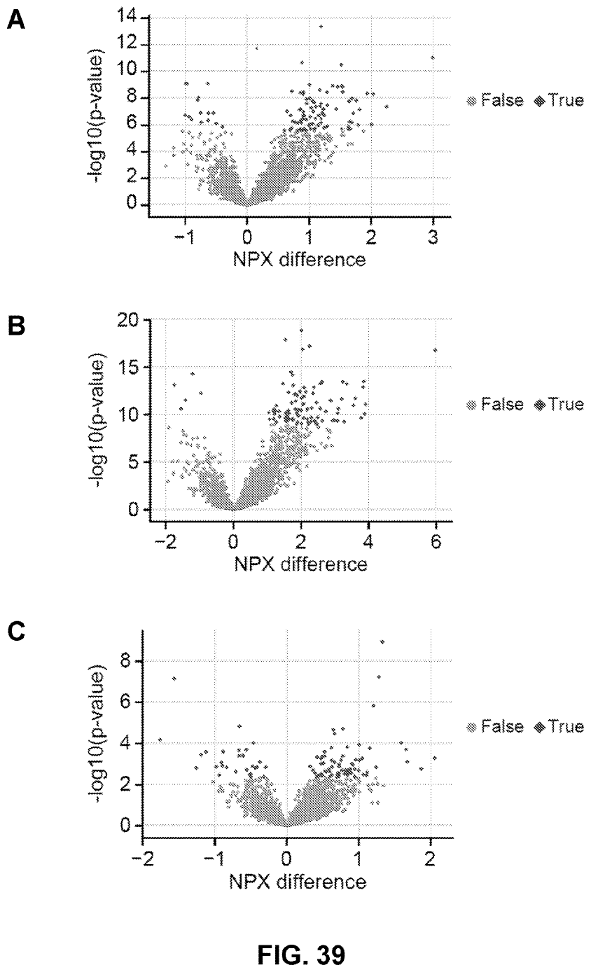

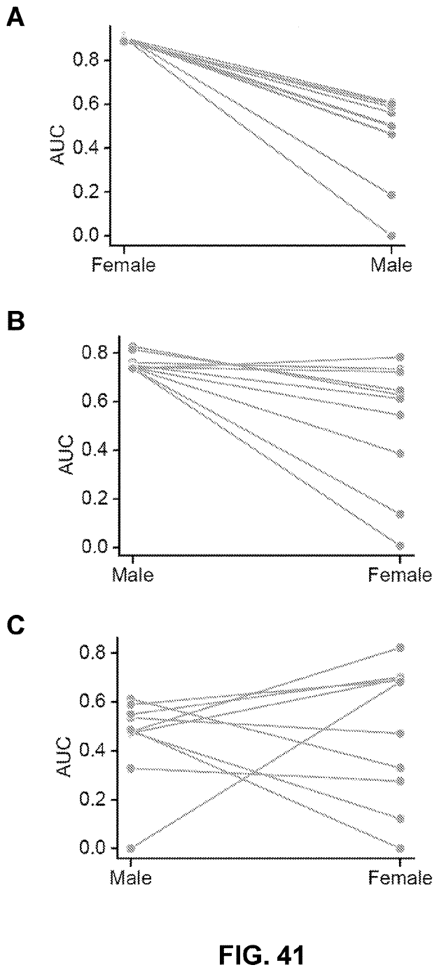

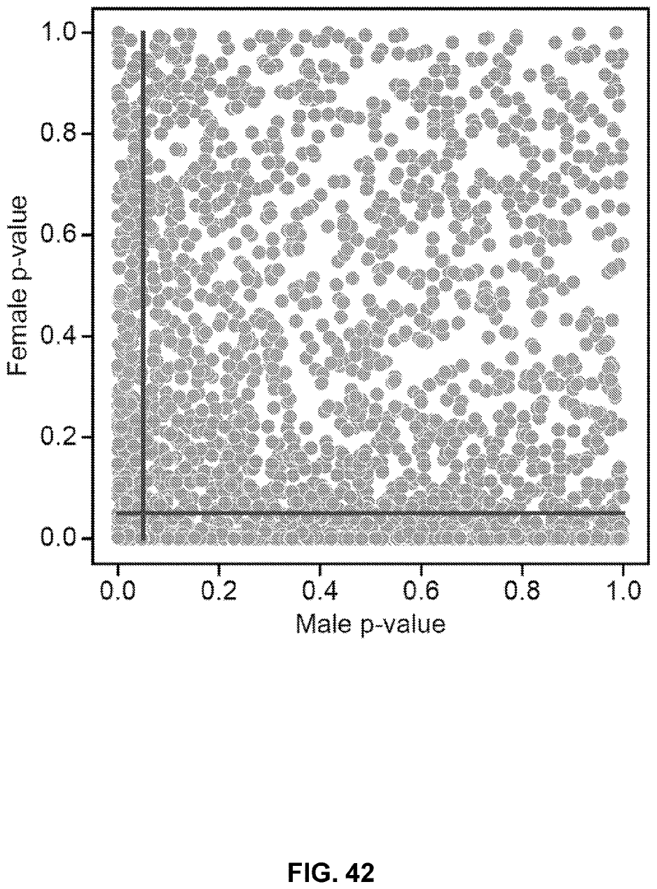

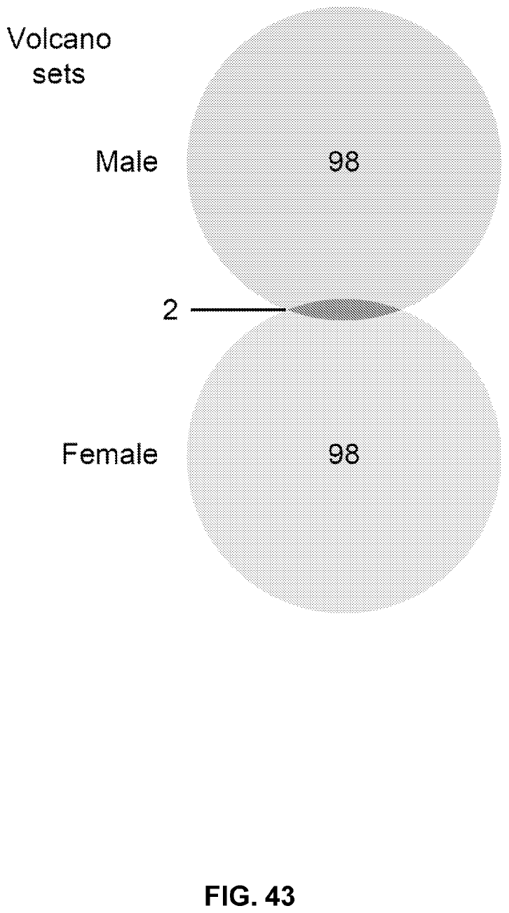

It was found that the proteins-cancer association varied significantly between male and females (see FIGS. 39-43). A simple comparison between cancer and normal samples for men and women showed a poor correlation across protein profiles. Many proteins with a p-value of less than 0.001 in one sex showed no significant difference in the other one. An analysis of volcano plots showed minimal overlap between 100 proteins selected based on their differential expression and their p-value. An analysis of the top ten proteins using AUC showed the order of importance of the proteins varied between males and females. The ten proteins with the best AUC to differentiate cancer from normal in male samples perform poorly in females and vice versa. Thus, the remainder of the analyses on male and female samples were performed separately to capture the sex specific patterns.

Finding the best sex-specific sets to identify a cancer was performed in two steps. At the first step, the protein signature of any cancer (pan cancer/any cancer classifier) was detected to classify any cancer from normal. In the second step, the tissue of origin of cancer and cancer subtypes (i.e., small cell and non-small cell cancers of lung, and cervical and endometrial cancers of uterine) was identified.

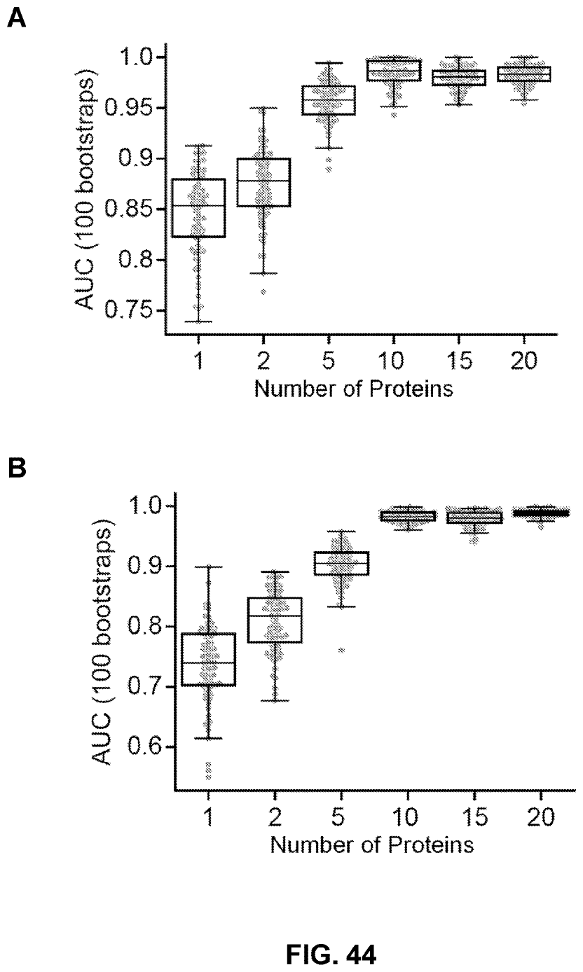

For each step, several numbers of proteins were tested. The model's performance by AUC increased quickly with the addition of more proteins, but after ten proteins in any cancer model, no more improvement in AUC was observed (see FIG. 44, Panels A and B).

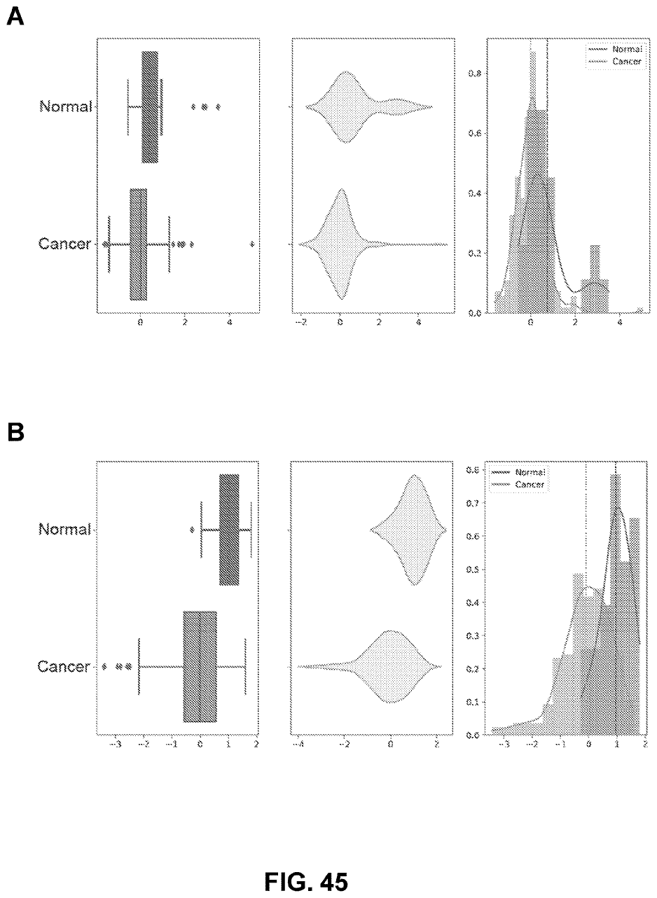

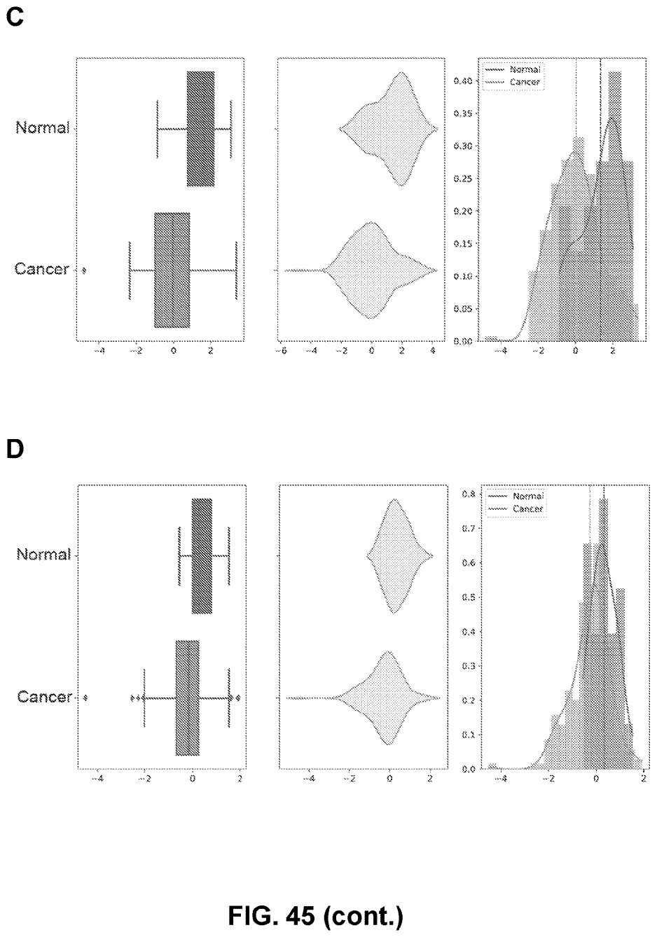

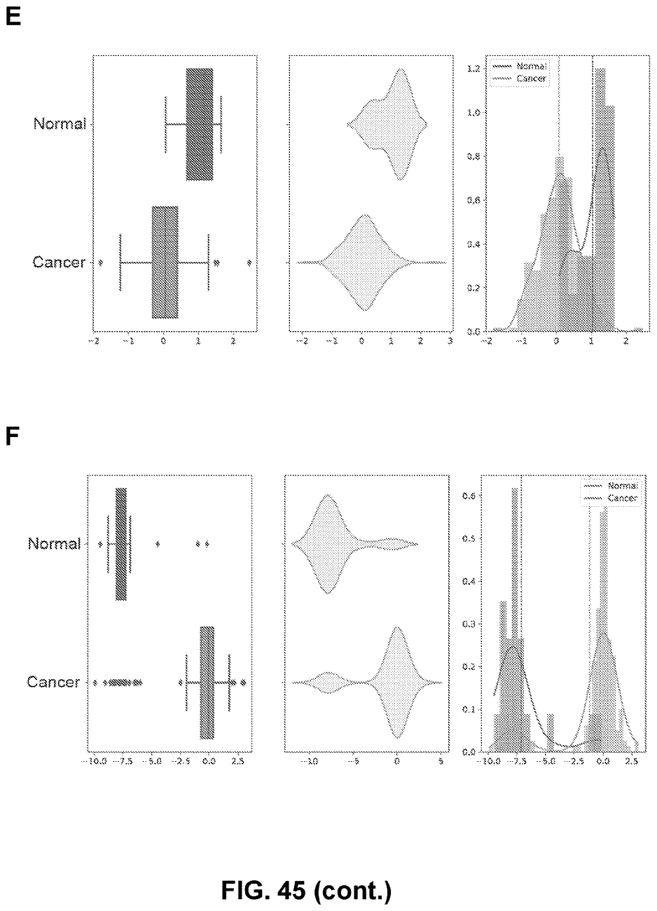

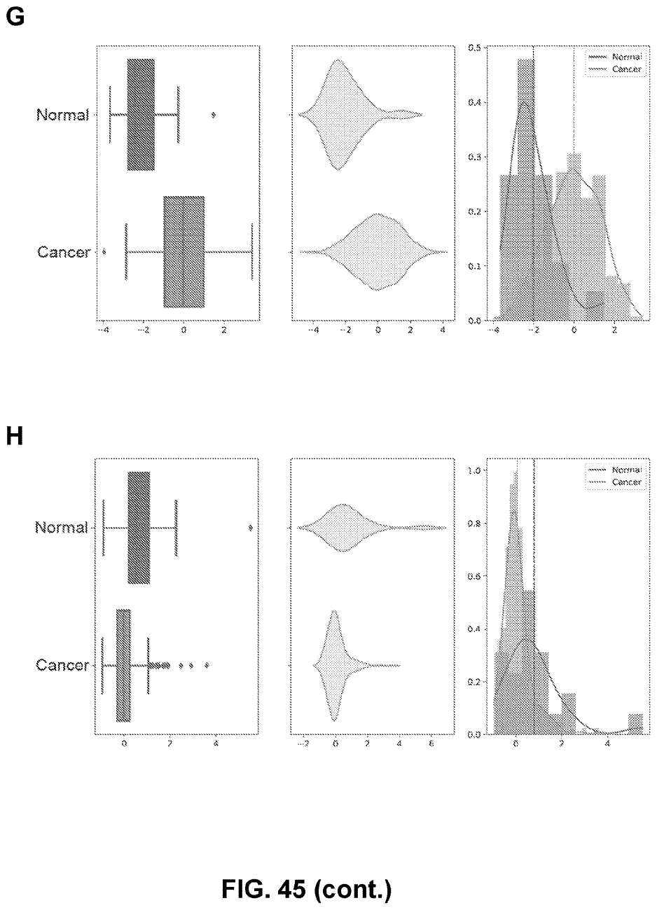

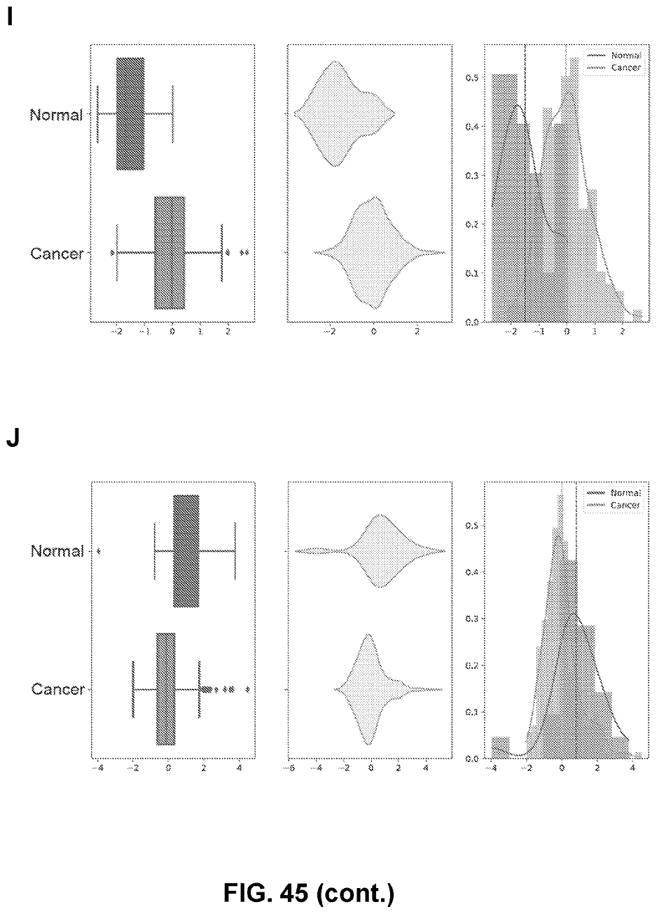

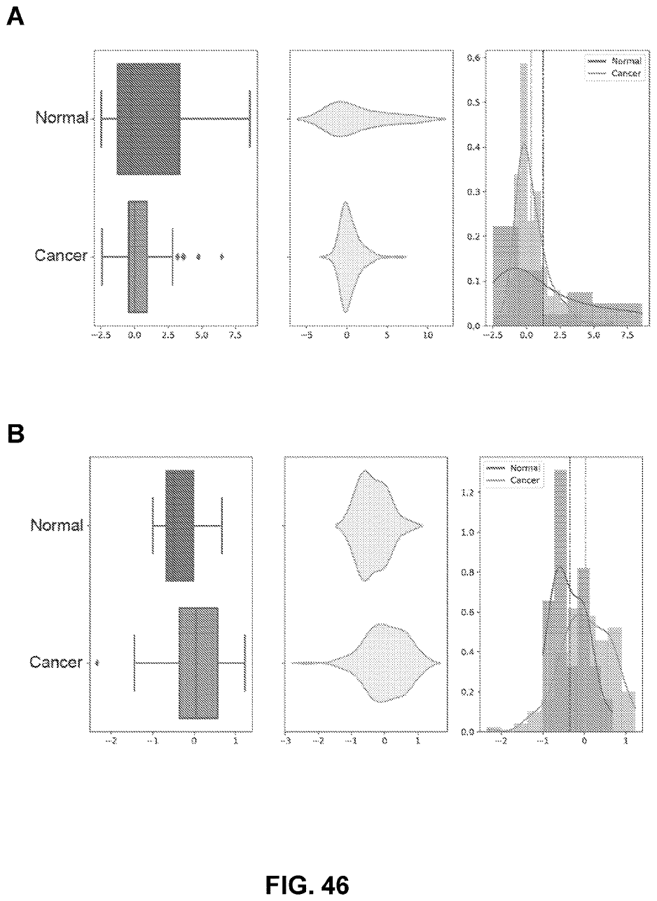

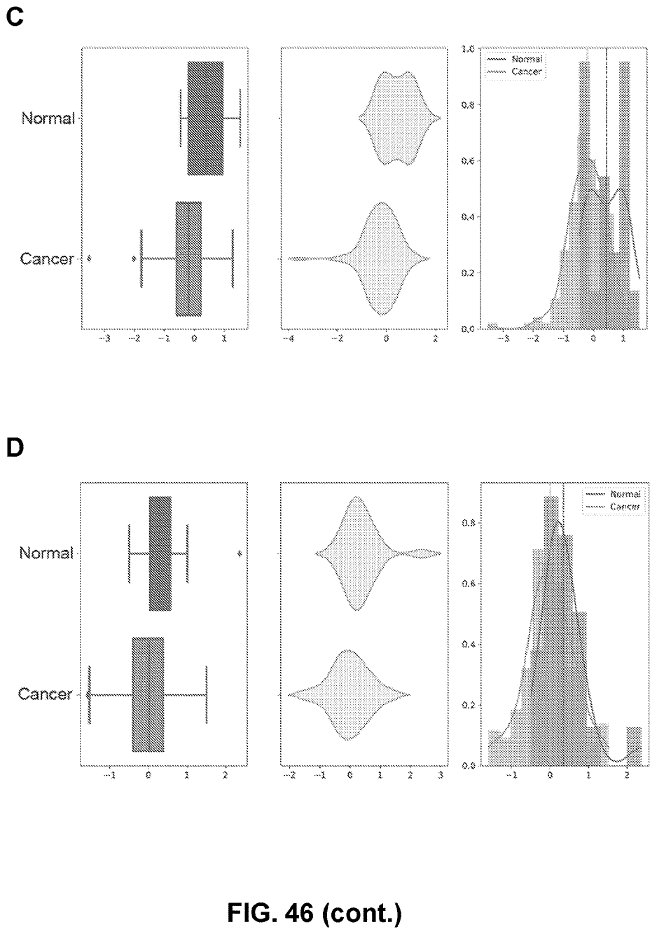

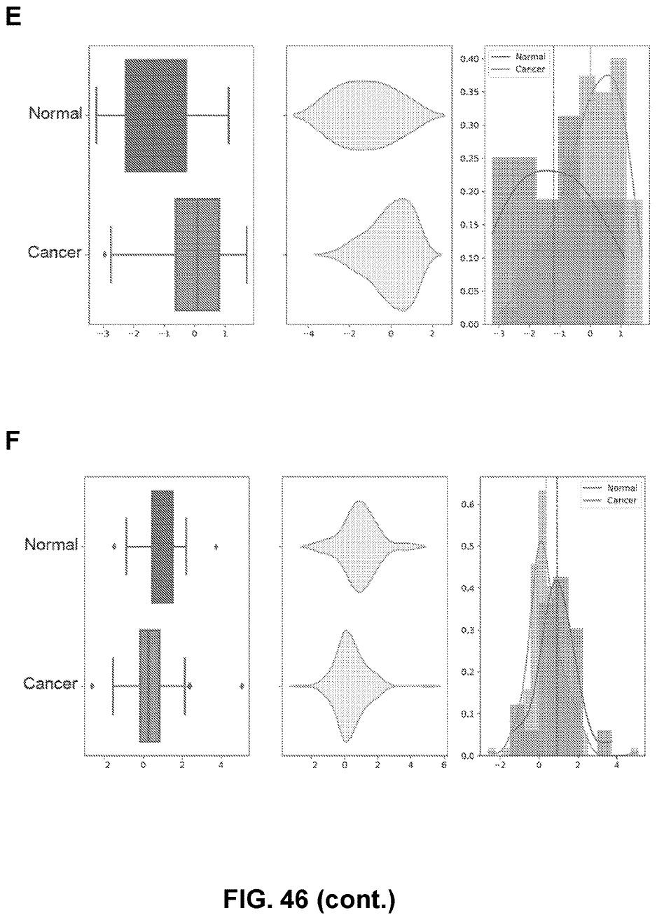

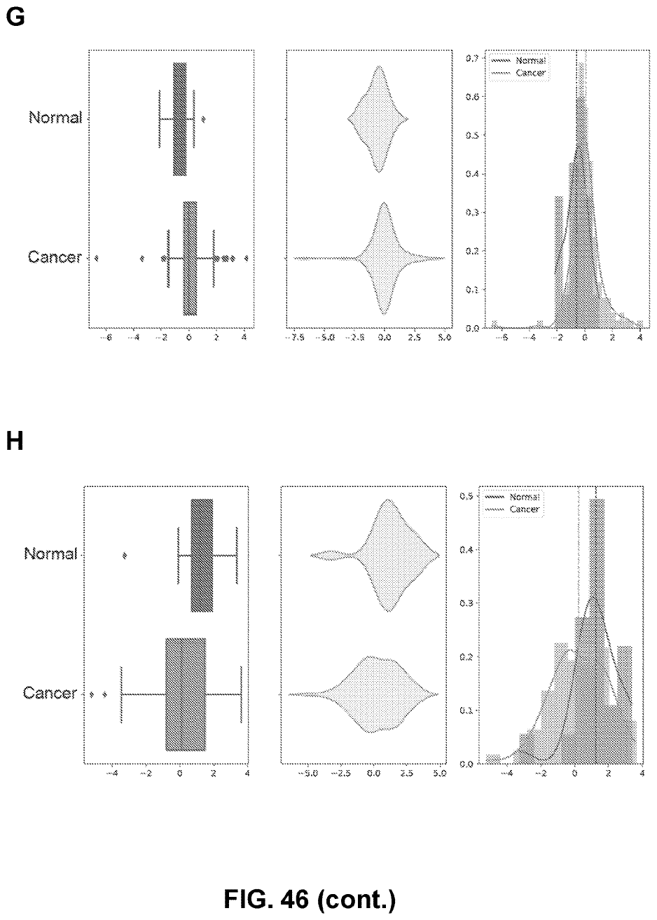

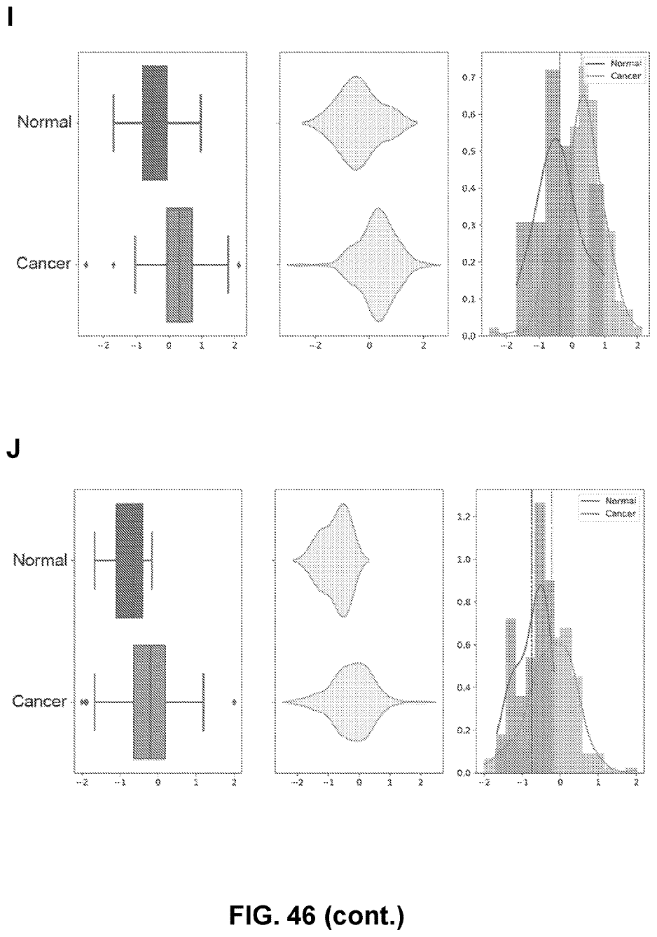

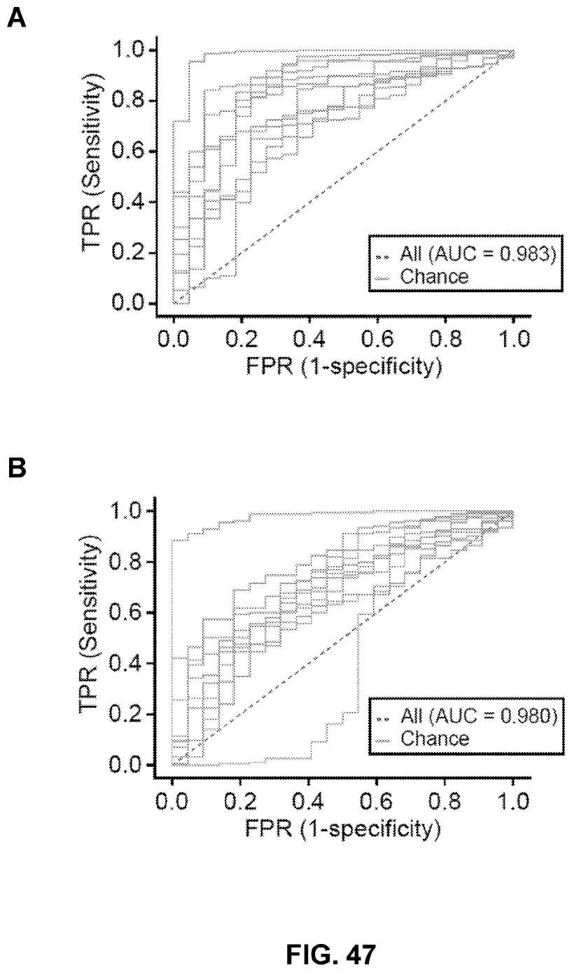

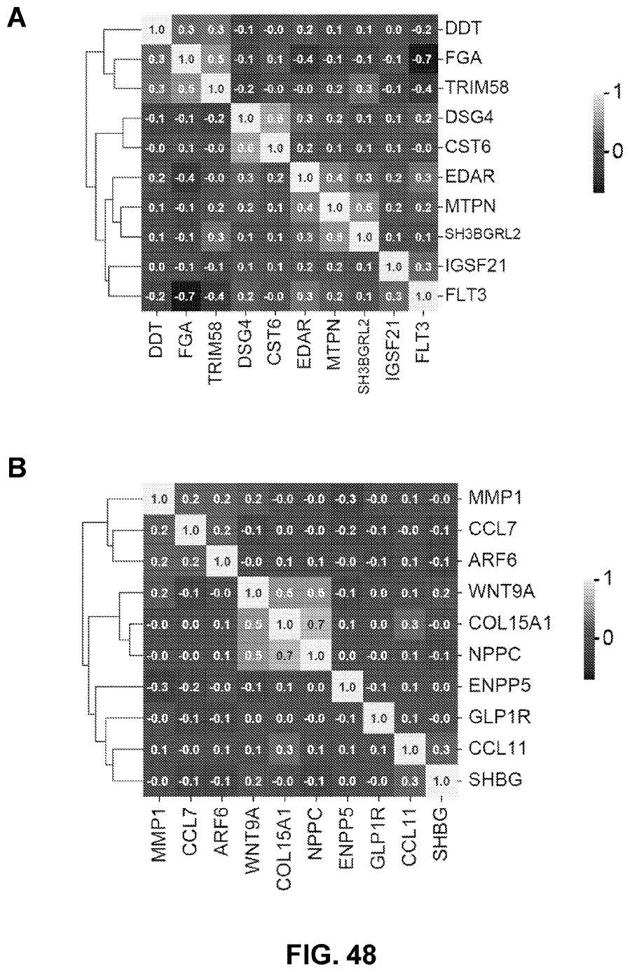

The final pan-cancer detection panels for male and female each consisted of ten proteins that were differentially expressed among normal and cancer plasma samples (see FIGS. 45 and 46: Tables 101.B.4 and 101.C.4). Each protein of the panel alone had a low to medium detection accuracy but when assessed in combination with other proteins as a panel they achieved a very high accuracy in detection of early-stage cancers (see FIGS. 47 and 48). The proteins in the panel showed low to medium correlation indicating each protein contributing new information and presenting a different pathway to the panel.

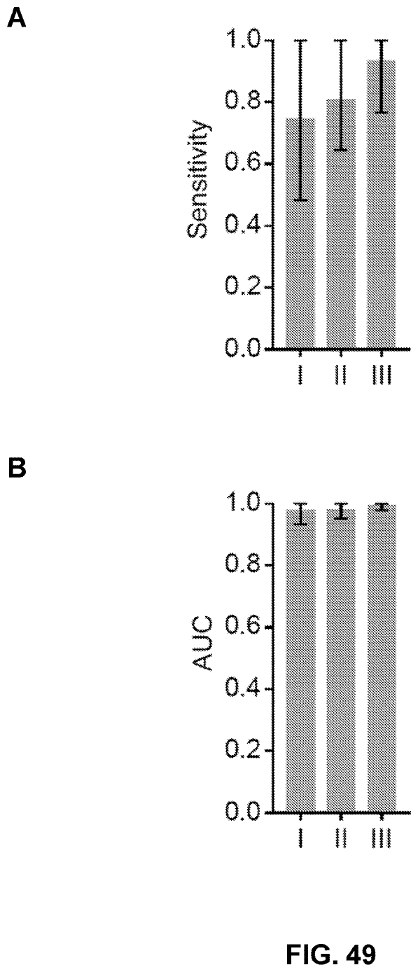

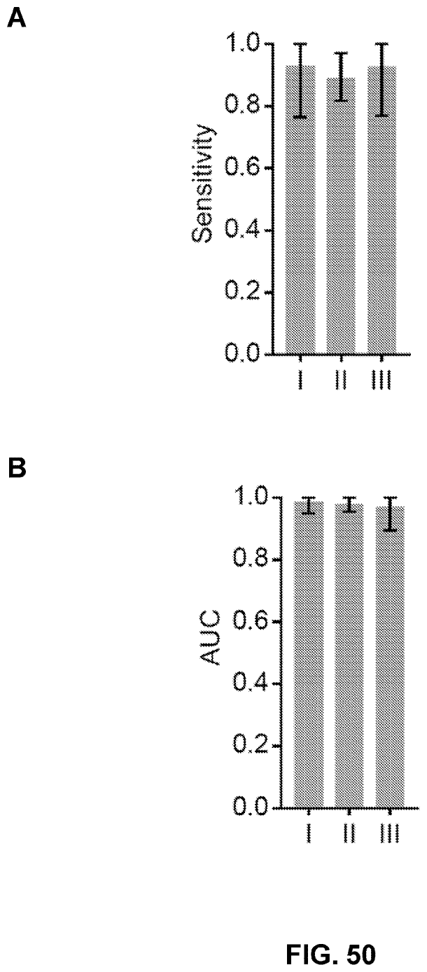

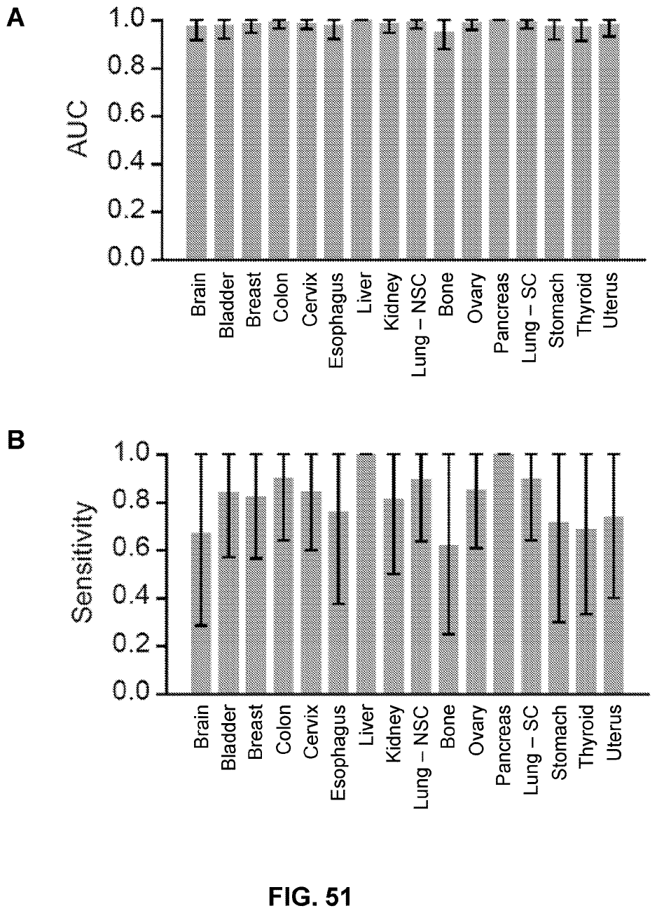

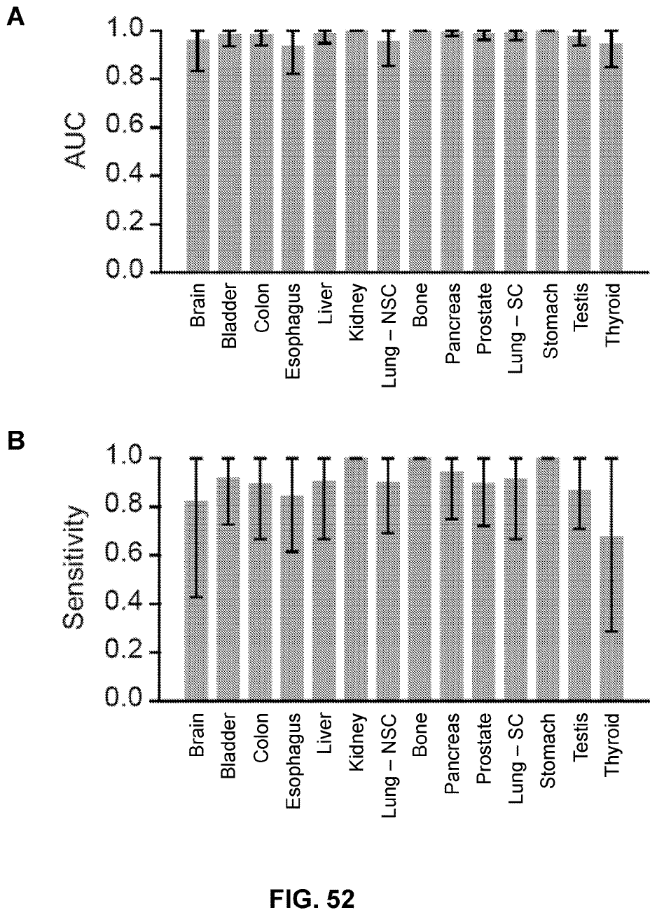

Overall, at the detection step, the protein panels showed high sensitivity and specificity among females and males. At the specificity of 99%, the overall sensitivity of the test was 91% among females and 86% among males (see FIG. 49, Panel A, and FIG. 50, Panel A). High accuracy was observed across all stages of cancer among females and males (see FIG. 49, Panel B, and FIG. 50, Panel B, respectively). At stage I and at the specificity of 99%, the panel was able to identify 87% of cancers among females and 89% of cancers among males. The performance of the panels varied across cancers (see FIGS. 51 and 52). Overall, some cancers (e.g., cancers of the esophagus, liver, kidney, pancreas, and lung—small cell) were easier to detect both among females and males. On the other hand, the detection of cancers like brain or bone was more challenging but achieved with high accuracy after optimization.

To evaluate the performance of the cancer set to detect any cancer, one cancer at a time was excluded and the fitted model was evaluated on the other cancers by the algorithm above on the excluded (unseen) cancer. The analysis showed an acceptable performance with an AUC of, on average, above 0.9 for an unseen cancer. The cancer-out model has better performance on unseen colon and lung cancer but a lower performance to detect unseen brain cancer (astrocytoma) and bone cancer (osteosarcoma) and classify them as “any cancer”. This shows that the protein set correlates very well with the cancer and serves as a cancer detection test.

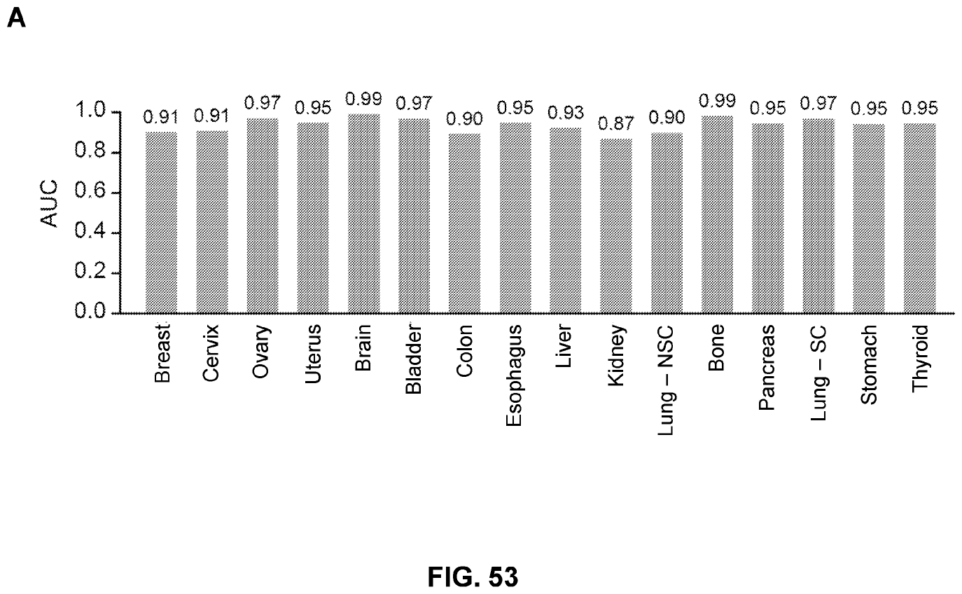

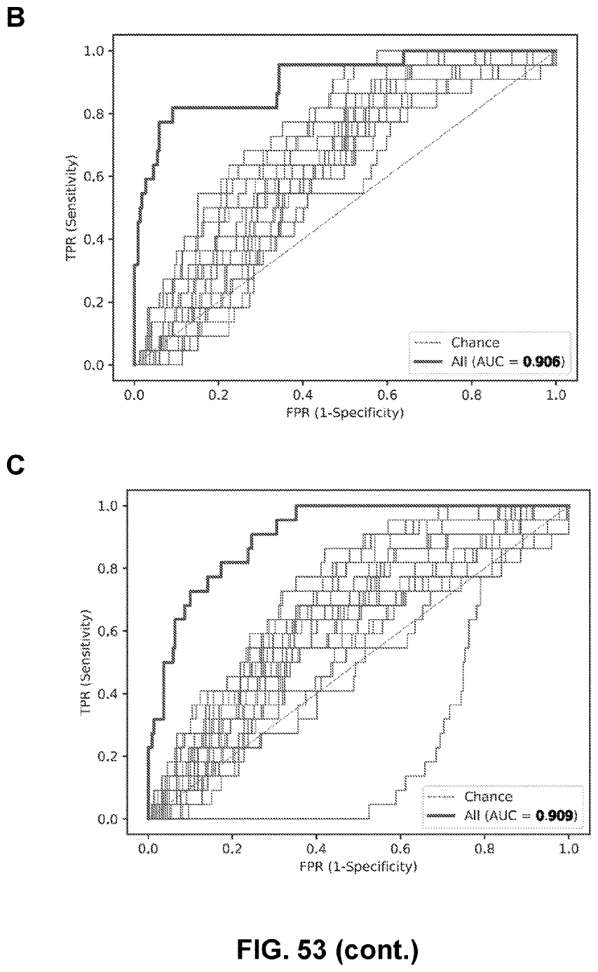

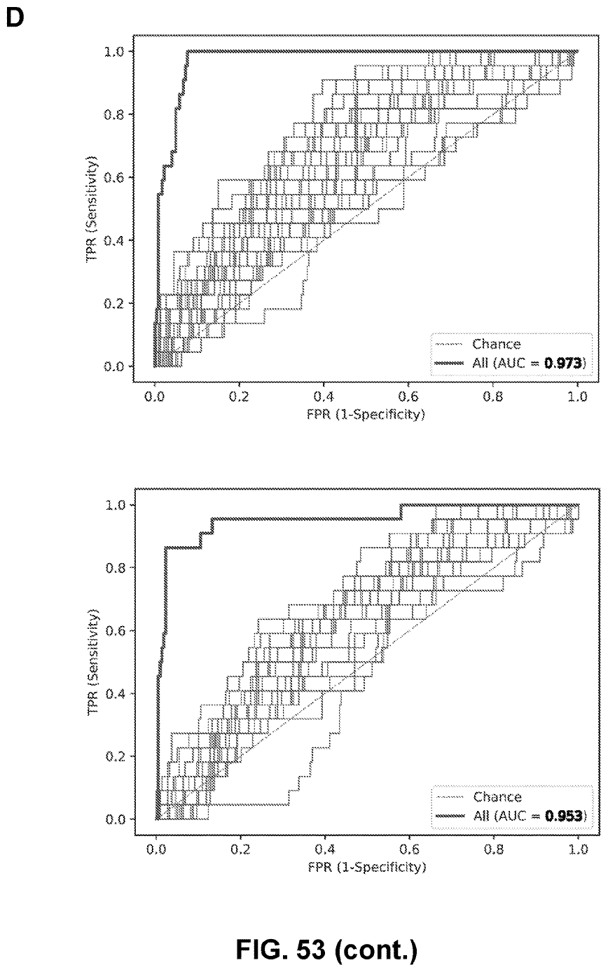

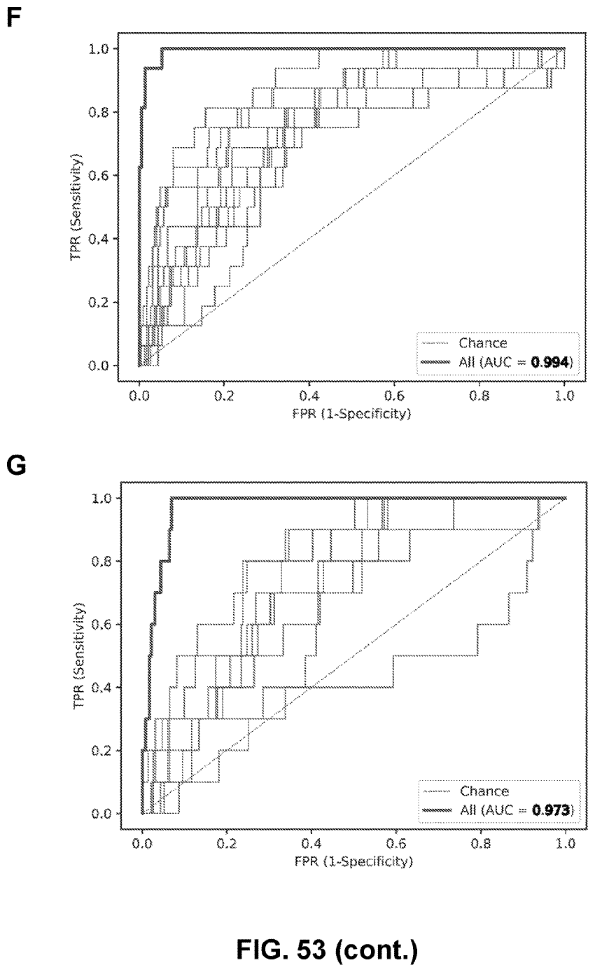

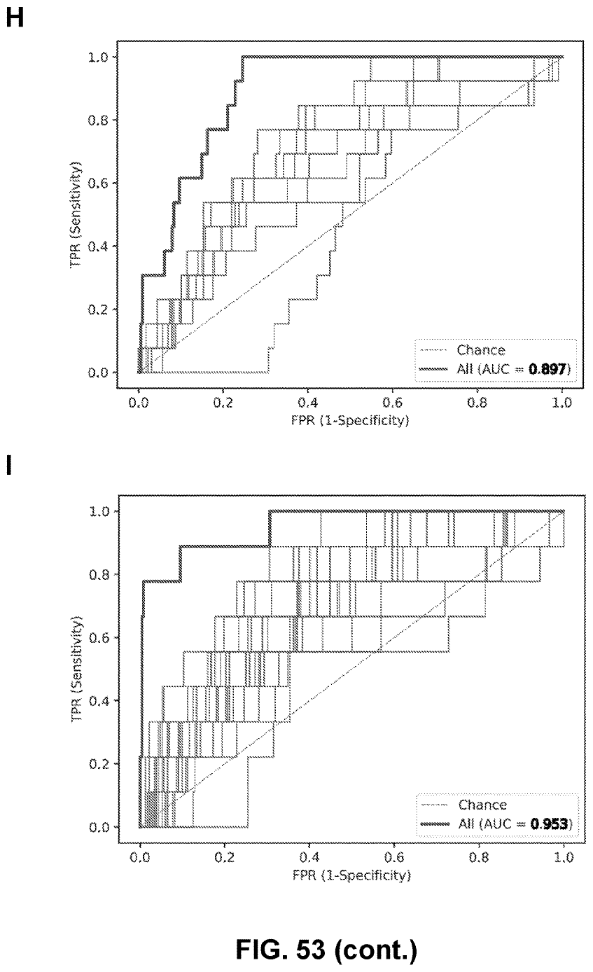

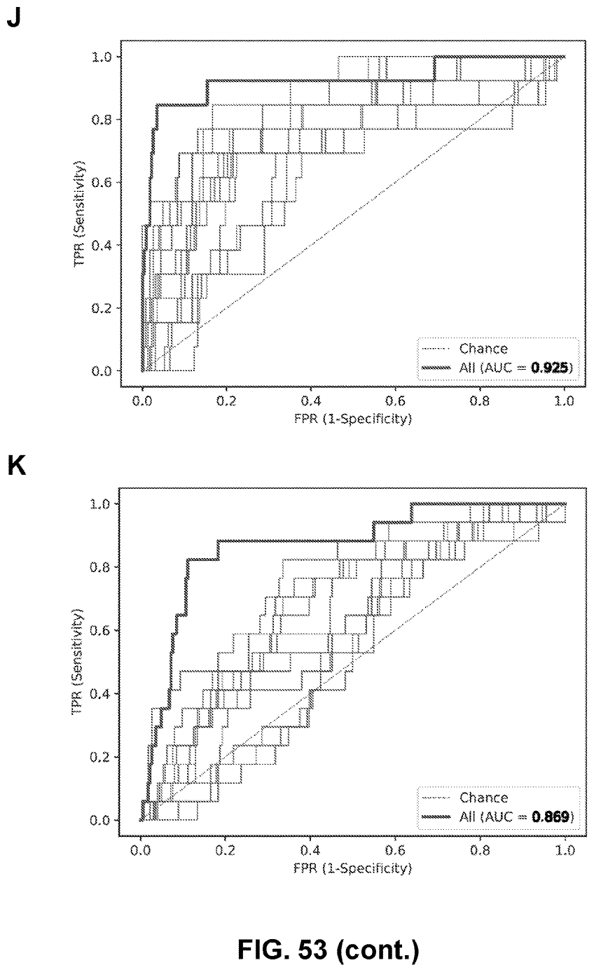

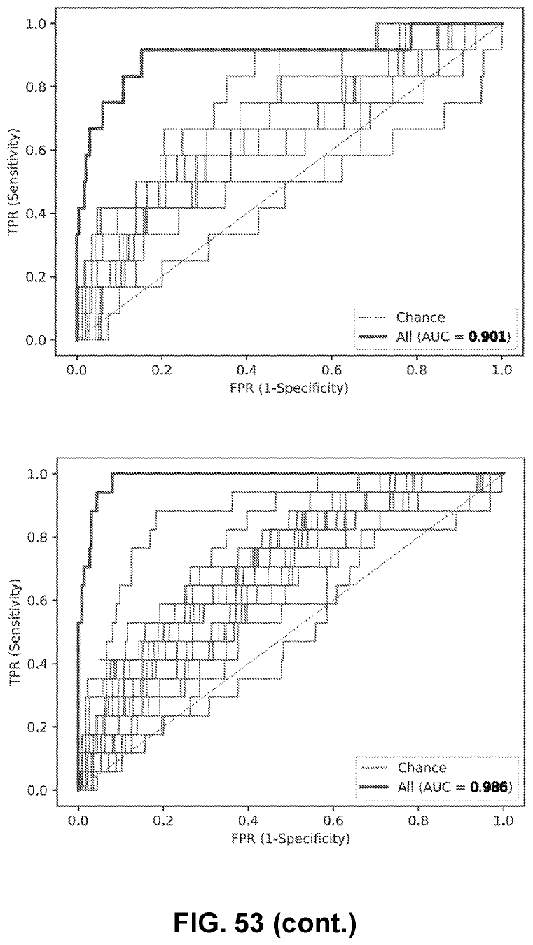

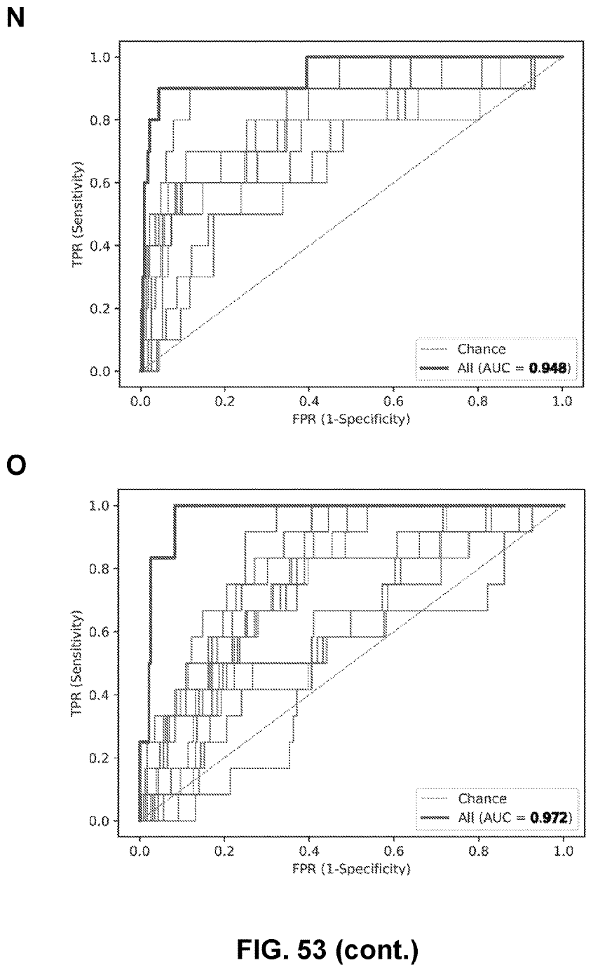

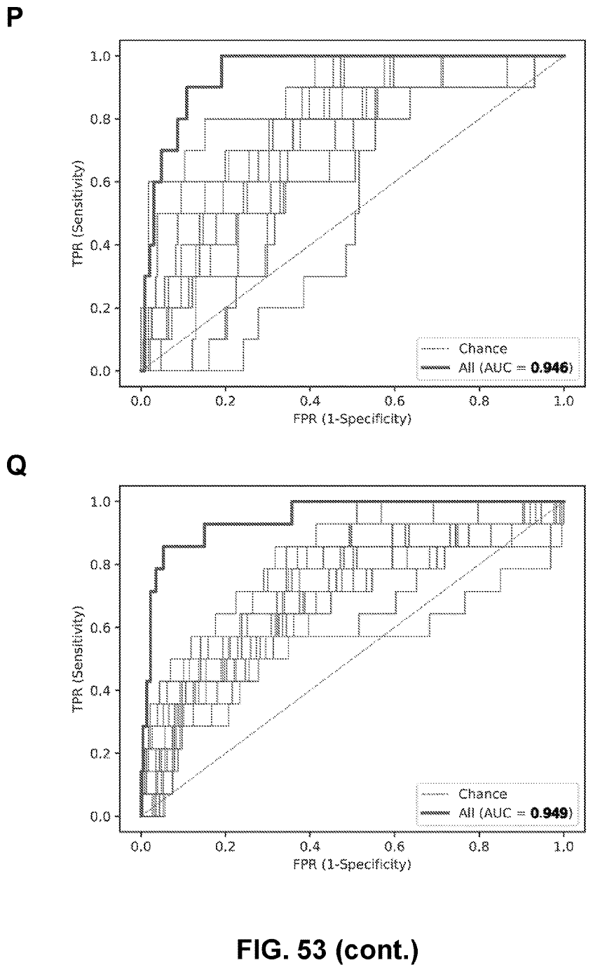

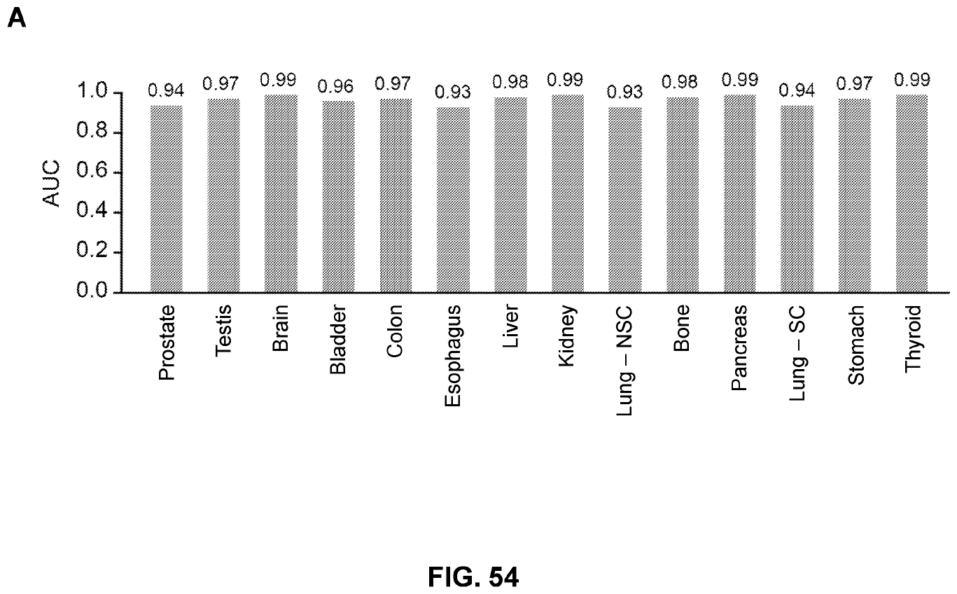

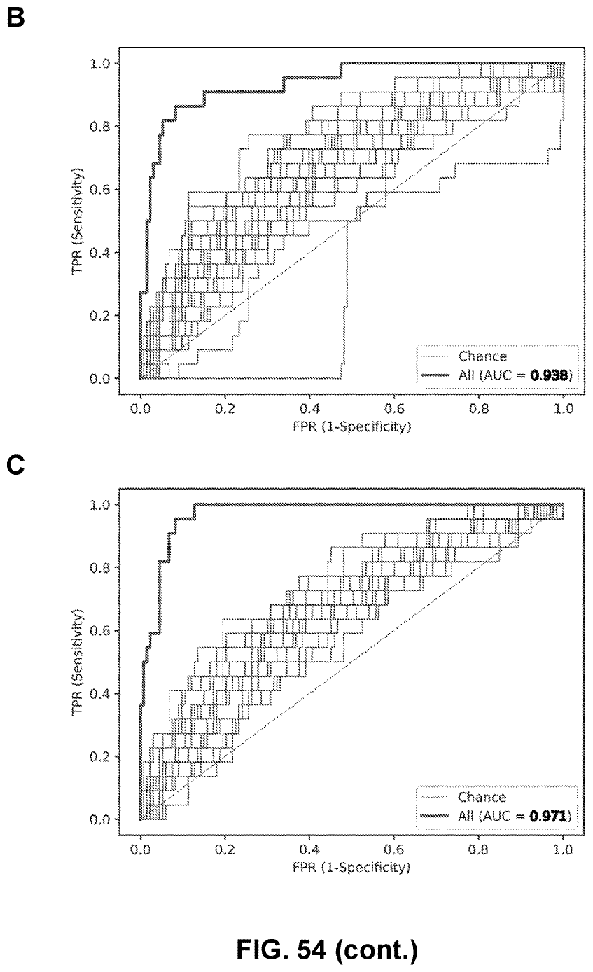

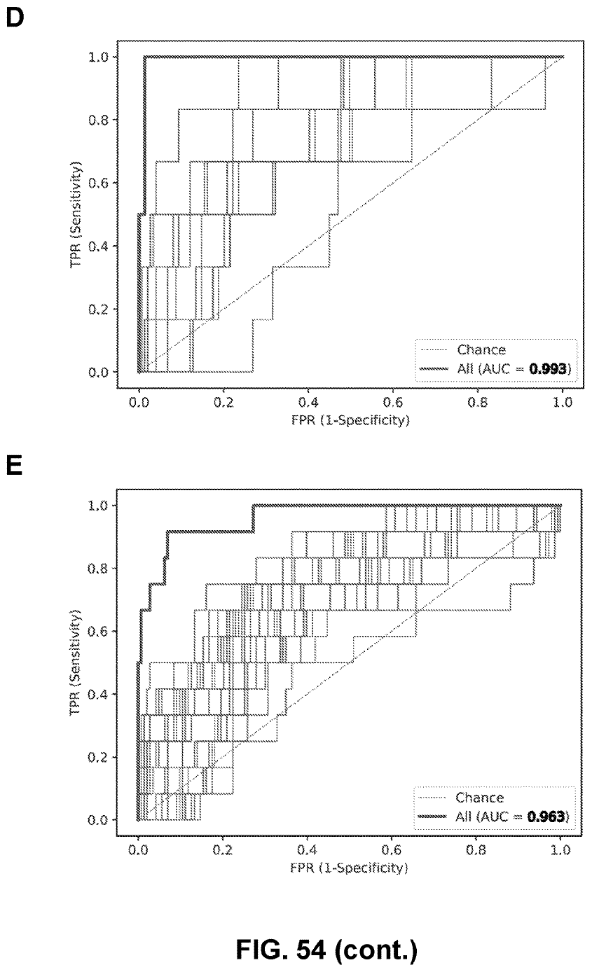

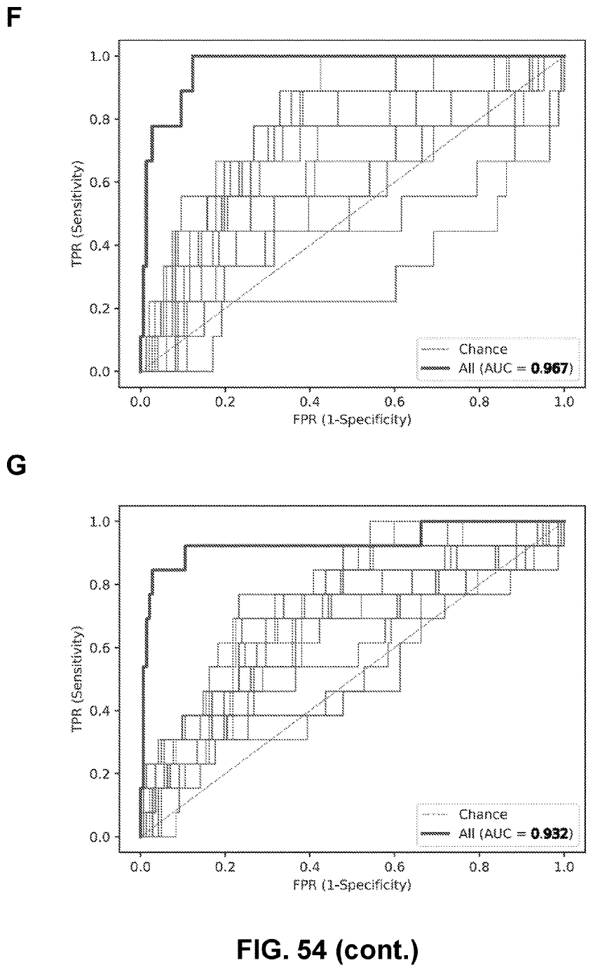

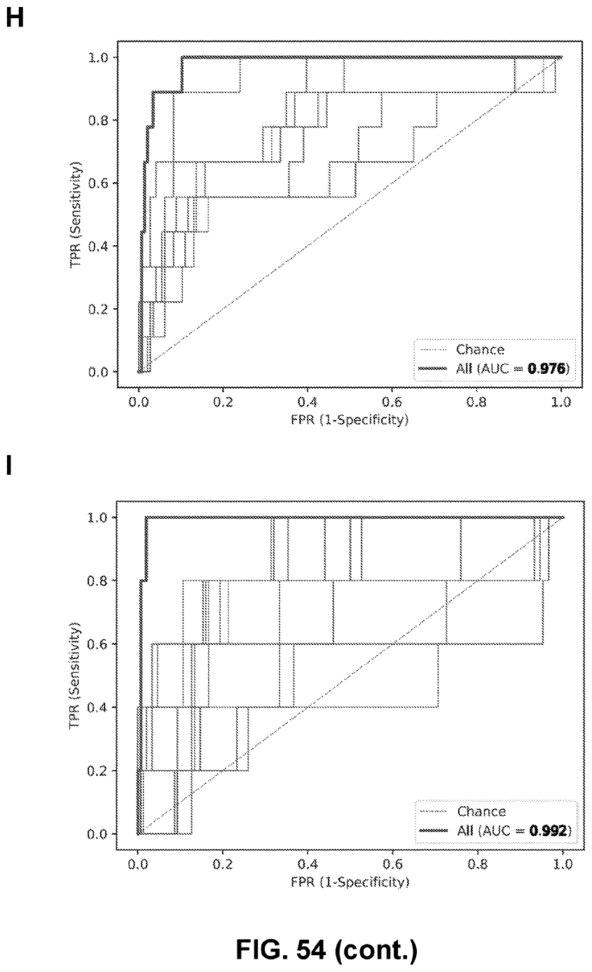

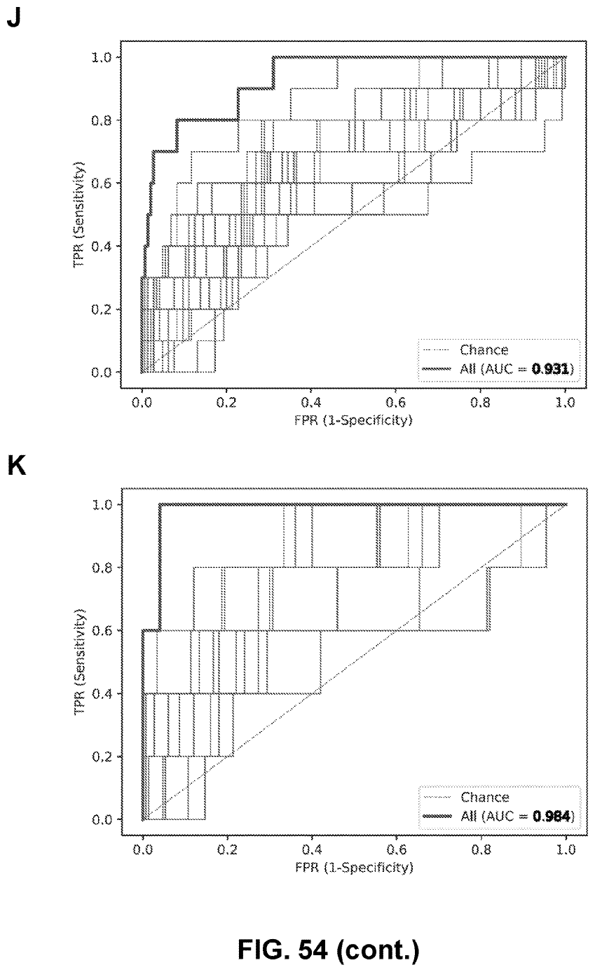

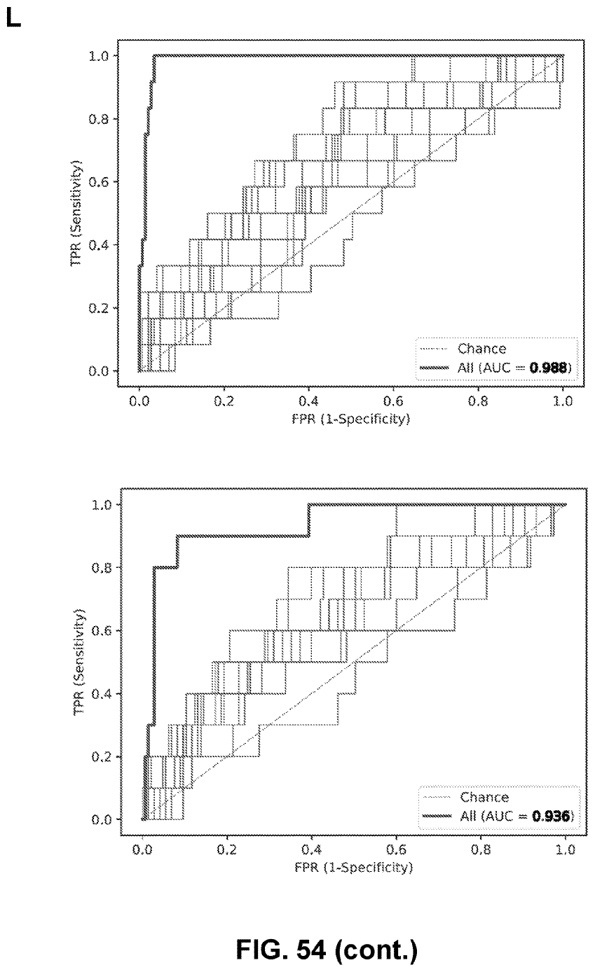

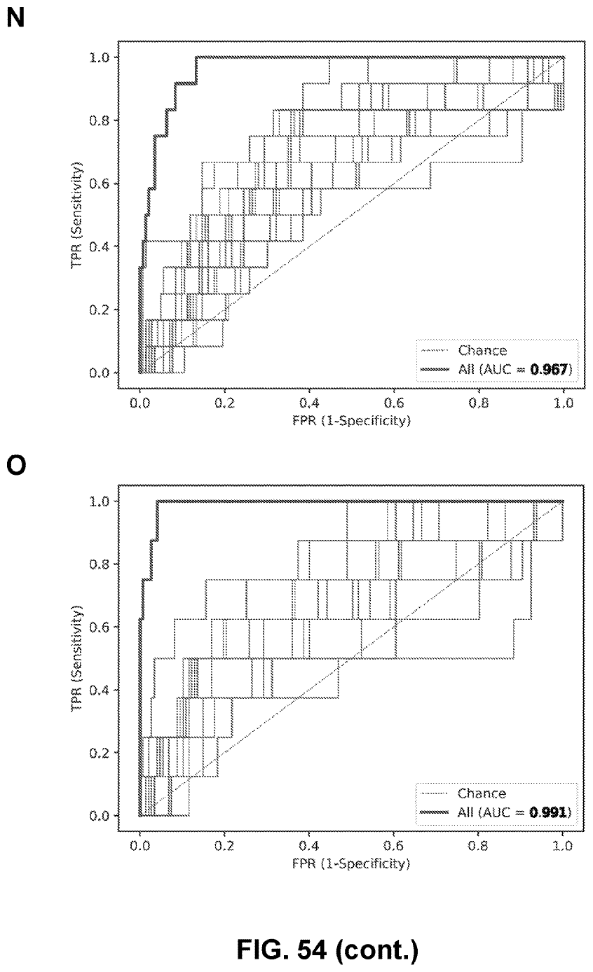

For the localization panels, a phenomenon similar to the pan-cancer detection panel was observed, where the addition of proteins to the panel no longer led to improved AUC. However, since the performance of different predictive models for specific cancers plateaued at different numbers, an algorithm was employed to select the proteins-cancer pairs for additional proteins if they improved overall AUC. Thus, different numbers of proteins were picked as the best set by the algorithm. Moreover, some proteins were helpful in localizing more than one cancer. Panels of 190 proteins were identified for females and males, in which each protein is identified as being capable of indicating cancer associated with a particular tissue(s). These female and male panels are shown in Table 251 and 252, respectively. However, the highest performance was observed at the level of 150 proteins (see FIGS. 53 and 54; see also Tables 253 and 254 for the female and male panels, respectively).

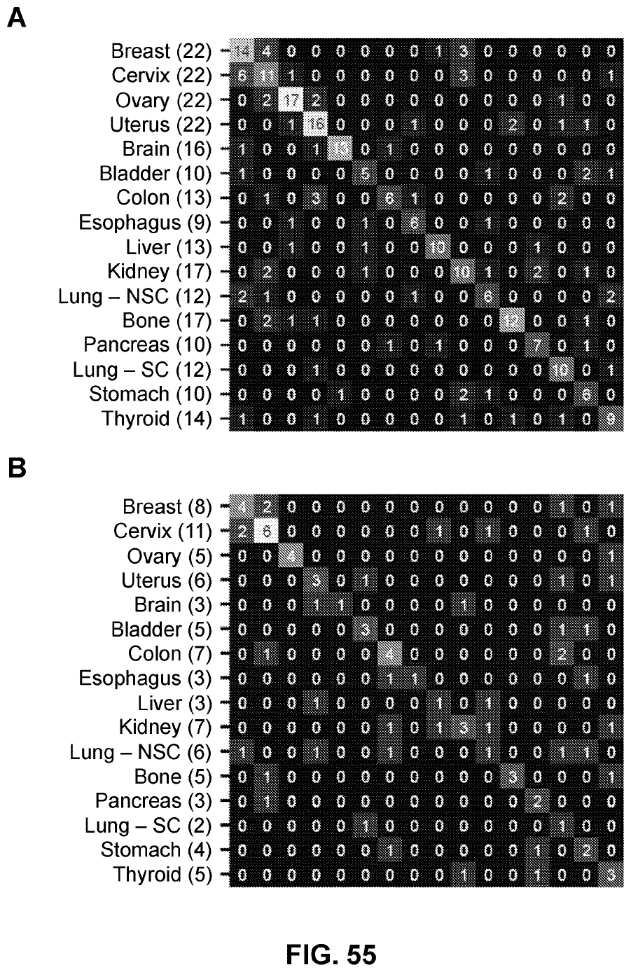

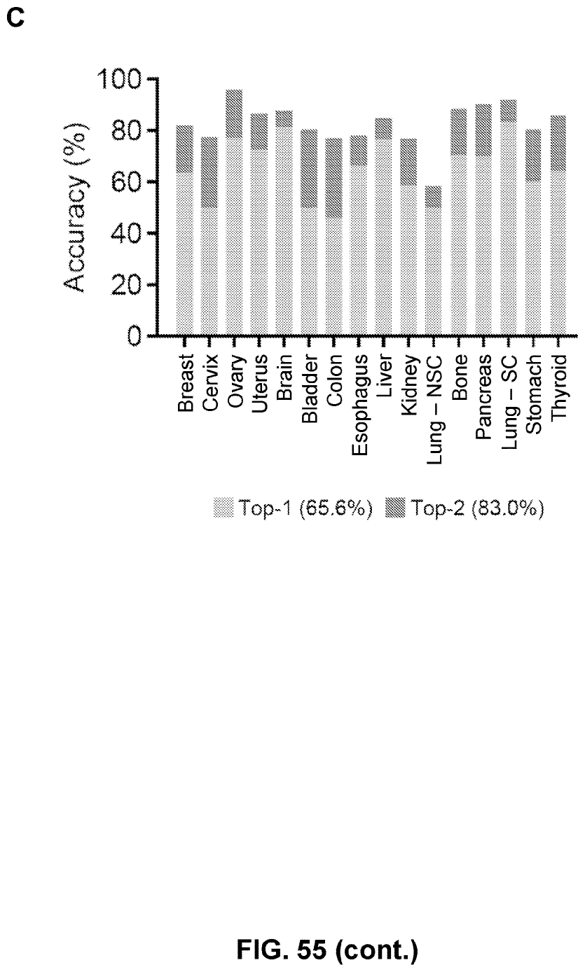

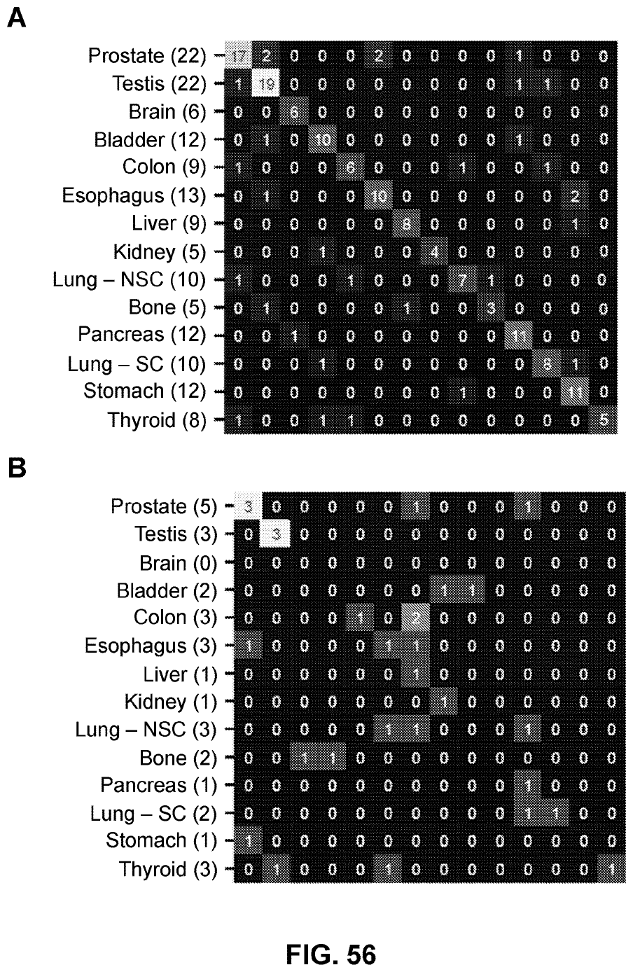

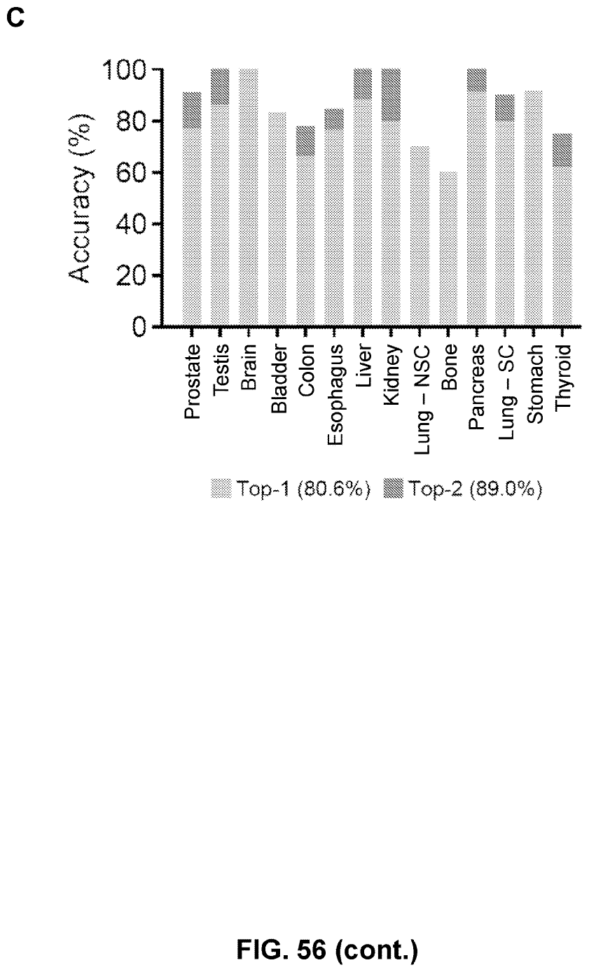

For the 150-protein localization panels, each sample was fed into separate cancer versus other cancers and the prediction probability for that cancer was calculated (see FIGS. 55 and 56). The top two highest probability was used to identify the tissue of origin. The number of proteins allocated to each cancer, which are shown in Tables 2 and 3, was selected in a way that optimized the overall performance of the test. In females the highest and the lowest number of proteins were allocated to ovarian cancer and bladder cancer, respectively, while in males the highest number of proteins was allocated to bladder cancer and the lowest number of proteins were allocated to liver cancer.

The overall performance of the test was evaluated by its ability to correctly classify the samples at the detection and localization level (see FIG. 55, Panel B, and FIG. 56. Panel B). Overall, at the specificity of 99% our sex-specific test showed the detection sensitivity of 86% for male and 91% for female. The localization accuracy of the tests was 89% for males and 77% for females. For each cancer type, the accuracy as determined by AUC achieved by the allocated proteins was at least 0.86 in the female panel and at least 0.93 om the male panel (see Tables 2 and 3).

Through the analysis of 18 cancers and normal plasma samples, it was found that only a few cancers can be uniquely identified by up-regulated proteins, which are typically preferred as biomarkers. Many cancers showed much higher specificity using downregulated proteins rather than just upregulated proteins.

The evaluation of the plasma concentrations of proteins that were selected showed that almost all the proteins were in the low abundance group. The critical proteins such as CCLI 1 required no dilution, compared with high abundance proteins such as FGA which require 100,000-fold dilution to be detected reliably by proximity technology. This highlights the importance of low-concentration proteins to see precancerous states and early stages where the tumor has little systemic impact and generated footprints.

### Example 3

Analyses were performed to identify additional pan-cancer proteins and tissue-specific cancer proteins of the present invention. Plasma samples were collected from 18 early-stage solid tumors, including cancers of the bladder, bone, brain, breast, cervix, colon, esophagus, kidney, liver, lung—small cell, lung—non-small cell, ovary, pancreas, prostate, stomach, testis, thyroid, and uterus. Additionally, plasma samples were obtained from healthy individuals representing a diverse group of blood donors. These donors varied in body mass index, levels of physical activity, and smoking habits, ensuring a broad representation of health profiles. Only samples from cancer patients with a diagnosis confirmed by a pathologist and who were treatment naïve were included in the study.

### Protein Measurement

Liquid biopsy offers the possibility to assess the state of health of specific tissues in the body by detecting their released molecules in easily accessible body fluids such as blood. The approach offers a means to screen for disease processes anywhere in the body, and it represents a simple and practical alternative to taking biopsies of specific organs for molecular analysis.

While any protein measurement technique could have been used, including ELISA, CLIA, IHC, liquid-bead immunoassays, mass spectrometry, aptamer-based assays, RPPA, etc., a proximity extension assay (PEA) was employed to evaluate proteins in plasma. In PEA, each protein was recognized by two antibodies for proper detection. In proximity assays, each of the two antibodies was conjugated to one of two different DNA oligonucleotides, and the reagents were incubated with the samples in solution. The proximity reactions underwent a dilution step. Oligonucleotides on pairs of antibodies that remain in proximity by virtue of having bound the same protein molecule then underwent DNA ligation (proximity ligation assay) or DNA polymerization (proximity extension assay). The effect of the ligation or polymerization reactions was to create amplifiable reporter DNA strands for sensitive readout via, for example, real-time PCR or next-generation sequencing, and the assays could be performed in high multiplex. By constructing the assays so that only proper pairs of antibodies can yield detection signals, but no other combination of antibodies, the detection of many different proteins in parallel was possible without eroding detection specificity by reactions of noncognate pairs.

The analytical performance of the panels was validated for sensitivity, dynamic range, specificity, precision, and scalability. The analytical measuring range was defined by the LLOQ and ULOQ and reported in pg/mL. The high dose hook effect (a state of antigen excess relative to the reagent antibodies resulting in falsely lower values) was also determined for each analyte.

All assays were thoroughly validated for precision (repeatability and reproducibility). Intra-assay variation (within-run) was calculated as the mean CV for individual samples, within each separate run during the validation studies. Inter-assay variation (between-runs) was calculated as the mean CV, for the same individual samples, among separate runs during the validation studies.

Each protein analyte was addressed by a matched pair of antibodies, coupled to unique, partially complementary oligonucleotides and measured by quantitative real-time PCR. Validation of the readout specificity for all of the panels was carried out using a simple, sequential approach in which pools of protein analytes were tested with all antibody probe pairs in the panel.

### Feature Selection

Proteins were used to create features that could be used for the classification of samples. The proteins were categorized based on their concentration or their patterns of change detected by different statistical or machine-learning techniques to create new features.

Machine learning and statistical analyses techniques used to generate features and the final score for each cancer were included but not limited to the following concepts and methods: supervised learning concepts that may include AODE: artificial neural network, such as Backpropagation, Auto encoders, Hopfield networks, Boltzmann machines, Restricted Boltzmann Machines, and Spiking neural networks; Bayesian statistics, such as Bayesian network and Bayesian knowledge base; case-based reasoning; Gaussian process regression; gene expression programming; GMDH; inductive logic programming; instance-based learning; lazy learning; learning Automata; learning vector quantization; logistic model tree; minimum message length (decision trees, decision graphs, etc.), such as nearest neighbor algorithm and analogical modeling; probability approximately correct learning (PAC) learning; ripple down rules, a knowledge acquisition methodology; symbolic machine learning algorithms; support vector machines: random forests; ensembles of classifiers, such as bootstrap aggregating (bagging) and boosting (meta-algorithm): ordinal classification; IFN; conditional random field; ANOVA; linear classifiers, such as Fisher's linear discriminant, linear regression, logistic regression, multinomial logistic regression, naive Bayes classifier, Perceptron, support vector machines; quadratic classifiers; k-nearest neighbor: boosting: decision trees, such as C4.5, random forests, ID3, CART, SLIQ SPRINT; Bayesian networks, such as Naive Bayes; and Hidden Markov models, cUnsupervised learning concepts may include; expectation-maximization algorithm; vector quantization; generative topographic map: information bottleneck method; artificial neural network, such as self-organizing map; association rule learning, such as Apriori algorithm. Eclat algorithm, and FP growth algorithm; hierarchical clusterings such as single linkage clustering and conceptual clustering; cluster analysis, such as K-means algorithm, fuzzy clustering, DBSCAN, and OPTICS algorithm; and outlier detection, such as local outlier factor. Semi-supervised learning concepts may include: generative models; low-density separation: graph-based methods, and co-training. Reinforcement learning concepts may include temporal difference learning; Q-learning, learning automata, and SARSA. Deep learning concepts may include deep belief networks; deep Boltzmann machines; deep convolutional neural networks; deep recurrent neural networks; and hierarchical temporal memory.

### Cancer Detection Proteins

One or more features were fed into one or more computation models. The classifiers were used to calculate a score for the patient. The scores of different classifiers were combined to identify the patient as having specific cancer or not. The computational model only selected protein or protein combinations that could generate a ROC curve of greater than or equal to 0.6.

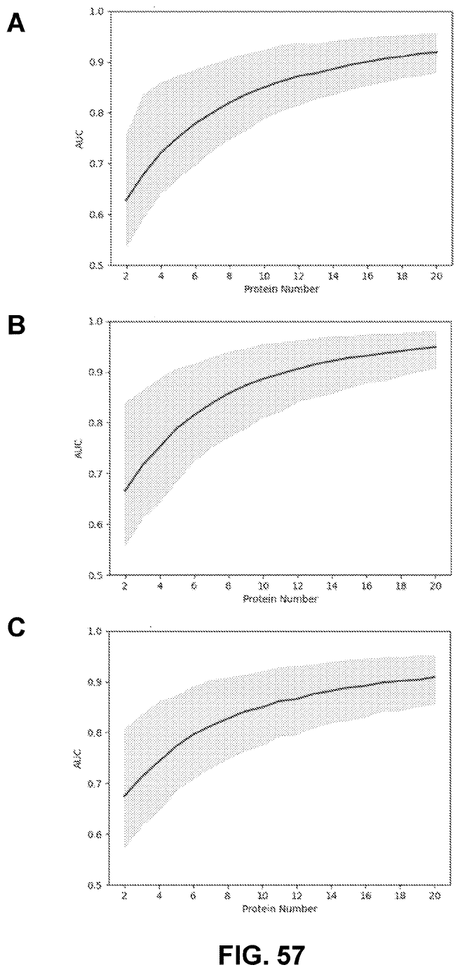

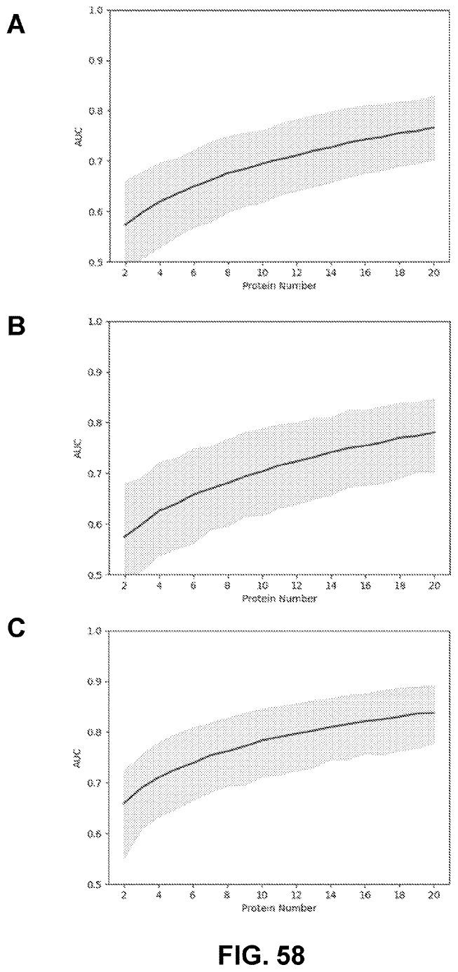

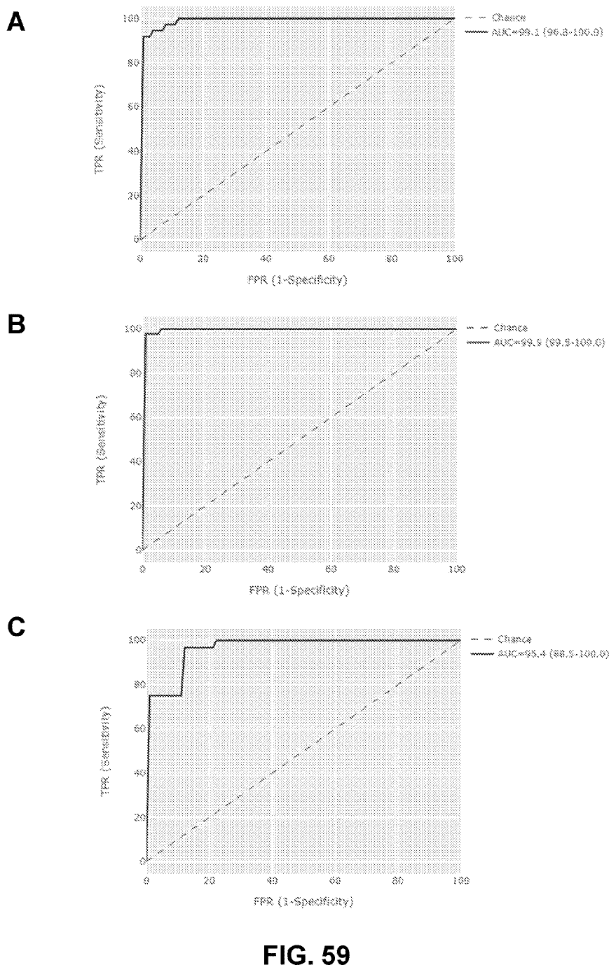

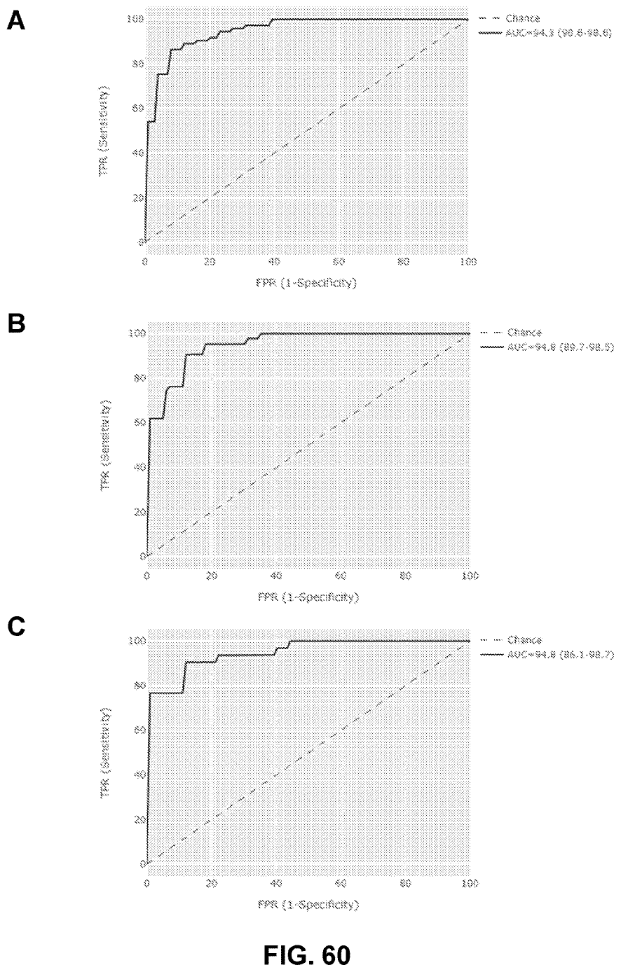

Tissue-specific cancer detection proteins were aggregated to create the pan-cancer detection proteins of Table 1001 that can be used in a pan-cancer detection analysis to detect whether a subject has cancer. Table 1001 also provides individual AUC values for each of the proteins, indicating the accuracy at which each protein can detect whether cancer is present in the subject. That any combination of two or more proteins from these tables can detect whether the subject has cancer is demonstrated in FIGS. 57 and 58, which shows that the accuracy is over 0.6 when any two proteins through any 20 proteins are randomly selected, when two different classifiers were used in the analysis. The protein panels also showed high sensitivity and specificity, as shown in FIGS. 59 and 60. The accuracy (AUC) of the pan-cancer detection panels of Tables 1001.A.1, 1001.B.1, and 1001.C.1 in detecting any cancer in mixed-gender, females, and males, respectively are shown for each classifier in Table 4.

### Cancer Localization

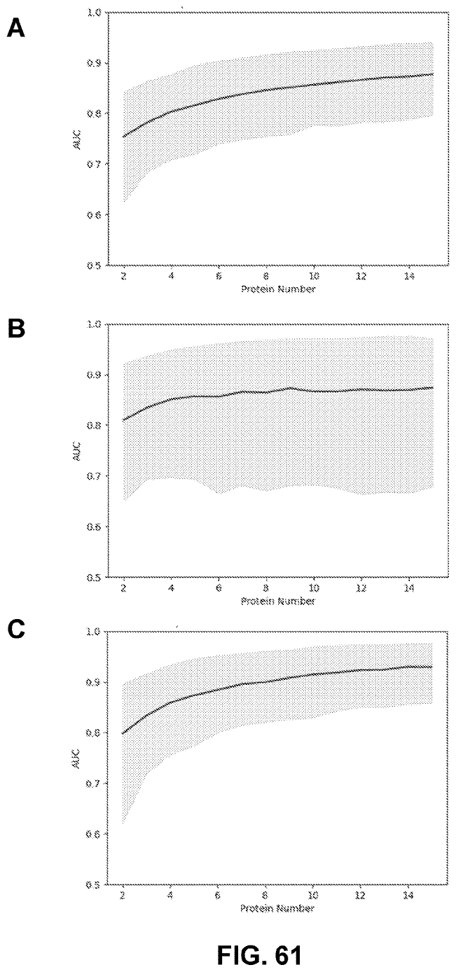

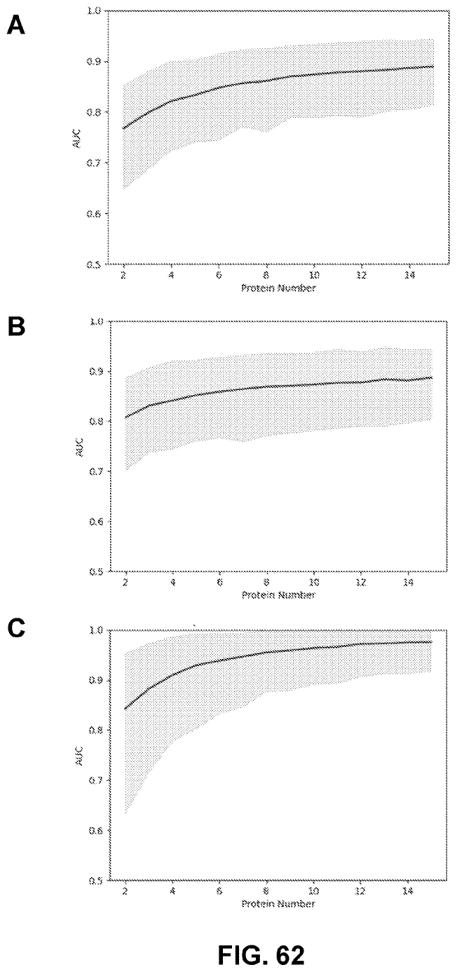

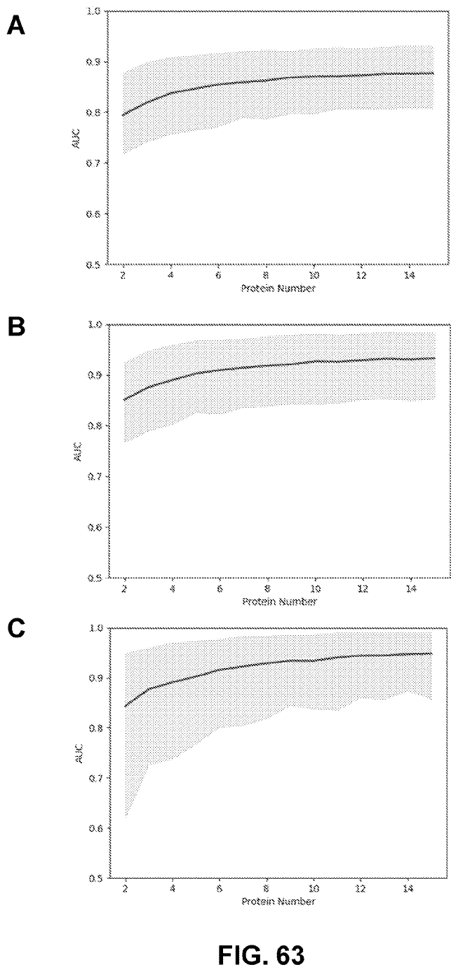

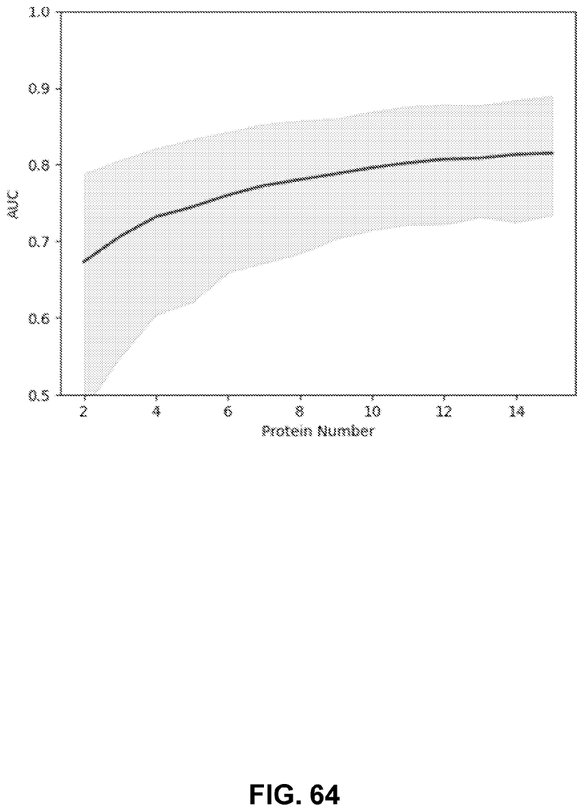

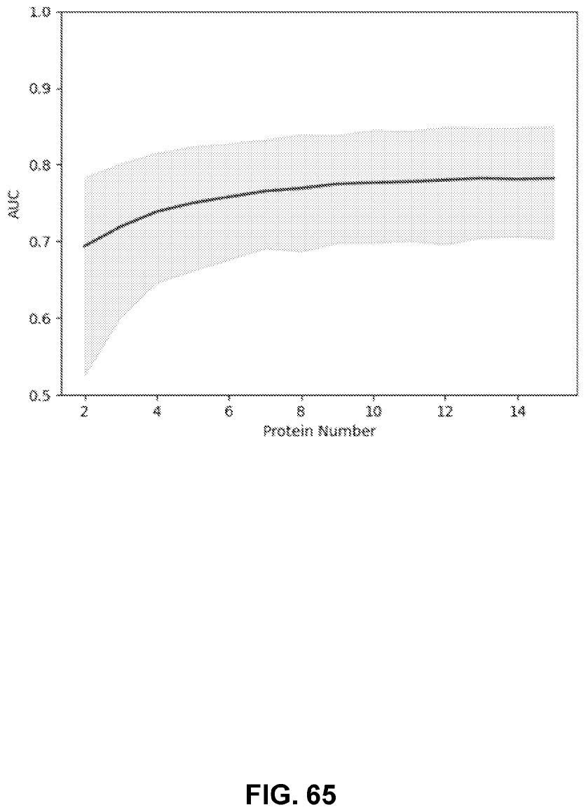

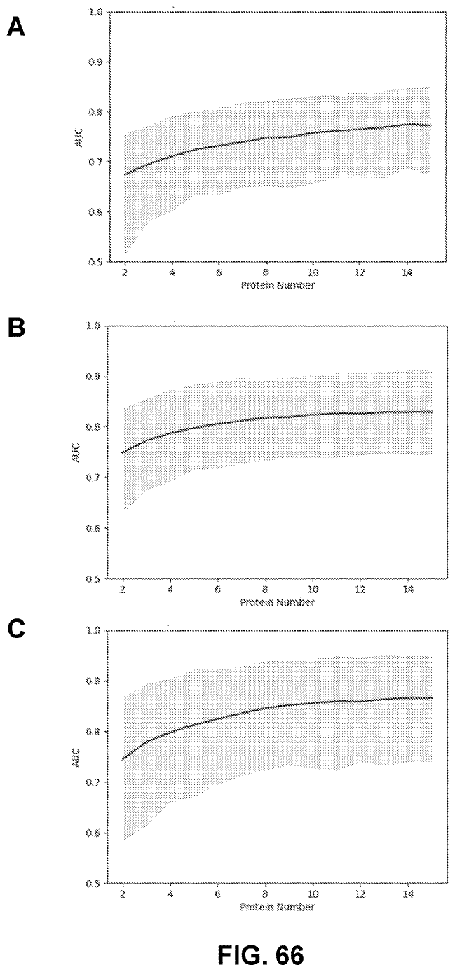

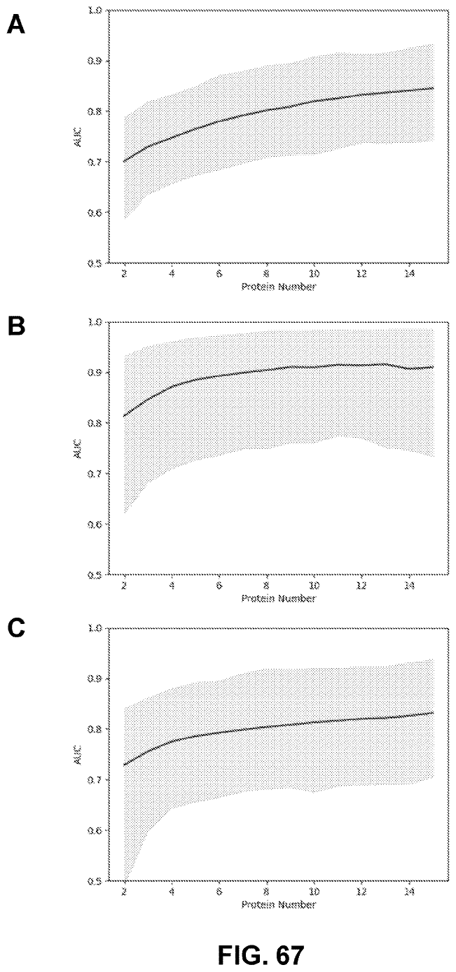

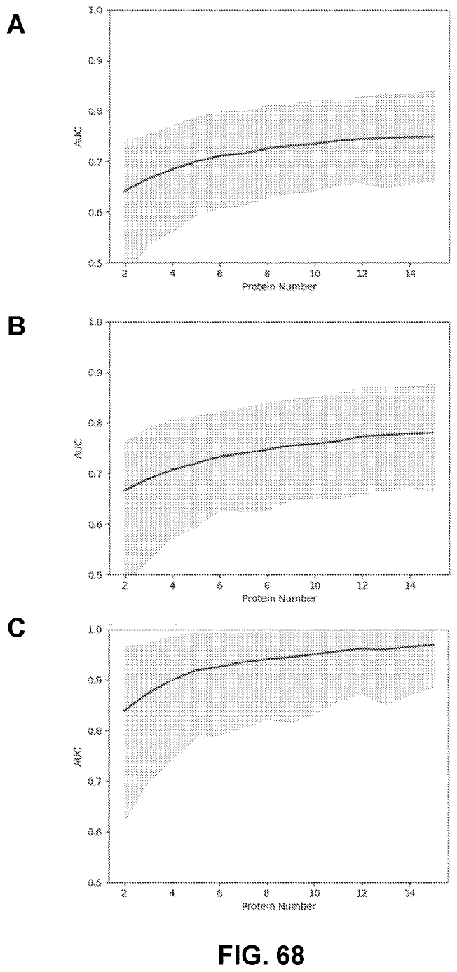

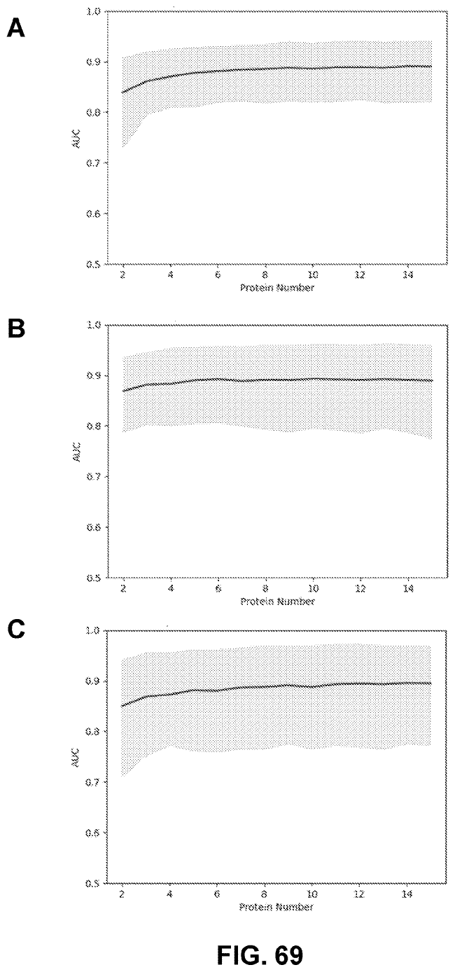

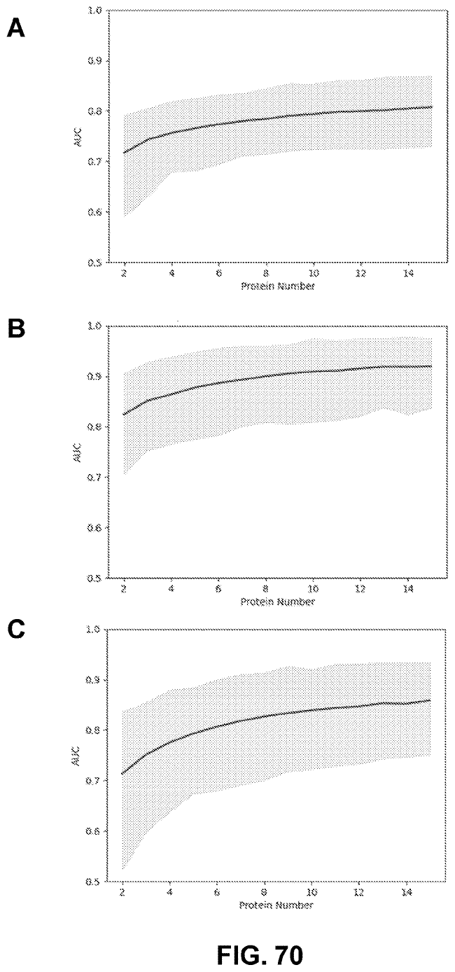

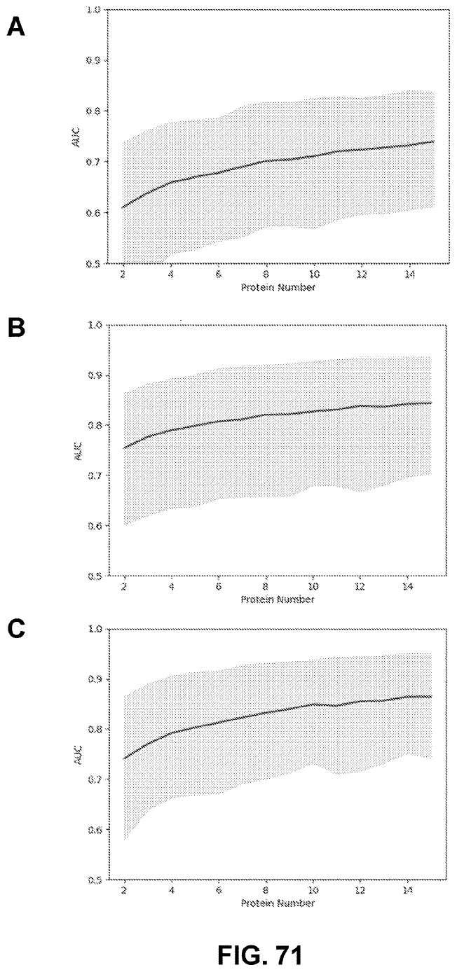

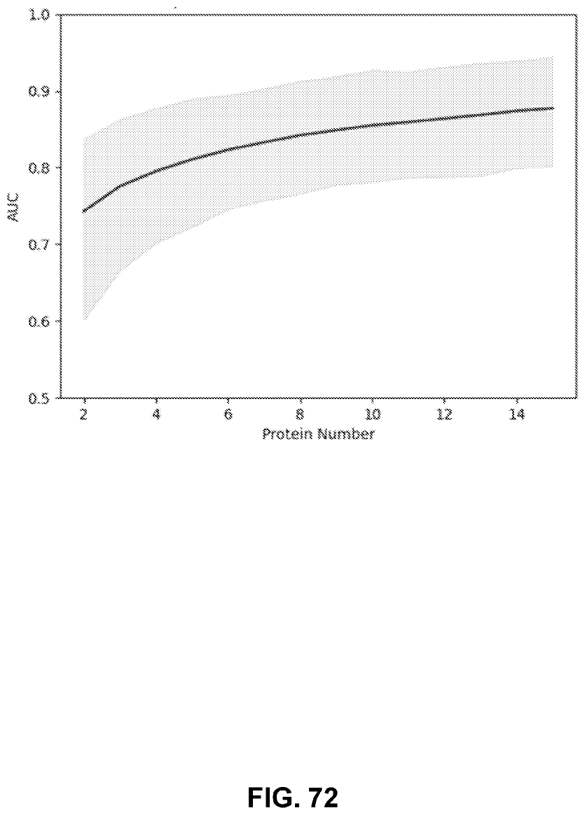

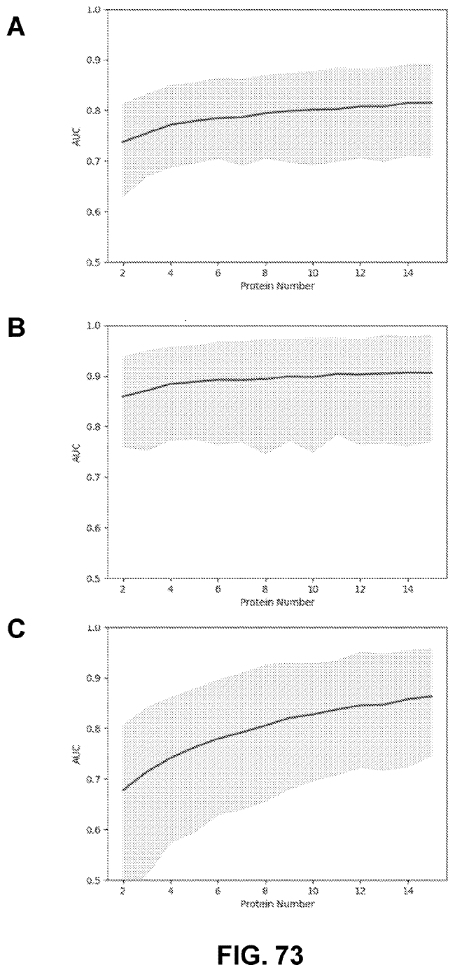

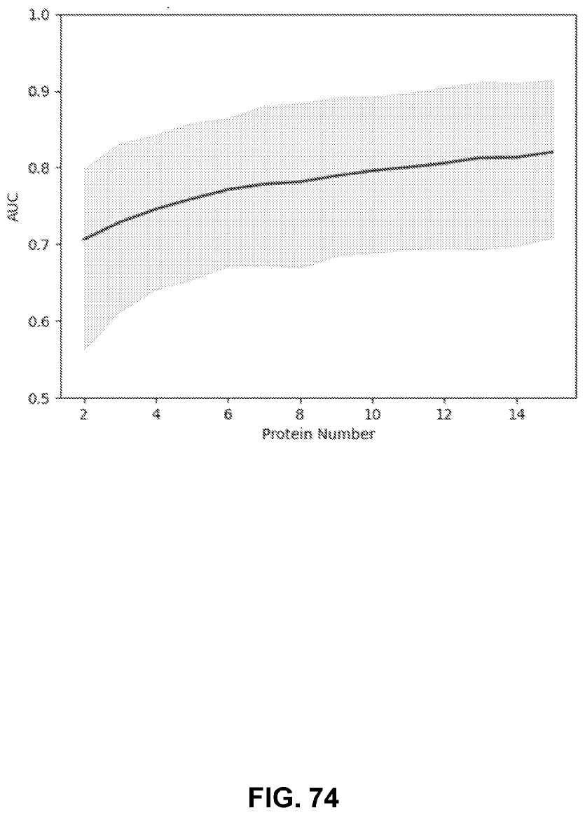

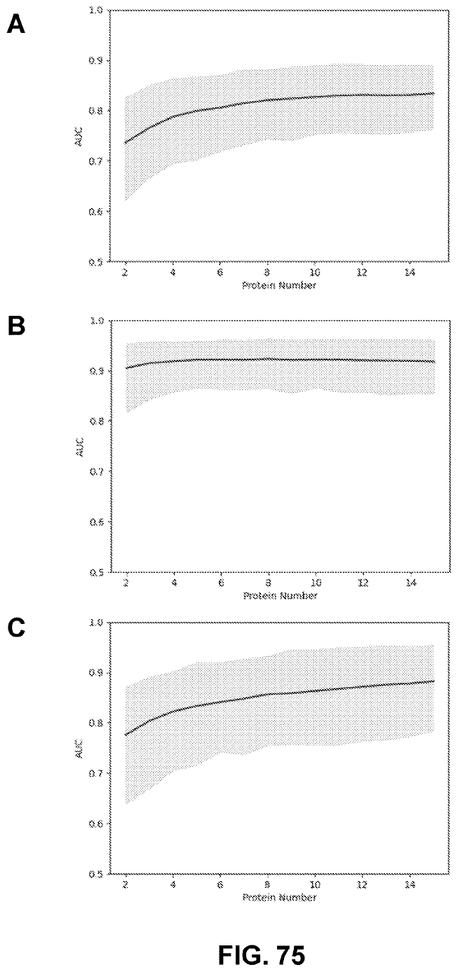

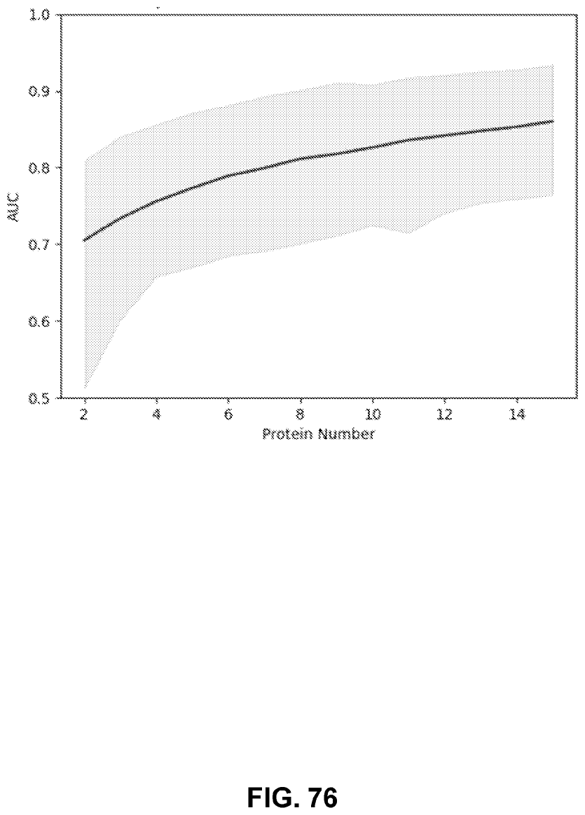

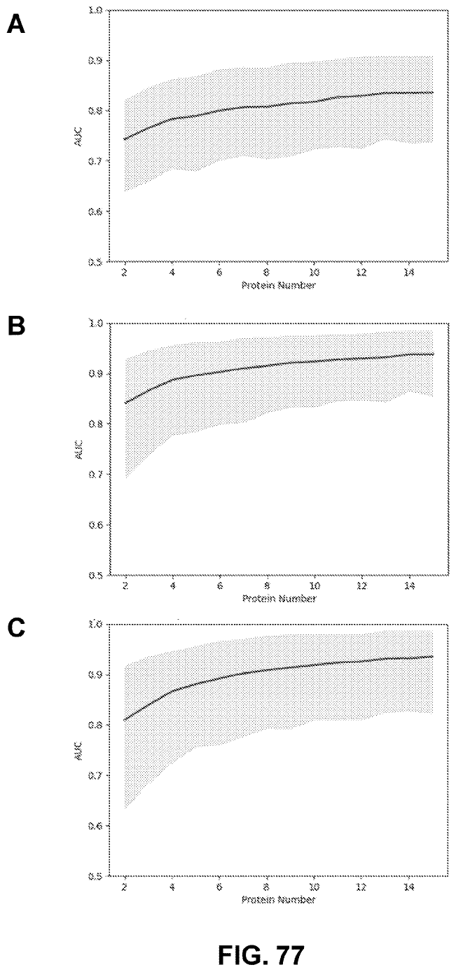

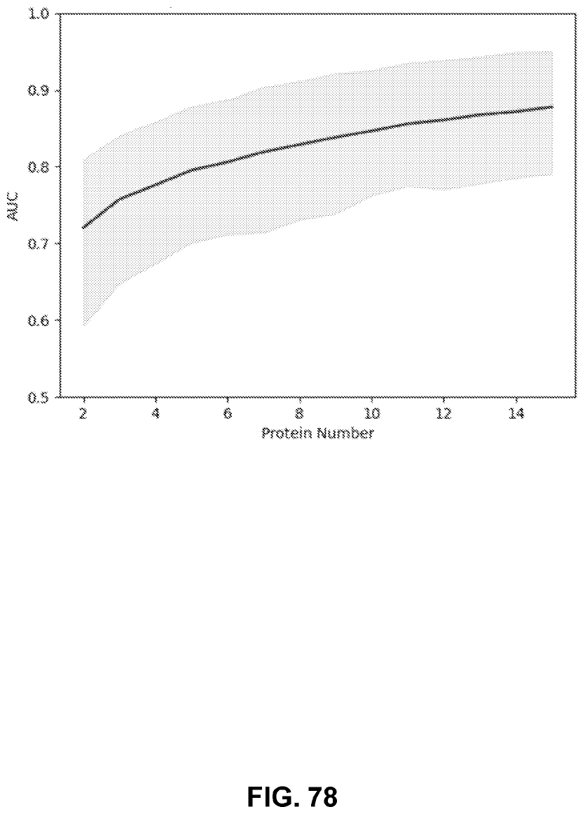

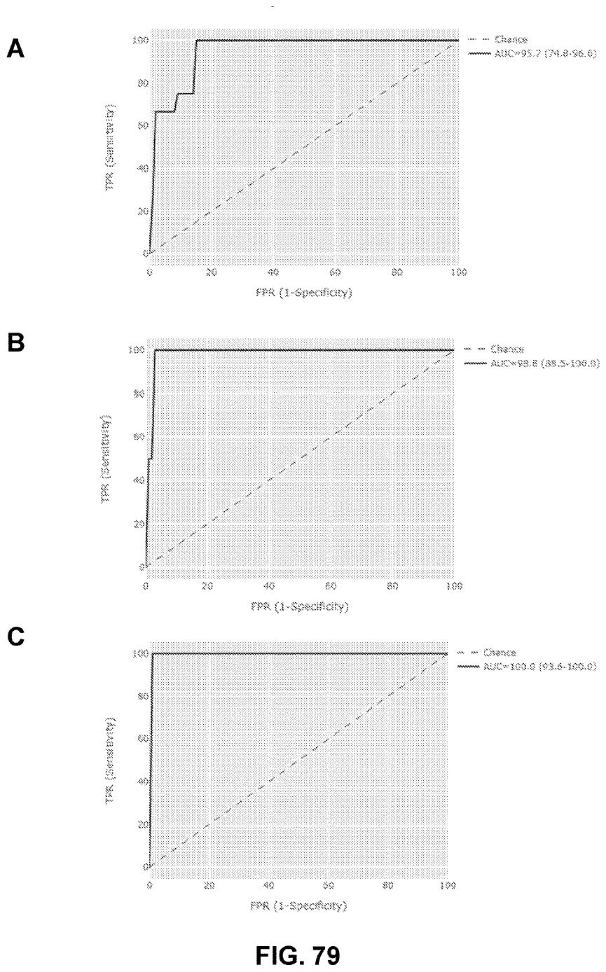

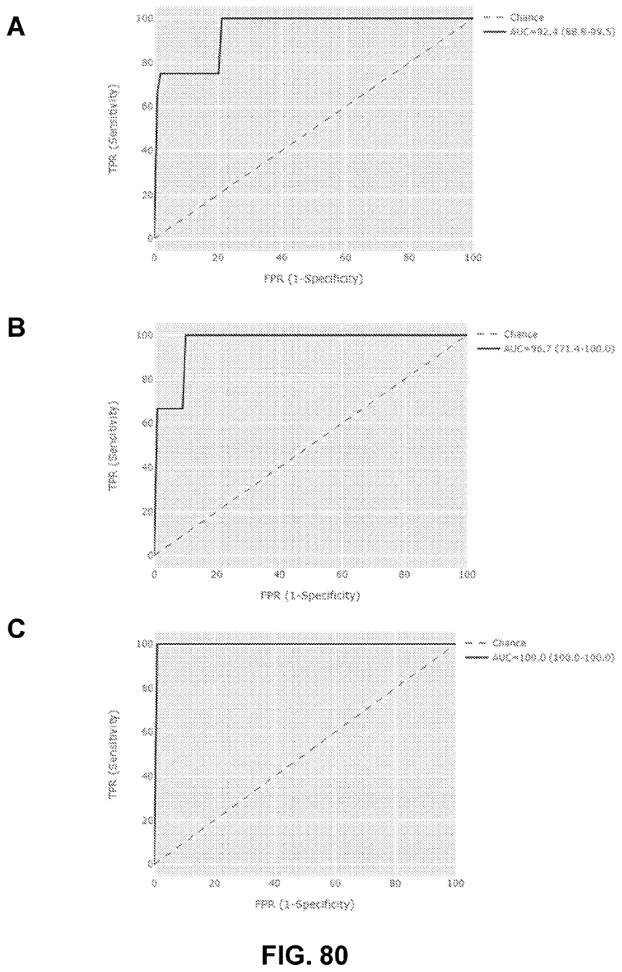

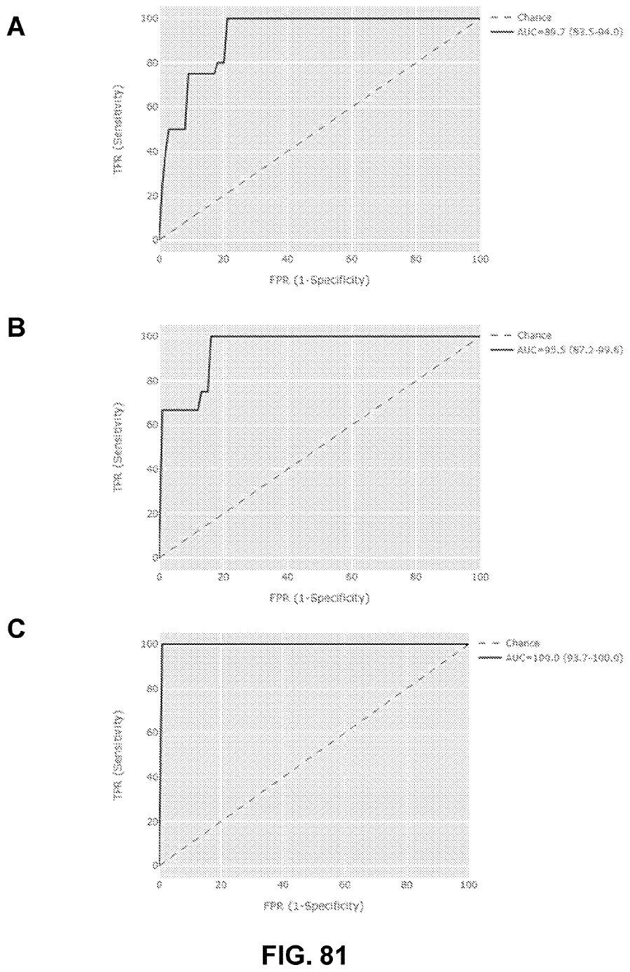

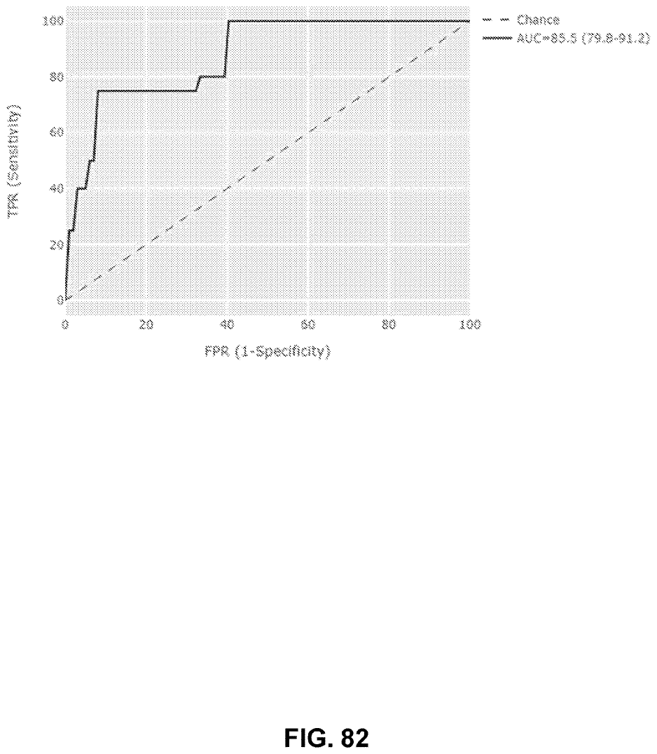

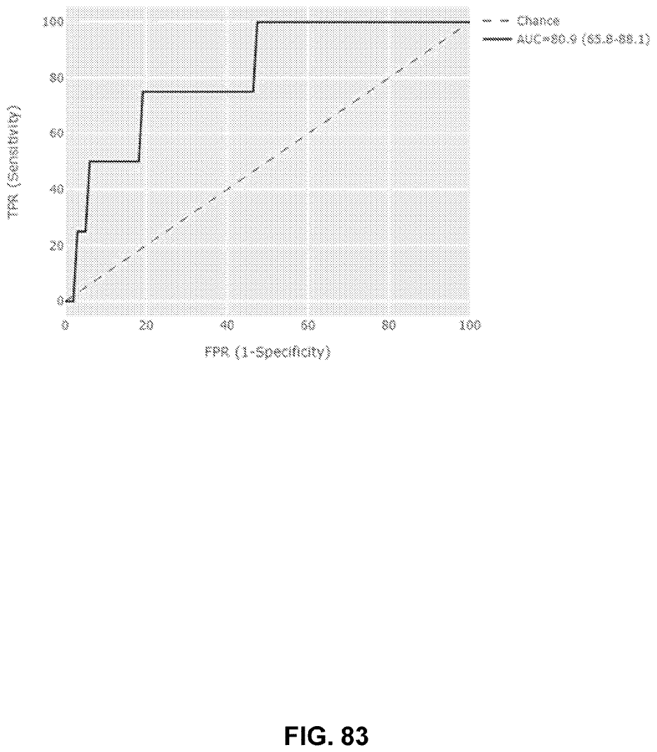

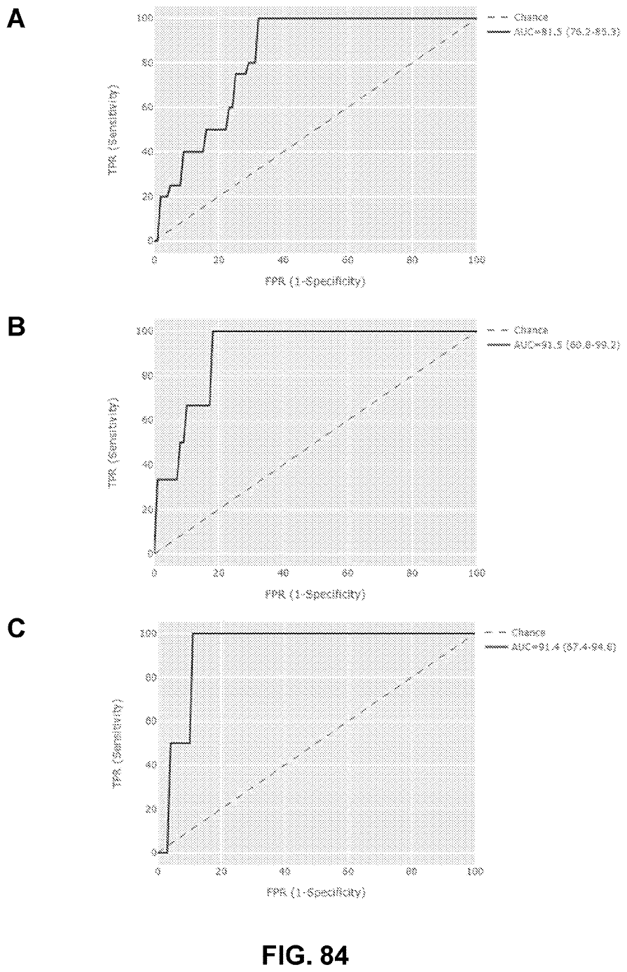

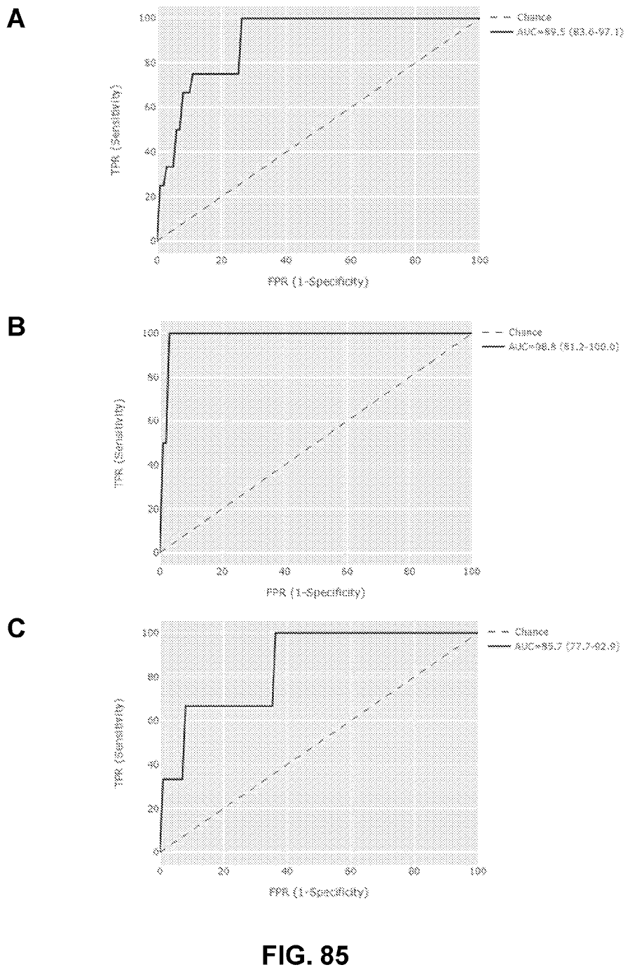

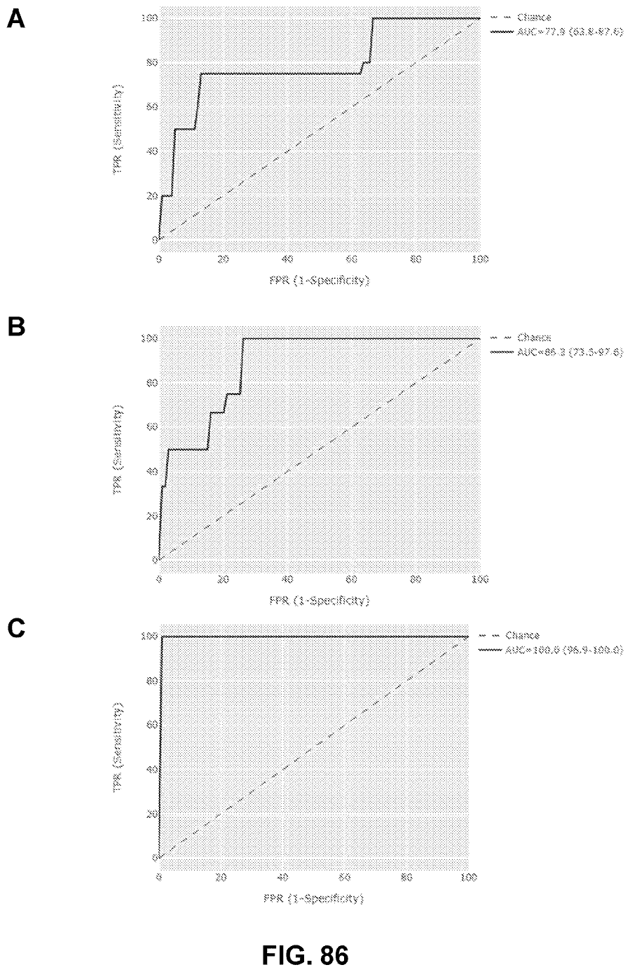

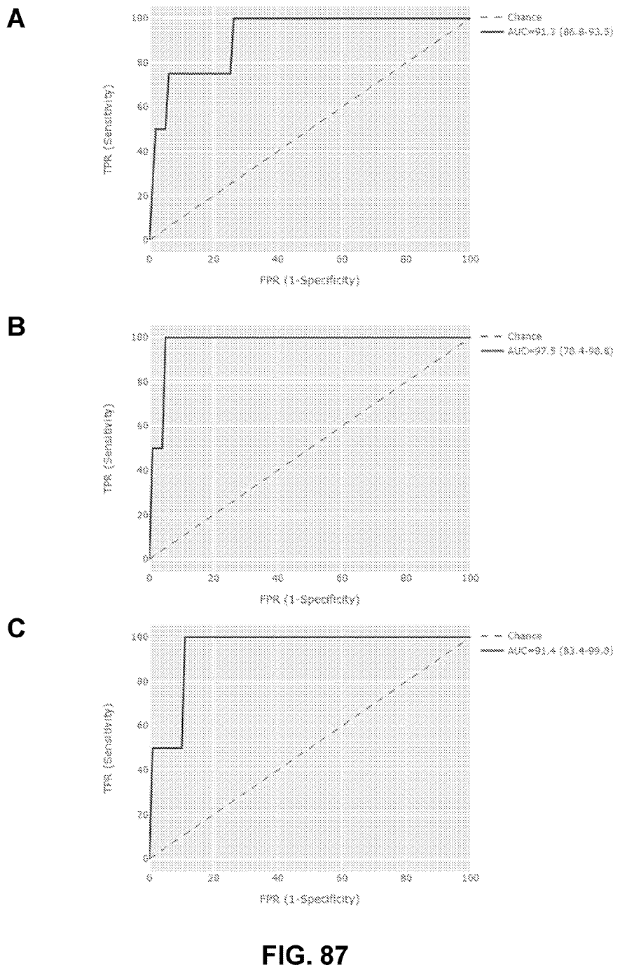

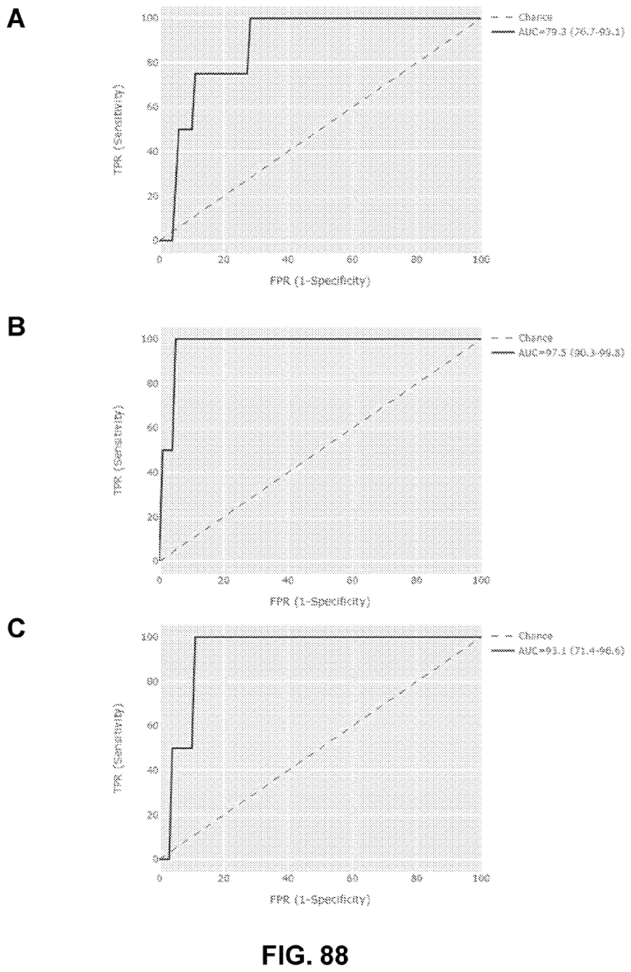

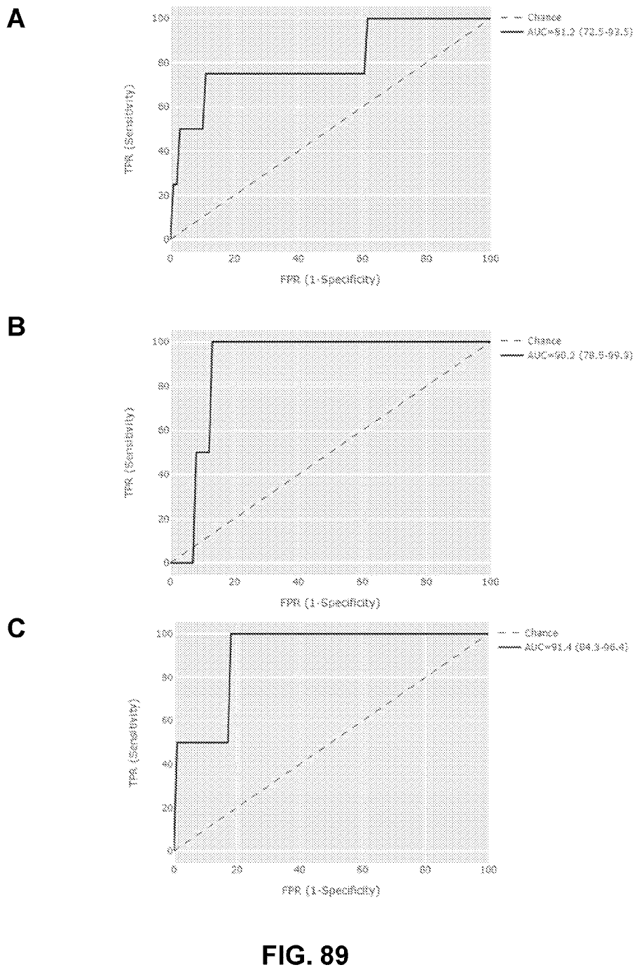

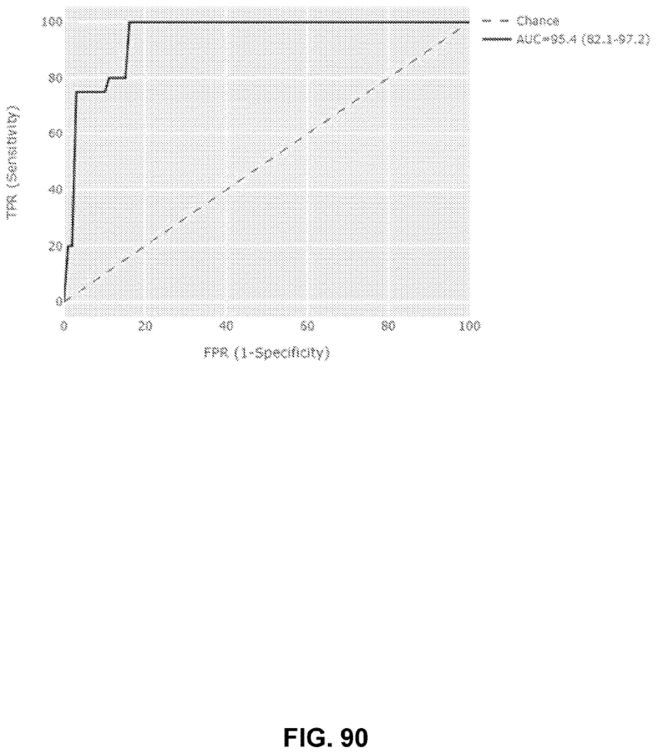

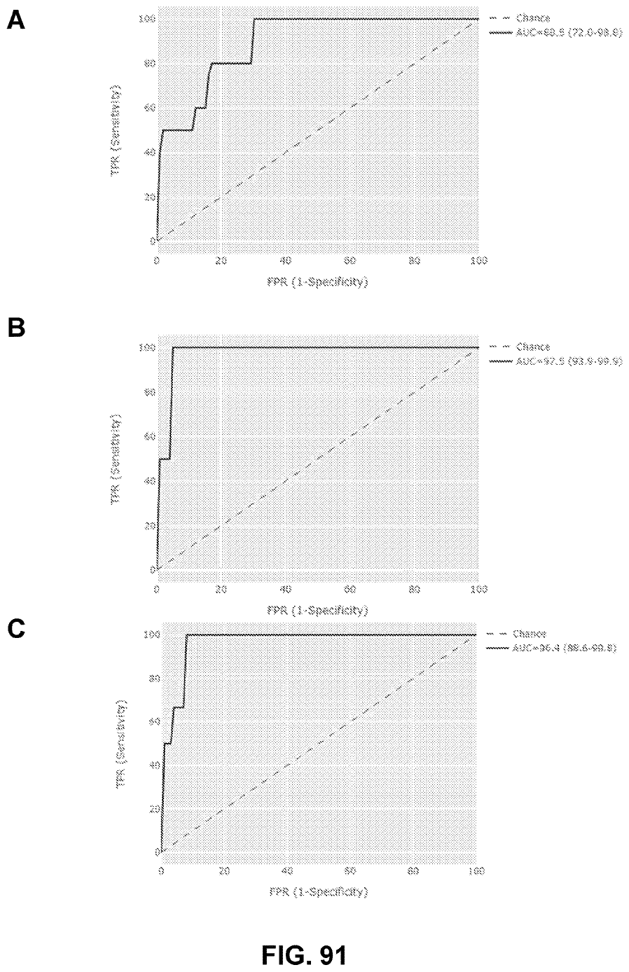

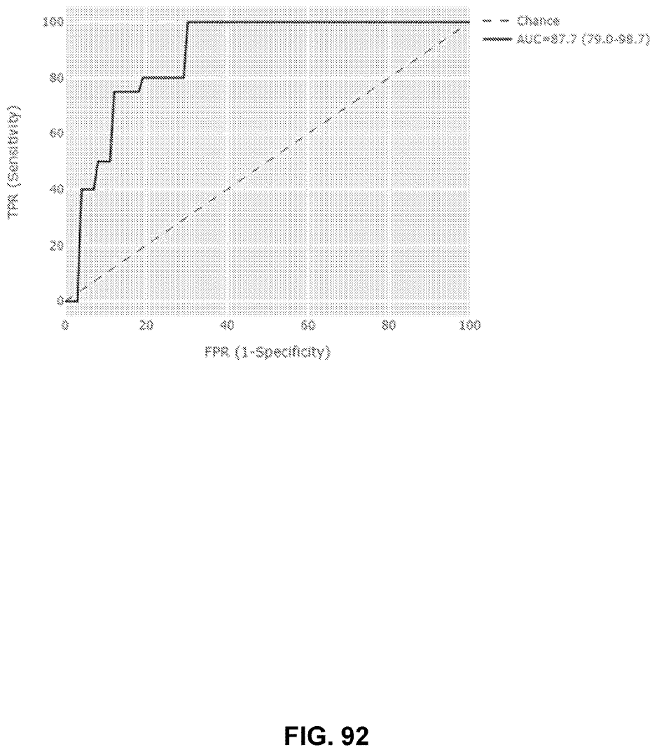

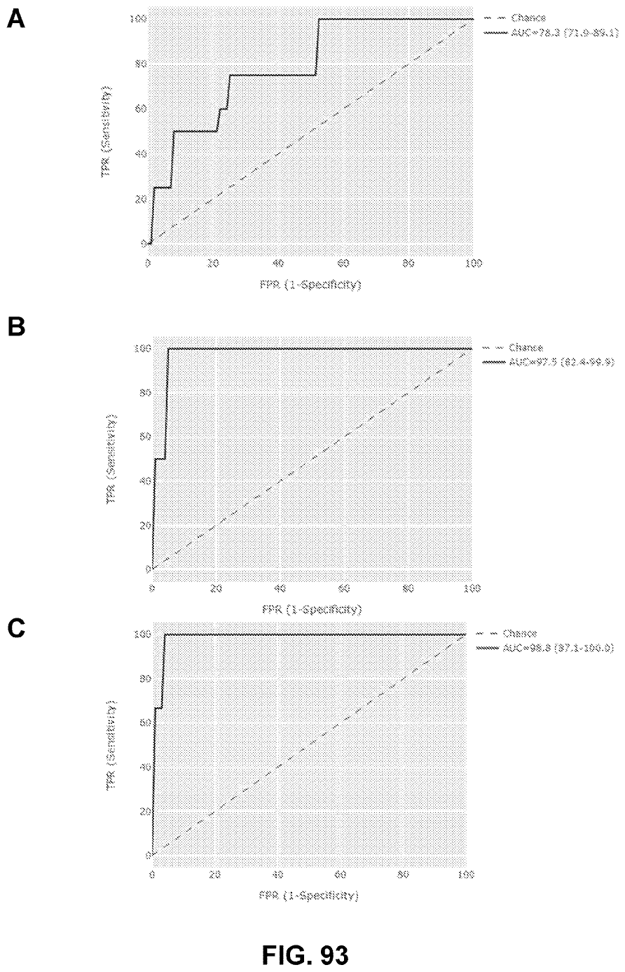

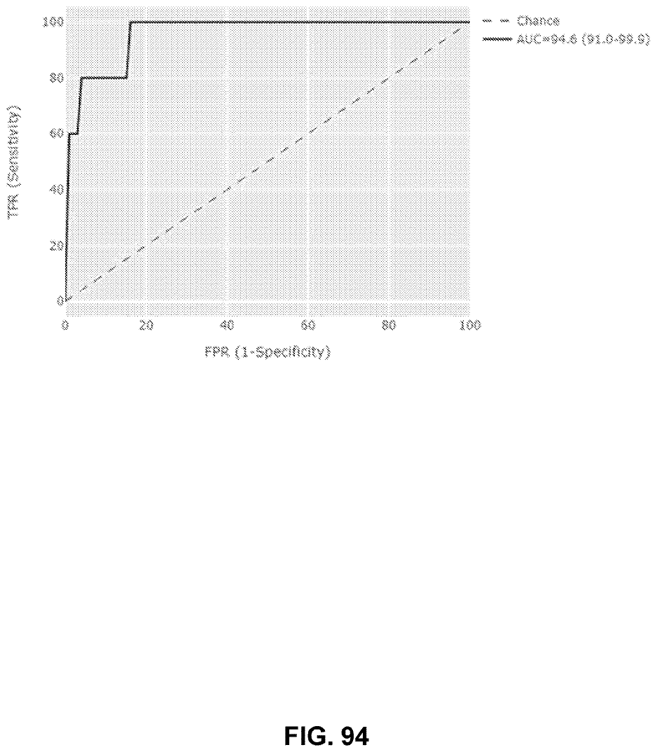

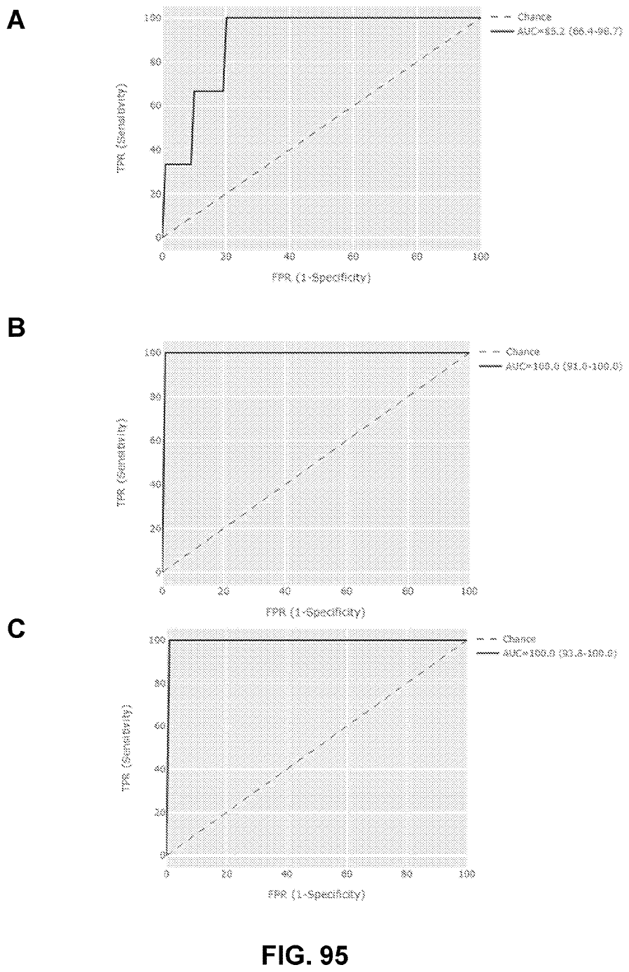

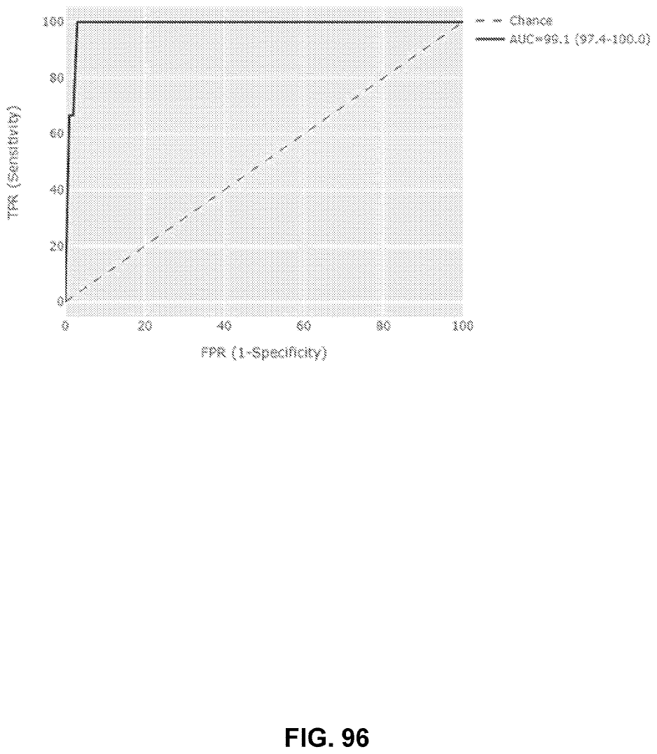

One or more features were fed into one or more computation models. The features could be similar or different from what was used in determining cancer versus non-cancer status. The classifiers were used to calculate a score for the patient for every possible cancer type, including cancers of the bladder, bone, brain, breast, cervix, colon, esophagus, kidney, liver, lung—small cell, lung—non-small cell, ovary, pancreas, prostate, stomach, testis, thyroid, and uterine. The computational model only selected protein or protein combinations that could generate a ROC curve of greater than or equal to 0.6. The scores are combined to discriminate the cancer type. The resulting tissue-specific cancer localization proteins are shown in Tables 2001-2018, corresponding to detection proteins of cancer associated with bladder, bone, brain, breast, cervix, colon, esophagus, kidney, liver, lung—small cell, lung—non-small cell, ovary, pancreas, prostate, stomach, testis, thyroid, and uterine, respectively. Tables 2001-2018 also provide individual AUC values for each of the proteins, indicating the accuracy at which each protein can detect the corresponding cancer that is associated with a tissue. That any combination of two or more proteins from these tables can detect whether the subject has the specific cancer is demonstrated in FIGS. 61-78, which shows that the accuracy is over 0.6 when any two proteins through any 20 proteins are randomly selected. Further, as shown in, as shown in FIGS. 79-96, the protein panels also showed high sensitivity and specificity. The accuracy (AUC) of the particular panels for detecting specified cancers in mixed-gender, females, and/or males are shown in Table 5.

Lengthy table referenced here
US20260196349A1-20260709-T00001
Please refer to the end of the specification for access instructions.

Lengthy table referenced here
US20260196349A1-20260709-T00002
Please refer to the end of the specification for access instructions.

Lengthy table referenced here
US20260196349A1-20260709-T00003
Please refer to the end of the specification for access instructions.

Lengthy table referenced here
US20260196349A1-20260709-T00004
Please refer to the end of the specification for access instructions.

Lengthy table referenced here
US20260196349A1-20260709-T00005
Please refer to the end of the specification for access instructions.

Lengthy table referenced here
US20260196349A1-20260709-T00006
Please refer to the end of the specification for access instructions.

Lengthy table referenced here
US20260196349A1-20260709-T00007
Please refer to the end of the specification for access instructions.

Lengthy table referenced here
US20260196349A1-20260709-T00008
Please refer to the end of the specification for access instructions.

Lengthy table referenced here
US20260196349A1-20260709-T00009
Please refer to the end of the specification for access instructions.

Lengthy table referenced here
US20260196349A1-20260709-T00010
Please refer to the end of the specification for access instructions.

Lengthy table referenced here
US20260196349A1-20260709-T00011
Please refer to the end of the specification for access instructions.

Lengthy table referenced here
US20260196349A1-20260709-T00012
Please refer to the end of the specification for access instructions.

Lengthy table referenced here
US20260196349A1-20260709-T00013
Please refer to the end of the specification for access instructions.

Lengthy table referenced here
US20260196349A1-20260709-T00014
Please refer to the end of the specification for access instructions.

Lengthy table referenced here
US20260196349A1-20260709-T00015
Please refer to the end of the specification for access instructions.

Lengthy table referenced here
US20260196349A1-20260709-T00016
Please refer to the end of the specification for access instructions.

Lengthy table referenced here
US20260196349A1-20260709-T00017
Please refer to the end of the specification for access instructions.

Lengthy table referenced here
US20260196349A1-20260709-T00018
Please refer to the end of the specification for access instructions.

Lengthy table referenced here
US20260196349A1-20260709-T00019
Please refer to the end of the specification for access instructions.

Lengthy table referenced here
US20260196349A1-20260709-T00020
Please refer to the end of the specification for access instructions.

Lengthy table referenced here
US20260196349A1-20260709-T00021
Please refer to the end of the specification for access instructions.

Lengthy table referenced here
US20260196349A1-20260709-T00022
Please refer to the end of the specification for access instructions.

Lengthy table referenced here
US20260196349A1-20260709-T00023
Please refer to the end of the specification for access instructions.

Lengthy table referenced here
US20260196349A1-20260709-T00024
Please refer to the end of the specification for access instructions.

Lengthy table referenced here
US20260196349A1-20260709-T00025
Please refer to the end of the specification for access instructions.

Lengthy table referenced here
US20260196349A1-20260709-T00026
Please refer to the end of the specification for access instructions.

Lengthy table referenced here
US20260196349A1-20260709-T00027
Please refer to the end of the specification for access instructions.

Lengthy table referenced here
US20260196349A1-20260709-T00028
Please refer to the end of the specification for access instructions.

Lengthy table referenced here
US20260196349A1-20260709-T00029
Please refer to the end of the specification for access instructions.

Lengthy table referenced here
US20260196349A1-20260709-T00030
Please refer to the end of the specification for access instructions.

Lengthy table referenced here
US20260196349A1-20260709-T00031
Please refer to the end of the specification for access instructions.

Lengthy table referenced here
US20260196349A1-20260709-T00032
Please refer to the end of the specification for access instructions.

Lengthy table referenced here
US20260196349A1-20260709-T00033
Please refer to the end of the specification for access instructions.

Lengthy table referenced here
US20260196349A1-20260709-T00034
Please refer to the end of the specification for access instructions.

Lengthy table referenced here
US20260196349A1-20260709-T00035
Please refer to the end of the specification for access instructions.

Lengthy table referenced here
US20260196349A1-20260709-T00036
Please refer to the end of the specification for access instructions.

Lengthy table referenced here
US20260196349A1-20260709-T00037
Please refer to the end of the specification for access instructions.

Lengthy table referenced here
US20260196349A1-20260709-T00038
Please refer to the end of the specification for access instructions.

Lengthy table referenced here
US20260196349A1-20260709-T00039
Please refer to the end of the specification for access instructions.

Lengthy table referenced here
US20260196349A1-20260709-T00040
Please refer to the end of the specification for access instructions.

Lengthy table referenced here
US20260196349A1-20260709-T00041
Please refer to the end of the specification for access instructions.

Lengthy table referenced here
US20260196349A1-20260709-T00042
Please refer to the end of the specification for access instructions.

Lengthy table referenced here
US20260196349A1-20260709-T00043
Please refer to the end of the specification for access instructions.

Lengthy table referenced here
US20260196349A1-20260709-T00044
Please refer to the end of the specification for access instructions.

Lengthy table referenced here
US20260196349A1-20260709-T00045
Please refer to the end of the specification for access instructions.

Lengthy table referenced here
US20260196349A1-20260709-T00046
Please refer to the end of the specification for access instructions.

Lengthy table referenced here
US20260196349A1-20260709-T00047
Please refer to the end of the specification for access instructions.

Lengthy table referenced here
US20260196349A1-20260709-T00048
Please refer to the end of the specification for access instructions.

Lengthy table referenced here
US20260196349A1-20260709-T00049
Please refer to the end of the specification for access instructions.

Lengthy table referenced here
US20260196349A1-20260709-T00050
Please refer to the end of the specification for access instructions.

Lengthy table referenced here
US20260196349A1-20260709-T00051
Please refer to the end of the specification for access instructions.

Lengthy table referenced here
US20260196349A1-20260709-T00052
Please refer to the end of the specification for access instructions.

Lengthy table referenced here
US20260196349A1-20260709-T00053
Please refer to the end of the specification for access instructions.

Lengthy table referenced here
US20260196349A1-20260709-T00054
Please refer to the end of the specification for access instructions.

Lengthy table referenced here
US20260196349A1-20260709-T00055
Please refer to the end of the specification for access instructions.

Lengthy table referenced here
US20260196349A1-20260709-T00056
Please refer to the end of the specification for access instructions.

Lengthy table referenced here
US20260196349A1-20260709-T00057
Please refer to the end of the specification for access instructions.

Lengthy table referenced here
US20260196349A1-20260709-T00058
Please refer to the end of the specification for access instructions.

Lengthy table referenced here
US20260196349A1-20260709-T00059
Please refer to the end of the specification for access instructions.

Lengthy table referenced here
US20260196349A1-20260709-T00060
Please refer to the end of the specification for access instructions.

Lengthy table referenced here
US20260196349A1-20260709-T00061
Please refer to the end of the specification for access instructions.

Lengthy table referenced here
US20260196349A1-20260709-T00062
Please refer to the end of the specification for access instructions.

Lengthy table referenced here
US20260196349A1-20260709-T00063
Please refer to the end of the specification for access instructions.

Lengthy table referenced here
US20260196349A1-20260709-T00064
Please refer to the end of the specification for access instructions.

Lengthy table referenced here
US20260196349A1-20260709-T00065
Please refer to the end of the specification for access instructions.

### EMBODIMENTS

Select embodiments of the present invention are as follows:

Embodiment 1. A method of evaluating a subject for cancer comprising:
(I) determining in a biological sample from the subject a concentration of one or more proteins selected from Table 101 or selected from Table 1001;
(II) determining in a biological sample from the subject a concentration of one or more proteins selected from each of Tables 201-218, or selected from each of Tables 2001-2018; or determining in a biological sample from the subject a concentration of each protein from Table 251, 252, 253, or 254;
thereby evaluating the subject for cancer,
wherein the sample has been separated from the subject at the time of the determination in (I) or (II).

Embodiment 2. The method of Embodiment 1, wherein (I) is performed prior to (II).

Embodiment 3. The method of Embodiment 1, wherein the performance of (I) overlaps with the performance of (II).

Embodiment 4. The method of any one of Embodiments 1-3, further comprising, in (I), applying a classifier to the concentration of the one or more proteins that identifies whether the concentration of the one or more proteins selected from Table 101 or selected from Table 1001 is indicative of the subject having cancer.

Embodiment 5. The method of any one of Embodiments 1-4, further comprising, in (II), applying a classifier to the concentration of the one or more proteins that identifies whether the concentration of one two or more proteins selected from each of Tables 201-218, or selected from each of Tables 2001-2018 is indicative of tissue with which the cancer is associated: or applying a classifier to the concentration of each protein that identifies whether the concentration of the proteins of Table 251, 252, 253, or 254 is indicative of tissue with which the cancer is associated.

Embodiment 6. The method of any one of Embodiments 1-5, further comprising administering a treatment to the subject.

Embodiment 7. The method of any one of Embodiments 1-6, wherein the cancer comprises a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 8. The method of any one of Embodiments 1-6, wherein the cancer comprises bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

Embodiment 9. A method of treating cancer in a subject, comprising
(a) acquiring results from the method of any one of Embodiments 1-5; and
(b) administering a treatment to the subject.

Embodiment 10. The method of Embodiment 9, wherein the treatment is responsive to the results acquired in (a).

Embodiment 11. The method of Embodiment 9 or 10, wherein (a) comprises
(I) determining in a biological sample from the subject a concentration of two or more proteins selected from Table 101 or selected from Table 1001, and applying a classifier to the concentration of the two or more proteins to identify that the subject has cancer; and
(II) determining in a biological sample from the subject a concentration of two or more proteins selected from each of Tables 201-218, or selected from each of Tables 2001-2018, and applying a classifier to the concentration of the two or more proteins to identify with which tissue the cancer is associated; or determining in a biological sample from the subject a concentration of each protein of Table 251, 252, 253, or 254, and applying a classifier to the concentration to identify with which tissue the cancer is associated.

Embodiment 12. A method of treating cancer in a subject, comprising
(a) acquiring results from an evaluation of the subject that determined to which tissue the cancer is associated; and
(b) administering a treatment to the subject,
wherein the evaluation comprises:
   (I) determining in a biological sample from the subject a concentration of one or more proteins selected from Table 101 or selected from Table 1001, and applying a classifier to the concentration of the one or more proteins to identify that the subject has cancer;
   (II) determining in a biological sample from the subject a concentration of one or more proteins selected from each of Tables 201-218, or selected from each of Tables 2001-2018, and applying a classifier to the concentration of the one or more proteins to identify with which tissue the cancer is associated; or determining in a biological sample from the subject a concentration of each of the proteins of Tables 251, 252, 253, or 254, and applying a classifier to the concentration of each protein to identify with which tissue the cancer is associated.

Embodiment 13. The method of any one of Embodiments 9-12, wherein the results in (a) are acquired from a third party.

Embodiment 14. The method of any one of Embodiments 1-13, wherein the cancer is early-stage.

Embodiment 15. The method of any one of Embodiments 1-14, wherein in (I), the one or more proteins selected from Table 101 are selected from Table 101.A.1, 101.A.2, 101.A.3, or 101.A.4.

Embodiment 16. The method of any one of Embodiments 1-14, wherein in (I), the one or more proteins selected from Table 1001 are selected from Table 1001.A.1, 1001.A.2, 1001.A.3, or 1001.A.4.

Embodiment 17. The method of any one of Embodiments 1-14, wherein in (I), the one or more proteins selected from Table 101 are selected from Table 101.B.1, 101.B.2, 101.B.3, or 101.B.4.

Embodiment 18. The method of any one of Embodiments 1-14, wherein in (I), the one or more proteins selected from Table 1001 are selected from Table 1001.B.1, 1001.B.2, 1001.B.3, or 1001.B.4.

Embodiment 19. The method of Embodiment 17 or 18, wherein the subject is female.

Embodiment 20. The method of any one of Embodiments 1-14, wherein in (I), the one or more proteins selected from Table 101 are selected from Table 101.C.1, 101.C.2, 101.C.3, or 101.C.4.

Embodiment 21. The method of any one of Embodiments 1-14, wherein the one or more proteins selected from Table 1001 are selected from Table 1001.C.1, 1001.C.2, 1001.C.3, or 1001.C.4.

Embodiment 22. The method of Embodiment 20 or 21, wherein the subject is male.

Embodiment 23. The method of any one of Embodiments 1-22, wherein, in (I), two or more proteins are selected from Table 101 or selected from Table 1001.

Embodiment 24. The method of Embodiment 23, wherein, in (I), three or more proteins are selected from Table 101 or selected from Table 1001.

Embodiment 25. The method of Embodiment 24, wherein, in (I), five or more proteins are selected from Table 101 or selected from Table 1001.

Embodiment 26. The method of Embodiment 25, wherein, in (I), ten or more proteins are selected from Table 101 or selected from Table 1001.

Embodiment 27. The method of any one of Embodiments 1-26, wherein, in (I), no more than about 300 proteins are selected from Table 101 or selected from Table 1001.

Embodiment 28. The method of Embodiment 27, wherein, in (I), no more than about 200 proteins are selected from Table 101 or selected from Table 1001.

Embodiment 29. The method of Embodiment 28, wherein, in (I), no more than about 100 proteins are selected from Table 101 or selected from Table 1001.

Embodiment 30. The method of Embodiment 29, wherein, in (I), no more than about 50 proteins are selected from Table 101 or selected from Table 1001.

Embodiment 31. The method of Embodiment 30, wherein, in (I), no more than about 25 proteins are selected from Table 101 or selected from Table 1001.

Embodiment 32. The method of any one of Embodiments 1-14, wherein, in (I), the one or more proteins selected from Table 101 is every protein of Table 101.A.1, 101.A.2, 101.A.3, or 101.A.4.

Embodiment 33. The method of any one of Embodiments 1-14, wherein, in (I), the one or more proteins selected from Table 1001 is every protein of Table 1001.A.1, 1001.A.2, 1001.A.3, or 1001.A.4.

Embodiment 34. The method of Embodiment 19, wherein, in (I), the one or more proteins selected from Table 101 is every protein of Table 101.B.1, 101.B.2, 101.B.3, or 101.B.4.

Embodiment 35. The method of Embodiment 19, wherein, in (I), the one or more proteins selected from Table 1001 is every protein of Table 1001.B.1, 1001.B.2, 1001.B.3, or 1001.B.4.

Embodiment 36. The method of Embodiment 22, wherein, in (I), the one or more proteins selected from Table 101 is every protein of Table 101.C.1, 101.C.2, 101.C.3, or 101.C.4.

Embodiment 37. The method of Embodiment 22, wherein, in (I), the one or more proteins selected from Table 1001 is every protein of Table 1001.C.1, 1001.C.2, 1001.C.3, or 1001.C.4.

Embodiment 38. The method of any one of Embodiments 1-37, wherein in (II), the one or more proteins selected from Table 201 are selected from Table 201.A.1 or 201.A.2.

Embodiment 39. The method of any one of Embodiments 1-37, wherein in (II), the one or more proteins selected from Table 2001 are selected from Table 2001.A.1, 2001.A.2, or 2001.A.3.

Embodiment 40. The method of any one of Embodiments 1-37, wherein in (II), the one or more proteins selected from Table 201 are selected from Table 201.B.1, 201.B.2, 201.B.3, or 201.B.4.

Embodiment 41. The method of any one of Embodiments 1-37, wherein in (II), the one or more proteins selected from Table 2001 are selected from Table 2001.B.1, 2001.B.2, or 2001.B.3.

Embodiment 42. The method of Embodiment 40 or 41, wherein the subject is female.

Embodiment 43. The method of any one of Embodiments 1-37, wherein in (II), the one or more proteins selected from Table 201 are selected from Table 201.C.1, 201.C.2, 201.C.3, or 201.C.4.

Embodiment 44. The method of any one of Embodiments 1-37, wherein in (II), the one or more proteins selected from Table 2001 are selected from Table 2001.C.1, 2001.C.2, or 2001.C.3.

Embodiment 45. The method of Embodiment 43 or 44, wherein the subject is male.

Embodiment 46. The method of any one of Embodiments 1-45, wherein, in (II), two or more proteins are selected from Table 201 or selected from Table 2001.

Embodiment 47. The method of Embodiment 46, wherein, in (II), three or more proteins are selected from Table 201 or selected from Table 2001.

Embodiment 48. The method of Embodiment 47, wherein, in (II), five or more proteins are selected from Table 201 or selected from Table 2001.

Embodiment 49. The method of Embodiment 48, wherein, in (II), ten or more proteins are selected from Table 201 or selected from Table 2001.

Embodiment 50. The method of any one of Embodiments 1-49, wherein, in (II), no more than about 300 proteins are selected from Table 201 or selected from Table 2001.

Embodiment 51. The method of Embodiment 50, wherein, in (II), no more than about 200 proteins are selected from Table 201 or selected from Table 2001.

Embodiment 52. The method of Embodiment 51, wherein, in (II), no more than about 100 proteins are selected from Table 201 or selected from Table 2001.

Embodiment 53. The method of Embodiment 52, wherein, in (II), no more than about 50 proteins are selected from Table 201 or selected from Table 2001.

Embodiment 54. The method of Embodiment 53, wherein, in (II), no more than about 25 proteins are selected from Table 201 or selected from Table 2001.

Embodiment 55. The method of any one of Embodiments 1-37, wherein, in (II), the one or more proteins selected from Table 201 is every protein of Table 201.A.1 or 201.A.2.

Embodiment 56. The method of any one of Embodiments 1-37, wherein, in (II), the one or more proteins selected from Table 2001 is every protein of Table 2001.A.1, 2001.A.2, or 2001.A.3.

Embodiment 57. The method of Embodiment 42, wherein, in (II), the one or more proteins selected from Table 201 is every protein of Table 201.B.1, 201.B.2, 201.B.3, or 201.B.4.

Embodiment 58. The method of Embodiment 42, wherein, in (II), the one or more proteins selected from Table 2001 is every protein of Table 2001.B.1, 2001.B.2, or 2001.B.3.

Embodiment 59. The method of Embodiment 45, wherein, in (II), the one or more proteins selected from Table 201 is every protein of Table 201.C.1, 201.C.2, 201.C.3, or 201.C.4.

Embodiment 60. The method of Embodiment 45, wherein, in (II), the one or more proteins selected from Table 2001 is every protein of Table 2001.C.1, 2001.C.2, or 2001.C.3.

Embodiment 61. The method of any one of Embodiments 1-60, wherein in (II), the one or more proteins selected from Table 202 are selected from Table 202.A.1 or 202.A.2.

Embodiment 62. The method of any one of Embodiments 1-60, wherein in (II), the one or more proteins selected from Table 2002 are selected from Table 2002.A.1, 2002.A.2, or 2002.A.3.

Embodiment 63. The method of any one of Embodiments 1-60, wherein in (II), the one or more proteins selected from Table 202 are selected from Table 202.B.1, 202.B.2, 202.B.3, or 202.B.4.

Embodiment 64. The method of any one of Embodiments 1-60, wherein in (II), the one or more proteins selected from Table 2002 are selected from Table 2002.B.1, 2002.B.2, or 2002.B.3.

Embodiment 65. The method of Embodiment 63 or 64, wherein the subject is female.

Embodiment 66. The method of any one of Embodiments 1-60, wherein in (II), the one or more proteins selected from Table 202 are selected from Table 202.C.1, 202.C.2, or 202.C.3.

Embodiment 67. The method of any one of Embodiments 1-60, wherein in (II), the one or more proteins selected from Table 2002 are selected from Table 2002.C.1, 2002.C.2, or 2002.C.3.

Embodiment 68. The method of Embodiment 66 or 67, wherein the subject is male.

Embodiment 69. The method of any one of Embodiments 1-68, wherein, in (II), two or more proteins are selected from Table 202 or selected from Table 2002.

Embodiment 70. The method of Embodiment 69, wherein, in (II), three or more proteins are selected from Table 202 or selected from Table 2002.

Embodiment 71. The method of Embodiment 70, wherein, in (II), five or more proteins are selected from Table 202 or selected from Table 2002.

Embodiment 72. The method of Embodiment 71, wherein, in (II), ten or more proteins are selected from Table 202 or selected from Table 2002.

Embodiment 73. The method of any one of Embodiments 1-72, wherein, in (II), no more than about 300 proteins are selected from Table 202 or selected from Table 2002.

Embodiment 74. The method of Embodiment 73, wherein, in (II), no more than about 200 proteins are selected from Table 202 or selected from Table 2002.

Embodiment 75. The method of Embodiment 74, wherein, in (II), no more than about 100 proteins are selected from Table 202 or selected from Table 2002.

Embodiment 76. The method of Embodiment 75, wherein, in (II), no more than about 50 proteins are selected from Table 202 or selected from Table 2002.

Embodiment 77. The method of Embodiment 76, wherein, in (II), no more than about 25 proteins are selected from Table 202 or selected from Table 2002.

Embodiment 78. The method of any one of Embodiments 1-60, wherein, in (II), the one or more proteins selected from Table 202 is every protein of Table 202.A.1 or 202.A.2.

Embodiment 79. The method of any one of Embodiments 1-60, wherein, in (II), the one or more proteins selected from Table 2002 is every protein of Table 2002.A.1, 2002.A.2, or 2002.A.3.

Embodiment 80. The method of Embodiment 65, wherein, in (II), the one or more proteins selected from Table 202 is every protein of Table 202.B.1, 202.B.2, 202.B.3, or 202.B.4.

Embodiment 81. The method of Embodiment 65, wherein, in (II), the one or more proteins selected from Table 2002 is every protein of Table 2002.B.1, 2002.B.2, or 2002.B.3.

Embodiment 82. The method of Embodiment 68, wherein, in (II), the one or more proteins selected from Table 202 is every protein of Table 202.C.1, 202.C.2, or 202.C.3.

Embodiment 83. The method of Embodiment 68, wherein, in (II), the one or more proteins selected from Table 2002 is every protein of Table 2002.C.1, 2002.C.2, or 2002.C.3.

Embodiment 84. The method of any one of Embodiments 1-83, wherein in (II), the one or more proteins selected from Table 203 are selected from Table 203.A.1 or 203.A.2.

Embodiment 85. The method of any one of Embodiments 1-83, wherein in (II), the one or more proteins selected from Table 2003 are selected from Table 2003.A.1, 2003.A.2, or 2003.A.3.

Embodiment 86. The method of any one of Embodiments 1-83, wherein in (II), the one or more proteins selected from Table 203 are selected from Table 203.B.1, 203.B.2, 203.B.3, or 203.B.4.

Embodiment 87. The method of any one of Embodiments 1-83, wherein in (II), the one or more proteins selected from Table 2003 are selected from Table 2003.B.1, 2003.B.2, or 2003.B.3.

Embodiment 88. The method of Embodiment 86 or 87, wherein the subject is female.

Embodiment 89. The method of any one of Embodiments 1-83, wherein in (II), the one or more proteins selected from Table 203 are selected from Table 203.C.1, 203.C.2, 203.C.3, or 204.C.4.

Embodiment 90. The method of any one of Embodiments 1-83, wherein in (II), the one or more proteins selected from Table 2003 are selected from Table 2003.C.1, 2003.C.2, or 2003.C.3.

Embodiment 91. The method of Embodiment 89 or 90, wherein the subject is male.

Embodiment 92. The method of any one of Embodiments 1-91, wherein, in (II), two or more proteins are selected from Table 203 or selected from Table 2003.

Embodiment 93. The method of Embodiment 92, wherein, in (II), three or more proteins are selected from Table 203 or selected from Table 2003.

Embodiment 94. The method of Embodiment 93, wherein, in (II), five or more proteins are selected from Table 203 or selected from Table 2003.

Embodiment 95. The method of Embodiment 94, wherein, in (II), ten or more proteins are selected from Table 203 or selected from Table 2003.

Embodiment 96. The method of any one of Embodiments 1-95, wherein, in (II), no more than about 300 proteins are selected from Table 203 or selected from Table 2003.

Embodiment 97. The method of Embodiment 96, wherein, in (II), no more than about 200 proteins are selected from Table 203 or selected from Table 2003.

Embodiment 98. The method of Embodiment 97, wherein, in (II), no more than about 100 proteins are selected from Table 203 or selected from Table 2003.

Embodiment 99. The method of Embodiment 98, wherein, in (II), no more than about 50 proteins are selected from Table 203 or selected from Table 2003.

Embodiment 100. The method of Embodiment 99, wherein, in (II), no more than about 25 proteins are selected from Table 203 or selected from Table 2003.

Embodiment 101. The method of any one of Embodiments 1-83, wherein, in (II), the one or more proteins selected from Table 203 is every protein of Table 203.A.1 or 203.A.2.

Embodiment 102. The method of any one of Embodiments 1-83, wherein, in (II), the one or more proteins selected from Table 2003 is every protein of Table 2003.A.1, 2003.A.2, or 2003.A.3.

Embodiment 103. The method of Embodiment 88, wherein, in (II), the one or more proteins selected from Table 203 is every protein of Table 203.B.1, 203.B.2, 203.B.3, or 203.B.4.

Embodiment 104. The method of Embodiment 88, wherein, in (II), the one or more proteins selected from Table 2003 is every protein of Table 2003.B.1, 2003.B.2, or 2003.B.3.

Embodiment 105. The method of Embodiment 91, wherein, in (II), the one or more proteins selected from Table 203 is every protein of Table 203.C.1, 203.C.2, 203.C.3, or 203.C.4.

Embodiment 106. The method of Embodiment 91, wherein, in (II), the one or more proteins selected from Table 2003 is every protein of Table 2003.C.1, 2003.C.2, or 2003.C.3.

Embodiment 107. The method of any one of Embodiments 1-106, wherein in (II), the one or more proteins selected from Table 204 are selected from Table 204.A.1 or 204.A.2.

Embodiment 108. The method of any one of Embodiments 1-106, wherein in (II), the one or more proteins selected from Table 2004 are selected from Table 2004.A.1.

Embodiment 109. The method of any one of Embodiments 1-106, wherein in (II), the one or more proteins selected from Table 204 are selected from Table 204.B.1, 204.B.2, 204.B.3, or 204.B.4.

Embodiment 110. The method of any one of Embodiments 1-106, wherein in (II), the one or more proteins selected from Table 2004 are selected from Table 2004.A.1, 2004.A.2, or 2004.A.3.

Embodiment 111. The method of Embodiment 109 or 110, wherein the subject is female.

Embodiment 112. The method of any one of Embodiments 1-111, wherein, in (II), two or more proteins are selected from Table 204 or selected from Table 2004.

Embodiment 113. The method of Embodiment 112, wherein, in (II), three or more proteins are selected from Table 204 or selected from Table 2004.

Embodiment 114. The method of Embodiment 113, wherein, in (II), five or more proteins are selected from Table 204 or selected from Table 2004.

Embodiment 115. The method of Embodiment 114, wherein, in (II), ten or more proteins are selected from Table 204 or selected from Table 2004.

Embodiment 116. The method of any one of Embodiments 1-115, wherein, in (II), no more than about 300 proteins are selected from Table 204 or selected from Table 2004.

Embodiment 117. The method of Embodiment 116, wherein, in (II), no more than about 200 proteins are selected from Table 204 or selected from Table 2004.

Embodiment 118. The method of Embodiment 117, wherein, in (II), no more than about 100 proteins are selected from Table 204 or selected from Table 2004.

Embodiment 119. The method of Embodiment 118, wherein, in (II), no more than about 50 proteins are selected from Table 204 or selected from Table 2004.

Embodiment 120. The method of Embodiment 119, wherein, in (II), no more than about 25 proteins are selected from Table 204 or selected from Table 2004.

Embodiment 121. The method of any one of Embodiments 1-106, wherein, in (II), the one or more proteins selected from Table 204 is every protein of Table 204.A.1 or 204.A.2.

Embodiment 122. The method of any one of Embodiments 1-106, wherein, in (II), the one or more proteins selected from Table 2004 is every protein of Table 2004.A.1.

Embodiment 123. The method of Embodiment 111, wherein, in (II), the one or more proteins selected from Table 204 is every protein of Table 204.B.1, 204.B.2, 204.B.3, or 204.B.4.

Embodiment 124. The method of Embodiment 111, wherein, in (II), the one or more proteins selected from Table 2004 is every protein of Table 2004.A.1, 2004.A.2, or 2004.A.3.

Embodiment 125. The method of any one of Embodiments 1-124, wherein in (II), the one or more proteins selected from Table 205 are selected from Table 205.A.1 or 205.A.2.

Embodiment 126. The method of any one of Embodiments 1-124, wherein in (II), the one or more proteins selected from Table 2005 are selected from Table 2005.A.1.

Embodiment 127. The method of any one of Embodiments 1-124, wherein in (II), the one or more proteins selected from Table 205 are selected from Table 205.B.1, 205.B.2, 205.B.3, or 205.B.4.

Embodiment 128. The method of any one of Embodiments 1-124, wherein in (II), the one or more proteins selected from Table 2005 are selected from Table 2005.A.1, 2005.A.2, or 2005.A.3.

Embodiment 129. The method of Embodiment 127 or 128, wherein the subject is female.

Embodiment 130. The method of any one of Embodiments 1-129, wherein, in (II), two or more proteins are selected from Table 205 or selected from Table 2005.

Embodiment 131. The method of Embodiment 130, wherein, in (II), three or more proteins are selected from Table 205 or selected from Table 2005.

Embodiment 132. The method of Embodiment 131, wherein, in (II), five or more proteins are selected from Table 205 or selected from Table 2005.

Embodiment 133. The method of Embodiment 132, wherein, in (II), ten or more proteins are selected from Table 205 or selected from Table 2005.

Embodiment 134. The method of any one of Embodiments 1-133, wherein, in (II), no more than about 300 proteins are selected from Table 205 or selected from Table 2005.

Embodiment 135. The method of Embodiment 134, wherein, in (II), no more than about 200 proteins are selected from Table 205 or selected from Table 2005.

Embodiment 136. The method of Embodiment 135, wherein, in (II), no more than about 100 proteins are selected from Table 205 or selected from Table 2005.

Embodiment 137. The method of Embodiment 136, wherein, in (II), no more than about 50 proteins are selected from Table 205 or selected from Table 2005.

Embodiment 138. The method of Embodiment 137, wherein, in (II), no more than about 25 proteins are selected from Table 205 or selected from Table 2005.

Embodiment 139. The method of any one of Embodiments 1-124, wherein, in (II), the one or more proteins selected from Table 205 is every protein of Table 205.A.1 or 205.A.2.

Embodiment 140. The method of any one of Embodiments 1-124, wherein, in (II), the one or more proteins selected from Table 2005 is every protein of Table 2005.A.1.

Embodiment 141. The method of Embodiment 129, wherein, in (II), the one or more proteins selected from Table 205 is every protein of Table 205.B.1, 205.B.2, 205.B.3, or 205.B.4.

Embodiment 142. The method of Embodiment 129, wherein, in (II), the one or more proteins selected from Table 2005 is every protein of Table 2005.A.1, 2005.A.2, or 2005.A.3.

Embodiment 143. The method of any one of Embodiments 1-142, wherein in (II), the one or more proteins selected from Table 206 are selected from Table 206.A.1 or 206.A.2.

Embodiment 144. The method of any one of Embodiments 1-142, wherein in (II), the one or more proteins selected from Table 2006 are selected from Table 2006.A.1, 2006.A.2, or 2006.A.3.

Embodiment 145. The method of any one of Embodiments 1-142, wherein in (II), the one or more proteins selected from Table 206 are selected from Table 206.B.1, 206.B.2, 206.B.3, or 206.B.4.

Embodiment 146. The method of any one of Embodiments 1-142, wherein in (II), the one or more proteins selected from Table 2006 are selected from Table 2006.B.1, 2006.B.2, or 2006.B.3.

Embodiment 147. The method of Embodiment 145 or 146, wherein the subject is female.

Embodiment 148. The method of any one of Embodiments 1-142, wherein in (II), the one or more proteins selected from Table 206 are selected from Table 206.C.1, 206.C.2, or 206.C.3.

Embodiment 149. The method of any one of Embodiments 1-142, wherein in (II), the one or more proteins selected from Table 2006 are selected from Table 2006.C.1, 2006.C.2, or 2006.C.3.

Embodiment 150. The method of Embodiment 148 or 149, wherein the subject is male.

Embodiment 151. The method of any one of Embodiments 1-150, wherein, in (II), two or more proteins are selected from Table 206 or selected from Table 2006.

Embodiment 152. The method of Embodiment 151, wherein, in (II), three or more proteins are selected from Table 206 or selected from Table 2006.

Embodiment 153. The method of Embodiment 152, wherein, in (II), five or more proteins are selected from Table 206 or selected from Table 2006.

Embodiment 154. The method of Embodiment 153, wherein, in (II), ten or more proteins are selected from Table 206 or selected from Table 2006.

Embodiment 155. The method of any one of Embodiments 1-154, wherein, in (II), no more than about 300 proteins are selected from Table 206 or selected from Table 2006.

Embodiment 156. The method of Embodiment 155, wherein, in (II), no more than about 200 proteins are selected from Table 206 or selected from Table 2006.

Embodiment 157. The method of Embodiment 156, wherein, in (II), no more than about 100 proteins are selected from Table 206 or selected from Table 2006.

Embodiment 158. The method of Embodiment 157, wherein, in (II), no more than about 50 proteins are selected from Table 206 or selected from Table 2006.

Embodiment 159. The method of Embodiment 158, wherein, in (II), no more than about 25 proteins are selected from Table 206 or selected from Table 2006.

Embodiment 160. The method of any one of Embodiments 1-142, wherein, in (II), the one or more proteins selected from Table 206 is every protein of Table 206.A.1 or 206.A.2.

Embodiment 161. The method of any one of Embodiments 1-142, wherein, in (II), the one or more proteins selected from Table 2006 is every protein of Table 2006.A.1, 2006.A.2, or 2006.A.3.

Embodiment 162. The method of Embodiment 147, wherein, in (II), the one or more proteins selected from Table 206 is every protein of Table 206.B.1, 206.B.2, 206.B.3, or 206.B.4.

Embodiment 163. The method of Embodiment 147, wherein, in (II), the one or more proteins selected from Table 2006 is every protein of Table 2006.B.1, 2006.B.2, or 2006.B.3.

Embodiment 164. The method of Embodiment 150, wherein, in (II), the one or more proteins selected from Table 206 is every protein of Table 206.C.1, 206.C.2, or 206.C.3.

Embodiment 165. The method of Embodiment 150, wherein, in (II), the one or more proteins selected from Table 2006 is every protein of Table 2006.C.1, 2006.C.2, or 2006.C.3.

Embodiment 166. The method of any one of Embodiments 1-165, wherein in (II), the one or more proteins selected from Table 207 are selected from Table 207.A.1 or 207.A.2.

Embodiment 167. The method of any one of Embodiments 1-165, wherein in (II), the one or more proteins selected from Table 2007 are selected from Table 2007.A.1, 2007.A.2, or 2007.A.3.

Embodiment 168. The method of any one of Embodiments 1-165, wherein in (II), the one or more proteins selected from Table 207 are selected from Table 207.B.1, 207.B.2, 207.B.3, or 207.B.4.

Embodiment 169. The method of any one of Embodiments 1-165, wherein in (II), the one or more proteins selected from Table 2007 are selected from Table 2007.B.1, 2007.B.2, or 2007.B.3.

Embodiment 170. The method of Embodiment 168 or 169, wherein the subject is female.

Embodiment 171. The method of any one of Embodiments 1-165, wherein in (II), the one or more proteins selected from Table 207 are selected from Table 207.C.1, 207.C.2, or 207.C.3.

Embodiment 172. The method of any one of Embodiments 1-165, wherein in (II), the one or more proteins selected from Table 2007 are selected from Table 2007.C.1, 2007.C.2, or 2007.C.3.

Embodiment 173. The method of Embodiment 171 or 172, wherein the subject is male.

Embodiment 174. The method of any one of Embodiments 1-173, wherein, in (II), two or more proteins are selected from Table 207 or selected from Table 2007.

Embodiment 175. The method of Embodiment 174, wherein, in (II), three or more proteins are selected from Table 207 or selected from Table 2007.

Embodiment 176. The method of Embodiment 175, wherein, in (II), five or more proteins are selected from Table 207 or selected from Table 2007.

Embodiment 177. The method of Embodiment 176, wherein, in (II), ten or more proteins are selected from Table 207 or selected from Table 2007.

Embodiment 178. The method of any one of Embodiments 1-177, wherein, in (II), no more than about 300 proteins are selected from Table 207 or selected from Table 2007.

Embodiment 179. The method of Embodiment 178, wherein, in (II), no more than about 200 proteins are selected from Table 207 or selected from Table 2007.

Embodiment 180. The method of Embodiment 179, wherein, in (II), no more than about 100 proteins are selected from Table 207 or selected from Table 2007.

Embodiment 181. The method of Embodiment 180, wherein, in (II), no more than about 50 proteins are selected from Table 207 or selected from Table 2007.

Embodiment 182. The method of Embodiment 181, wherein, in (II), no more than about 25 proteins are selected from Table 207 or selected from Table 2007.

Embodiment 183. The method of any one of Embodiments 1-165, wherein, in (II), the one or more proteins selected from Table 207 is every protein of Table 207.A.1 or 207.A.2.

Embodiment 184. The method of any one of Embodiments 1-165, wherein, in (II), the one or more proteins selected from Table 2007 is every protein of Table 2007.A.1, 2007.A.2, or 2007.A.3.

Embodiment 185. The method of Embodiment 170, wherein, in (II), the one or more proteins selected from Table 207 is every protein of Table 207.B.1, 207.B.2, 207.B.3, or 207.B.4.

Embodiment 186. The method of Embodiment 170, wherein, in (II), the one or more proteins selected from Table 2007 is every protein of Table 2007.B.1, 2007.B.2, or 2007.B.3.

Embodiment 187. The method of Embodiment 173, wherein, in (II), the one or more proteins selected from Table 207 is every protein of Table 207.C.1, 207.C.2, or 207.C.3.

Embodiment 188. The method of Embodiment 173, wherein, in (II), the one or more proteins selected from Table 2007 is every protein of Table 2007.C.1, 2007.C.2, or 2007.C.3.

Embodiment 189. The method of any one of Embodiments 1-188, wherein in (II), the one or more proteins selected from Table 208 are selected from Table 208.A.1 or 208.A.2.

Embodiment 190. The method of any one of Embodiments 1-188, wherein in (II), the one or more proteins selected from Table 2008 are selected from Table 2008.A.1, 2008.A.2, or 2008.A.3.

Embodiment 191. The method of any one of Embodiments 1-188, wherein in (II), the one or more proteins selected from Table 208 are selected from Table 208.B.1, 208.B.2, 208.B.3, or 208.B.4.

Embodiment 192. The method of any one of Embodiments 1-188, wherein in (II), the one or more proteins selected from Table 2008 are selected from Table 2008.B.1, 2008.B.2, or 2008.B.3.

Embodiment 193. The method of Embodiment 191 or 192, wherein the subject is female.

Embodiment 194. The method of any one of Embodiments 1-188, wherein in (II), the one or more proteins selected from Table 208 are selected from Table 208.C.1, 208.C.2, 208.C.3, or 208.C.4.

Embodiment 195. The method of any one of Embodiments 1-188, wherein in (II), the one or more proteins selected from Table 2008 are selected from Table 2008.C.1, 2008.C.2, or 2008.C.3.

Embodiment 196. The method of Embodiment 194 or 195, wherein the subject is male.

Embodiment 197. The method of any one of Embodiments 1-196, wherein, in (II), two or more proteins are selected from Table 208 or selected from Table 2008.

Embodiment 198. The method of Embodiment 197, wherein, in (II), three or more proteins are selected from Table 208 or selected from Table 2008.

Embodiment 199. The method of Embodiment 198, wherein, in (II), five or more proteins are selected from Table 208 or selected from Table 2008.

Embodiment 200. The method of Embodiment 199, wherein, in (II), ten or more proteins are selected from Table 208 or selected from Table 2008.

Embodiment 201. The method of any one of Embodiments 1-200, wherein, in (II), no more than about 300 proteins are selected from Table 208 or selected from Table 2008.

Embodiment 202. The method of Embodiment 201, wherein, in (II), no more than about 200 proteins are selected from Table 208 or selected from Table 2008.

Embodiment 203. The method of Embodiment 202, wherein, in (II), no more than about 100 proteins are selected from Table 208 or selected from Table 2008.

Embodiment 204. The method of Embodiment 203, wherein, in (II), no more than about 50 proteins are selected from Table 208 or selected from Table 2008.

Embodiment 205. The method of Embodiment 204, wherein, in (II), no more than about 25 proteins are selected from Table 208 or selected from Table 2008.

Embodiment 206. The method of any one of Embodiments 1-188, wherein, in (II), the one or more proteins selected from Table 208 is every protein of Table 208.A.1 or 208.A.2.

Embodiment 207. The method of any one of Embodiments 1-188, wherein, in (II), the one or more proteins selected from Table 2008 is every protein of Table 2008.A.1, 2008.A.2, or 2008.A.3.

Embodiment 208. The method of Embodiment 193, wherein, in (II), the one or more proteins selected from Table 208 is every protein of Table 208.B.1, 208.B.2, 208.B.3, or 208.B.4.

Embodiment 209. The method of Embodiment 193, wherein, in (II), the one or more proteins selected from Table 2008 is every protein of Table 2008.B.1, 2008.B.2, or 2008.B.3.

Embodiment 210. The method of Embodiment 196, wherein, in (II), the one or more proteins selected from Table 208 is every protein of Table 208.C.1, 208.C.2, 208.C.3, or 208.C.4.

Embodiment 211. The method of Embodiment 196, wherein, in (II), the one or more proteins selected from Table 2008 is every protein of Table 2008.C.1, 2008.C.2, or 2008.C.3.

Embodiment 212. The method of any one of Embodiments 1-211, wherein in (II), the one or more proteins selected from Table 209 are selected from Table 209.A.1 or 209.A.2.

Embodiment 213. The method of any one of Embodiments 1-211 wherein in (II), the one or more proteins selected from Table 2009 are selected from Table 2009.A.1, 2009.A.2, or 2009.A.3.

Embodiment 214. The method of any one of Embodiments 1-211, wherein in (II), the one or more proteins selected from Table 209 are selected from Table 209.B.1, 209.B.2, 209.B.3, or 209.B.4.

Embodiment 215. The method of any one of Embodiments 1-211, wherein in (II), the one or more proteins selected from Table 2009 are selected from Table 2009.B.1, 2009.B.2, or 2009.B.3.

Embodiment 216. The method of Embodiment 214 or 215, wherein the subject is female.

Embodiment 217. The method of any one of Embodiments 1-211, wherein in (II), the one or more proteins selected from Table 209 are selected from Table 209.C.1, 209.C.2, 209.C.3, or 209.C.4.

Embodiment 218. The method of any one of Embodiments 1-211, wherein in (II), the one or more proteins selected from Table 2009 are selected from Table 2009.C.1, 2009.C.2, or 2009.C.3.

Embodiment 219. The method of Embodiment 217 or 218., wherein the subject is male.

Embodiment 220. The method of any one of Embodiments 1-219, wherein, in (II), two or more proteins are selected from Table 209 or selected from Table 2009.

Embodiment 221. The method of Embodiment 220, wherein, in (II), three or more proteins are selected from Table 209 or selected from Table 2009.

Embodiment 222. The method of Embodiment 221, wherein, in (II), five or more proteins are selected from Table 209 or selected from Table 2009.

Embodiment 223. The method of Embodiment 222, wherein, in (II), ten or more proteins are selected from Table 209 or selected from Table 2009.

Embodiment 224. The method of any one of Embodiments 1-223, wherein, in (II), no more than about 300 proteins are selected from Table 209 or selected from Table 2009.

Embodiment 225. The method of Embodiment 224, wherein, in (II), no more than about 200 proteins are selected from Table 209 or selected from Table 2009.

Embodiment 226. The method of Embodiment 225, wherein, in (II), no more than about 100 proteins are selected from Table 209 or selected from Table 2009.

Embodiment 227. The method of Embodiment 226, wherein, in (II), no more than about 50 proteins are selected from Table 209 or selected from Table 2009.

Embodiment 228. The method of Embodiment 227, wherein, in (II), no more than about 25 proteins are selected from Table 209 or selected from Table 2009.

Embodiment 229. The method of any one of Embodiments 1-211, wherein, in (II), the one or more proteins selected from Table 209 is every protein of Table 209.A.1 or 209.A.2.

Embodiment 230. The method of any one of Embodiments 1-211, wherein, in (II), the one or more proteins selected from Table 2009 is every protein of Table 2009.A.1, 2009.A.2, or 2009.A.3.

Embodiment 231. The method of Embodiment 216, wherein, in (II), the one or more proteins selected from Table 209 is every protein of Table 209.B.1, 209.B.2, 209.B.3, or 209.B.4.

Embodiment 232. The method of Embodiment 216, wherein, in (II), the one or more proteins selected from Table 2009 is every protein of Table 2009.B.1, 2009.B.2, or 2009.B.3.

Embodiment 233. The method of Embodiment 219, wherein, in (II), the one or more proteins selected from Table 209 is every protein of Table 209.C.1, 209.C.2, 209.C.3, or 209.C.4.

Embodiment 234. The method of Embodiment 219, wherein, in (II), the one or more proteins selected from Table 2009 is every protein of Table 2009.C.1, 2009.C.2, or 2009.C.3.

Embodiment 235. The method of any one of Embodiments 1-234, wherein in (II), the one or more proteins selected from Table 210 are selected from Table 210.A.1 or 210.A.2.

Embodiment 236. The method of any one of Embodiments 1-234 wherein in (II), the one or more proteins selected from Table 2010 are selected from Table 2010.A.1, 2010.A.2, or 2010.A.3.

Embodiment 237. The method of any one of Embodiments 1-234, wherein in (II), the one or more proteins selected from Table 210 are selected from Table 210.B.1, 210.B.2, 210.B.3, or 210.B.4.

Embodiment 238. The method of any one of Embodiments 1-234, wherein in (II), the one or more proteins selected from Table 2010 are selected from Table 2010.B.1, 2010.B.2, or 2010.B.3.

Embodiment 239. The method of Embodiment 237 or 238, wherein the subject is female.

Embodiment 240. The method of any one of Embodiments 1-234, wherein in (II), the one or more proteins selected from Table 210 are selected from Table 210.C.1, 210.C.2, 210.C.3, or 210.C.4.

Embodiment 241. The method of any one of Embodiments 1-234, wherein in (II), the one or more proteins selected from Table 2010 are selected from Table 2010.C.1, 2010.C.2, or 2010.C.3.

Embodiment 242. The method of Embodiment 240 or 241, wherein the subject is male.

Embodiment 243. The method of any one of Embodiments 1-242, wherein, in (II), two or more proteins are selected from Table 210 or selected from Table 2010.

Embodiment 244. The method of Embodiment 243, wherein, in (II), three or more proteins are selected from Table 210 or selected from Table 2010.

Embodiment 245. The method of Embodiment 244, wherein, in (II), five or more proteins are selected from Table 210 or selected from Table 2010.

Embodiment 246. The method of Embodiment 245, wherein, in (II), ten or more proteins are selected from Table 210 or selected from Table 2010.

Embodiment 247. The method of any one of Embodiments 1-246, wherein, in (II), no more than about 300 proteins are selected from Table 210 or selected from Table 2010.

Embodiment 248. The method of Embodiment 247, wherein, in (II), no more than about 200 proteins are selected from Table 210 or selected from Table 2010.

Embodiment 249. The method of Embodiment 248, wherein, in (II), no more than about 100 proteins are selected from Table 210 or selected from Table 2010.

Embodiment 250. The method of Embodiment 249, wherein, in (II), no more than about 50 proteins are selected from Table 210 or selected from Table 2010.

Embodiment 251. The method of Embodiment 250, wherein, in (II), no more than about 25 proteins are selected from Table 210 or selected from Table 2010.

Embodiment 252. The method of any one of Embodiments 1-234, wherein, in (II), the one or more proteins selected from Table 210 is every protein of Table 210.A.1 or 210.A.2.

Embodiment 253. The method of any one of Embodiments 1-234, wherein, in (II), the one or more proteins selected from Table 2010 is every protein of Table 2010.A.1, 2010.A.2, or 2010.A.3.

Embodiment 254. The method of Embodiment 239, wherein, in (II), the one or more proteins selected from Table 210 is every protein of Table 210.B.1, 210.B.2, 210.B.3, or 210.B.4.

Embodiment 255. The method of Embodiment 239, wherein, in (II), the one or more proteins selected from Table 2010 is every protein of Table 2010.B.1, 2010.B.2, or 2010.B.3.

Embodiment 256. The method of Embodiment 242, wherein, in (II), the one or more proteins selected from Table 210 is every protein of Table 210.C.1, 210.C.2, 210.C.3, or 210.C.4.

Embodiment 257. The method of Embodiment 242, wherein, in (II), the one or more proteins selected from Table 2010 is every protein of Table 2010.C.1, 2010.C.2, or 2010.C.3.

Embodiment 258. The method of any one of Embodiments 1-257, wherein in (II), the one or more proteins selected from Table 211 are selected from Table 211.A.1 or 211.A.2.

Embodiment 259. The method of any one of Embodiments 1-257 wherein in (II), the one or more proteins selected from Table 2011 are selected from Table 2011.A.1 or 2011.A.2.

Embodiment 260. The method of any one of Embodiments 1-257, wherein in (II), the one or more proteins selected from Table 211 are selected from Table 211.B.1, 211.B.2, 211.B.3, or 211.B.4.

Embodiment 261. The method of any one of Embodiments 1-257, wherein in (II), the one or more proteins selected from Table 2011 are selected from Table 2011.B.1, 2011.B.2, or 2011.B.3.

Embodiment 262. The method of Embodiment 260 or 261, wherein the subject is female.

Embodiment 263. The method of any one of Embodiments 1-257, wherein in (II), the one or more proteins selected from Table 211 are selected from Table 211.C.1, 211.C.2, or 211.C.3.

Embodiment 264. The method of any one of Embodiments 1-257, wherein in (II), the one or more proteins selected from Table 2011 are selected from Table 2011.C.1, 2011.C.2, or 2011.C.3.

Embodiment 265. The method of Embodiment 263 or 264, wherein the subject is male.

Embodiment 266. The method of any one of Embodiments 1-265, wherein, in (II), two or more proteins are selected from Table 211 or selected from Table 2011.

Embodiment 267. The method of Embodiment 266, wherein, in (II), three or more proteins are selected from Table 211 or selected from Table 2011.

Embodiment 268. The method of Embodiment 267, wherein, in (II), five or more proteins are selected from Table 211 or selected from Table 2011.

Embodiment 269. The method of Embodiment 268, wherein, in (II), ten or more proteins are selected from Table 211 or selected from Table 2011.

Embodiment 270. The method of any one of Embodiments 1-269, wherein, in (II), no more than about 300 proteins are selected from Table 211 or selected from Table 2011.

Embodiment 271. The method of Embodiment 270, wherein, in (II), no more than about 200 proteins are selected from Table 211 or selected from Table 2011.

Embodiment 272. The method of Embodiment 271, wherein, in (II), no more than about 100 proteins are selected from Table 211 or selected from Table 2011.

Embodiment 273. The method of Embodiment 272, wherein, in (II), no more than about 50 proteins are selected from Table 211 or selected from Table 2011.

Embodiment 274. The method of Embodiment 273, wherein, in (II), no more than about 25 proteins are selected from Table 211 or selected from Table 2011.

Embodiment 275. The method of any one of Embodiments 1-257, wherein, in (II), the one or more proteins selected from Table 211 is every protein of Table 211.A.1 or 211.A.2.

Embodiment 276. The method of any one of Embodiments 1-257, wherein, in (II), the one or more proteins selected from Table 2011 is every protein of Table 2011.A.1 2011.A.2.

Embodiment 277. The method of Embodiment 262, wherein, in (II), the one or more proteins selected from Table 211 is every protein of Table 211.B.1, 211.B.2, 211.B.3, or 211.B.4.

Embodiment 278. The method of Embodiment 262, wherein, in (II), the one or more proteins selected from Table 2011 is every protein of Table 2011.B.1, 2011.B.2, or 2011.B.3.

Embodiment 279. The method of Embodiment 265, wherein, in (II), the one or more proteins selected from Table 211 is every protein of Table 211.C.1, 211.C.2, or 211.C.3.

Embodiment 280. The method of Embodiment 265, wherein, in (II), the one or more proteins selected from Table 2011 is every protein of Table 2011.C.1, 2011.C.2, or 2011.C.3.

Embodiment 281. The method of any one of Embodiments 1-280, wherein in (II), the one or more proteins selected from Table 212 are selected from Table 212.A.1 or 212.A.2.

Embodiment 282. The method of any one of Embodiments 1-280, wherein in (II), the one or more proteins selected from Table 2012 are selected from Table 2012.A.1.

Embodiment 283. The method of any one of Embodiments 1-280, wherein in (II), the one or more proteins selected from Table 212 are selected from Table 212.B.1, 212.B.2, 212.B.3, or 212.B.4.

Embodiment 284. The method of any one of Embodiments 1-280, wherein in (II), the one or more proteins selected from Table 2012 are selected from Table 2012.A.1, 2012.A.2, or 2012.A.3.

Embodiment 285. The method of Embodiment 283 or 284, wherein the subject is female.

Embodiment 286. The method of any one of Embodiments 1-285, wherein, in (II), two or more proteins are selected from Table 212 or selected from Table 2012.

Embodiment 287. The method of Embodiment 286, wherein, in (II), three or more proteins are selected from Table 212 or selected from Table 2012.

Embodiment 288. The method of Embodiment 287, wherein, in (II), five or more proteins are selected from Table 212 or selected from Table 2012.

Embodiment 289. The method of Embodiment 288, wherein, in (II), ten or more proteins are selected from Table 212 or selected from Table 2012.

Embodiment 290. The method of any one of Embodiments 1-289, wherein, in (II), no more than about 300 proteins are selected from Table 212 or selected from Table 2012.

Embodiment 291. The method of Embodiment 290, wherein, in (II), no more than about 200 proteins are selected from Table 212 or selected from Table 2012.

Embodiment 292. The method of Embodiment 291, wherein, in (II), no more than about 100 proteins are selected from Table 212 or selected from Table 2012.

Embodiment 293. The method of Embodiment 292, wherein, in (II), no more than about 50 proteins are selected from Table 212 or selected from Table 2012.

Embodiment 294. The method of Embodiment 293, wherein, in (II), no more than about 25 proteins are selected from Table 212 or selected from Table 2012.

Embodiment 295. The method of any one of Embodiments 1-280, wherein, in (II), the one or more proteins selected from Table 212 is every protein of Table 212.A.1 or 212.A.2.

Embodiment 296. The method of any one of Embodiments 1-280, wherein, in (II), the one or more proteins selected from Table 2012 is every protein of Table 2012.A.1.

Embodiment 297. The method of Embodiment 285, wherein, in (II), the one or more proteins selected from Table 212 is every protein of Table 212.B.1, 212.B.2, 212.B.3, or 212.B.4.

Embodiment 298. The method of Embodiment 285, wherein, in (II), the one or more proteins selected from Table 2012 is every protein of Table 2012.A.1, 2012.A.2, or 2012.A.3.

Embodiment 299. The method of any one of Embodiments 1-298, wherein in (II), the one or more proteins selected from Table 213 are selected from Table 213.A.1 or 213.A.2.

Embodiment 300. The method of any one of Embodiments 1-298 wherein in (II), the one or more proteins selected from Table 2013 are selected from Table 2013.A.1, 2013.A.2, or 2013.A.3.

Embodiment 301. The method of any one of Embodiments 1-298, wherein in (II), the one or more proteins selected from Table 213 are selected from Table 213.B.1, 213.B.2, or 213.B.3.

Embodiment 302. The method of any one of Embodiments 1-298, wherein in (II), the one or more proteins selected from Table 2013 are selected from Table 2013.B.1, 2013.B.2, or 2013.B.3.

Embodiment 303. The method of Embodiment 301 or 302, wherein the subject is female.

Embodiment 304. The method of any one of Embodiments 1-298, wherein in (II), the one or more proteins selected from Table 213 are selected from Table 213.C.1, 213.C.2, or 213.C.3.

Embodiment 305. The method of any one of Embodiments 1-298, wherein in (II), the one or more proteins selected from Table 2013 are selected from Table 2013.C.1, 2013.C.2, or 2013.C.3.

Embodiment 306. The method of Embodiment 304 or 305, wherein the subject is male.

Embodiment 307. The method of any one of Embodiments 1-306, wherein, in (II), two or more proteins are selected from Table 213 or selected from Table 2013.

Embodiment 308. The method of Embodiment 307, wherein, in (II), three or more proteins are selected from Table 213 or selected from Table 2013.

Embodiment 309. The method of Embodiment 308, wherein, in (II), five or more proteins are selected from Table 213 or selected from Table 2013.

Embodiment 310. The method of Embodiment 309, wherein, in (II), ten or more proteins are selected from Table 213 or selected from Table 2013.

Embodiment 311. The method of any one of Embodiments 1-310, wherein, in (II), no more than about 300 proteins are selected from Table 213 or selected from Table 2013.

Embodiment 312. The method of Embodiment 311, wherein, in (II), no more than about 200 proteins are selected from Table 213 or selected from Table 2013.

Embodiment 313. The method of Embodiment 312, wherein, in (II), no more than about 100 proteins are selected from Table 213 or selected from Table 2013.

Embodiment 314. The method of Embodiment 313, wherein, in (II), no more than about 50 proteins are selected from Table 213 or selected from Table 2013.

Embodiment 315. The method of Embodiment 314, wherein, in (II), no more than about 25 proteins are selected from Table 213 or selected from Table 2013.

Embodiment 316. The method of any one of Embodiments 1-298, wherein, in (II), the one or more proteins selected from Table 213 is every protein of Table 213.A.1 or 213.A.2.

Embodiment 317. The method of any one of Embodiments 1-298, wherein, in (II), the one or more proteins selected from Table 2013 is every protein of Table 2013.A.1, 2013.A.2, or 2013.A.3.

Embodiment 318. The method of Embodiment 303, wherein, in (II), the one or more proteins selected from Table 213 is every protein of Table 213.B.1, 213.B.2, or 213.B.3.

Embodiment 319. The method of Embodiment 303, wherein, in (II), the one or more proteins selected from Table 2013 is every protein of Table 2013.B.1, 2013.B.2, or 2013.B.3.

Embodiment 320. The method of Embodiment 306, wherein, in (II), the one or more proteins selected from Table 213 is every protein of Table 213.C.1, 213.C.2, or 213.C.3.

Embodiment 321. The method of Embodiment 306, wherein, in (II), the one or more proteins selected from Table 2013 is every protein of Table 2013.C.1, 2013.C.2, or 2013.C.3.

Embodiment 322. The method of any one of Embodiments 1-321, wherein in (II), the one or more proteins selected from Table 214 are selected from Table 214.A.1 or 214.A.2.

Embodiment 323. The method of any one of Embodiments 1-321, wherein in (II), the one or more proteins selected from Table 2014 are selected from Table 2014.A.1.

Embodiment 324. The method of any one of Embodiments 1-321, wherein in (II), the one or more proteins selected from Table 214 are selected from Table 214.B.1, 214.B.2, 214.B.3, or 214.B.4.

Embodiment 325. The method of any one of Embodiments 1-321, wherein in (II), the one or more proteins selected from Table 2014 are selected from Table 2014.A.1, 2014.A.2, or 2014.A.3.

Embodiment 326. The method of Embodiment 324 or 325, wherein the subject is male.

Embodiment 327. The method of any one of Embodiments 1-326, wherein, in (II), two or more proteins are selected from Table 214 or selected from Table 2014.

Embodiment 328. The method of Embodiment 327, wherein, in (II), three or more proteins are selected from Table 214 or selected from Table 2014.

Embodiment 329. The method of Embodiment 328, wherein, in (II), five or more proteins are selected from Table 214 or selected from Table 2014.

Embodiment 330. The method of Embodiment 329, wherein, in (II), ten or more proteins are selected from Table 214 or selected from Table 2014.

Embodiment 331. The method of any one of Embodiments 1-330, wherein, in (II), no more than about 300 proteins are selected from Table 214 or selected from Table 2014.

Embodiment 332. The method of Embodiment 331, wherein, in (II), no more than about 200 proteins are selected from Table 214 or selected from Table 2014.

Embodiment 333. The method of Embodiment 332, wherein, in (II), no more than about 100 proteins are selected from Table 214 or selected from Table 2014.

Embodiment 334. The method of Embodiment 333, wherein, in (II), no more than about 50 proteins are selected from Table 214 or selected from Table 2014.

Embodiment 335. The method of Embodiment 334, wherein, in (II), no more than about 25 proteins are selected from Table 214 or selected from Table 2014.

Embodiment 336. The method of any one of Embodiments 1-321, wherein, in (II), the one or more proteins selected from Table 214 is every protein of Table 214.A.1 or 214.A.2.

Embodiment 337. The method of any one of Embodiments 1-321, wherein, in (II), the one or more proteins selected from Table 2014 is every protein of Table 2014.A.1.

Embodiment 338. The method of Embodiment 326, wherein, in (II), the one or more proteins selected from Table 214 is every protein of Table 214.B.1, 214.B.2, 214.B.3, or 214.B.4.

Embodiment 339. The method of Embodiment 326, wherein, in (II), the one or more proteins selected from Table 2014 is every protein of Table 2014.A.1, 2014.A.2, or 2014.A.3.

Embodiment 340. The method of any one of Embodiments 1-339, wherein in (II), the one or more proteins selected from Table 215 are selected from Table 215.A.1 or 215.A.2.

Embodiment 341. The method of any one of Embodiments 1-339 wherein in (II), the one or more proteins selected from Table 2015 are selected from Table 2015.A.1, 2015.A.2, or 2015.A.3.

Embodiment 342. The method of any one of Embodiments 1-339, wherein in (II), the one or more proteins selected from Table 215 are selected from Table 215.B.1, 215.B.2, 215.B.3, or 215.B.4.

Embodiment 343. The method of any one of Embodiments 1-339, wherein in (II), the one or more proteins selected from Table 2015 are selected from Table 2015.B.1, 2015.B.2, or 2015.B.3.

Embodiment 344. The method of Embodiment 342 or 343, wherein the subject is female.

Embodiment 345. The method of any one of Embodiments 1-339, wherein in (II), the one or more proteins selected from Table 215 are selected from Table 215.C.1, 215.C.2, 215.C.3, or 215.C.4.

Embodiment 346. The method of any one of Embodiments 1-339, wherein in (II), the one or more proteins selected from Table 2015 are selected from Table 2015.C.1, 2015.C.2, or 2015.C.3.

Embodiment 347. The method of Embodiment 345 or 346, wherein the subject is male.

Embodiment 348. The method of any one of Embodiments 1-347, wherein, in (II), two or more proteins are selected from Table 215 or selected from Table 2015.

Embodiment 349. The method of Embodiment 348, wherein, in (II), three or more proteins are selected from Table 215 or selected from Table 2015.

Embodiment 350. The method of Embodiment 349, wherein, in (II), five or more proteins are selected from Table 215 or selected from Table 2015.

Embodiment 351. The method of Embodiment 350, wherein, in (II), ten or more proteins are selected from Table 215 or selected from Table 2015.

Embodiment 352. The method of any one of Embodiments 1-351, wherein, in (II), no more than about 300 proteins are selected from Table 215 or selected from Table 2015.

Embodiment 353. The method of Embodiment 352, wherein, in (II), no more than about 200 proteins are selected from Table 215 or selected from Table 2015.

Embodiment 354. The method of Embodiment 353, wherein, in (II), no more than about 100 proteins are selected from Table 215 or selected from Table 2015.

Embodiment 355. The method of Embodiment 354, wherein, in (II), no more than about 50 proteins are selected from Table 215 or selected from Table 2015.

Embodiment 356. The method of Embodiment 355, wherein, in (II), no more than about 25 proteins are selected from Table 215 or selected from Table 2015.

Embodiment 357. The method of any one of Embodiments 1-339, wherein, in (II), the one or more proteins selected from Table 215 is every protein of Table 215.A.1 or 215.A.2.

Embodiment 358. The method of any one of Embodiments 1-339, wherein, in (II), the one or more proteins selected from Table 2015 is every protein of Table 2015.A.1, 2015.A.2, or 2015.A.3.

Embodiment 359. The method of Embodiment 344, wherein, in (II), the one or more proteins selected from Table 215 is every protein of Table 215.B.1, 215.B.2, 215.B.3, or 215.B.4.

Embodiment 360. The method of Embodiment 344, wherein, in (II), the one or more proteins selected from Table 2015 is every protein of Table 2015.B.1, 2015.1B.2, or 2015.1B.3.

Embodiment 361. The method of Embodiment 347, wherein, in (II), the one or more proteins selected from Table 215 is every protein of Table 215.C.1, 215.C.2, 215.C.3, or 215.C.4.

Embodiment 362. The method of Embodiment 347, wherein, in (II), the one or more proteins selected from Table 2015 is every protein of Table 2015.C.1, 2015.C.2, or 2015.C.3.

Embodiment 363. The method of any one of Embodiments 1-362, wherein in (II), the one or more proteins selected from Table 216 are selected from Table 216.A.1 or 216.A.2.

Embodiment 364. The method of any one of Embodiments 1-362, wherein in (II), the one or more proteins selected from Table 2016 are selected from Table 2016.A.1.

Embodiment 365. The method of any one of Embodiments 1-362, wherein in (II), the one or more proteins selected from Table 216 are selected from Table 216.B.1, 216.B.2, 216.B.3, or 216.B.4.

Embodiment 366. The method of any one of Embodiments 1-362, wherein in (II), the one or more proteins selected from Table 2016 are selected from Table 2016.A.1, 2016.A.2, or 2016.A.3.

Embodiment 367. The method of Embodiment 365 or 366, wherein the subject is male.

Embodiment 368. The method of any one of Embodiments 1-367, wherein, in (II), two or more proteins are selected from Table 216 or selected from Table 2016.

Embodiment 369. The method of Embodiment 368, wherein, in (II), three or more proteins are selected from Table 216 or selected from Table 2016.

Embodiment 370. The method of Embodiment 369, wherein, in (II), five or more proteins are selected from Table 216 or selected from Table 2016.

Embodiment 371. The method of Embodiment 370, wherein, in (II), ten or more proteins are selected from Table 216 or selected from Table 2016.

Embodiment 372. The method of any one of Embodiments 1-371, wherein, in (II), no more than about 300 proteins are selected from Table 216 or selected from Table 2016.

Embodiment 373. The method of Embodiment 372, wherein, in (II), no more than about 200 proteins are selected from Table 216 or selected from Table 2016.

Embodiment 374. The method of Embodiment 373, wherein, in (II), no more than about 100 proteins are selected from Table 216 or selected from Table 2016.

Embodiment 375. The method of Embodiment 374, wherein, in (II), no more than about 50 proteins are selected from Table 216 or selected from Table 2016.

Embodiment 376. The method of Embodiment 375, wherein, in (II), no more than about 25 proteins are selected from Table 216 or selected from Table 2016.

Embodiment 377. The method of any one of Embodiments 1-362, wherein, in (II), the one or more proteins selected from Table 216 is every protein of Table 216.A.1 or 216.A.2.

Embodiment 378. The method of any one of Embodiments 1-362, wherein, in (II), the one or more proteins selected from Table 2016 is every protein of Table 2016.A.1.

Embodiment 379. The method of Embodiment 367, wherein, in (II), the one or more proteins selected from Table 216 is every protein of Table 216.B.1, 216.B.2, 216.B.3, or 216.B.4.

Embodiment 380. The method of Embodiment 367, wherein, in (II), the one or more proteins selected from Table 2016 is every protein of Table 2016.A.1, 2016.A.2, or 2016.A.3.

Embodiment 381. The method of any one of Embodiments 1-380, wherein in (II), the one or more proteins selected from Table 217 are selected from Table 217.A.1 or 217.A.2.

Embodiment 382. The method of any one of Embodiments 1-380 wherein in (II), the one or more proteins selected from Table 2017 are selected from Table 2017.A.1, 2017.A.2, or 2017.A.3.

Embodiment 383. The method of any one of Embodiments 1-380, wherein in (II), the one or more proteins selected from Table 217 are selected from Table 217.B.1, 217.B.2, 217.B.3, or 217.B.4.

Embodiment 384. The method of any one of Embodiments 1-380, wherein in (II), the one or more proteins selected from Table 2017 are selected from Table 2017.B.1, 2017.B.2, or 2017.B.3.

Embodiment 385. The method of Embodiment 383 or 384, wherein the subject is female.

Embodiment 386. The method of any one of Embodiments 1-380, wherein in (II), the one or more proteins selected from Table 217 are selected from Table 217.C.1, 217.C.2, 217.C.3, or 217.C.4.

Embodiment 387. The method of any one of Embodiments 1-380, wherein in (II), the one or more proteins selected from Table 2017 are selected from Table 2017.C.1, 2017.C.2, or 2017.C.3.

Embodiment 388. The method of Embodiment 386 or 387, wherein the subject is male.

Embodiment 389. The method of any one of Embodiments 1-388, wherein, in (II), two or more proteins are selected from Table 217 or selected from Table 2017.

Embodiment 390. The method of Embodiment 389, wherein, in (II), three or more proteins are selected from Table 217 or selected from Table 2017.

Embodiment 391. The method of Embodiment 390, wherein, in (II), five or more proteins are selected from Table 217 or selected from Table 2017.

Embodiment 392. The method of Embodiment 391, wherein, in (II), ten or more proteins are selected from Table 217 or selected from Table 2017.

Embodiment 393. The method of any one of Embodiments 1-392, wherein, in (II), no more than about 300 proteins are selected from Table 217 or selected from Table 2017.

Embodiment 394. The method of Embodiment 393, wherein, in (II), no more than about 200 proteins are selected from Table 217 or selected from Table 2017.

Embodiment 395. The method of Embodiment 394, wherein, in (II), no more than about 100 proteins are selected from Table 217 or selected from Table 2017.

Embodiment 396. The method of Embodiment 395, wherein, in (II), no more than about 50 proteins are selected from Table 217 or selected from Table 2017.

Embodiment 397. The method of Embodiment 396, wherein, in (II), no more than about 25 proteins are selected from Table 217 or selected from Table 2017.

Embodiment 398. The method of any one of Embodiments 1-380, wherein, in (II), the one or more proteins selected from Table 217 is every protein of Table 217.A.1 or 217.A.2.

Embodiment 399. The method of any one of Embodiments 1-380, wherein, in (II), the one or more proteins selected from Table 2017 is every protein of Table 2017.A.1, 2017.A.2, or 2017.A.3.

Embodiment 400. The method of Embodiment 385, wherein, in (II), the one or more proteins selected from Table 217 is every protein of Table 217.B.1, 217.B.2, 217.B.3, or 217.B.4.

Embodiment 401. The method of Embodiment 385, wherein, in (II), the one or more proteins selected from Table 2017 is every protein of Table 2017.B.1, 2017.B.2, or 2017.B.3.

Embodiment 402. The method of Embodiment 388, wherein, in (II), the one or more proteins selected from Table 217 is every protein of Table 217.C.1, 217.C.2, 217.C.3, or 217.C.4.

Embodiment 403. The method of Embodiment 388, wherein, in (II), the one or more proteins selected from Table 2017 is every protein of Table 2017.C.1, 2017.C.2, or 2017.C.3.

Embodiment 404. The method of any one of Embodiments 1-403, wherein in (II), the one or more proteins selected from Table 218, are selected from Table 218.A.1 or 218.A.2.

Embodiment 405. The method of any one of Embodiments 1-403, wherein in (II), the one or more proteins selected from Table 2018 are selected from Table 2018.A.1.

Embodiment 406. The method of any one of Embodiments 1-403, wherein in (II), the one or more proteins selected from Table 218, are selected from Table 218.B.1, 218.B.2, 218.B.3, or 218.B.4.

Embodiment 407. The method of any one of Embodiments 1-403, wherein in (II), the one or more proteins selected from Table 2018 are selected from Table 2018.A.1, 2018.A.2, or 2018.A.3.

Embodiment 408. The method of Embodiment 406 or 407, wherein the subject is female.

Embodiment 409. The method of any one of Embodiments 1-408, wherein, in (II), two or more proteins are selected from Table 218, or selected from Table 2018.

Embodiment 410. The method of Embodiment 409, wherein, in (II), three or more proteins are selected from Table 218, or selected from Table 2018.

Embodiment 411. The method of Embodiment 410, wherein, in (II), five or more proteins are selected from Table 218, or selected from Table 2018.

Embodiment 412. The method of Embodiment 411, wherein, in (II), ten or more proteins are selected from Table 218, or selected from Table 2018.

Embodiment 413. The method of any one of Embodiments 1-412, wherein, in (II), no more than about 300 proteins are selected from Table 218, or selected from Table 2018.

Embodiment 414. The method of Embodiment 413, wherein, in (II), no more than about 200 proteins are selected from Table 218, or selected from Table 2018.

Embodiment 415. The method of Embodiment 414, wherein, in (II), no more than about 100 proteins are selected from Table 218, or selected from Table 2018.

Embodiment 416. The method of Embodiment 415, wherein, in (II), no more than about 50 proteins are selected from Table 218, or selected from Table 2018.

Embodiment 417. The method of Embodiment 416, wherein, in (II), no more than about 25 proteins are selected from Table 218, or selected from Table 2018.

Embodiment 418. The method of any one of Embodiments 1-403, wherein, in (II), the one or more proteins selected from Table 218, is every protein of Table 218.A.1 or 218.A.2.

Embodiment 419. The method of any one of Embodiments 1-403, wherein, in (II), the one or more proteins selected from Table 2018 is every protein of Table 2018.A.1.

Embodiment 420. The method of Embodiment 408, wherein, in (II), the one or more proteins selected from Table 218, is every protein of Table 218.B.1, 218.B.2, 218.B.3, or 218.B.4.

Embodiment 421. The method of Embodiment 408, wherein, in (II), the one or more proteins selected from Table 2018 is every protein of Table 2018.A.1, 2018.A.2, or 2018.A.3.

Embodiment 422. The method of any one of Embodiments 1-37, wherein in (II), the concentration of each protein is from Table 251, 252, 253, or 254.

Embodiment 423. The method of Embodiment 422, wherein the concentration of each protein is from Table 251.

Embodiment 424. The method of Embodiment 423, wherein the subject is female.

Embodiment 425. The method of Embodiment 422, wherein the concentration of each protein is from Table 252.

Embodiment 426. The method of Embodiment 425, wherein the subject is female.

Embodiment 427. The method of Embodiment 422, wherein the concentration of each protein is from Table 253.

Embodiment 428. The method of Embodiment 427, wherein the subject is male.

Embodiment 429. The method of Embodiment 422, wherein the concentration of each protein is from Table 254.

Embodiment 430. The method of Embodiment 429, wherein the subject is male.

Embodiment 431. The method of any one of Embodiments 422-430, wherein the cancer is early-stage.

Embodiment 432. A method of evaluating a subject for a cancer associated with a tissue selected from bladder, bone, brain, breast, cervix, colon, esophagus, kidney, liver, lung—small cell (SC), lung—non-small cell (NSC), ovary, pancreas, prostate, stomach, testis, thyroid, and uterus, the method comprising:
determining in a biological sample from the subject a concentration of one or more proteins selected from Tables 301-318 that respectively correspond to said tissues:
thereby evaluating the subject for cancer.

Embodiment 433. The method of Embodiment 432, further comprising applying a classifier to the concentration of the one or more proteins that identifies whether the concentration of the one or more proteins is indicative of the subject having cancer associated with the tissue.

Embodiment 434. The method of Embodiment 432 or 433, further comprising administering a treatment to the subject.

Embodiment 435. The method of any one of Embodiments 432-434, wherein the subject is evaluated for a cancer selected from breast, colon, SC lung, NSC lung, ovary, or pancreas, and the one or more proteins are selected from Tables 304, 306, 310, 311, 312, or 313, respectively.

Embodiment 436. A method of treating cancer in a subject, comprising
(a) acquiring results from the method of any one of Embodiments 432-435; and
(b) administering a treatment to the subject.

Embodiment 437. The method of Embodiment 436, wherein the treatment is responsive to the results acquired in (a).

Embodiment 438. The method of Embodiment 436 or 437, wherein (a) comprises:
(i) determining in a biological sample from the subject a concentration of one or more proteins selected from Tables 301-318 that respectively correspond to said tissues; and
(ii) applying a classifier to the concentration to identify with which tissue the cancer is associated.

Embodiment 439. A method of treating cancer in a subject, wherein the cancer is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising:
(a) acquiring results from an evaluation of the subject that determined the subject has cancer associated with the tissue:
(b) administering a treatment to the subject,
wherein the evaluation comprises:
   (i) determining in a biological sample from the subject a concentration of one or more proteins selected from Tables 301-318 that respectively correspond to said tissues; and
   (ii) applying a classifier to the concentration to identify with which tissue the cancer is associated.

Embodiment 440. The method of any one of Embodiments 436-439, wherein the results in (a) are acquired from a third party.

Embodiment 441. The method of any one of Embodiments 432-440, wherein the cancer is associated with the bladder or is bladder cancer.

Embodiment 442. The method of Embodiment 441, wherein the one or more proteins are selected from Table 301.A.1.

Embodiment 443. The method of Embodiment 441, wherein the one or more proteins are selected from Table 301.A.2.

Embodiment 444. The method of Embodiment 441, wherein the one or more proteins are selected from Table 301.B.1.

Embodiment 445. The method of Embodiment 444, wherein the subject is female.

Embodiment 446. The method of Embodiment 441, wherein the one or more proteins are selected from Table 301.B.2.

Embodiment 447. The method of Embodiment 446, wherein the subject is female.

Embodiment 448. The method of Embodiment 441, wherein the one or more proteins are selected from Table 301.C.1.

Embodiment 449. The method of Embodiment 448, wherein the subject is male.

Embodiment 450. The method of Embodiment 441, wherein the one or more proteins are selected from Table 301.C.2.

Embodiment 451. The method of Embodiment 450, wherein the subject is male.

Embodiment 452. The method of any one of Embodiments 441-451, wherein two or more proteins are selected from Table 301.

Embodiment 453. The method of Embodiment 452, wherein three or more proteins are selected from Table 301.

Embodiment 454. The method of Embodiment 453, wherein five or more proteins are selected from Table 301.

Embodiment 455. The method of Embodiment 454, wherein ten or more proteins are selected from Table 301.

Embodiment 456. The method of any one of Embodiments 441-455, wherein no more than about 300 proteins are selected from Table 301.

Embodiment 457. The method of Embodiment 456, wherein no more than about 200 proteins are selected from Table 301.

Embodiment 458. The method of Embodiment 457, wherein no more than about 100 proteins are selected from Table 301.

Embodiment 459. The method of Embodiment 458, wherein no more than about 50 proteins are selected from Table 301.

Embodiment 460. The method of Embodiment 459, wherein no more than about 25 proteins are selected from Table 301.

Embodiment 461. The method of any one of Embodiments 432-440, wherein the cancer is associated with bone or is bone cancer.

Embodiment 462. The method of Embodiment 461, wherein the one or more proteins are selected from Table 302.A.1.

Embodiment 463. The method of Embodiment 461, wherein the one or more proteins are selected from Table 302.A.2.

Embodiment 464. The method of Embodiment 461, wherein the one or more proteins are selected from Table 302.B.1.

Embodiment 465. The method of Embodiment 464, wherein the subject is female.

Embodiment 466. The method of Embodiment 461, wherein the one or more proteins are selected from Table 302.B.2.

Embodiment 467. The method of Embodiment 466, wherein the subject is female.

Embodiment 468. The method of Embodiment 461, wherein the one or more proteins are selected from Table 302.C.1.

Embodiment 469. The method of Embodiment 468, wherein the subject is male.

Embodiment 470. The method of Embodiment 461, wherein the one or more proteins are selected from Table 302.C.2.

Embodiment 471. The method of Embodiment 470, wherein the subject is male.

Embodiment 472. The method of any one of Embodiments 461-471, wherein two or more proteins are selected from Table 302.

Embodiment 473. The method of Embodiment 472, wherein three or more proteins are selected from Table 302.

Embodiment 474. The method of Embodiment 473, wherein five or more proteins are selected from Table 302.

Embodiment 475. The method of Embodiment 474, wherein ten or more proteins are selected from Table 302.

Embodiment 476. The method of any one of Embodiments 461-475, wherein no more than about 300 proteins are selected from Table 302.

Embodiment 477. The method of Embodiment 476, wherein no more than about 200 proteins are selected from Table 302.

Embodiment 478. The method of Embodiment 477, wherein no more than about 100 proteins are selected from Table 302.

Embodiment 479. The method of Embodiment 478, wherein no more than about 50 proteins are selected from Table 302.

Embodiment 480. The method of Embodiment 479, wherein no more than about 25 proteins are selected from Table 302.

Embodiment 481. The method of any one of Embodiments 461-480, wherein the cancer is osteosarcoma.

Embodiment 482. The method of any one of Embodiments 432-440, wherein the cancer is associated with the brain or is brain cancer.

Embodiment 483. The method of Embodiment 482, wherein the one or more proteins are selected from Table 303.A.1.

Embodiment 484. The method of Embodiment 482, wherein the one or more proteins are selected from Table 303.A.2.

Embodiment 485. The method of Embodiment 482, wherein the one or more proteins are selected from Table 303.B.1.

Embodiment 486. The method of Embodiment 485, wherein the subject is female.

Embodiment 487. The method of Embodiment 482, wherein the one or more proteins selected from Table 303.B.2.

Embodiment 488. The method of Embodiment 487, wherein the subject is female.

Embodiment 489. The method of Embodiment 482, wherein the one or more proteins are selected from Table 303.C.1.

Embodiment 490. The method of Embodiment 489, wherein the subject is male.

Embodiment 491. The method of Embodiment 482, wherein the one or more proteins are selected from Table 303.C.2.

Embodiment 492. The method of Embodiment 491, wherein the subject is male.

Embodiment 493. The method of any one of Embodiments 482-492, wherein two or more proteins are selected from Table 303.

Embodiment 494. The method of Embodiment 493, wherein three or more proteins are selected from Table 303.

Embodiment 495. The method of Embodiment 494, wherein five or more proteins are selected from Table 303.

Embodiment 496. The method of Embodiment 495, wherein ten or more proteins are selected from Table 303.

Embodiment 497. The method of any one of Embodiments 482-496, wherein no more than about 300 proteins are selected from Table 303.

Embodiment 498. The method of Embodiment 497, wherein no more than about 200 proteins are selected from Table 303.

Embodiment 499. The method of Embodiment 498, wherein no more than about 100 proteins are selected from Table 303.

Embodiment 500. The method of Embodiment 499, wherein no more than about 50 proteins are selected from Table 303.

Embodiment 501. The method of Embodiment 500, wherein no more than about 25 proteins are selected from Table 303.

Embodiment 502. The method of any one of Embodiments 482-501, wherein the cancer is astrocytoma.

Embodiment 503. The method of any one of Embodiments 432-440, wherein the cancer is associated with the breast or is breast cancer.

Embodiment 504. The method of Embodiment 503, wherein the one or more proteins are selected from Table 304.A.1.

Embodiment 505. The method of Embodiment 503, wherein the one or more proteins are selected from Table 304.A.2.

Embodiment 506. The method of Embodiment 503, wherein the one or more proteins are selected from Table 304.B.1.

Embodiment 507. The method of Embodiment 506, wherein the subject is female.

Embodiment 508. The method of Embodiment 503, wherein the one or more proteins selected from Table 304.B.2.

Embodiment 509. The method of Embodiment 508, wherein the subject is female.

Embodiment 510. The method of any one of Embodiments 503-509, wherein two or more proteins are selected from Table 304.

Embodiment 511. The method of Embodiment 510, wherein three or more proteins are selected from Table 304.

Embodiment 512. The method of Embodiment 511, wherein five or more proteins are selected from Table 304.

Embodiment 513. The method of Embodiment 512, wherein ten or more proteins are selected from Table 304.

Embodiment 514. The method of any one of Embodiments 503-513, wherein no more than about 300 proteins are selected from Table 304.

Embodiment 515. The method of Embodiment 514, wherein no more than about 200 proteins are selected from Table 304.

Embodiment 516. The method of Embodiment 515, wherein no more than about 100 proteins are selected from Table 304.

Embodiment 517. The method of Embodiment 516, wherein no more than about 50 proteins are selected from Table 304.

Embodiment 518. The method of Embodiment 517, wherein no more than about 25 proteins are selected from Table 304.

Embodiment 519. The method of any one of Embodiments 432-440, wherein the cancer is associated with the cervix or is cervical cancer.

Embodiment 520. The method of Embodiment 519, wherein the one or more proteins are selected from Table 305.A.1.

Embodiment 521. The method of Embodiment 519, wherein the one or more proteins are selected from Table 305.A.2.

Embodiment 522. The method of Embodiment 519, wherein the one or more proteins are selected from Table 305.B.1.

Embodiment 523. The method of Embodiment 522, wherein the subject is female.

Embodiment 524. The method of Embodiment 519, wherein the one or more proteins selected from Table 305.B.2.

Embodiment 525. The method of Embodiment 524, wherein the subject is female.

Embodiment 526. The method of any one of Embodiments 519-525, wherein two or more proteins are selected from Table 305.

Embodiment 527. The method of Embodiment 526, wherein three or more proteins are selected from Table 305.

Embodiment 528. The method of Embodiment 527, wherein five or more proteins are selected from Table 305.

Embodiment 529. The method of Embodiment 528, wherein ten or more proteins are selected from Table 305.

Embodiment 530. The method of any one of Embodiments 519-529, wherein no more than about 300 proteins are selected from Table 305.

Embodiment 531. The method of Embodiment 530, wherein no more than about 200 proteins are selected from Table 305.

Embodiment 532. The method of Embodiment 531, wherein no more than about 100 proteins are selected from Table 305.

Embodiment 533. The method of Embodiment 532, wherein no more than about 50 proteins are selected from Table 305.

Embodiment 534. The method of Embodiment 533, wherein no more than about 25 proteins are selected from Table 305.

Embodiment 535. The method of any one of Embodiments 432-440, wherein the cancer is associated with the colon or is colorectal cancer.

Embodiment 536. The method of Embodiment 535, wherein the one or more proteins are selected from Table 306.A.1.

Embodiment 537. The method of Embodiment 535, wherein the one or more proteins are selected from Table 306.A.2.

Embodiment 538. The method of Embodiment 535, wherein the one or more proteins are selected from Table 306.B.1.

Embodiment 539. The method of Embodiment 538, wherein the subject is female.

Embodiment 540. The method of Embodiment 535, wherein the one or more proteins selected from Table 306.B.2.

Embodiment 541. The method of Embodiment 540, wherein the subject is female.

Embodiment 542. The method of Embodiment 535, wherein the one or more proteins are selected from Table 306.C.1.

Embodiment 543. The method of Embodiment 542, wherein the subject is male.

Embodiment 544. The method of Embodiment 535, wherein the one or more proteins are selected from Table 306.C.2.

Embodiment 545. The method of Embodiment 544, wherein the subject is male.

Embodiment 546. The method of any one of Embodiments 535-545, wherein two or more proteins are selected from Table 306.

Embodiment 547. The method of Embodiment 546, wherein three or more proteins are selected from Table 306.

Embodiment 548. The method of Embodiment 547, wherein five or more proteins are selected from Table 306.

Embodiment 549. The method of Embodiment 548, wherein ten or more proteins are selected from Table 306.

Embodiment 550. The method of any one of Embodiments 535-549, wherein no more than about 300 proteins are selected from Table 306.

Embodiment 551. The method of Embodiment 550, wherein no more than about 200 proteins are selected from Table 306.

Embodiment 552. The method of Embodiment 551, wherein no more than about 100 proteins are selected from Table 306.

Embodiment 553. The method of Embodiment 552, wherein no more than about 50 proteins are selected from Table 306.

Embodiment 554. The method of Embodiment 553, wherein no more than about 25 proteins are selected from Table 306.

Embodiment 555. The method of any one of Embodiments 432-440, wherein the cancer is associated with the esophagus or is esophageal cancer.

Embodiment 556. The method of Embodiment 555, wherein the one or more proteins are selected from Table 307.A.1.

Embodiment 557. The method of Embodiment 555, wherein the one or more proteins are selected from Table 307.A.2.

Embodiment 558. The method of Embodiment 555, wherein the one or more proteins are selected from Table 307.B.1.

Embodiment 559. The method of Embodiment 558, wherein the subject is female.

Embodiment 560. The method of Embodiment 555, wherein the one or more proteins selected from Table 307.B.2.

Embodiment 561. The method of Embodiment 560, wherein the subject is female.

Embodiment 562. The method of Embodiment 555, wherein the one or more proteins are selected from Table 307.C.1.

Embodiment 563. The method of Embodiment 562, wherein the subject is male.

Embodiment 564. The method of Embodiment 555, wherein the one or more proteins are selected from Table 307.C.2.

Embodiment 565. The method of Embodiment 564, wherein the subject is male.

Embodiment 566. The method of any one of Embodiments 555-565, wherein two or more proteins are selected from Table 307.

Embodiment 567. The method of Embodiment 566, wherein three or more proteins are selected from Table 307.

Embodiment 568. The method of Embodiment 567, wherein five or more proteins are selected from Table 307.

Embodiment 569. The method of Embodiment 568, wherein ten or more proteins are selected from Table 307.

Embodiment 570. The method of any one of Embodiments 555-569, wherein no more than about 300 proteins are selected from Table 307.

Embodiment 571. The method of Embodiment 570, wherein no more than about 200 proteins are selected from Table 307.

Embodiment 572. The method of Embodiment 571, wherein no more than about 100 proteins are selected from Table 307.

Embodiment 573. The method of Embodiment 572, wherein no more than about 50 proteins are selected from Table 307.

Embodiment 574. The method of Embodiment 573, wherein no more than about 25 proteins are selected from Table 307.

Embodiment 575. The method of any one of Embodiments 432-440, wherein the cancer is associated with the kidney or is kidney cancer.

Embodiment 576. The method of Embodiment 575, wherein the one or more proteins are selected from Table 308.A.1.

Embodiment 577. The method of Embodiment 575, wherein the one or more proteins are selected from Table 308.A.2.

Embodiment 578. The method of Embodiment 575, wherein the one or more proteins are selected from Table 308.B.1.

Embodiment 579. The method of Embodiment 578, wherein the subject is female.

Embodiment 580. The method of Embodiment 575, wherein the one or more proteins are selected from Table 308.B.2.

Embodiment 581. The method of Embodiment 580, wherein the subject is female.

Embodiment 582. The method of Embodiment 575, wherein the one or more proteins are selected from Table 308.C.1.

Embodiment 583. The method of Embodiment 582, wherein the subject is male.

Embodiment 584. The method of Embodiment 575, wherein the one or more proteins are selected from Table 308.C.2.

Embodiment 585. The method of Embodiment 584, wherein the subject is male.

Embodiment 586. The method of any one of Embodiments 575-585, wherein two or more proteins are selected from Table 308.

Embodiment 587. The method of Embodiment 586, wherein three or more proteins are selected from Table 308.

Embodiment 588. The method of Embodiment 587, wherein five or more proteins are selected from Table 308.

Embodiment 589. The method of Embodiment 588, wherein ten or more proteins are selected from Table 308.

Embodiment 590. The method of any one of Embodiments 575-589, wherein no more than about 300 proteins are selected from Table 308.

Embodiment 591. The method of Embodiment 590, wherein no more than about 200 proteins are selected from Table 308.

Embodiment 592. The method of Embodiment 591, wherein no more than about 100 proteins are selected from Table 308.

Embodiment 593. The method of Embodiment 592, wherein no more than about 50 proteins are selected from Table 308.

Embodiment 594. The method of Embodiment 593, wherein no more than about 25 proteins are selected from Table 308.

Embodiment 595. The method of any one of Embodiments 432-440, wherein the cancer is associated with the liver or is liver cancer.

Embodiment 596. The method of Embodiment 595, wherein the one or more proteins are selected from Table 309.A.1.

Embodiment 597. The method of Embodiment 595, wherein the one or more proteins are selected from Table 309.A.2.

Embodiment 598. The method of Embodiment 595, wherein the one or more proteins are selected from Table 309.B.1.

Embodiment 599. The method of Embodiment 598, wherein the subject is female.

Embodiment 600. The method of Embodiment 595, wherein the one or more proteins selected from Table 309.B.2.

Embodiment 601. The method of Embodiment 600, wherein the subject is female.

Embodiment 602. The method of Embodiment 595, wherein the one or more proteins are selected from Table 309.C.1.

Embodiment 603. The method of Embodiment 602, wherein the subject is male.

Embodiment 604. The method of Embodiment 595, wherein the one or more proteins are selected from Table 309.C.2.

Embodiment 605. The method of Embodiment 604, wherein the subject is male.

Embodiment 606. The method of any one of Embodiments 595-605, wherein two or more proteins are selected from Table 309.

Embodiment 607. The method of Embodiment 606, wherein three or more proteins are selected from Table 309.

Embodiment 608. The method of Embodiment 607, wherein five or more proteins are selected from Table 309.

Embodiment 609. The method of Embodiment 608, wherein ten or more proteins are selected from Table 309.

Embodiment 610. The method of any one of Embodiments 595-609, wherein no more than about 300 proteins are selected from Table 309.

Embodiment 611. The method of Embodiment 610, wherein no more than about 200 proteins are selected from Table 309.

Embodiment 612. The method of Embodiment 611, wherein no more than about 100 proteins are selected from Table 309.

Embodiment 613. The method of Embodiment 612, wherein no more than about 50 proteins are selected from Table 309.

Embodiment 614. The method of Embodiment 613, wherein no more than about 25 proteins are selected from Table 309.

Embodiment 615. The method of any one of Embodiments 432-440, wherein the cancer is associated with the lung—small cell (SC) or is SC lung cancer.

Embodiment 616. The method of Embodiment 615, wherein the one or more proteins are selected from Table 310.A.1.

Embodiment 617. The method of Embodiment 615, wherein the one or more proteins are selected from Table 310.A.2.

Embodiment 618. The method of Embodiment 615, wherein the one or more proteins are selected from Table 310.B.1.

Embodiment 619. The method of Embodiment 618, wherein the subject is female.

Embodiment 620. The method of Embodiment 615, wherein the one or more proteins selected from Table 310.B.2.

Embodiment 621. The method of Embodiment 620, wherein the subject is female.

Embodiment 622. The method of Embodiment 615, wherein the one or more proteins are selected from Table 310.C.1.

Embodiment 623. The method of Embodiment 622, wherein the subject is male.

Embodiment 624. The method of Embodiment 615, wherein the one or more proteins are selected from Table 310.C.2.

Embodiment 625. The method of Embodiment 624, wherein the subject is male.

Embodiment 626. The method of any one of Embodiments 615-625, wherein two or more proteins are selected from Table 310.

Embodiment 627. The method of Embodiment 626, wherein three or more proteins are selected from Table 310.

Embodiment 628. The method of Embodiment 627, wherein five or more proteins are selected from Table 310.

Embodiment 629. The method of Embodiment 628, wherein ten or more proteins are selected from Table 310.

Embodiment 630. The method of any one of Embodiments 615-629, wherein no more than about 300 proteins are selected from Table 310.

Embodiment 631. The method of Embodiment 630, wherein no more than about 200 proteins are selected from Table 310.

Embodiment 632. The method of Embodiment 631, wherein no more than about 100 proteins are selected from Table 310.

Embodiment 633. The method of Embodiment 632, wherein no more than about 50 proteins are selected from Table 310.

Embodiment 634. The method of Embodiment 633, wherein no more than about 25 proteins are selected from Table 310.

Embodiment 635. The method of any one of Embodiments 432-440, wherein the cancer is associated with the lung—non-small cell (NSC) or is NSC lung cancer.

Embodiment 636. The method of Embodiment 635, wherein the one or more proteins are selected from Table 311.A.1.

Embodiment 637. The method of Embodiment 635, wherein the one or more proteins are selected from Table 311.A.2.

Embodiment 638. The method of Embodiment 635, wherein the one or more proteins are selected from Table 311.B.1.

Embodiment 639. The method of Embodiment 638, wherein the subject is female.

Embodiment 640. The method of Embodiment 635, wherein the one or more proteins are selected from Table 311.B.2.

Embodiment 641. The method of Embodiment 640, wherein the subject is female.

Embodiment 642. The method of Embodiment 635, wherein the one or more proteins are selected from Table 311.C.1.

Embodiment 643. The method of Embodiment 642, wherein the subject is male.

Embodiment 644. The method of Embodiment 635, wherein the one or more proteins are selected from Table 311.C.2.

Embodiment 645. The method of Embodiment 644, wherein the subject is male.

Embodiment 646. The method of any one of Embodiments 635-645, wherein two or more proteins are selected from Table 311.

Embodiment 647. The method of Embodiment 646, wherein three or more proteins are selected from Table 311.

Embodiment 648. The method of Embodiment 647, wherein five or more proteins are selected from Table 311.

Embodiment 649. The method of Embodiment 648, wherein ten or more proteins are selected from Table 311.

Embodiment 650. The method of any one of Embodiments 635-649, wherein no more than about 300 proteins are selected from Table 311.

Embodiment 651. The method of Embodiment 650, wherein no more than about 200 proteins are selected from Table 311.

Embodiment 652. The method of Embodiment 651, wherein no more than about 100 proteins are selected from Table 311.

Embodiment 653. The method of Embodiment 652, wherein no more than about 50 proteins are selected from Table 311.

Embodiment 654. The method of Embodiment 653, wherein no more than about 25 proteins are selected from Table 311.

Embodiment 655. The method of any one of Embodiments 432-440, wherein the cancer is associated with the ovary or is ovarian cancer.

Embodiment 656. The method of Embodiment 655, wherein the one or more proteins are selected from Table 312.A.1.

Embodiment 657. The method of Embodiment 655, wherein the one or more proteins are selected from Table 312.A.2.

Embodiment 658. The method of Embodiment 655, wherein the one or more proteins are selected from Table 312.B.1.

Embodiment 659. The method of Embodiment 658, wherein the subject is female.

Embodiment 660. The method of Embodiment 655, wherein the one or more proteins selected from Table 312.B.2.

Embodiment 661. The method of Embodiment 660, wherein the subject is female.

Embodiment 662. The method of any one of Embodiments 655-661, wherein two or more proteins are selected from Table 312.

Embodiment 663. The method of Embodiment 662, wherein three or more proteins are selected from Table 312.

Embodiment 664. The method of Embodiment 663, wherein five or more proteins are selected from Table 312.

Embodiment 665. The method of Embodiment 664, wherein ten or more proteins are selected from Table 312.

Embodiment 666. The method of any one of Embodiments 655-665, wherein no more than about 300 proteins are selected from Table 312.

Embodiment 667. The method of Embodiment 666, wherein no more than about 200 proteins are selected from Table 312.

Embodiment 668. The method of Embodiment 661, wherein no more than about 100 proteins are selected from Table 312.

Embodiment 669. The method of Embodiment 662, wherein no more than about 50 proteins are selected from Table 312.

Embodiment 670. The method of Embodiment 663, wherein no more than about 25 proteins are selected from Table 312.

Embodiment 671. The method of any one of Embodiments 432-440, wherein the cancer is associated with the pancreas.

Embodiment 672. The method of Embodiment 671, wherein the one or more proteins are selected from Table 313.A.1.

Embodiment 673. The method of Embodiment 671, wherein the one or more proteins are selected from Table 313.A.2.

Embodiment 674. The method of Embodiment 671, wherein the one or more proteins are selected from Table 313.B.1.

Embodiment 675. The method of Embodiment 674, wherein the subject is female.

Embodiment 676. The method of Embodiment 671, wherein the one or more proteins selected from Table 313.B.2.

Embodiment 677. The method of Embodiment 676, wherein the subject is female.

Embodiment 678. The method of Embodiment 671, wherein the one or more proteins are selected from Table 313.C.1.

Embodiment 679. The method of Embodiment 678, wherein the subject is male.

Embodiment 680. The method of Embodiment 671, wherein the one or more proteins are selected from Table 313.C.2.

Embodiment 681. The method of Embodiment 680, wherein the subject is male.

Embodiment 682. The method of any one of Embodiments 671-681, wherein two or more proteins are selected from Table 313.

Embodiment 683. The method of Embodiment 682, wherein three or more proteins are selected from Table 313.

Embodiment 684. The method of Embodiment 683, wherein five or more proteins are selected from Table 313.

Embodiment 685. The method of Embodiment 684, wherein ten or more proteins are selected from Table 313.

Embodiment 686. The method of any one of Embodiments 671-685, wherein no more than about 300 proteins are selected from Table 313.

Embodiment 687. The method of Embodiment 686, wherein no more than about 200 proteins are selected from Table 313.

Embodiment 688. The method of Embodiment 687, wherein no more than about 100 proteins are selected from Table 313.

Embodiment 689. The method of Embodiment 688, wherein no more than about 50 proteins are selected from Table 313.

Embodiment 690. The method of Embodiment 689, wherein no more than about 25 proteins are selected from Table 313.

Embodiment 691. The method of any one of Embodiments 432-440, wherein the cancer is associated with the prostate.

Embodiment 692. The method of Embodiment 691, wherein the one or more proteins are selected from Table 314.A.1.

Embodiment 693. The method of Embodiment 691, wherein the one or more proteins are selected from Table 314.A.2.

Embodiment 694. The method of Embodiment 691, wherein the one or more proteins are selected from Table 314.B.1.

Embodiment 695. The method of Embodiment 694, wherein the subject is male.

Embodiment 696. The method of Embodiment 691, wherein the one or more proteins selected from Table 314.B.2.

Embodiment 697. The method of Embodiment 696, wherein the subject is male.

Embodiment 698. The method of any one of Embodiments 691-697, wherein two or more proteins are selected from Table 314.

Embodiment 699. The method of Embodiment 698, wherein three or more proteins are selected from Table 314.

Embodiment 700. The method of Embodiment 699, wherein five or more proteins are selected from Table 314.

Embodiment 701. The method of Embodiment 700, wherein ten or more proteins are selected from Table 314.

Embodiment 702. The method of any one of Embodiments 691-701, wherein no more than about 300 proteins are selected from Table 314.

Embodiment 703. The method of Embodiment 702, wherein no more than about 200 proteins are selected from Table 314.

Embodiment 704. The method of Embodiment 703, wherein no more than about 100 proteins are selected from Table 314.

Embodiment 705. The method of Embodiment 704, wherein no more than about 50 proteins are selected from Table 314.

Embodiment 706. The method of Embodiment 705, wherein no more than about 25 proteins are selected from Table 314.

Embodiment 707. The method of any one of Embodiments 432-440, wherein the cancer is associated with the stomach.

Embodiment 708. The method of Embodiment 707, wherein the one or more proteins are selected from Table 315.A.1.

Embodiment 709. The method of Embodiment 707, wherein the one or more proteins are selected from Table 315.A.2.

Embodiment 710. The method of Embodiment 707, wherein the one or more proteins are selected from Table 315.B.1.

Embodiment 711. The method of Embodiment 710, wherein the subject is female.

Embodiment 712. The method of Embodiment 707, wherein the one or more proteins are selected from Table 315.B.2.

Embodiment 713. The method of Embodiment 712, wherein the subject is female.

Embodiment 714. The method of Embodiment 707, wherein the one or more proteins are selected from Table 315.C.1.

Embodiment 715. The method of Embodiment 714, wherein the subject is male.

Embodiment 716. The method of Embodiment 707, wherein the one or more proteins are selected from Table 315.C.2.

Embodiment 717. The method of Embodiment 716, wherein the subject is male.

Embodiment 718. The method of any one of Embodiments 707-717, wherein two or more proteins are selected from Table 315.

Embodiment 719. The method of Embodiment 718, wherein three or more proteins are selected from Table 315.

Embodiment 720. The method of Embodiment 719, wherein five or more proteins are selected from Table 315.

Embodiment 721. The method of Embodiment 720, wherein ten or more proteins are selected from Table 315.

Embodiment 722. The method of any one of Embodiments 707-721, wherein no more than about 300 proteins are selected from Table 315.

Embodiment 723. The method of Embodiment 722, wherein no more than about 200 proteins are selected from Table 315.

Embodiment 724. The method of Embodiment 723, wherein no more than about 100 proteins are selected from Table 315.

Embodiment 725. The method of Embodiment 724, wherein no more than about 50 proteins are selected from Table 315.

Embodiment 726. The method of Embodiment 725, wherein no more than about 25 proteins are selected from Table 315.

Embodiment 727. The method of any one of Embodiments 432-440, wherein the cancer is associated with the testis.

Embodiment 728. The method of Embodiment 727, wherein the one or more proteins are selected from Table 316.A.1.

Embodiment 729. The method of Embodiment 727, wherein the one or more proteins are selected from Table 316.A.2.

Embodiment 730. The method of Embodiment 727, wherein the one or more proteins are selected from Table 316.B.1.

Embodiment 731. The method of Embodiment 730, wherein the subject is male.

Embodiment 732. The method of Embodiment 727, wherein the one or more proteins are selected from Table 316.B.2.

Embodiment 733. The method of Embodiment 732, wherein the subject is male.

Embodiment 734. The method of any one of Embodiments 727-733, wherein two or more proteins are selected from Table 316.

Embodiment 735. The method of Embodiment 734, wherein three or more proteins are selected from Table 316.

Embodiment 736. The method of Embodiment 736, wherein five or more proteins are selected from Table 316.

Embodiment 737. The method of Embodiment 737, wherein ten or more proteins are selected from Table 316.

Embodiment 738. The method of any one of Embodiments 727-737, wherein no more than about 300 proteins are selected from Table 316.

Embodiment 739. The method of Embodiment 738, wherein no more than about 200 proteins are selected from Table 316.

Embodiment 740. The method of Embodiment 739, wherein no more than about 100 proteins are selected from Table 316.

Embodiment 741. The method of Embodiment 740, wherein no more than about 50 proteins are selected from Table 316.

Embodiment 742. The method of Embodiment 741, wherein no more than about 25 proteins are selected from Table 316.

Embodiment 743. The method of any one of Embodiments 432-440, wherein the cancer is associated with the thyroid.

Embodiment 744. The method of Embodiment 743, wherein the one or more proteins are selected from Table 317.A.1.

Embodiment 745. The method of Embodiment 743, wherein the one or more proteins are selected from Table 317.A.2.

Embodiment 746. The method of Embodiment 743, wherein the one or more proteins are selected from Table 317.B.1.

Embodiment 747. The method of Embodiment 746, wherein the subject is female.

Embodiment 748. The method of Embodiment 743, wherein the one or more proteins selected from Table 317.B.2.

Embodiment 749. The method of Embodiment 748, wherein the subject is female.

Embodiment 750. The method of Embodiment 743, wherein the one or more proteins are selected from Table 317.C.1.

Embodiment 751. The method of Embodiment 750, wherein the subject is male.

Embodiment 752. The method of Embodiment 743, wherein the one or more proteins are selected from Table 317.C.2.

Embodiment 753. The method of Embodiment 752, wherein the subject is male.

Embodiment 754. The method of any one of Embodiments 743-753, wherein two or more proteins are selected from Table 317.

Embodiment 755. The method of Embodiment 754, wherein three or more proteins are selected from Table 317.

Embodiment 756. The method of Embodiment 755, wherein five or more proteins are selected from Table 317.

Embodiment 757. The method of Embodiment 756, wherein ten or more proteins are selected from Table 317.

Embodiment 758. The method of any one of Embodiments 743-757, wherein no more than about 300 proteins are selected from Table 317.

Embodiment 759. The method of Embodiment 758, wherein no more than about 200 proteins are selected from Table 317.

Embodiment 760. The method of Embodiment 759, wherein no more than about 100 proteins are selected from Table 317.

Embodiment 761. The method of Embodiment 760, wherein no more than about 50 proteins are selected from Table 317.

Embodiment 762. The method of Embodiment 761, wherein no more than about 25 proteins are selected from Table 317.

Embodiment 763. The method of any one of Embodiments 432-440, wherein the cancer is associated with the uterus.

Embodiment 764. The method of Embodiment 763, wherein the one or more proteins are selected from Table 318.A.1.

Embodiment 765. The method of Embodiment 763, wherein the one or more proteins are selected from Table 318.A.2.

Embodiment 766. The method of Embodiment 763, wherein the one or more proteins are selected from Table 318.B.1.

Embodiment 767. The method of Embodiment 766, wherein the subject is female.

Embodiment 768. The method of Embodiment 763, wherein the one or more proteins selected from Table 318.B.2.

Embodiment 769. The method of Embodiment 768, wherein the subject is female.

Embodiment 770. The method of any one of Embodiments 763-769, wherein two or more proteins are selected from Table 318.

Embodiment 771. The method of Embodiment 770, wherein three or more proteins are selected from Table 318.

Embodiment 772. The method of Embodiment 771, wherein five or more proteins are selected from Table 318.

Embodiment 773. The method of Embodiment 772, wherein ten or more proteins are selected from Table 318.

Embodiment 774. The method of any one of Embodiments 763-773, wherein no more than about 300 proteins are selected from Table 318.

Embodiment 775. The method of Embodiment 774, wherein no more than about 200 proteins are selected from Table 318.

Embodiment 776. The method of Embodiment 775, wherein no more than about 100 proteins are selected from Table 318.

Embodiment 777. The method of Embodiment 776, wherein no more than about 50 proteins are selected from Table 318.

Embodiment 778. The method of Embodiment 777, wherein no more than about 25 proteins are selected from Table 318.

Embodiment 779. The method of any one of Embodiments 432-778, wherein the cancer is early-stage.

Embodiment 780. The method of any one of Embodiments 1-779, wherein the subject is asymptomatic for cancer.

Embodiment 781. The method of Embodiment 780, wherein the subject is asymptomatic for a cancer selected from a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 782. The method of Embodiment 780, wherein the subject is asymptomatic of a cancer comprising bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

Embodiment 783. A method of evaluating a subject who is suspected of having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising: determining in a biological sample from the subject a concentration of one or more proteins selected from Tables 201-218, or selected from Tables 2001-2018 that respectively correspond to said tissues; or determining in a biological sample from the subject a concentration of each protein from Table 251, 252, 253, or 254; thereby evaluating the subject for cancer.

Embodiment 784. The method of Embodiment 783, further comprising applying a classifier to the concentration of the one or more proteins that identifies whether the concentration of the one or more proteins is indicative of the subject having cancer associated with the tissue: or applying a classifier to the concentration of each protein that identifies whether the concentration of the proteins of Table 251, 252, 253, or 254 is indicative of the subject having cancer associated with the tissue.

Embodiment 785. The method of Embodiment 783 or 784, further comprising administering a treatment to the subject.

Embodiment 786. A method of treating cancer in a subject, comprising
(a) acquiring results from the method of any one of Embodiments 783-785; and
(b) administering a treatment to the subject.

Embodiment 787. The method of Embodiment 786, wherein the treatment is responsive to the results acquired in (a).

Embodiment 788. The method of Embodiment 786 or 787, wherein (a) comprises:
determining in a biological sample from the subject a concentration of one or more proteins selected from Tables 201-218, that respectively correspond to said tissues, and (applying a classifier to the concentration of the one or more proteins to identify with which tissue the cancer is associated; or
determining in a biological sample from the subject a concentration of each protein from Table 251, 252, 253, or 254, and applying a classifier to the concentration of each protein to identify with which tissue the cancer is associated.

Embodiment 789. A method of treating cancer in a subject who is suspected of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising:
(a) acquiring results from an evaluation of the subject that determined the subject has cancer associated with the tissue;
(b) administering a treatment to the subject,
wherein the evaluation comprises:
   (i) determining in a biological sample from the subject a concentration of one or more proteins selected from Tables 201-218, or selected from Tables 2001-2018 that respectively correspond to said tissues, and (ii) applying a classifier to the concentration of the one or more proteins to identify with which tissue the cancer is associated' or
   (i) determining in a biological sample from the subject a concentration of each protein from Table 251, 252, 253, or 254, and applying a classifier to the concentration of each protein to identify with which tissue the cancer is associated.

Embodiment 790. The method of any one of Embodiments 786-789, wherein the results in (a) are acquired from a third party.

Embodiment 791. The method of any one of Embodiments 783-790, wherein the subject is suspected of having a cancer associated with the bladder or of having bladder cancer.

Embodiment 792. The method of Embodiment 791, wherein the one or more proteins are selected from Table 201 or Table 2001.

Embodiment 793. The method of Embodiment 792, wherein the one or more proteins are selected from Table 201.A.1, 201.A.2, 2001.A.1, 2001.A.2, or 2001.A.3.

Embodiment 794. The method of Embodiment 792, wherein the one or more proteins are selected from Table 201.B.1, 201.B.2, 201.B.3, 201.B.4, 2001.B.1, 2001.B.2, or 2001.B.3.

Embodiment 795. The method of Embodiment 794, wherein the subject is female.

Embodiment 796. The method of Embodiment 792, wherein the one or more proteins are selected from Table 201.C.1, 201.C.2, 201.C.3, 201.C.4, 2001.C.1, 2001.C.2, or 2001.C.3.

Embodiment 797. The method of Embodiment 796, wherein the subject is male.

Embodiment 798. The method of any one of Embodiments 792-797, wherein two or more proteins are selected.

Embodiment 799. The method of Embodiment 798, wherein three or more proteins are selected.

Embodiment 800. The method of Embodiment 799, wherein five or more proteins are selected.

Embodiment 801. The method of Embodiment 800, wherein ten or more proteins are selected.

Embodiment 802. The method of any one of Embodiments 792-801, wherein no more than about 300 proteins are selected.

Embodiment 803. The method of Embodiment 802, wherein no more than about 200 proteins are selected.

Embodiment 804. The method of Embodiment 803, wherein no more than about 100 proteins are selected.

Embodiment 805. The method of Embodiment 804, wherein no more than about 50 proteins are selected.

Embodiment 806. The method of Embodiment 805, wherein no more than about 25 proteins are selected.

Embodiment 807. The method of Embodiment 792, wherein the one or more proteins is every protein of Table 201.A.1, 201.A.2, 2001.A.1, 2001.A.2, or 2001.A.3.

Embodiment 808. The method of Embodiment 792, wherein the one or more proteins is every protein of Table 201.B.1, 201.B.2, 201.B.3, 201.B.4, 2001.B.1, 2001.B.2, or 2001.B.3.

Embodiment 809. The method of Embodiment 808, wherein the subject is female.

Embodiment 810. The method of Embodiment 792, wherein the one or more proteins is every protein Table 201.C.1, 201.C.2, 201.C.3, 201.C.4, 2001.C.1, 2001.C.2, or 2001.C.3.

Embodiment 811. The method of Embodiment 810, wherein the subject is male.

Embodiment 812. The method of any one of Embodiments 791-811, wherein the cancer is early-stage.

Embodiment 813. The method of any one of Embodiments 783-789, wherein the subject is suspected of having a cancer associated with bone or of having one cancer.

Embodiment 814. The method of Embodiment 813, wherein the subject is suspected of having an osteosarcoma.

Embodiment 815. The method of Embodiment 813 or 814, wherein the one or more proteins are selected from Table 202 or Table 2002.

Embodiment 816. The method of Embodiment 815, wherein the one or more proteins are selected from Table 202.A.1, 202.A.2, 2002.A.1, 2002.A.2, or 2002.A.3.

Embodiment 817. The method of Embodiment 815, wherein the one or more proteins are selected from Table 202.B.1, 202.B.2, 202.B.3, 202.B.4, 2002.B.1, 2002.B.2, or 2002.B.3.

Embodiment 818. The method of Embodiment 817, wherein the subject is female.

Embodiment 819. The method of Embodiment 815, wherein the one or more proteins are selected from Table 202.C.1, 202.C.2, 202.C.3, 2002.C.1, 2002.C.2, or 2002.C.3.

Embodiment 820. The method of Embodiment 819, wherein the subject is male.

Embodiment 821. The method of any one of Embodiments 815-820, wherein two or more proteins are selected.

Embodiment 822. The method of Embodiment 821, wherein three or more proteins are selected.

Embodiment 823. The method of Embodiment 822, wherein five or more proteins are selected.

Embodiment 824. The method of Embodiment 823, wherein ten or more proteins are selected.

Embodiment 825. The method of any one of Embodiments 815-820, wherein no more than about 300 proteins are selected.

Embodiment 826. The method of Embodiment 825, wherein no more than about 200 proteins are selected.

Embodiment 827. The method of Embodiment 826, wherein no more than about 100 proteins are selected.

Embodiment 828. The method of Embodiment 827, wherein no more than about 50 proteins are selected.

Embodiment 829. The method of Embodiment 828, wherein no more than about 25 proteins are selected.

Embodiment 830. The method of Embodiment 815, wherein the one or more proteins is every protein of Table 202.A.1, 202.A.2, 2002.A.1, 2002.A.2, or 2002.A.3.

Embodiment 831. The method of Embodiment 815, wherein the one or more proteins is every protein of Table 202.B.1, 202.B.2, 202.B.3, 202.B.4, 2002.B.1, 2002.B.2, or 2002.B.3.

Embodiment 832. The method of Embodiment 831, wherein the subject is female.

Embodiment 833. The method of Embodiment 815, wherein the one or more proteins is every protein Table 202.C.1, 202.C.2, 202.C.3, 2002.C.1, 2002.C.2, or 2002.C.3.

Embodiment 834. The method of Embodiment 833, wherein the subject is male.

Embodiment 835. The method of any one of Embodiments 813-834, wherein the cancer is early-stage.

Embodiment 836. The method of any one of Embodiments 783-789, wherein the subject is suspected of having a cancer associated with the brain or of having brain cancer.

Embodiment 837. The method of Embodiment 836, wherein the subject is suspected of having an astrocytoma.

Embodiment 838. The method of Embodiment 836 or 837, wherein the one or more proteins are selected from Table 203 or Table 2003.

Embodiment 839. The method of Embodiment 838, wherein the one or more proteins are selected from Table 203.A.1, 203.A.2, 2003.A.1, 2003.A.2, or 2003.A.3.

Embodiment 840. The method of Embodiment 838, wherein the one or more proteins are selected from Table 203.B.1, 203.B.2, 203.B.3, 203.B.4, 2003.B.1, 2003.B.2, or 2003.B.3.

Embodiment 841. The method of Embodiment 840, wherein the subject is female.

Embodiment 842. The method of Embodiment 838, wherein the one or more proteins are selected from Table 203.C.1, 203.C.2, 203.C.3, 203.C.4, 2003.C.1, 2003.C.2, or 2003.C.3.

Embodiment 843. The method of Embodiment 842, wherein the subject is male.

Embodiment 844. The method of any one of Embodiments 838-843, wherein two or more proteins are selected.

Embodiment 845. The method of Embodiment 844, wherein three or more proteins are selected.

Embodiment 846. The method of Embodiment 845, wherein five or more proteins are selected.

Embodiment 847. The method of Embodiment 846, wherein ten or more proteins are selected.

Embodiment 848. The method of any one of Embodiments 838-847, wherein no more than about 300 proteins are selected.

Embodiment 849. The method of Embodiment 848, wherein no more than about 200 proteins are selected.

Embodiment 850. The method of Embodiment 849, wherein no more than about 100 proteins are selected.

Embodiment 851. The method of Embodiment 850, wherein no more than about 50 proteins are selected.

Embodiment 852. The method of Embodiment 851, wherein no more than about 25 proteins are selected.

Embodiment 853. The method of Embodiment 838, wherein the one or more proteins is every protein of Table 203.A.1, 203.A.2, 2003.A.1, 2003.A.2, or 2003.A.3.

Embodiment 854. The method of Embodiment 838, wherein the one or more proteins is every protein of Table 203.B.1, 203.B.2, 203.B.3, 203.B.4, 2003.B.1, 2003.B.2, or 2003.B.3.

Embodiment 855. The method of Embodiment 854, wherein the subject is female.

Embodiment 856. The method of Embodiment 838, wherein the one or more proteins is every protein Table 203.C.1, 203.C.2, 203.C.3, 203.C.4, 2003.C.1, 2003.C.2, or 2003.C.3.

Embodiment 857. The method of Embodiment 856, wherein the subject is male.

Embodiment 858. The method of any one of Embodiments 836-857, wherein the cancer is early-stage.

Embodiment 859. The method of any one of Embodiments 783-789, wherein the subject is suspected of having a cancer associated with the breast or of having breast cancer.

Embodiment 860. The method of Embodiment 859, wherein the one or more proteins are selected from Table 204 or Table 2004.

Embodiment 861. The method of Embodiment 860, wherein the one or more proteins are selected from Table 204.A.1, 204.A.2, 2004.A.1, 2004.A.2, or 2004.A.3.

Embodiment 862. The method of Embodiment 860, wherein the one or more proteins are selected from Table 204.B.1, 204.B.2, 204.B.3, 204.B.4, 2004.A.1, 2004.A.2, or 2004.A.3.

Embodiment 863. The method of Embodiment 862, wherein the subject is female.

Embodiment 864. The method of any one of Embodiments 860-863, wherein two or more proteins are selected.

Embodiment 865. The method of Embodiment 864, wherein three or more proteins are selected.

Embodiment 866. The method of Embodiment 865, wherein five or more proteins are selected.

Embodiment 867. The method of Embodiment 866, wherein ten or more proteins are selected.

Embodiment 868. The method of any one of Embodiments 860-867, wherein no more than about 300 proteins are selected.

Embodiment 869. The method of Embodiment 868, wherein no more than about 200 proteins are selected.

Embodiment 870. The method of Embodiment 869, wherein no more than about 100 proteins are selected.

Embodiment 871. The method of Embodiment 870, wherein no more than about 50 proteins are selected.

Embodiment 872. The method of Embodiment 871, wherein no more than about 25 proteins are selected.

Embodiment 873. The method of Embodiment 860, wherein the one or more proteins is every protein of Table 204.A.1, 204.A.2, 2004.A.1, 2004.A.2, or 2004.A.3.

Embodiment 874. The method of Embodiment 860, wherein the one or more proteins is every protein of Table 204.B.1, 204.B.2, 204.B.3, 204.B.4, 2004.A.1, 2004.A.2, or 2004.A.3.

Embodiment 875. The method of Embodiment 874, wherein the subject is female.

Embodiment 876. The method of any one of Embodiments 859-875, wherein the cancer is early-stage.

Embodiment 877. The method of any one of Embodiments 783-789, wherein the subject is suspected of having a cancer associated with the cervix or of having cervical cancer.

Embodiment 878. The method of Embodiment 877, wherein the one or more proteins are selected from Table 205 or Table 2005.

Embodiment 879. The method of Embodiment 878, wherein the one or more proteins are selected from Table 205.A.1, 205.A.2, 2005.A.1, 2005.A.2, or 2005.A.3.

Embodiment 880. The method of Embodiment 878, wherein the one or more proteins are selected from Table 205.B.1, 205.B.2, 205.B.3, 205.B.4, 2005.A.1, 2005.A.2, or 2005.A.3.

Embodiment 881. The method of Embodiment 880, wherein the subject is female.

Embodiment 882. The method of any one of Embodiments 878-881, wherein two or more proteins are selected.

Embodiment 883. The method of Embodiment 882, wherein three or more proteins are selected.

Embodiment 884. The method of Embodiment 883, wherein five or more proteins are selected.

Embodiment 885. The method of Embodiment 884, wherein ten or more proteins are selected.

Embodiment 886. The method of any one of Embodiments 878-885, wherein no more than about 300 proteins are selected.

Embodiment 887. The method of Embodiment 886, wherein no more than about 200 proteins are selected.

Embodiment 888. The method of Embodiment 887, wherein no more than about 100 proteins are selected.

Embodiment 889. The method of Embodiment 888, wherein no more than about 50 proteins are selected.

Embodiment 890. The method of Embodiment 889, wherein no more than about 25 proteins are selected.

Embodiment 891. The method of Embodiment 878, wherein the one or more proteins is every protein of Table 205.A.1, 205.A.2, 2005.A.1, 2005.A.2, or 2005.A.3.

Embodiment 892. The method of Embodiment 878, wherein the one or more proteins is every protein of Table 205.B.1, 205.B.2, 205.B.3, 205.B.4, 2005.A.1, 2005.A.2, or 2005.A.3.

Embodiment 893. The method of Embodiment 892, wherein the subject is female.

Embodiment 894. The method of any one of Embodiments 877-893, wherein the cancer is early-stage.

Embodiment 895. The method of any one of Embodiments 783-789, wherein the subject is suspected of having a cancer associated with the colon or of having colorectal cancer.

Embodiment 896. The method of Embodiment 895, wherein the one or more proteins are selected from Table 206 or Table 2006.

Embodiment 897. The method of Embodiment 896, wherein the one or more proteins are selected from Table 206.A.1, 206.A.2, 2006.A.1, 2006.A.2, or 2006.A.3.

Embodiment 898. The method of Embodiment 896, wherein the one or more proteins are selected from Table 206.B.1, 206.B.2, 206.B.3, 206.B.4, 2006.B.1, 2006.B.2, or 2006.B.3.

Embodiment 899. The method of Embodiment 898, wherein the subject is female.

Embodiment 900. The method of Embodiment 896, wherein the one or more proteins are selected from Table 206.C.1, 206.C.2, 206.C.3, 2006.C.1, 2006.C.2, or 2006.C.3.

Embodiment 901. The method of Embodiment 900, wherein the subject is male.

Embodiment 902. The method of any one of Embodiments 896-901, wherein two or more proteins are selected.

Embodiment 903. The method of Embodiment 902, wherein three or more proteins are selected.

Embodiment 904. The method of Embodiment 903, wherein five or more proteins are selected.

Embodiment 905. The method of Embodiment 904, wherein ten or more proteins are selected.

Embodiment 906. The method of any one of Embodiments 896-905, wherein no more than about 300 proteins are selected.

Embodiment 907. The method of Embodiment 906, wherein no more than about 200 proteins are selected.

Embodiment 908. The method of Embodiment 907, wherein no more than about 100 proteins are selected.

Embodiment 909. The method of Embodiment 908, wherein no more than about 50 proteins are selected.

Embodiment 910. The method of Embodiment 909, wherein no more than about 25 proteins are selected.

Embodiment 911. The method of Embodiment 896, wherein the one or more proteins is every protein of Table 206.A.1, 206.A.2, 2006.A.1, 2006.A.2, or 2006.A.3.

Embodiment 912. The method of Embodiment 896, wherein the one or more proteins is every protein of Table 206.B.1, 206.B.2, 206.B.3, 206.B.4, 2006.B.1, 2006.B.2, or 2006.B.3.

Embodiment 913. The method of Embodiment 912, wherein the subject is female.

Embodiment 914. The method of Embodiment 896, wherein the one or more proteins is every protein Table 206.C.1, 206.C.2, 206.C.3, 2006.C.1, 2006.C.2, or 2006.C.3.

Embodiment 915. The method of Embodiment 914, wherein the subject is male.

Embodiment 916. The method of any one of Embodiments 895-915, wherein the cancer is early-stage.

Embodiment 917. The method of any one of Embodiments 783-789, wherein the subject is suspected of having a cancer associated with the esophagus or of esophageal cancer.

Embodiment 918. The method of Embodiment 917, wherein the one or more proteins are selected from Table 207 or Table 2007.

Embodiment 919. The method of Embodiment 918, wherein the one or more proteins are selected from Table 207.A.1, 207.A.2, 2007.A.1, 2007.A.2, or 2007.A.3.

Embodiment 920. The method of Embodiment 918, wherein the one or more proteins are selected from Table 207.B.1, 207.B.2, 207.B.3, 207.B.4, 2007.B.1, 2007.B.2, or 2007.B.3.

Embodiment 921. The method of Embodiment 920, wherein the subject is female.

Embodiment 922. The method of Embodiment 918, wherein the one or more proteins are selected from Table 207.C.1, 207.C.2, 207.C.3, 2007.C.1, 2007.C.2, or 2007.C.3.

Embodiment 923. The method of Embodiment 922, wherein the subject is male.

Embodiment 924. The method of any one of Embodiments 918-923, wherein two or more proteins are selected.

Embodiment 925. The method of Embodiment 924, wherein three or more proteins are selected.

Embodiment 926. The method of Embodiment 925, wherein five or more proteins are selected.

Embodiment 927. The method of Embodiment 926, wherein ten or more proteins are selected.

Embodiment 928. The method of any one of Embodiments 918-927, wherein no more than about 300 proteins are selected.

Embodiment 929. The method of Embodiment 928, wherein no more than about 200 proteins are selected.

Embodiment 930. The method of Embodiment 929, wherein no more than about 100 proteins are selected.

Embodiment 931. The method of Embodiment 930, wherein no more than about 50 proteins are selected.

Embodiment 932. The method of Embodiment 931, wherein no more than about 25 proteins are selected.

Embodiment 933. The method of Embodiment 918, wherein the one or more proteins is every protein of Table 207.A.1, 207.A.2, 2007.A.1, 2007.A.2, or 2007.A.3.

Embodiment 934. The method of Embodiment 918, wherein the one or more proteins is every protein of Table 207.B.1, 207.B.2, 207.B.3, 207.B.4, 2007.B.1, 2007.B.2, or 2007.B.3.

Embodiment 935. The method of Embodiment 934, wherein the subject is female.

Embodiment 936. The method of Embodiment 918, wherein the one or more proteins is every protein Table 207.C.1, 207.C.2, 207.C.3, 2007.C.1, 2007.C.2, or 2007.C.3.

Embodiment 937. The method of Embodiment 936, wherein the subject is male.

Embodiment 938. The method of any one of Embodiments 917-937, wherein the cancer is early-stage.

Embodiment 939. The method of any one of Embodiments 783-789, wherein the subject is suspected of having a cancer associated with the kidney or of having kidney cancer.

Embodiment 940. The method of Embodiment 939, wherein the one or more proteins are selected from Table 208 or Table 2008.

Embodiment 941. The method of Embodiment 940, wherein the one or more proteins are selected from Table 208.A.1, 208.A.2, 2008.A.1, 2008.A.2, or 2008.A.3.

Embodiment 942. The method of Embodiment 940, wherein the one or more proteins are selected from Table 208.B.1, 208.B.2, 208.B.3, 208.B.4, 2008.B.1, 2008.B.2, or 2008.B.3.

Embodiment 943. The method of Embodiment 942, wherein the subject is female.

Embodiment 944. The method of Embodiment 940, wherein the one or more proteins are selected from Table 208.C.1, 208.C.2, 208.C.3, 208.C.4, 2008.C.1, 2008.C.2, or 2008.C.3.

Embodiment 945. The method of Embodiment 944, wherein the subject is male.

Embodiment 946. The method of any one of Embodiments 940-945, wherein two or more proteins are selected.

Embodiment 947. The method of Embodiment 946, wherein three or more proteins are selected.

Embodiment 948. The method of Embodiment 947, wherein five or more proteins are selected.

Embodiment 949. The method of Embodiment 948, wherein ten or more proteins are selected.

Embodiment 950. The method of any one of Embodiments 940-949, wherein no more than about 300 proteins are selected.

Embodiment 951. The method of Embodiment 950, wherein no more than about 200 proteins are selected.

Embodiment 952. The method of Embodiment 951, wherein no more than about 100 proteins are selected.

Embodiment 953. The method of Embodiment 952, wherein no more than about 50 proteins are selected.

Embodiment 954. The method of Embodiment 953, wherein no more than about 25 proteins are selected.

Embodiment 955. The method of Embodiment 940, wherein the one or more proteins is every protein of Table 208.A.1, 208.A.2, 2008.A.1, 2008.A.2, or 2008.A.3.

Embodiment 956. The method of Embodiment 940, wherein the one or more proteins is every protein of Table 208.B.1, 208.B.2, 208.B.3, 208.B.4, 2008.B.1, 2008.B.2, or 2008.B.3.

Embodiment 957. The method of Embodiment 956, wherein the subject is female.

Embodiment 958. The method of Embodiment 940, wherein the one or more proteins is every protein Table 208.C.1, 208.C.2, 208.C.3, 208.C.4, 2008.C.1, 2008.C.2, or 2008.C.3.

Embodiment 959. The method of Embodiment 958, wherein the subject is male.

Embodiment 960. The method of any one of Embodiments 939-959, wherein the cancer is early-stage.

Embodiment 961. The method of any one of Embodiments 783-789, wherein the subject is suspected of having a cancer associated with the liver or of having liver cancer.

Embodiment 962. The method of Embodiment 961, wherein the one or more proteins are selected from Table 209 or Table 2009.

Embodiment 963. The method of Embodiment 962, wherein the one or more proteins are selected from Table 209.A.1, 209.A.2, 2009.A.1, 2009.A.2, or 2009.A.3.

Embodiment 964. The method of Embodiment 962, wherein the one or more proteins are selected from Table 209.B.1, 209.B.2, 209.B.3, 209.B.4, 2009.B.1, 2009.B.2, or 2009.B.3.

Embodiment 965. The method of Embodiment 964, wherein the subject is female.

Embodiment 966. The method of Embodiment 962, wherein the one or more proteins are selected from Table 209.C.1, 209.C.2, 209.C.3, 209.C.4, 2009.C.1, 2009.C.2, or 2009.C.3.

Embodiment 967. The method of Embodiment 966, wherein the subject is male.

Embodiment 968. The method of any one of Embodiments 962-967, wherein two or more proteins are selected.

Embodiment 969. The method of Embodiment 968, wherein three or more proteins are selected.

Embodiment 970. The method of Embodiment 969, wherein five or more proteins are selected.

Embodiment 971. The method of Embodiment 970, wherein ten or more proteins are selected.

Embodiment 972. The method of any one of Embodiments 962-971, wherein no more than about 300 proteins are selected.

Embodiment 973. The method of Embodiment 972, wherein no more than about 200 proteins are selected.

Embodiment 974. The method of Embodiment 973, wherein no more than about 100 proteins are selected.

Embodiment 975. The method of Embodiment 974, wherein no more than about 50 proteins are selected.

Embodiment 976. The method of Embodiment 975, wherein no more than about 25 proteins are selected.

Embodiment 977. The method of Embodiment 962, wherein the one or more proteins is every protein of Table 209.A.1, 209.A.2, 2009.A.1, 2009.A.2, or 2009.A.3.

Embodiment 978. The method of Embodiment 962, wherein the one or more proteins is every protein of Table 209.B.1, 209.B.2, 209.B.3, 209.B.4, 2009.B.1, 2009.B.2, or 2009.B.3.

Embodiment 979. The method of Embodiment 978, wherein the subject is female.

Embodiment 980. The method of Embodiment 962, wherein the one or more proteins is every protein Table 209.C.1, 209.C.2, 209.C.3, 209.C.4, 2009.C.1, 2009.C.2, or 2009.C.3.

Embodiment 981. The method of Embodiment 980, wherein the subject is male.

Embodiment 982. The method of any one of Embodiments 961-981, wherein the cancer is early-stage.

Embodiment 983. The method of any one of Embodiments 783-789, wherein the subject is suspected of having a cancer associated with the lung—small cell or of having small cell lung cancer.

Embodiment 984. The method of Embodiment 983, wherein the one or more proteins are selected from Table 210 or Table 2010.

Embodiment 985. The method of Embodiment 984, wherein the one or more proteins are selected from Table 210.A.1, 210.A.2, 2010.A.1, 2010.A.2, or 2010.A.3.

Embodiment 986. The method of Embodiment 984, wherein the one or more proteins are selected from Table 210.B.1, 210.B.2, 210.B.3, 210.B.4, 2010.B.1, 2010.B.2, or 2010.B.3.

Embodiment 987. The method of Embodiment 986, wherein the subject is female.

Embodiment 988. The method of Embodiment 984, wherein the one or more proteins are selected from Table 210.C.1, 210.C.2, 210.C.3, 210.C.4, 2010.C.1, 2010.C.2, or 2010.C.3.

Embodiment 989. The method of Embodiment 988, wherein the subject is male.

Embodiment 990. The method of any one of Embodiments 984-989, wherein two or more proteins are selected.

Embodiment 991. The method of Embodiment 990, wherein three or more proteins are selected.

Embodiment 992. The method of Embodiment 991, wherein five or more proteins are selected.

Embodiment 993. The method of Embodiment 992, wherein ten or more proteins are selected.

Embodiment 994. The method of any one of Embodiments 984-993, wherein no more than about 300 proteins are selected.

Embodiment 995. The method of Embodiment 994, wherein no more than about 200 proteins are selected.

Embodiment 996. The method of Embodiment 995, wherein no more than about 100 proteins are selected.

Embodiment 997. The method of Embodiment 996, wherein no more than about 50 proteins are selected.

Embodiment 998. The method of Embodiment 997, wherein no more than about 25 proteins are selected.

Embodiment 999. The method of Embodiment 984, wherein the one or more proteins is every protein of Table 210.A.1, 210.A.2, 2010.A.1, 2010.A.2, or 2010.A.3.

Embodiment 1000. The method of Embodiment 984, wherein the one or more proteins is every protein of Table 210.B.1, 210.B.2, 210.B.3, 210.B.4, 2010.B.1, 2010.B.2, or 2010.B.3.

Embodiment 1001. The method of Embodiment 1000, wherein the subject is female.

Embodiment 1002. The method of Embodiment 984, wherein the one or more proteins is every protein Table 210.C.1, 210.C.2, 210.C.3, 210.C.4, 2010.C.1, 2010.C.2, or 2010.C.3.

Embodiment 1003. The method of Embodiment 1002, wherein the subject is male.

Embodiment 1004. The method of any one of Embodiments 983-1003, wherein the cancer is early-stage.

Embodiment 1005. The method of any one of Embodiments 783-789, wherein the subject is suspected of having a cancer associated with the lung—non-small cell or of having non-small cell lung cancer.

Embodiment 1006. The method of Embodiment 1005, wherein the one or more proteins are selected from Table 211 or Table 2011.

Embodiment 1007. The method of Embodiment 1006, wherein the one or more proteins are selected from Table 211.A.1, 211.A.2, 2011.A.1, or 2011.A.2.

Embodiment 1008. The method of Embodiment 1006, wherein the one or more proteins are selected from Table 211.B.1, 211.B.2, 211.B.3, 211.B.4, 2011.B.1, 2011.B.2, or 2011.B.3.

Embodiment 1009. The method of Embodiment 1008, wherein the subject is female.

Embodiment 1010. The method of Embodiment 1006, wherein the one or more proteins are selected from Table 211.C.1, 211.C.2, 211.C.3, 2011.C.1, 2011.C.2, or 2011.C.3.

Embodiment 1011. The method of Embodiment 1010, wherein the subject is male.

Embodiment 1012. The method of any one of Embodiments 1006-1011, wherein two or more proteins are selected.

Embodiment 1013. The method of Embodiment 1012, wherein three or more proteins are selected.

Embodiment 1014. The method of Embodiment 1013, wherein five or more proteins are selected.

Embodiment 1015. The method of Embodiment 1014, wherein ten or more proteins are selected.

Embodiment 1016. The method of any one of Embodiments 1006-1015, wherein no more than about 300 proteins are selected.

Embodiment 1017. The method of Embodiment 1016, wherein no more than about 200 proteins are selected.

Embodiment 1018. The method of Embodiment 1017, wherein no more than about 100 proteins are selected.

Embodiment 1019. The method of Embodiment 1018, wherein no more than about 50 proteins are selected.

Embodiment 1020. The method of Embodiment 1019, wherein no more than about 25 proteins are selected.

Embodiment 1021. The method of Embodiment 1006, wherein the one or more proteins is every protein of Table 211.A.1, 211.A.2, 2011.A.1, or 2011.A.2.

Embodiment 1022. The method of Embodiment 1006, wherein the one or more proteins is every protein of Table 211.B.1, 211.B.2, 211.B.3, 211.B.4, 2011.B.1, 2011.B.2, or 2011.B.3.

Embodiment 1023. The method of Embodiment 1022, wherein the subject is female.

Embodiment 1024. The method of Embodiment 1006, wherein the one or more proteins is every protein Table 211.C.1, 211.C.2, 211.C.3, 2011.C.1, 2011.C.2, or 2011.C.3.

Embodiment 1025. The method of Embodiment 1024, wherein the subject is male.

Embodiment 1026. The method of any one of Embodiments 1005-1025, wherein the cancer is early-stage.

Embodiment 1027. The method of any one of Embodiments 783-789, wherein the subject is suspected of having a cancer associated with the ovary or of having ovarian cancer.

Embodiment 1028. The method of Embodiment 1027, wherein the one or more proteins are selected from Table 212 or Table 2012.

Embodiment 1029. The method of Embodiment 1028, wherein the one or more proteins are selected from Table 212.A.1, 212.A.2, 2012.A.1, 2012.A.2, or 2012.A.3.

Embodiment 1030. The method of Embodiment 1028, wherein the one or more proteins are selected from Table 212.B.1, 212.B.2, 212.B.3, 212.B.4, 2012.A.1, 2012.A.2, or 2012.A.3.

Embodiment 1031. The method of Embodiment 1030, wherein the subject is female.

Embodiment 1032. The method of any one of Embodiments 1028-1031, wherein two or more proteins are selected.

Embodiment 1033. The method of Embodiment 1032, wherein three or more proteins are selected.

Embodiment 1034. The method of Embodiment 1033, wherein five or more proteins are selected.

Embodiment 1035. The method of Embodiment 1034, wherein ten or more proteins are selected.

Embodiment 1036. The method of any one of Embodiments 1028-1035, wherein no more than about 300 proteins are selected.

Embodiment 1037. The method of Embodiment 1036, wherein no more than about 200 proteins are selected.

Embodiment 1038. The method of Embodiment 1037, wherein no more than about 100 proteins are selected.

Embodiment 1039. The method of Embodiment 1038, wherein no more than about 50 proteins are selected.

Embodiment 1040. The method of Embodiment 1039, wherein no more than about 25 proteins are selected.

Embodiment 1041. The method of Embodiment 1028, wherein the one or more proteins is every protein of Table 212.A.1, 212.A.2, 2012.A.1, 2012.A.2, or 2012.A.3.

Embodiment 1042. The method of Embodiment 1028, wherein the one or more proteins is every protein of Table 212.B.1, 212.B.2, 212.B.3, 212.B.4, 2012.A.1, 2012.A.2, or 2012.A.3.

Embodiment 1043. The method of Embodiment 1042, wherein the subject is female.

Embodiment 1044. The method of any one of Embodiments 1027-1043, wherein the cancer is early-stage.

Embodiment 1045. The method of any one of Embodiments 783-789, wherein the subject is suspected of having a cancer associated with the pancreas or of having pancreatic cancer.

Embodiment 1046. The method of Embodiment 1045, wherein the one or more proteins are selected from Table 213 or Table 2013.

Embodiment 1047. The method of Embodiment 1046, wherein the one or more proteins are selected from Table 213.A.1, 213.A.2, 2013.A.1, 2013.A.2, or 2013.A.3.

Embodiment 1048. The method of Embodiment 1046, wherein the one or more proteins are selected from Table 213.B.1, 213.B.2, 213.B.3, 2013.B.1, 2013.B.2, or 2013.B.3.

Embodiment 1049. The method of Embodiment 1048, wherein the subject is female.

Embodiment 1050. The method of Embodiment 1046, wherein the one or more proteins are selected from Table 213.C.1, 213.C.2, 213.C.3, 2013.C.1, 2013.C.2, or 2013.C.3.

Embodiment 1051. The method of Embodiment 1050, wherein the subject is male.

Embodiment 1052. The method of any one of Embodiments 1046-1051, wherein two or more proteins are selected.

Embodiment 1053. The method of Embodiment 1052, wherein three or more proteins are selected.

Embodiment 1054. The method of Embodiment 1053, wherein five or more proteins are selected.

Embodiment 1055. The method of Embodiment 1054, wherein ten or more proteins are selected.

Embodiment 1056. The method of any one of Embodiments 1046-1055, wherein no more than about 300 proteins are selected.

Embodiment 1057. The method of Embodiment 1056, wherein no more than about 200 proteins are selected.

Embodiment 1058. The method of Embodiment 1057, wherein no more than about 100 proteins are selected.

Embodiment 1059. The method of Embodiment 1058, wherein no more than about 50 proteins are selected.

Embodiment 1060. The method of Embodiment 1059, wherein no more than about 25 proteins are selected.

Embodiment 1061. The method of Embodiment 1046, wherein the one or more proteins is every protein of Table 213.A.1, 213.A.2, 2013.A.1, 2013.A.2, or 2013.A.3.

Embodiment 1062. The method of Embodiment 1046, wherein the one or more proteins is every protein of Table 213.B.1, 213.B.2, 213.B.3, 2013.B.1, 2013.B.2, or 2013.B.3.

Embodiment 1063. The method of Embodiment 1062, wherein the subject is female.

Embodiment 1064. The method of Embodiment 1046, wherein the one or more proteins is every protein Table 213.C.1, 213.C.2, 213.C.3, 2013.C.1, 2013.C.2, or 2013.C.3.

Embodiment 1065. The method of Embodiment 1064, wherein the subject is male.

Embodiment 1066. The method of any one of Embodiments 1045-1065, wherein the cancer is early-stage.

Embodiment 1067. The method of any one of Embodiments 783-789, wherein the subject is suspected of having a cancer associated with the prostate or of having prostate cancer.

Embodiment 1068. The method of Embodiment 1067, wherein the one or more proteins are selected from Table 214 or Table 2014.

Embodiment 1069. The method of Embodiment 1068, wherein the one or more proteins are selected from Table 214.A.1, 214.A.2, 2014.A.1, 2014.A.2, or 2014.A.3.

Embodiment 1070. The method of Embodiment 1068, wherein the one or more proteins are selected from Table 214.B.1, 214.B.2, 214.B.3, 214.B.4, 2014.A.1, 2014.A.2, or 2014.A.3.

Embodiment 1071. The method of Embodiment 1070, wherein the subject is male.

Embodiment 1072. The method of any one of Embodiments 1068-1071, wherein two or more proteins are selected.

Embodiment 1073. The method of Embodiment 1072, wherein three or more proteins are selected.

Embodiment 1074. The method of Embodiment 1073, wherein five or more proteins are selected.

Embodiment 1075. The method of Embodiment 1074, wherein ten or more proteins are selected.

Embodiment 1076. The method of any one of Embodiments 1068-1075, wherein no more than about 300 proteins are selected.

Embodiment 1077. The method of Embodiment 1076, wherein no more than about 200 proteins are selected.

Embodiment 1078. The method of Embodiment 1077, wherein no more than about 100 proteins are selected.

Embodiment 1079. The method of Embodiment 1078, wherein no more than about 50 proteins are selected.

Embodiment 1080. The method of Embodiment 1079, wherein no more than about 25 proteins are selected.

Embodiment 1081. The method of Embodiment 1068, wherein the one or more proteins is every protein of Table 214.A.1, 214.A.2, 2014.A.1, 2014.A.2, or 2014.A.3.

Embodiment 1082. The method of Embodiment 1068, wherein the one or more proteins is every protein of Table 214.B.1, 214.B.2, 214.B.3, 214.B.4, 2014.A.1, 2014.A.2, or 2014.A.3.

Embodiment 1083. The method of Embodiment 1082, wherein the subject is male.

Embodiment 1084. The method of any one of Embodiments 1067-811, wherein the cancer is early-stage.

Embodiment 1085. The method of any one of Embodiments 783-789, wherein the subject is suspected of having a cancer associated with the stomach or of having stomach cancer.

Embodiment 1086. The method of Embodiment 1085, wherein the one or more proteins are selected from Table 215 or Table 2015.

Embodiment 1087. The method of Embodiment 1086, wherein the one or more proteins are selected from Table 215.A.1, 215.A.2, 2015.A.1, 2015.A.2, or 2015.A.3.

Embodiment 1088. The method of Embodiment 1086, wherein the one or more proteins are selected from Table 215.B.1, 215.B.2, 215.B.3, 215.B.4, 2015.B.1, 2015.B.2, or 2015.B.3.

Embodiment 1089. The method of Embodiment 1088, wherein the subject is female.

Embodiment 1090. The method of Embodiment 1086, wherein the one or more proteins are selected from Table 215.C.1, 215.C.2, 215.C.3, 215.C.4, 2015.C.1, 2015.C.2, or 2015.C.3.

Embodiment 1091. The method of Embodiment 1090, wherein the subject is male.

Embodiment 1092. The method of any one of Embodiments 1086-1091, wherein two or more proteins are selected.

Embodiment 1093. The method of Embodiment 1092, wherein three or more proteins are selected.

Embodiment 1094. The method of Embodiment 1093, wherein five or more proteins are selected.

Embodiment 1095. The method of Embodiment 1094, wherein ten or more proteins are selected.

Embodiment 1096. The method of any one of Embodiments 1086-1095, wherein no more than about 300 proteins are selected.

Embodiment 1097. The method of Embodiment 1096, wherein no more than about 200 proteins are selected.

Embodiment 1098. The method of Embodiment 1097, wherein no more than about 100 proteins are selected.

Embodiment 1099. The method of Embodiment 1098, wherein no more than about 50 proteins are selected.

Embodiment 1100. The method of Embodiment 1099, wherein no more than about 25 proteins are selected.

Embodiment 1101. The method of Embodiment 1086, wherein the one or more proteins is every protein of Table 215.A.1, 215.A.2, 2015.A.1, 2015.A.2, or 2015.A.3.

Embodiment 1102. The method of Embodiment 1086, wherein the one or more proteins is every protein of Table 215.B.1, 215.B.2, 215.B.3, 215.B.4, 2015.B.1, 2015.B.2, or 2015.B.3.

Embodiment 1103. The method of Embodiment 1102, wherein the subject is female.

Embodiment 1104. The method of Embodiment 1086, wherein the one or more proteins is every protein Table 215.C.1, 215.C.2, 215.C.3, 215.C.4, 2015.C.1, 2015.C.2, or 2015.C.3.

Embodiment 1105. The method of Embodiment 1104, wherein the subject is male.

Embodiment 1106. The method of any one of Embodiments 1085-1105, wherein the cancer is early-stage.

Embodiment 1107. The method of any one of Embodiments 783-789, wherein the subject is suspected of having a cancer associated with the testis or of having testicular cancer.

Embodiment 1108. The method of Embodiment 1107, wherein the one or more proteins are selected from Table 216 or Table 2016.

Embodiment 1109. The method of Embodiment 1108, wherein the one or more proteins are selected from Table 216.A.1, 216.A.2, 2016.A.1, 2016.A.2, or 2016.A.3.

Embodiment 1110. The method of Embodiment 1108, wherein the one or more proteins are selected from Table 216.B.1, 216.B.2, 216.B.3, 216.B.4, 2016.A.1, 2016.A.2, or 2016.A.3.

Embodiment 1111. The method of Embodiment 1110, wherein the subject is male.

Embodiment 1112. The method of any one of Embodiments 1108-1111, wherein two or more proteins are selected.

Embodiment 1113. The method of Embodiment 1112, wherein three or more proteins are selected.

Embodiment 1114. The method of Embodiment 1113, wherein five or more proteins are selected.

Embodiment 1115. The method of Embodiment 1114, wherein ten or more proteins are selected.

Embodiment 1116. The method of any one of Embodiments 1108-1115, wherein no more than about 300 proteins are selected.

Embodiment 1117. The method of Embodiment 1116, wherein no more than about 200 proteins are selected.

Embodiment 1118. The method of Embodiment 1117, wherein no more than about 100 proteins are selected.

Embodiment 1119. The method of Embodiment 1118, wherein no more than about 50 proteins are selected.

Embodiment 1120. The method of Embodiment 1119, wherein no more than about 25 proteins are selected.

Embodiment 1121. The method of Embodiment 1108, wherein the one or more proteins is every protein of Table 216.A.1, 216.A.2, 2016.A.1, 2016.A.2, or 2016.A.3.

Embodiment 1122. The method of Embodiment 1108, wherein the one or more proteins is every protein of Table 216.B.1, 216.11.2, 216.1B.3, 216.B.4, 2016.A.1, 2016.A.2, or 2016.A.3.

Embodiment 1123. The method of Embodiment 1122, wherein the subject is male.

Embodiment 1124. The method of any one of Embodiments 1107-1123, wherein the cancer is early-stage.

Embodiment 1125. The method of any one of Embodiments 783-789, wherein the subject is suspected of having a cancer associated with the thyroid.

Embodiment 1126. The method of Embodiment 1125, wherein the one or more proteins are selected from Table 217 or Table 2017.

Embodiment 1127. The method of Embodiment 1126, wherein the one or more proteins are selected from Table 217.A.1, 217.A.2, 2017.A.1, 2017.A.2, or 2017.A.3.

Embodiment 1128. The method of Embodiment 1126, wherein the one or more proteins are selected from Table 217.B.1, 217.1B.2, 217.B.3, 217.B.4, 2017.11.1, 2017.11.2, or 2017.B.3.

Embodiment 1129. The method of Embodiment 1128, wherein the subject is female.

Embodiment 1130. The method of Embodiment 1126, wherein the one or more proteins are selected from Table 217.C.1, 217.C.2, 217.C.3, 217.C.4, 2017.C.1, 2017.C.2, or 2017.C.3.

Embodiment 1131. The method of Embodiment 1130, wherein the subject is male.

Embodiment 1132. The method of any one of Embodiments 1126-1131, wherein two or more proteins are selected.

Embodiment 1133. The method of Embodiment 1132, wherein three or more proteins are selected.

Embodiment 1134. The method of Embodiment 1133, wherein five or more proteins are selected.

Embodiment 1135. The method of Embodiment 1134, wherein ten or more proteins are selected.

Embodiment 1136. The method of any one of Embodiments 1126-1135, wherein no more than about 300 proteins are selected.

Embodiment 1137. The method of Embodiment 1136, wherein no more than about 200 proteins are selected.

Embodiment 1138. The method of Embodiment 1137, wherein no more than about 100 proteins are selected.

Embodiment 1139. The method of Embodiment 1138, wherein no more than about 50 proteins are selected.

Embodiment 1140. The method of Embodiment 1139, wherein no more than about 25 proteins are selected.

Embodiment 1141. The method of Embodiment 1126, wherein the one or more proteins is every protein of Table 217.A.1, 217.A.2, 2017.A.1, 2017.A.2, or 2017.A.3.

Embodiment 1142. The method of Embodiment 1126, wherein the one or more proteins is every protein of Table 217.B.1, 217.B.2, 217.B.3, 217.B.4, 2017.B.1, 2017.B.2, or 2017.B.3.

Embodiment 1143. The method of Embodiment 1142, wherein the subject is female.

Embodiment 1144. The method of Embodiment 1126, wherein the one or more proteins is every protein Table 217.C.1, 217.C.2, 217.C.3, 217.C.4, 2017.C.1, 2017.C.2, or 2017.C.3.

Embodiment 1145. The method of Embodiment 1144, wherein the subject is male.

Embodiment 1146. The method of any one of Embodiments 1125-1145, wherein the cancer is early-stage.

Embodiment 1147. The method of any one of Embodiments 783-789, wherein the subject is suspected of having a cancer associated with the uterus or of having uterine cancer.

Embodiment 1148. The method of Embodiment 1147, wherein the one or more proteins are selected from Table 218, or Table 2018.

Embodiment 1149. The method of Embodiment 1148, wherein the one or more proteins are selected from Table 218.A.1, 218.A.2, 2018.A.1, 2018.A.2, or 2018.A.3.

Embodiment 1150. The method of Embodiment 1148, wherein the one or more proteins are selected from Table 218.B.1, 218.B.2, 218.B.3, 218.B.4, 2018.A.1, 2018.A.2, or 2018.A.3.

Embodiment 1151. The method of Embodiment 1150, wherein the subject is female.

Embodiment 1152. The method of any one of Embodiments 1148-1151, wherein two or more proteins are selected.

Embodiment 1153. The method of Embodiment 1152, wherein three or more proteins are selected.

Embodiment 1154. The method of Embodiment 1153, wherein five or more proteins are selected.

Embodiment 1155. The method of Embodiment 1154, wherein ten or more proteins are selected.

Embodiment 1156. The method of any one of Embodiments 1148-1155, wherein no more than about 300 proteins are selected.

Embodiment 1157. The method of Embodiment 1156, wherein no more than about 200 proteins are selected.

Embodiment 1158. The method of Embodiment 1157, wherein no more than about 100 proteins are selected.

Embodiment 1159. The method of Embodiment 1158, wherein no more than about 50 proteins are selected.

Embodiment 1160. The method of Embodiment 1159, wherein no more than about 25 proteins are selected.

Embodiment 1161. The method of Embodiment 1148, wherein the one or more proteins is every protein of Table 218.A.1, 218.A.2, 2018.A.1, 2018.A.2, or 2018.A.3.

Embodiment 1162. The method of Embodiment 1148, wherein the one or more proteins is every protein of Table 218.B.1, 218.B.2, 218.B.3, 218.B.4, 2018.A.1, 2018.A.2, or 2018.A.3.

Embodiment 1163. The method of Embodiment 1162, wherein the subject is female.

Embodiment 1164. The method of any one of Embodiments 1147-1163, wherein the cancer is early-stage.

Embodiment 1165. The method of any one of Embodiments 783-790, comprising determining in a biological sample from the subject a concentration of each protein from Table 251, 252, 253, or 254.

Embodiment 1166. The method of Embodiment 1165, wherein the concentration of each protein is from Table 251.

Embodiment 1167. The method of Embodiment 1166, wherein the subject is female.

Embodiment 1168. The method of Embodiment 1165, wherein the concentration of each protein is from Table 252.

Embodiment 1169. The method of Embodiment 1168, wherein the subject is female.

Embodiment 1170. The method of Embodiment 1165, wherein the concentration of each protein is from Table 253.

Embodiment 1171. The method of Embodiment 1170, wherein the subject is male.

Embodiment 1172. The method of Embodiment 1165, wherein the concentration of each protein is from Table 254.

Embodiment 1173. The method of Embodiment 1172, wherein the subject is male.

Embodiment 1174. The method of any one of Embodiments 1165-1173, wherein the cancer is early-stage.

Embodiment 1175. The method of any one of Embodiments 783-1174, wherein the subject is symptomatic of a cancer.

Embodiment 1176. The method of any one of Embodiments 1-1175, wherein the sample is selected from a plasma sample, urine sample, saliva sample, cerebrospinal fluid (CSF) sample, sweat sample, and tear sample.

Embodiment 1177. The method of any one of Embodiments 1-1175, wherein the number of proteins for which the concentration is determined is sufficient to achieve an area-under-the-curve (AUC) of a ROC curve of at least about 0.6.

Embodiment 1178. The method of Embodiment 1177, wherein the number of proteins for which the concentration is determined is sufficient to achieve an AUC of a ROC curve of at least about 0.7.

Embodiment 1179. The method of Embodiment 1178, wherein the number of proteins for which the concentration is determined is sufficient to achieve an AUC of a ROC curve of at least about 0.8.

Embodiment 1180. The method of Embodiment 1179, wherein the number of proteins for which the concentration is determined is sufficient to achieve an AUC of a ROC curve of at least about 0.9.

Embodiment 1181. The method of any one of Embodiments 1-1180, wherein the concentration of the two or more proteins is determined by one or more assays.

Embodiment 1182. A method of measuring amounts of proteins in a subject, the method comprising determining individual amounts of one or more proteins selected from Table 101 or selected from Table 1001.

Embodiment 1183. A method of measuring amounts of proteins in a subject, the method comprising determining individual amounts of one or more proteins selected from any of Tables 201-218., 2001-2018, 251, 252, 253, or 254.

Embodiment 1184. A method of measuring amounts of proteins in a subject, the method comprising determining individual amounts of each protein of Table 251, 252, 253, or 254.

Embodiment 1185. A method of measuring amounts of proteins in a subject, the method comprising determining individual amounts of one or more proteins selected from any of Tables 301-318.

Embodiment 1186. A method of evaluating a treatment for cancer in a subject, the method comprising:
(a) administering a treatment for cancer to the subject, and
(b) determining in a biological sample from the subject a concentration of one or more proteins, wherein the one or more proteins are selected from any of Tables 101, 1001, 201-218., 2001-2018, or 301-318: or determining in a biological sample from the subject a concentration of each protein of Table 251, 252, 253, or 254;
thereby evaluating the subject for cancer.

Embodiment 1187. A method of evaluating the efficacy of a treatment, comprising
(a) administering a treatment for cancer to the subject, and
(b) determining in a biological sample from the subject a concentration of one or more proteins, wherein the one or more proteins are selected from any of Tables 101, 1001, 201-218, 2001-2018, or 301-318; or determining in a biological sample from the subject a concentration of each protein of Table 251, 252, 253, or 254;
thereby evaluating the subject for cancer.

Embodiment 1188. A method of treating cancer in a subject, comprising
(a) administering a treatment for cancer, and
(b) determining in a biological sample from the subject a concentration of one or more proteins to evaluate the efficacy of the treatment, wherein the one or more proteins are selected from any of Tables 101, 1001, 201-218., 2001-2018, or 301-318; or determining in a biological sample from the subject a concentration of each protein of Table 251, 252, 253, or 254.

Embodiment 1189. A method of adjusting a treatment for cancer, comprising
(a) administering a treatment for cancer, and
(b) determining in a biological sample from the subject a concentration of one or more proteins, wherein the one or more proteins are selected from any of Tables 101, 1001, 201-218., 2001-2018, or 301-318; or determining in a biological sample from the subject a concentration of each protein of Table 251, 252, 253, or 254.

Embodiment 1190. A method of treating cancer in a subject, comprising
(a) administering a treatment for cancer, and
(b) determining in a biological sample from the subject a concentration of one or more proteins to evaluate whether the treatment requires adjustment, wherein the one or more proteins are selected from any of Tables 101, 1001, 201-218., 2001-2018, or 301-318; or determining in a biological sample from the subject a concentration of each protein of Table 251, 252, 253, or 254.

Embodiment 1191. A method of monitoring for cancer recurrence in a subject, comprising
(a) administering a treatment for cancer, and
(b) determining in a biological sample from the subject a concentration of one or more proteins to evaluate whether the treatment requires adjustment, wherein the one or more proteins are selected from any of Tables 101, 1001, 201-218., 2001-2018, or 301-318; or determining in a biological sample from the subject a concentration of each protein of Table 251, 252, 253, or 254.

Embodiment 1192. The method of Embodiment 1191, further comprising administering an adjusted treatment when it is determined that treatment requires adjustment.

Embodiment 1193. A method of treating cancer in a subject, comprising
(a) administering a treatment for cancer, and
(b) determining in a biological sample from the subject a concentration of one or more proteins to evaluate whether cancer is recurring, wherein the one or more proteins are selected from any of Tables 101, 1001, 201-218., 2001-2018, or 301-318; or determining in a biological sample from the subject a concentration of each protein of Table 251, 252, 253, or 254.

Embodiment 1194. The method of Embodiment 1193, further comprising administering a second treatment when it is determined that the cancer is recurring.

Embodiment 1195. The method of any one of Embodiments 1186-1194, wherein the one or more proteins are selected from Table 101.

Embodiment 1196. The method of Embodiment 1195, further comprising applying a classifier to the concentration of the one or more proteins that identifies whether the concentration of the one or more proteins is indicative of the subject having any cancer.

Embodiment 1197. The method of any one of Embodiments 1186-1194, wherein the one or more proteins are selected from Table 1001.

Embodiment 1198. The method of Embodiment 1197, further comprising applying a classifier to the concentration of the one or more proteins that identifies whether the concentration of the one or more proteins is indicative of the subject having any cancer.

Embodiment 1199. The method of Embodiment 1186-1194, wherein the one or more proteins are selected from Tables 201-218.

Embodiment 1200. The method of Embodiment 1199, further comprising applying a classifier to the concentration of the one or more proteins selected from Tables 201-218, that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 1201. The method of Embodiment 1186-1194, wherein the one or more proteins are selected from Tables 2001-2018.

Embodiment 1202. The method of Embodiment 1201, further comprising applying a classifier to the concentration of the one or more proteins selected from Tables 2001-2018 that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 1203. The method of Embodiment 1186-1194, wherein the one or more proteins are selected from Tables 301-318.

Embodiment 1204. The method of Embodiment 1203, further comprising applying a classifier to the concentration of the one or more proteins selected from Tables 301-318 that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 1205. The method of Embodiment 1186-1194, wherein the concentration is of each protein from Table 251, 252, 253, or 254.

Embodiment 1206. The method of Embodiment 1205, further comprising applying a classifier to the concentration of each protein from Table 251, 252, 253, or 254 that identifies whether the concentration is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

Embodiment 1207. The method of any one of Embodiments 1186-1206, wherein the administration of the treatment in (a) is performed by a third party.

Embodiment 1208. The method of any one of Embodiments 1186-1206, wherein the determination in a biological sample from the subject a concentration of one or more proteins in (b) is performed by a third party.

Embodiment 1209. The method of any one of Embodiments 1186-1204, 1207, or 1208, wherein two or more proteins are selected from any of Tables 101, 1001, 201-218., 2001-2018, or 301-318.

Embodiment 1210. The method of Embodiment 1209, wherein three or more proteins are selected from any of Tables 101, 1001, 201-218., 2001-2018, or 301-318.

Embodiment 1211. The method of Embodiment 1210, wherein five or more proteins are selected from any of Tables 101, 1001, 201-218., 2001-2018, or 301-318.

Embodiment 1212. The method of Embodiment 1211, wherein ten or more proteins are selected from any of Tables 101, 1001, 201-218., 2001-2018, or 301-318.

Embodiment 1213. The method of any one of Embodiments 1186-1204 or 1207-1212, wherein no more than about 300 proteins are selected from any of Tables 101, 1001, 201-218., 2001-2018, or 301-318.

Embodiment 1214. The method of Embodiment 1213, wherein no more than about 200 proteins are selected from any of Tables 101, 1001, 201-218., 2001-2018, or 301-318.

Embodiment 1215. The method of Embodiment 1214, wherein no more than about 100 proteins are selected from any of Tables 101, 1001, 201-218., 2001-2018, or 301-318.

Embodiment 1216. The method of Embodiment 1215, wherein no more than about 50 proteins are selected from any of Tables 101, 1001, 201-218., 2001-2018, or 301-318.

Embodiment 1217. The method of Embodiment 1216, wherein no more than about 25 proteins are selected from any of Tables 101, 1001, 201-218., 2001-2018, or 301-318.

Embodiment 1218. The method of any of Embodiments 38, 55, 793, or 807, wherein the one or more proteins or wherein every protein is selected from Table 201.A.1.

Embodiment 1219. The method of any of Embodiments 38, 55, 793, or 807, wherein the one or more proteins or wherein every protein is selected from Table 201.A.2.

Embodiment 1220. The method of any of Embodiments 40, 57, 794, or 808, wherein the one or more proteins or wherein every protein is selected from Table 201.B.1.

Embodiment 1221. The method of any of Embodiments 40, 57, 794, or 808, wherein the one or more proteins or wherein every protein is selected from Table 201.B.2.

Embodiment 1222. The method of any of Embodiments 40, 57, 794, or 808, wherein the one or more proteins or wherein every protein is selected from Table 201.B.3.

Embodiment 1223. The method of any of Embodiments 40, 57, 794, or 808, wherein the one or more proteins or wherein every protein is selected from Table 201.B.4.

Embodiment 1224. The method of any of Embodiments 43, 59, 796, or 810, wherein the one or more proteins or wherein every protein is selected from Table 201.C.1.

Embodiment 1225. The method of any of Embodiments 43, 59, 796, or 810, wherein the one or more proteins or wherein every protein is selected from Table 201.C.2.

Embodiment 1226. The method of any of Embodiments 43, 59, 796, or 810, wherein the one or more proteins or wherein every protein is selected from Table 201.C.3.

Embodiment 1227. The method of any of Embodiments 43, 59, 796, or 810, wherein the one or more proteins or wherein even protein is selected from Table 201.C.4.

Embodiment 1228. The method of any of Embodiments 39, 56, 793, or 807, wherein the one or more proteins or wherein every protein is selected from Table 2001.A.1.

Embodiment 1229. The method of any of Embodiments 39, 56, 793, or 807, wherein the one or more proteins or wherein every protein is selected from Table 2001.A.2.

Embodiment 1230. The method of any of Embodiments 39, 56, 793, or 807, wherein the one or more proteins or wherein every protein is selected from Table 2001.A.3.

Embodiment 1231. The method of any of Embodiments 41, 58, 794, or 808, wherein the one or more proteins or wherein every protein is selected from Table 2001.B.1.

Embodiment 1232. The method of any of Embodiments 41, 58, 794, or 808, wherein the one or more proteins or wherein every protein is selected from Table 2001.B.2.

Embodiment 1233. The method of any of Embodiments 41, 58, 794, or 808, wherein the one or more proteins or wherein every protein is selected from Table 2001.B.3.

Embodiment 1234. The method of any of Embodiments 44, 60, 796, or 810, wherein the one or more proteins or wherein every protein is selected from Table 2001.C.1.

Embodiment 1235. The method of any of Embodiments 44, 60, 796, or 810, wherein the one or more proteins or wherein every protein is selected from Table 2001.C.2.

Embodiment 1236. The method of any of Embodiments 44, 60, 796, or 810, wherein the one or more proteins or wherein every protein is selected from Table 2001.C.3.

Embodiment 1237. The method of any of Embodiments 61, 78, 816, or 830, wherein the one or more proteins or wherein every protein is selected from Table 202.A.1.

Embodiment 1238. The method of any of Embodiments 61, 78, 816, or 830, wherein the one or more proteins or wherein every protein is selected from Table 202.A.2.

Embodiment 1239. The method of any of Embodiments 63, 80, 817, or 831, wherein the one or more proteins or wherein every protein is selected from Table 202.B.1.

Embodiment 1240. The method of any of Embodiments 63, 80, 817, or 831, wherein the one or more proteins or wherein every protein is selected from Table 202.B.2.

Embodiment 1241. The method of any of Embodiments 63, 80, 817, or 831, wherein the one or more proteins or wherein every protein is selected from Table 202.1B.3.

Embodiment 1242. The method of any of Embodiments 63, 80, 817, or 831, wherein the one or more proteins or wherein every protein is selected from Table 202.1B.4.

Embodiment 1243. The method of any of Embodiments 66, 82, 819, or 833, wherein the one or more proteins or wherein every protein is selected from Table 202.C.1.

Embodiment 1244. The method of any of Embodiments 66, 82, 819, or 833, wherein the one or more proteins or wherein every protein is selected from Table 202.C.2.

Embodiment 1245. The method of any of Embodiments 66, 82, 819, or 833, wherein the one or more proteins or wherein every protein is selected from Table 202.C.3.

Embodiment 1246. The method of any of Embodiments 62, 79, 816, or 830, wherein the one or more proteins or wherein every protein is selected from Table 2002.A.1.

Embodiment 1247. The method of any of Embodiments 62, 79, 816, or 830, wherein the one or more proteins or wherein every protein is selected from Table 2002.A.2.

Embodiment 1248. The method of any of Embodiments 62, 79, 816, or 830, wherein the one or more proteins or wherein every protein is selected from Table 2002.A.3.

Embodiment 1249. The method of any of Embodiments 64, 81, 817, or 831, wherein the one or more proteins or wherein every protein is selected from Table 2002.1B.1.

Embodiment 1250. The method of any of Embodiments 64, 81, 817, or 831, wherein the one or more proteins or wherein every protein is selected from Table 2002.1B.2.

Embodiment 1251. The method of any of Embodiments 64, 81, 817, or 831, wherein the one or more proteins or wherein every protein is selected from Table 2002.B.3.

Embodiment 1252. The method of any of Embodiments 67, 83, 819, or 833, wherein the one or more proteins or wherein every protein is selected from Table 2002.C.1.

Embodiment 1253. The method of any of Embodiments 67, 83, 819, or 833, wherein the one or more proteins or wherein every protein is selected from Table 2002.C.2.

Embodiment 1254. The method of any of Embodiments 67, 83, 819, or 833, wherein the one or more proteins or wherein every protein is selected from Table 2002.C.3.

Embodiment 1255. The method of any of Embodiments 84, 101, 839, or 853, wherein the one or more proteins or wherein every protein is selected from Table 203.A.1.

Embodiment 1256. The method of any of Embodiments 84, 101, 839, or 853, wherein the one or more proteins or wherein every protein is selected from Table 203.A.2.

Embodiment 1257. The method of any of Embodiments 86, 103, 840, or 854, wherein the one or more proteins or wherein every protein is selected from Table 203.B.1.

Embodiment 1258. The method of any of Embodiments 86, 103, 840, or 854, wherein the one or more proteins or wherein every protein is selected from Table 203.1.2.

Embodiment 1259. The method of any of Embodiments 86, 103, 840, or 854, wherein the one or more proteins or wherein every protein is selected from Table 203.B.3.

Embodiment 1260. The method of any of Embodiments 86, 103, 840, or 854, wherein the one or more proteins or wherein every protein is selected from Table 203.1B.4.

Embodiment 1261. The method of any of Embodiments 89, 105, 842, or 856, wherein the one or more proteins or wherein every protein is selected from Table 203.C.1.

Embodiment 1262. The method of any of Embodiments 89, 105, 842, or 856, wherein the one or more proteins or wherein every protein is selected from Table 203.C.2.

Embodiment 1263. The method of any of Embodiments 89, 105, 842, or 856, wherein the one or more proteins or wherein every protein is selected from Table 203.C.3.

Embodiment 1264. The method of any of Embodiments 89, 105, 842, or 856, wherein the one or more proteins or wherein every protein is selected from Table 203.C.4.

Embodiment 1265. The method of any of Embodiments 85, 102, 839, or 853, wherein the one or more proteins or wherein every protein is selected from Table 2003.A.1.

Embodiment 1266. The method of any of Embodiments 85, 102, 839, or 853, wherein the one or more proteins or wherein every protein is selected from Table 2003.A.2.

Embodiment 1267. The method of any of Embodiments 85, 102, 839, or 853, wherein the one or more proteins or wherein every protein is selected from Table 2003.A.3.

Embodiment 1268. The method of any of Embodiments 87, 104, 840, or 854, wherein the one or more proteins or wherein every protein is selected from Table 2003.B.1.

Embodiment 1269. The method of any of Embodiments 87, 104, 840, or 854, wherein the one or more proteins or wherein every protein is selected from Table 2003.B.2.

Embodiment 1270. The method of any of Embodiments 87, 104, 840, or 854, wherein the one or more proteins or wherein every protein is selected from Table 2003.B.3.

Embodiment 1271. The method of any of Embodiments 90, 106, 842, or 856, wherein the one or more proteins or wherein every protein is selected from Table 2003.C.1.

Embodiment 1272. The method of any of Embodiments 90, 106, 842, or 856, wherein the one or more proteins or wherein every protein is selected from Table 2003.C.2.

Embodiment 1273. The method of any of Embodiments 90, 106, 842, or 856, wherein the one or more proteins or wherein every protein is selected from Table 2003.C.3.

Embodiment 1274. The method of any of Embodiments 107, 121, 861, or 873, wherein the one or more proteins or wherein every protein is selected from Table 204.A.1.

Embodiment 1275. The method of any of Embodiments 107, 121, 861, or 873, wherein the one or more proteins or wherein every protein is selected from Table 204.A.2.

Embodiment 1276. The method of any of Embodiments 109, 123, 862, or 874, wherein the one or more proteins or wherein every protein is selected from Table 204.B.1.

Embodiment 1277. The method of any of Embodiments 109, 123, 862, or 874, wherein the one or more proteins or wherein every protein is selected from Table 204.1.2.

Embodiment 1278. The method of any of Embodiments 109, 123, 862, or 874, wherein the one or more proteins or wherein every protein is selected from Table 204.B.3.

Embodiment 1279. The method of any of Embodiments 109, 123, 862, or 874, wherein the one or more proteins or wherein every protein is selected from Table 204.B.4.

Embodiment 1280. The method of any of Embodiments 108, 110, 122, 124, 861, 862, 873, or 874, wherein the one or more proteins or wherein every protein is selected from Table 2004.A.1.

Embodiment 1281. The method of any of Embodiments 110, 124, 861, 862, 873, or 874, wherein the one or more proteins or wherein every protein is selected from Table 2004.A.2.

Embodiment 1282. The method of any of Embodiments 110, 124, 861, 862, 873, or 874, wherein the one or more proteins or wherein every protein is selected from Table 2004.A.3.

Embodiment 1283. The method of any of Embodiments 125, 139, 879, or 891, wherein the one or more proteins or wherein every protein is selected from Table 205.A.1.

Embodiment 1284. The method of any of Embodiments 125, 139, 879, or 891, wherein the one or more proteins or wherein every protein is selected from Table 205.A.2.

Embodiment 1285. The method of any of Embodiments 127, 141, 880, or 892, wherein the one or more proteins or wherein every protein is selected from Table 205.B.1.

Embodiment 1286. The method of any of Embodiments 127, 141, 880, or 892, wherein the one or more proteins or wherein every protein is selected from Table 205.B.2.

Embodiment 1287. The method of any of Embodiments 127, 141, 880, or 892, wherein the one or more proteins or wherein every protein is selected from Table 205.1B3.

Embodiment 1288. The method of any of Embodiments 127, 141, 880, or 892, wherein the one or more proteins or wherein every protein is selected from Table 205.B.4.

Embodiment 1289. The method of any of Embodiments 126, 128, 140, 142, 879, 880, 891, or 892, wherein the one or more proteins or wherein every protein is selected from Table 2005.A.1.

Embodiment 1290. The method of any of Embodiments 128, 142, 879, 880, 891, or 892, wherein the one or more proteins or wherein every protein is selected from Table 2005.A.2.

Embodiment 1291. The method of any of Embodiments 128, 142, 879, 880, 891, or 892, wherein the one or more proteins or wherein every protein is selected from Table 2005.A.3.

Embodiment 1292. The method of any of Embodiments 143, 160, 897, or 911, wherein the one or more proteins or wherein every protein is selected from Table 206.A.1.

Embodiment 1293. The method of any of Embodiments 143, 160, 897, or 911, wherein the one or more proteins or wherein every protein is selected from Table 206.A.2.

Embodiment 1294. The method of any of Embodiments 145, 162, 898, or 912, wherein the one or more proteins or wherein every protein is selected from Table 206.B.1.

Embodiment 1295. The method of any of Embodiments 145, 162, 898, or 912, wherein the one or more proteins or wherein every protein is selected from Table 206.1B.2.

Embodiment 1296. The method of any of Embodiments 145, 162, 898, or 912, wherein the one or more proteins or wherein every protein is selected from Table 206.1B.3.

Embodiment 1297. The method of any of Embodiments 145, 162, 898, or 912, wherein the one or more proteins or wherein every protein is selected from Table 206.1B.4.

Embodiment 1298. The method of any of Embodiments 148, 164, 900, or 914, wherein the one or more proteins or wherein every protein is selected from Table 206.C.1.

Embodiment 1299. The method of any of Embodiments 148, 164, 900, or 914, wherein the one or more proteins or wherein every protein is selected from Table 206.C.2.

Embodiment 1300. The method of any of Embodiments 148, 164, 900, or 914, wherein the one or more proteins or wherein every protein is selected from Table 206.C.3.

Embodiment 1301. The method of any of Embodiments 144, 161, 897, or 911, wherein the one or more proteins or wherein every protein is selected from Table 2006.A.1.

Embodiment 1302. The method of any of Embodiments 144, 161, 897, or 911, wherein the one or more proteins or wherein every protein is selected from Table 2006.A.2.

Embodiment 1303. The method of any of Embodiments 144, 161, 897, or 911, wherein the one or more proteins or wherein every protein is selected from Table 2006.A.3.

Embodiment 1304. The method of any of Embodiments 146, 163, 898, or 912, wherein the one or more proteins or wherein every protein is selected from Table 2006.B.1.

Embodiment 1305. The method of any of Embodiments 146, 163, 898, or 912, wherein the one or more proteins or wherein every protein is selected from Table 2006.B.2.

Embodiment 1306. The method of any of Embodiments 146, 163, 898, or 912, wherein the one or more proteins or wherein every protein is selected from Table 2006.B.3.

Embodiment 1307. The method of any of Embodiments 149, 165, 900, or 914, wherein the one or more proteins or wherein every protein is selected from Table 2006.C.1.

Embodiment 1308. The method of any of Embodiments 149, 165, 900, or 914, wherein the one or more proteins or wherein every protein is selected from Table 2006.C.2.

Embodiment 1309. The method of any of Embodiments 149, 165, 900, or 9140, wherein the one or more proteins or wherein every protein is selected from Table 2006.C.3.

Embodiment 1310. The method of any of Embodiments 166, 183, 919, or 933, wherein the one or more proteins or wherein every protein is selected from Table 207.A.1.

Embodiment 1311. The method of any of Embodiments 166, 183, 919, or 933, wherein the one or more proteins or wherein every protein is selected from Table 207.A.2.

Embodiment 1312. The method of any of Embodiments 168, 185, 920, or 934, wherein the one or more proteins or wherein every protein is selected from Table 207.B.1.

Embodiment 1313. The method of any of Embodiments 168, 185, 920, or 934, wherein the one or more proteins or wherein every protein is selected from Table 207.1B.2.

Embodiment 1314. The method of any of Embodiments 168, 185, 920, or 934, wherein the one or more proteins or wherein every protein is selected from Table 207.1B.3.

Embodiment 1315. The method of any of Embodiments 168, 185, 920, or 934, wherein the one or more proteins or wherein every protein is selected from Table 207.1B.4.

Embodiment 1316. The method of any of Embodiments 171, 187, 922, or 936, wherein the one or more proteins or wherein every protein is selected from Table 207.C.1.

Embodiment 1317. The method of any of Embodiments 171, 187, 922, or 936, wherein the one or more proteins or wherein every protein is selected from Table 207.C.2.

Embodiment 1318. The method of any of Embodiments 171, 187, 922, or 936, wherein the one or more proteins or wherein every protein is selected from Table 207.C.3.

Embodiment 1319. The method of any of Embodiments 167, 184, 919, or 933, wherein the one or more proteins or wherein every protein is selected from Table 2007.A.1.

Embodiment 1320. The method of any of Embodiments 167, 184, 919, or 933, wherein the one or more proteins or wherein every protein is selected from Table 2007.A.2.

Embodiment 1321. The method of any of Embodiments 167, 184, 919, or 933, wherein the one or more proteins or wherein every protein is selected from Table 2007.A.3.

Embodiment 1322. The method of any of Embodiments 169, 186, 920, or 934, wherein the one or more proteins or wherein every protein is selected from Table 2007.B.1.

Embodiment 1323. The method of any of Embodiments 169, 186, 920, or 934, wherein the one or more proteins or wherein every protein is selected from Table 2007.B.2.

Embodiment 1324. The method of any of Embodiments 169, 186, 920, or 934, wherein the one or more proteins or wherein every protein is selected from Table 2007.B.3.

Embodiment 1325. The method of any of Embodiments 172, 188, 922, or 936, wherein the one or more proteins or wherein every protein is selected from Table 2007.C.1.

Embodiment 1326. The method of any of Embodiments 172, 188, 922, or 936, wherein the one or more proteins or wherein every protein is selected from Table 2007.C.2.

Embodiment 1327. The method of any of Embodiments 172, 188, 922, or 936, wherein the one or more proteins or wherein every protein is selected from Table 2007.C.3.

Embodiment 1328. The method of any of Embodiments 189, 206, 941, or 955, wherein the one or more proteins or wherein every protein is selected from Table 208.A.1.

Embodiment 1329. The method of any of Embodiments 189, 206, 941, or 955, wherein the one or more proteins or wherein every protein is selected from Table 208.A.2.

Embodiment 1330. The method of any of Embodiments 191, 208, 942, or 956, wherein the one or more proteins or wherein every protein is selected from Table 208.1.1.

Embodiment 1331. The method of any of Embodiments 191, 208, 942, or 956, wherein the one or more proteins or wherein every protein is selected from Table 208.B.2.

Embodiment 1332. The method of any of Embodiments 191, 208, 942, or 956, wherein the one or more proteins or wherein every protein is selected from Table 208.B.3.

Embodiment 1333. The method of any of Embodiments 191, 208, 942, or 956, wherein the one or more proteins or wherein every protein is selected from Table 208.1.4.

Embodiment 1334. The method of any of Embodiments 194, 210, 944, or 958, wherein the one or more proteins or wherein every protein is selected from Table 208.C.1.

Embodiment 1335. The method of any of Embodiments 194, 210, 944, or 958, wherein the one or more proteins or wherein every protein is selected from Table 208.C.2.

Embodiment 1336. The method of any of Embodiments 194, 210, 944, or 958, wherein the one or more proteins or wherein every protein is selected from Table 208.C.3.

Embodiment 1337. The method of any of Embodiments 194, 210, 944, or 958, wherein the one or more proteins or wherein every protein is selected from Table 208.C.4.

Embodiment 1338. The method of any of Embodiments 190, 207, 941, or 955, wherein the one or more proteins or wherein every protein is selected from Table 2008.A.1.

Embodiment 1339. The method of any of Embodiments 190, 207, 941, or 955, wherein the one or more proteins or wherein every protein is selected from Table 2008.A.2.

Embodiment 1340. The method of any of Embodiments 190, 207, 941, or 955, wherein the one or more proteins or wherein every protein is selected from Table 2008.A.3.

Embodiment 1341. The method of any of Embodiments 192, 209, 942, or 956, wherein the one or more proteins or wherein every protein is selected from Table 2008.B.1.

Embodiment 1342. The method of any of Embodiments 192, 209, 942, or 956, wherein the one or more proteins or wherein every protein is selected from Table 2008.B.2.

Embodiment 1343. The method of any of Embodiments 192, 209, 942, or 956, wherein the one or more proteins or wherein every protein is selected from Table 2008.B.3.

Embodiment 1344. The method of any of Embodiments 195, 211, 944, or 958, wherein the one or more proteins or wherein every protein is selected from Table 2008.C.1.

Embodiment 1345. The method of any of Embodiments 195, 211, 944, or 958, wherein the one or more proteins or wherein every protein is selected from Table 2008.C.2.

Embodiment 1346. The method of any of Embodiments 195, 211, 944, or 958, wherein the one or more proteins or wherein every protein is selected from Table 2008.C.3.

Embodiment 1347. The method of any of Embodiments 212, 229, 963, or 977, wherein the one or more proteins or wherein every protein is selected from Table 209.A.1.

Embodiment 1348. The method of any of Embodiments 212, 229, 963, or 977, wherein the one or more proteins or wherein every protein is selected from Table 209.A.2.

Embodiment 1349. The method of any of Embodiments 214, 231, 964, or 978, wherein the one or more proteins or wherein every protein is selected from Table 209.B.1.

Embodiment 1350. The method of any of Embodiments 214, 231, 964, or 978, wherein the one or more proteins or wherein every protein is selected from Table 209.B.2.

Embodiment 1351. The method of any of Embodiments 214, 231, 964, or 978, wherein the one or more proteins or wherein every protein is selected from Table 209.B.3.

Embodiment 1352. The method of any of Embodiments 214, 231, 964, or 978, wherein the one or more proteins or wherein every protein is selected from Table 209.B.4.

Embodiment 1353. The method of any of Embodiments 217, 233, 966, or 980, wherein the one or more proteins or wherein every protein is selected from Table 209.C.1.

Embodiment 1354. The method of any of Embodiments 217, 233, 966, or 980, wherein the one or more proteins or wherein every protein is selected from Table 209.C.2.

Embodiment 1355. The method of any of Embodiments 217, 233, 966, or 980, wherein the one or more proteins or wherein every protein is selected from Table 209.C.3.

Embodiment 1356. The method of any of Embodiments 217, 233, 966, or 980, wherein the one or more proteins or wherein every protein is selected from Table 209.C.4.

Embodiment 1357. The method of any of Embodiments 213, 230, 963, or 977, wherein the one or more proteins or wherein every protein is selected from Table 2009.A.1.

Embodiment 1358. The method of any of Embodiments 213, 230, 963, or 977, wherein the one or more proteins or wherein every protein is selected from Table 2009.A.2.

Embodiment 1359. The method of any of Embodiments 213, 230, 963, or 977, wherein the one or more proteins or wherein every protein is selected from Table 2009.A.3.

Embodiment 1360. The method of any of Embodiments 215, 232, 964, or 978, wherein the one or more proteins or wherein every protein is selected from Table 2009.B.1.

Embodiment 1361. The method of any of Embodiments 215, 232, 964, or 978, wherein the one or more proteins or wherein every protein is selected from Table 2009.B.2.

Embodiment 1362. The method of any of Embodiments 215, 232, 964, or 978, wherein the one or more proteins or wherein every protein is selected from Table 2009.B.3.

Embodiment 1363. The method of any of Embodiments 218., 234, 966, or 980, wherein the one or more proteins or wherein every protein is selected from Table 2009.C.1.

Embodiment 1364. The method of any of Embodiments 218., 234, 966, or 980, wherein the one or more proteins or wherein every protein is selected from Table 2009.C.2.

Embodiment 1365. The method of any of Embodiments 218., 234, 966, or 980, wherein the one or more proteins or wherein every protein is selected from Table 2009.C.3.

Embodiment 1366. The method of any of Embodiments 235, 252, 985, or 999, wherein the one or more proteins or wherein every protein is selected from Table 210.A.1.

Embodiment 1367. The method of any of Embodiments 235, 252, 985, or 999, wherein the one or more proteins or wherein every protein is selected from Table 210.A.2.

Embodiment 1368. The method of any of Embodiments 237, 254, 986, or 1000, wherein the one or more proteins or wherein every protein is selected from Table 210.1B.1.

Embodiment 1369. The method of any of Embodiments 237, 254, 986, or 1000, wherein the one or more proteins or wherein every protein is selected from Table 210.1B.2.

Embodiment 1370. The method of any of Embodiments 237, 254, 986, or 1000, wherein the one or more proteins or wherein every protein is selected from Table 210.1B.3.

Embodiment 1371. The method of any of Embodiments 237, 254, 986, or 1000, wherein the one or more proteins or wherein every protein is selected from Table 210.B.4.

Embodiment 1372. The method of any of Embodiments 240, 256, 988, or 1002, wherein the one or more proteins or wherein every protein is selected from Table 210.C.1.

Embodiment 1373. The method of any of Embodiments 240, 256, 988, or 1002, wherein the one or more proteins or wherein every protein is selected from Table 210.C.2.

Embodiment 1374. The method of any of Embodiments 240, 256, 988, or 1002, wherein the one or more proteins or wherein every protein is selected from Table 210.C.3.

Embodiment 1375. The method of any of Embodiments 240, 256, 988, or 1002, wherein the one or more proteins or wherein every protein is selected from Table 210.C.4.

Embodiment 1376. The method of any of Embodiments 236, 253, 985, or 999, wherein the one or more proteins or wherein every protein is selected from Table 2010.A.1.

Embodiment 1377. The method of any of Embodiments 236, 253, 985, or 999, wherein the one or more proteins or wherein every protein is selected from Table 2010.A.2.

Embodiment 1378. The method of any of Embodiments 236, 253, 985, or 999, wherein the one or more proteins or wherein every protein is selected from Table 2010.A.3.

Embodiment 1379. The method of any of Embodiments 238, 255, 986, or 1000, wherein the one or more proteins or wherein every protein is selected from Table 2010.B.1.

Embodiment 1380. The method of any of Embodiments 238, 255, 986, or 1000, wherein the one or more proteins or wherein every protein is selected from Table 2010.1B.2.

Embodiment 1381. The method of any of Embodiments 238, 255, 986, or 1000, wherein the one or more proteins or wherein every protein is selected from Table 2010.B.3.

Embodiment 1382. The method of any of Embodiments 241, 257, 988, or 1002, wherein the one or more proteins or wherein every protein is selected from Table 2010.C.1.

Embodiment 1383. The method of any of Embodiments 241, 257, 988, or 1002, wherein the one or more proteins or wherein every protein is selected from Table 2010.C.2.

Embodiment 1384. The method of any of Embodiments 241, 257, 988, or 1002, wherein the one or more proteins or wherein every protein is selected from Table 2010.C.3.

Embodiment 1385. The method of any of Embodiments 258, 275, 1007, or 1021, wherein the one or more proteins or wherein every protein is selected from Table 211.A.1.

Embodiment 1386. The method of any of Embodiments 258, 275, 1007, or 1021, wherein the one or more proteins or wherein every protein is selected from Table 211.A.2.

Embodiment 1387. The method of any of Embodiments 260, 277, 1008, or 1022, wherein the one or more proteins or wherein every protein is selected from Table 211.B.1.

Embodiment 1388. The method of any of Embodiments 260, 277, 1008, or 1022, wherein the one or more proteins or wherein every protein is selected from Table 211.B.2.

Embodiment 1389. The method of any of Embodiments 260, 277, 1008, or 1022, wherein the one or more proteins or wherein every protein is selected from Table 211.B.3.

Embodiment 1390. The method of any of Embodiments 260, 277, 1008, or 1022, wherein the one or more proteins or wherein every protein is selected from Table 211.B.4.

Embodiment 1391. The method of any of Embodiments 263, 279, 1010, or 1024, wherein the one or more proteins or wherein every protein is selected from Table 211.C.1.

Embodiment 1392. The method of any of Embodiments 263, 279, 1010, or 1024, wherein the one or more proteins or wherein every protein is selected from Table 211.C.2.

Embodiment 1393. The method of any of Embodiments 263, 279, 1010, or 1024, wherein the one or more proteins or wherein every protein is selected from Table 211.C.3.

Embodiment 1394. The method of any of Embodiments 259, 276, 1007, or 1021, wherein the one or more proteins or wherein every protein is selected from Table 2011.A.1.

Embodiment 1395. The method of any of Embodiments 259, 276, 1007, or 1021, wherein the one or more proteins or wherein every protein is selected from Table 2011.A.2.

Embodiment 1396. The method of any of Embodiments 261, 278, 1008, or 1022, wherein the one or more proteins or wherein every protein is selected from Table 2011.B.1.

Embodiment 1397. The method of any of Embodiments 261, 278, 1008, or 1022, wherein the one or more proteins or wherein every protein is selected from Table 2011.B.2.

Embodiment 1398. The method of any of Embodiments 261, 278, 1008, or 1022, wherein the one or more proteins or wherein every protein is selected from Table 2011.B.3.

Embodiment 1399. The method of any of Embodiments 264, 280, 1010, or 1024, wherein the one or more proteins or wherein every protein is selected from Table 2011.C.1.

Embodiment 1400. The method of any of Embodiments 264, 280, 1010, or 1024, wherein the one or more proteins or wherein every protein is selected from Table 2011.C.2.

Embodiment 1401. The method of any of Embodiments 264, 280, 1010, or 1024, wherein the one or more proteins or wherein every protein is selected from Table 2011.C.3.

Embodiment 1402. The method of any of Embodiments 281, 295, 1029, or 1041, wherein the one or more proteins or wherein every protein is selected from Table 212.A.1.

Embodiment 1403. The method of any of Embodiments 281, 295, 1029, or 1041, wherein the one or more proteins or wherein every protein is selected from Table 212.A.2.

Embodiment 1404. The method of any of Embodiments 283, 297, 1030, or 1042, wherein the one or more proteins or wherein every protein is selected from Table 212.B.1.

Embodiment 1405. The method of any of Embodiments 283, 297, 1030, or 1042, wherein the one or more proteins or wherein every protein is selected from Table 212.1B.2.

Embodiment 1406. The method of any of Embodiments 283, 297, 1030, or 1042, wherein the one or more proteins or wherein every protein is selected from Table 212.11.3.

Embodiment 1407. The method of any of Embodiments 283, 297, 1030, or 1042, wherein the one or more proteins or wherein every protein is selected from Table 212.1B.4.

Embodiment 1408. The method of any of Embodiments 282, 284, 296, 298, 1029, 1030, 1041, or 1042, wherein the one or more proteins or wherein every protein is selected from Table 2012.A.1.

Embodiment 1409. The method of any of Embodiments 284, 298, 1029, 1030, 1041, or 1042, wherein the one or more proteins or wherein every protein is selected from Table 2012.A.2.

Embodiment 1410. The method of any of Embodiments 284, 298, 1029, 1030, 1041, or 1042, wherein the one or more proteins or wherein every protein is selected from Table 2012.A.3.

Embodiment 1411. The method of any of Embodiments 299, 316, 1047, or 1061, wherein the one or more proteins or wherein every protein is selected from Table 213.A.1.

Embodiment 1412. The method of any of Embodiments 299, 316, 1047, or 1061, wherein the one or more proteins or wherein every protein is selected from Table 213.A.2.

Embodiment 1413. The method of any of Embodiments 301, 318, 1048, or 1062, wherein the one or more proteins or wherein every protein is selected from Table 213.B.1.

Embodiment 1414. The method of any of Embodiments 301, 318, 1048, or 1062, wherein the one or more proteins or wherein every protein is selected from Table 213.1B.2.

Embodiment 1415. The method of any of Embodiments 301, 318, 1048, or 1062, wherein the one or more proteins or wherein every protein is selected from Table 213.1B.3.

Embodiment 1416. The method of any of Embodiments 304, 320, 1050, or 1064, wherein the one or more proteins or wherein every protein is selected from Table 213.C.1.

Embodiment 1417. The method of any of Embodiments 304, 320, 1050, or 1064, wherein the one or more proteins or wherein every protein is selected from Table 213.C.2.

Embodiment 1418. The method of any of Embodiments 304, 320, 1050, or 1064, wherein the one or more proteins or wherein every protein is selected from Table 213.C.3.

Embodiment 1419. The method of any of Embodiments 300, 317, 1047, or 1061, wherein the one or more proteins or wherein every protein is selected from Table 2013.A.1.

Embodiment 1420. The method of any of Embodiments 300, 317, 1047, or 1061, wherein the one or more proteins or wherein every protein is selected from Table 2013.A.2.

Embodiment 1421. The method of any of Embodiments 300, 317, 1047, or 1061, wherein the one or more proteins or wherein every protein is selected from Table 2013.A.3.

Embodiment 1422. The method of any of Embodiments 302, 319, 1048, or 1062, wherein the one or more proteins or wherein every protein is selected from Table 2013.B.1.

Embodiment 1423. The method of any of Embodiments 302, 319, 1048, or 1062, wherein the one or more proteins or wherein every protein is selected from Table 2013.1B.2.

Embodiment 1424. The method of any of Embodiments 302, 319, 1048, or 1062, wherein the one or more proteins or wherein every protein is selected from Table 2013.1B.3.

Embodiment 1425. The method of any of Embodiments 305, 321, 1050, or 1064, wherein the one or more proteins or wherein every protein is selected from Table 2013.C.1.

Embodiment 1426. The method of any of Embodiments 305, 321, 1050, or 1064, wherein the one or more proteins or wherein every protein is selected from Table 2013.C.2.

Embodiment 1427. The method of any of Embodiments 305, 321, 1050, or 1064, wherein the one or more proteins or wherein every protein is selected from Table 2013.C.3.

Embodiment 1428. The method of any of Embodiments 322, 336, 1069, or 1081, wherein the one or more proteins or wherein every protein is selected from Table 214.A.1.

Embodiment 1429. The method of any of Embodiments 322, 336, 1069, or 1081, wherein the one or more proteins or wherein every protein is selected from Table 214.A.2.

Embodiment 1430. The method of any of Embodiments 324, 338, 1070, or 1082, wherein the one or more proteins or wherein every protein is selected from Table 214.B.1.

Embodiment 1431. The method of any of Embodiments 324, 338, 1070, or 1082, wherein the one or more proteins or wherein every protein is selected from Table 214.1.2.

Embodiment 1432. The method of any of Embodiments 324, 338, 1070, or 1082, wherein the one or more proteins or wherein every protein is selected from Table 214.B.3.

Embodiment 1433. The method of any of Embodiments 324, 338, 1070, or 1082, wherein the one or more proteins or wherein every protein is selected from Table 214.1B.4.

Embodiment 1434. The method of any of Embodiments 323, 325, 337, 339, 1069, 1070, 1081, or 1082, wherein the one or more proteins or wherein every protein is selected from Table 2014.A.1.

Embodiment 1435. The method of any of Embodiments 325, 339, 1069, 1070, 1081, or 1082, wherein the one or more proteins or wherein every protein is selected from Table 2014.A.2.

Embodiment 1436. The method of any of Embodiments 325, 339, 1069, 1070, 1081, or 1082, wherein the one or more proteins or wherein every protein is selected from Table 2014.A.3.

Embodiment 1437. The method of any of Embodiments 340, 357, 1087, or 1101, wherein the one or more proteins or wherein every protein is selected from Table 215.A.1.

Embodiment 1438. The method of any of Embodiments 340, 357, 1087, or 1101, wherein the one or more proteins or wherein every protein is selected from Table 215.A.2.

Embodiment 1439. The method of any of Embodiments 342, 359, 1088, or 1102, wherein the one or more proteins or wherein every protein is selected from Table 215.B.1.

Embodiment 1440. The method of any of Embodiments 342, 359, 1088, or 1102, wherein the one or more proteins or wherein every protein is selected from Table 215.B.2.

Embodiment 1441. The method of any of Embodiments 342, 359, 1088, or 1102, wherein the one or more proteins or wherein every protein is selected from Table 215.B.3.

Embodiment 1442. The method of any of Embodiments 342, 359, 1088, or 1102, wherein the one or more proteins or wherein every protein is selected from Table 215.B.4.

Embodiment 1443. The method of any of Embodiments 345, 361, 1090, or 1104, wherein the one or more proteins or wherein every protein is selected from Table 215.C.1.

Embodiment 1444. The method of any of Embodiments 345, 361, 1090, or 1104, wherein the one or more proteins or wherein every protein is selected from Table 215.C.2.

Embodiment 1445. The method of any of Embodiments 345, 361, 1090, or 1104, wherein the one or more proteins or wherein every protein is selected from Table 215.C.3.

Embodiment 1446. The method of any of Embodiments 345, 361, 1090, or 1104, wherein the one or more proteins or wherein every protein is selected from Table 215.C.4.

Embodiment 1447. The method of any of Embodiments 341, 358, 1087, or 1101, wherein the one or more proteins or wherein every protein is selected from Table 2015.A.1.

Embodiment 1448. The method of any of Embodiments 341, 358, 1087, or 1101, wherein the one or more proteins or wherein every protein is selected from Table 2015.A.2.

Embodiment 1449. The method of any of Embodiments 341, 358, 1087, or 1101, wherein the one or more proteins or wherein every protein is selected from Table 2015.A.3.

Embodiment 1450. The method of any of Embodiments 343, 360, 1088, or 1102, wherein the one or more proteins or wherein every protein is selected from Table 2015.B.1.

Embodiment 1451. The method of any of Embodiments 343, 360, 1088, or 1102, wherein the one or more proteins or wherein every protein is selected from Table 2015.B.2.

Embodiment 1452. The method of any of Embodiments 343, 360, 1088, or 1102, wherein the one or more proteins or wherein every protein is selected from Table 2015.B.3.

Embodiment 1453. The method of any of Embodiments 346, 362, 1090, or 1104, wherein the one or more proteins or wherein every protein is selected from Table 2015.C.1.

Embodiment 1454. The method of any of Embodiments 346, 362, 1090, or 1104, wherein the one or more proteins or wherein every protein is selected from Table 2015.C.2.

Embodiment 1455. The method of any of Embodiments 346, 362, 1090, or 1104, wherein the one or more proteins or wherein every protein is selected from Table 2015.C.3.

Embodiment 1456. The method of any of Embodiments 363, 377, 1109, or 1121, wherein the one or more proteins or wherein every protein is selected from Table 216.A.1.

Embodiment 1457. The method of any of Embodiments 363, 377, 1109, or 1121, wherein the one or more proteins or wherein every protein is selected from Table 216.A.2.

Embodiment 1458. The method of any of Embodiments 365, 379, 1110, or 1122, wherein the one or more proteins or wherein every protein is selected from Table 216.B.1.

Embodiment 1459. The method of any of Embodiments 365, 379, 1110, or 1122, wherein the one or more proteins or wherein every protein is selected from Table 216.B.2.

Embodiment 1460. The method of any of Embodiments 365, 379, 1110, or 1122, wherein the one or more proteins or wherein every protein is selected from Table 216.B.3.

Embodiment 1461. The method of any of Embodiments 365, 379, 1110, or 1122, wherein the one or more proteins or wherein every protein is selected from Table 216.B.4.

Embodiment 1462. The method of any of Embodiments 364, 366, 378, 380, 1109, 1110, 1121, or 1122, wherein the one or more proteins or wherein every protein is selected from Table 2016.A.1.

Embodiment 1463. The method of any of Embodiments 366, 380, 1109, 1110, 1121, or 1122, wherein the one or more proteins or wherein every protein is selected from Table 2016.A.2.

Embodiment 1464. The method of any of Embodiments 366, 380, 1109, 1110, 1121, or 1122, wherein the one or more proteins or wherein every protein is selected from Table 2016.A.3.

Embodiment 1465. The method of any of Embodiments 381, 398, 1127, or 1141, wherein the one or more proteins or wherein every protein is selected from Table 217.A.1.

Embodiment 1466. The method of any of Embodiments 381, 398, 1127, or 1141, wherein the one or more proteins or wherein every protein is selected from Table 217.A.2.

Embodiment 1467. The method of any of Embodiments 383, 400, 1128, or 1142, wherein the one or more proteins or wherein every protein is selected from Table 217.B.1.

Embodiment 1468. The method of any of Embodiments 383, 400, 1128, or 1142, wherein the one or more proteins or wherein every protein is selected from Table 217.B.2.

Embodiment 1469. The method of any of Embodiments 383, 400, 1128, or 1142, wherein the one or more proteins or wherein every protein is selected from Table 217.1B3.

Embodiment 1470. The method of any of Embodiments 383, 400, 1128, or 1142, wherein the one or more proteins or wherein every protein is selected from Table 217.B.4.

Embodiment 1471. The method of any of Embodiments 386, 402, 1130, or 1144, wherein the one or more proteins or wherein every protein is selected from Table 217.C.1.

Embodiment 1472. The method of any of Embodiments 386, 402, 1130, or 1144, wherein the one or more proteins or wherein every protein is selected from Table 217.C.2.

Embodiment 1473. The method of any of Embodiments 386, 402, 1130, or 1144, wherein the one or more proteins or wherein every protein is selected from Table 217.C.3.

Embodiment 1474. The method of any of Embodiments 386, 402, 1130, or 1144, wherein the one or more proteins or wherein every protein is selected from Table 217.C.4.

Embodiment 1475. The method of any of Embodiments 382, 399, 1127, or 1141, wherein the one or more proteins or wherein every protein is selected from Table 2017.A.1.

Embodiment 1476. The method of any of Embodiments 382, 399, 1127, or 1141, wherein the one or more proteins or wherein every protein is selected from Table 2017.A.2.

Embodiment 1477. The method of any of Embodiments 382, 399, 1127, or 1141, wherein the one or more proteins or wherein every protein is selected from Table 2017.A.3.

Embodiment 1478. The method of any of Embodiments 384, 401, 1128, or 1142, wherein the one or more proteins or wherein every protein is selected from Table 2017.B.1.

Embodiment 1479. The method of any of Embodiments 384, 401, 1128, or 1142, wherein the one or more proteins or wherein every protein is selected from Table 2017.B.2.

Embodiment 1480. The method of any of Embodiments 384, 401, 1128, or 1142, wherein the one or more proteins or wherein every protein is selected from Table 2017.B.3.

Embodiment 1481. The method of any of Embodiments 387, 403, 1130, or 1144, wherein the one or more proteins or wherein every protein is selected from Table 2017.C.1.

Embodiment 1482. The method of any of Embodiments 387, 403, 1130, or 1144, wherein the one or more proteins or wherein every protein is selected from Table 2017.C.2.

Embodiment 1483. The method of any of Embodiments 387, 403, 1130, or 1144, wherein the one or more proteins or wherein every protein is selected from Table 2017.C.3.

Embodiment 1484. The method of any of Embodiments 404, 418, 1149, or 1161, wherein the one or more proteins or wherein every protein is selected from Table 218.A.1.

Embodiment 1485. The method of any of Embodiments 404, 418, 1149, or 1161, wherein the one or more proteins or wherein every protein is selected from Table 218.A.2.

Embodiment 1486. The method of any of Embodiments 406, 420, 1150, or 1162, wherein the one or more proteins or wherein every protein is selected from Table 218.B.1.

Embodiment 1487. The method of any of Embodiments 406, 420, 1150, or 1162, wherein the one or more proteins or wherein every protein is selected from Table 218.B.2.

Embodiment 1488. The method of any of Embodiments 406, 420, 1150, or 1162, wherein the one or more proteins or wherein every protein is selected from Table 218.B.3.

Embodiment 1489. The method of any of Embodiments 406, 420, 1150, or 1162, wherein the one or more proteins or wherein every protein is selected from Table 218.B.4.

Embodiment 1490. The method of any of Embodiments 405, 407, 419, 421, 1149, 1150, 1161, or 1162, wherein the one or more proteins or wherein every protein is selected from Table 2018.A.1.

Embodiment 1491. The method of any of Embodiments 407, 421, 1149, 1150, 1161, or 1162, wherein the one or more proteins or wherein every protein is selected from Table 2018.A.2.

Embodiment 1492. The method of any of Embodiments 407, 421, 1149, 1150, 1161, or 1162, wherein the one or more proteins or wherein every protein is selected from Table 2018.A.3.

Embodiment 1493. The method of Embodiment 15 or 32, wherein the one or more proteins or wherein every protein is selected from Table 101.A.1.

Embodiment 1494. The method of Embodiment 15 or 32, wherein the one or more proteins or wherein every protein is selected from Table 101.A.2.

Embodiment 1495. The method of Embodiment 15 or 32, wherein the one or more proteins or wherein every protein is selected from Table 101.A.3.

Embodiment 1496. The method of Embodiment 15 or 32, wherein the one or more proteins or wherein every protein is selected from Table 101.A.4.

Embodiment 1497. The method of Embodiment 17 or 34, wherein the one or more proteins or wherein every protein is selected from Table 101.B.1.

Embodiment 1498. The method of Embodiment 17 or 34, wherein the one or more proteins or wherein every protein is selected from Table 101.1B.2.

Embodiment 1499. The method of Embodiment 17 or 34, wherein the one or more proteins or wherein every protein is selected from Table 101.1B.3.

Embodiment 1500. The method of Embodiment 17 or 34, wherein the one or more proteins or wherein every protein is selected from Table 101.1B.4.

Embodiment 1501. The method of Embodiment 20 or 36, wherein the one or more proteins or wherein every protein is selected from Table 101.C.1.

Embodiment 1502. The method of Embodiment 20 or 36, wherein the one or more proteins or wherein every protein is selected from Table 101.C.2.

Embodiment 1503. The method of Embodiment 20 or 36, wherein the one or more proteins or wherein every protein is selected from Table 101.C.3.

Embodiment 1504. The method of Embodiment 20 or 36, wherein the one or more proteins or wherein every protein is selected from Table 101.C.4.

Embodiment 1505. The method of Embodiment 16 or 33, wherein the one or more proteins or wherein every protein is selected from Table 1001.A.1.

Embodiment 1506. The method of Embodiment 16 or 33, wherein the one or more proteins or wherein every protein is selected from Table 1001.A.2.

Embodiment 1507. The method of Embodiment 16 or 33, wherein the one or more proteins or wherein every protein is selected from Table 1001.A.3.

Embodiment 1508. The method of Embodiment 16 or 33, wherein the one or more proteins or wherein every protein is selected from Table 1001.A.4.

Embodiment 1509. The method of Embodiment 18 or 35, wherein the one or more proteins or wherein every protein is selected from Table 1001.B.1.

Embodiment 1510. The method of Embodiment 18 or 35, wherein the one or more proteins or wherein every protein is selected from Table 1001.B.2.

Embodiment 1511. The method of Embodiment 18 or 35, wherein the one or more proteins or wherein every protein is selected from Table 1001.B.3.

Embodiment 1512. The method of Embodiment 18 or 35, wherein the one or more proteins or wherein every protein is selected from Table 1001.B.4.

Embodiment 1513. The method of Embodiment 21 or 37, wherein the one or more proteins or wherein every protein is selected from Table 1001.C.1.

Embodiment 1514. The method of Embodiment 21 or 37, wherein the one or more proteins or wherein every protein is selected from Table 1001.C.2.

Embodiment 1515. The method of Embodiment 21 or 37, wherein the one or more proteins or wherein every protein is selected from Table 1001.C.3.

Embodiment 1516. The method of Embodiment 21 or 37, wherein the one or more proteins or wherein every protein is selected from Table 1001.C.4.

LENGTHY TABLES
The patent application contains a lengthy table section. A copy of the table is available in electronic form from the USPTO web site (https://seqdata.uspto.gov/docdetail?docId=US20260196349A1). An electronic copy of the table will also be available from the USPTO upon request and payment of the fee set forth in 37 CFR 1.19(b)(3).

1. A method of evaluating a subject for cancer comprising:
(I) determining in a biological sample from the subject a concentration of one or more proteins selected from Table 101 or selected from Table 1001;
(II) determining in a biological sample from the subject a concentration of one or more proteins selected from each of Tables 201-218 or selected from each of Tables 2001-2018; or determining in a biological sample from the subject a concentration of each protein from Table 251, 252, 253, or 254;
thereby evaluating the subject for cancer,
wherein the sample has been separated from the subject at the time of the determination in (I) or (II).

2. The method of claim 1, wherein (I) is performed prior to (II).

3. The method of claim 1, wherein the performance of (I) overlaps with the performance of (II).

4. The method of any one of claims 1-3, further comprising, in (I), applying a classifier to the concentration of the one or more proteins that identifies whether the concentration of the one or more proteins selected from Table 101 or selected from Table 1001 is indicative of the subject having cancer.

5. The method of any one of claims 1-4, further comprising, in (II), applying a classifier to the concentration of the one or more proteins that identifies whether the concentration of one two or more proteins selected from each of Tables 201-218 or selected from each of Tables 2001-2018 is indicative of tissue with which the cancer is associated; or applying a classifier to the concentration of each protein that identifies whether the concentration of the proteins of Table 251, 252, 253, or 254 is indicative of tissue with which the cancer is associated.

6. The method of any one of claims 1-5, further comprising administering a treatment to the subject.

7. The method of any one of claims 1-6, wherein the cancer comprises a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

8. The method of any one of claims 1-6, wherein the cancer comprises bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

9. A method of treating cancer in a subject, comprising
(a) acquiring results from the method of any one of claims 1-5; and
(b) administering a treatment to the subject.

10. The method of claim 9, wherein the treatment is responsive to the results acquired in (a).

11. The method of claim 9 or 10, wherein (a) comprises
(I) determining in a biological sample from the subject a concentration of two or more proteins selected from Table 101 or selected from Table 1001, and applying a classifier to the concentration of the two or more proteins to identify that the subject has cancer; and
(II) determining in a biological sample from the subject a concentration of two or more proteins selected from each of Tables 201-218 or selected from each of Tables 2001-2018, and applying a classifier to the concentration of the two or more proteins to identify with which tissue the cancer is associated: or determining in a biological sample from the subject a concentration of each protein of Table 251, 252, 253, or 254, and applying a classifier to the concentration to identify with which tissue the cancer is associated.

12. A method of treating cancer in a subject, comprising
(a) acquiring results from an evaluation of the subject that determined to which tissue the cancer is associated; and
(b) administering a treatment to the subject,
wherein the evaluation comprises:
(I) determining in a biological sample from the subject a concentration of one or more proteins selected from Table 101 or selected from Table 1001, and applying a classifier to the concentration of the one or more proteins to identify that the subject has cancer;
(II) determining in a biological sample from the subject a concentration of one or more proteins selected from each of Tables 201-218 or selected from each of Tables 2001-2018, and applying a classifier to the concentration of the one or more proteins to identify with which tissue the cancer is associated; or determining in a biological sample from the subject a concentration of each of the proteins of Tables 251, 252, 253, or 254, and applying a classifier to the concentration of each protein to identify with which tissue the cancer is associated.

13. The method of any one of claims 9-12, wherein the results in (a) are acquired from a third party.

14. The method of any one of claims 1-13, wherein the cancer is early-stage.

15. The method of any one of claims 1-14, wherein in (I), the one or more proteins selected from Table 101 are selected from Table 101A.1, 101.A.2, 101.A.3, 101.A.4, 101.B.1, 101.B.2, 101.B.3, 101.B.4, 101.C.1, 101.C.2, 101.C.3, or 101.C.4.

16. The method of any one of claims 1-14, wherein in (I), the one or more proteins selected from Table 1001 are selected from Table 1001.A.1, 1001.A.2, 1001.A.3, 1001.A.4, 1001.B.1, 1001.B.2, 1001.B.3, 1001.B.4, 1001.C.1, 1001.C.2, 1001.C.3, or 1001.C.4.

17. The method of claim 15 or 16, wherein the subject is female.

18. The method of claim 15 or 16, wherein the subject is male.

19. The method of any one of claims 1-14, wherein, in (I), the one or more proteins selected from Table 101 is every protein of Table 101.A.1, 101.A.2, 101.A.3, 101.A.4, 101.B.1, 101.B.2, 101.B.3, 101.B.4, 101.C.1, 101.C.2, 101.C.3, or 101.C.4.

20. The method of any one of claims 1-14, wherein, in (I), the one or more proteins selected from Table 1001 is every protein of Table 1001.A.1, 1001.A.2, 1001.A.3, 1001.A.4, 1001.B.1, 1001.B.2, 1001.B.3, 1001.B.4, 1001.C.1, 1001.C.2, 1001.C.3, or 1001.C.4.

21. The method of any one of claims 1-20, wherein in (II), the one or more proteins selected from Table 201 are selected from Table 201.A.1, 201.A.2, 201.B.1, 201.B.2, 201.B.3, 201.B.4, 201.C.1, 201.C.2, 201.C.3, or 201.C.4.

22. The method of any one of claims 1-20, wherein in (II), the one or more proteins selected from Table 2001 are selected from Table 2001.A.1, 2001.A.2, 2001.A.3, 2001.B.1, 2001.B.2, 2001.B.3, 2001.C.1, 2001.C.2, or 2001.C.3.

23. The method of claim 21 or 22, wherein the subject is female.

24. The method of claim 21 or 22, wherein the subject is male.

25. The method of any one of claims 1-20, wherein, in (II), the one or more proteins selected from Table 201 is every protein of Table 201.A.1, 201.A.2, 201.B.1, 201.B.2, 201.B.3, 201.B.4, 201.C.1, 201.C.2, 201.C.3, or 201.C.4.

26. The method of any one of claims 1-20, wherein, in (II), the one or more proteins selected from Table 2001 is every protein of Table 2001.A.1, 2001.A.2, 2001.A.3, 2001.B.1, 2001.B.2, 2001.B.3, 2001.C.1, 2001.C.2, or 2001.C.3.

27. The method of any one of claims 1-26, wherein in (II), the one or more proteins selected from Table 202 are selected from Table 202.A.1, 202.A.2, 202.B.1, 202.B.2, 202.B.3, 202.B.4, 202.C.1, 202.C.2, or 202.C.3.

28. The method of any one of claims 1-26, wherein in (II), the one or more proteins selected from Table 2002 are selected from Table 2002.A.1, 2002.A.2, 2002.A.3, 2002.B.1, 2002.B.2, 2002.B.3, 2002.C.1, 2002.C.2, or 2002.C.3.

29. The method of claim 27 or 28, wherein the subject is female.

30. The method of claim 27 or 28, wherein the subject is male.

31. The method of any one of claims 1-26, wherein, in (II), the one or more proteins selected from Table 202 is every protein of Table 202.A.1, 202.A.2, 202.B.1, 202.B.2, 202.B.3, 202.B.4, 202.C.1, 202.C.2, or 202.C.3.

32. The method of any one of claims 1-26, wherein, in (II), the one or more proteins selected from Table 2002 is every protein of Table 2002.A.1, 2002.A.2, 2002.A.3, 2002.B.1, 2002.B.2, 2002.B.3, 2002.C.1, 2002.C.2, or 2002.C.3.

33. The method of any one of claims 1-32, wherein in (II), the one or more proteins selected from Table 203 are selected from Table 203.A.1, 203.A.2, 203.B.1, 203.B.2, 203.B.3, 203.B.4, 203.C.1, 203.C.2, 203.C.3, or 204.C.4.

34. The method of any one of claims 1-32, wherein in (II), the one or more proteins selected from Table 2003 are selected from Table 2003.A.1, 2003.A.2, 2003.A.3, 2003.B.1, 2003.B.2, 2003.B.3, 2003.C.1, 2003.C.2, or 2003.C.3.

35. The method of claim 33 or 34, wherein the subject is female.

36. The method of claim 33 or 34, wherein the subject is male.

37. The method of any one of claims 1-32, wherein, in (II), the one or more proteins selected from Table 203 is every protein of Table 203.A.1, 203.A.2, 203.B.1, 203.B.2, 203.B.3, 203.B.4, 203.C.1, 203.C.2, 203.C.3, or 204.C.4.

38. The method of any one of claims 1-32, wherein, in (II), the one or more proteins selected from Table 2003 is every protein of Table 2003.A.1, 2003.A.2, 2003.A.3, 2003.B.1, 2003.B.2, 2003.B.3, 2003.C.1, 2003.C.2, or 2003.C.3.

39. The method of any one of claims 1-39, wherein in (II), the one or more proteins selected from Table 204 are selected from Table 204.A.1 204.A.2, 204.B.1, 204.B.2, 204.B.3, or 204.B.4.

40. The method of any one of claims 1-39, wherein in (II), the one or more proteins selected from Table 2004 are selected from Table 2004.A.1, 2004.A.2, or 2004.A.3.

41. The method of claim 39 or 40, wherein the subject is female.

42. The method of any one of claims 1-39, wherein, in (II), the one or more proteins selected from Table 204 is every protein of Table 204.A.1 204.A.2, 204.B.1, 204.B.2, 204.B.3, or 204.B.4.

43. The method of any one of claims 1-39, wherein, in (II), the one or more proteins selected from Table 2004 is every protein of Table 2004.A.1, 2004.A.2, or 2004.A.3.

44. The method of any one of claims 1-43, wherein in (II), the one or more proteins selected from Table 205 are selected from Table 205.A.1, 205.A.2, 205.B.1, 205.B.2, 205.B.3, or 205.B.4.

45. The method of any one of claims 1-43, wherein in (II), the one or more proteins selected from Table 2005 are selected from Table 2005.A.1, 2005.A.2, or 2005.A.3.

46. The method of claim 44 or 45, wherein the subject is female.

47. The method of any one of claims 1-43, wherein, in (II), the one or more proteins selected from Table 205 is every protein of Table 205.A.1, 205.A.2, 205.B.1, 205.B.2, 205.B.3, or 205.B.4.

48. The method of any one of claims 1-43, wherein, in (II), the one or more proteins selected from Table 2005 is every protein of Table 2005.A.1, 2005.A.2, or 2005.A.3.

49. The method of any one of claims 1-48, wherein in (II), the one or more proteins selected from Table 206 are selected from Table 206.A.1, 206.A.2, 206.B.1, 206.B.2, 206.B.3, 206.B.4, 206.C.1, 206.C.2, or 206.C.3.

50. The method of any one of claims 1-48, wherein in (II), the one or more proteins selected from Table 2006 are selected from Table 2006.A.1, 2006.A.2, 2006.A.3, 2006.B.1, 2006.B.2, 2006.B.3, 2006.C.1, 2006.C.2, or 2006.C.3.

51. The method of claim 49 or 50, wherein the subject is female.

52. The method of claim 49 or 50, wherein the subject is male.

53. The method of any one of claims 1-48, wherein, in (II), the one or more proteins selected from Table 206 is every protein of Table 206.A.1, 206.A.2, 206.B.1, 206.B.2, 206.B.3, 206.B.4, 206.C.1, 206.C.2, or 206.C.3.

54. The method of any one of claims 1-48, wherein, in (II), the one or more proteins selected from Table 2006 is every protein of Table 2006.A.1, 2006.A.2, 2006.A.3, 2006.B.1, 2006.B.2, 2006.B.3, 2006.C.1, 2006.C.2, or 2006.C.3.

55. The method of any one of claims 1-54, wherein in (II), the one or more proteins selected from Table 207 are selected from Table 207.A.1, 207.A.2, 207.B.1, 207.B.2, 207.B.3, 207.B.4, 207.C.1, 207.C.2, or 207.C.3.

56. The method of any one of claims 1-54, wherein in (II), the one or more proteins selected from Table 2007 are selected from Table 2007.A.1, 2007.A.2, 2007.A.3, 2007.B.1, 2007.B.2, 2007.B.3, 2007.C.1, 2007.C.2, or 2007.C.3.

57. The method of claim 55 or 56, wherein the subject is female.

58. The method of claim 55 or 56, wherein the subject is male.

59. The method of any one of claims 1-54, wherein, in (II), the one or more proteins selected from Table 207 is every protein of Table 207.A.1, 207.A.2, 207.B.1, 207.B.2, 207.B.3, 207.B.4, 207.C.1, 207.C.2, or 207.C.3.

60. The method of any one of claims 1-54, wherein, in (II), the one or more proteins selected from Table 2007 is every protein of Table 2007.A.1, 2007.A.2, 2007.A.3, 2007.B.1, 2007.B.2, 2007.B.3, 2007.C.1, 2007.C.2, or 2007.C.3.

61. The method of any one of claims 1-60, wherein in (II), the one or more proteins selected from Table 208 are selected from Table 208.A.1, 208.A.2, 208.B.1, 208.B.2, 208.B.3, 208.B.4, 208.C.1, 208.C.2, 208.C.3, or 208.C.4.

62. The method of any one of claims 1-60, wherein in (II), the one or more proteins selected from Table 2008 are selected from Table 2008.A.1, 2008.A.2, 2008.A.3, 2008.B.1, 2008.B.2, 2008.B.3, 2008.C.1, 2008.C.2, or 2008.C.3.

63. The method of claim 61 or 62, wherein the subject is female.

64. The method of claim 61 or 62, wherein the subject is male.

65. The method of any one of claims 1-60, wherein, in (II), the one or more proteins selected from Table 208 is every protein of Table 208.A.1, 208.A.2, 208.B.1, 208.B.2, 208.B.3, 208.B.4, 208.C.1, 208.C.2, 208.C.3, or 208.C.4.

66. The method of any one of claims 1-60, wherein, in (II), the one or more proteins selected from Table 2008 is every protein of Table 2008.A.1, 2008.A.2, 2008.A.3, 2008.B.1, 2008.B.2, 2008.B.3, 2008.C.1, 2008.C.2, or 2008.C.3.

67. The method of any one of claims 1-66, wherein in (II), the one or more proteins selected from Table 209 are selected from Table 209.A.1, 209.A.2, 209.B.1, 209.B.2, 209.B.3, 209.B.4, 209.C.1, 209.C.2, 209.C.3, or 209.C.4.

68. The method of any one of claims 1-66, wherein in (II), the one or more proteins selected from Table 2009 are selected from Table 2009.A.1, 2009.A.2, 2009.A.3, 2009.B.1, 2009.B.2, 2009.B.3, 2009.C.1, 2009.C.2, or 2009.C.3.

69. The method of claim 67 or 68, wherein the subject is female.

70. The method of claim 67 or 68, wherein the subject is male.

71. The method of any one of claims 1-66, wherein, in (II), the one or more proteins selected from Table 209 is every protein of Table 209.A.1, 209.A.2, 209.B.1, 209.B.2, 209.B.3, 209.B.4, 209.C.1, 209.C.2, 209.C.3, or 209.C.4.

72. The method of any one of claims 1-66, wherein, in (II), the one or more proteins selected from Table 2009 is every protein of Table 2009.A.1, 2009.A.2, 2009.A.3, 2009.B.1, 2009.B.2, 2009.B.3, 2009.C.1, 2009.C.2, or 2009.C.3.

73. The method of any one of claims 1-72, wherein in (II), the one or more proteins selected from Table 210 are selected from Table 210.A.1, 210.A.2, 210.B.1, 210.B.2, 210.B.3, 210.B.4, 210.C.1, 210.C.2, 210.C.3, or 210.C.4.

74. The method of any one of claims 1-72 wherein in (II), the one or more proteins selected from Table 2010 are selected from Table 2010.A.1, 2010.A.2, 2010.A.3, 2010.B.1, 2010.B.2, 2010.B.3, 2010.C.1, 2010.C.2, or 2010.C.3.

75. The method of claim 73 or 74, wherein the subject is female.

76. The method of claim 73 or 74, wherein the subject is male.

77. The method of any one of claims 1-72, wherein, in (II), the one or more proteins selected from Table 210 is every protein of Table 210.A.1, 210.A.2, 210.B.1, 210.B.2, 210.B.3, 210.B.4, 210.C.1, 210.C.2, 210.C.3, or 210.C.4.

78. The method of any one of claims 1-72, wherein, in (II), the one or more proteins selected from Table 2010 is every protein of Table 2010.A.1, 2010.A.2, 2010.A.3, 2010.B.1, 2010.B.2, 2010.B.3, 2010.C.1, 2010.C.2, or 2010.C.3.

79. The method of any one of claims 1-78, wherein in (II), the one or more proteins selected from Table 211 are selected from Table 211.A.1, 211.A.2, 211.B.1, 211.B.2, 211.B.3, 211.B.4, 211.C.1, 211.C.2, or 211.C.3.

80. The method of any one of claims 1-78, wherein in (II), the one or more proteins selected from Table 2011 are selected from Table 2011.A.1, 2011.A.2, 2011.B.1, 2011.B.2, 2011.B.3, 2011.C.1, 2011.C.2, or 2011.C.3.

81. The method of claim 79 or 80, wherein the subject is female.

82. The method of claim 79 or 80, wherein the subject is male.

83. The method of any one of claims 1-78, wherein, in (II), the one or more proteins selected from Table 211 is every protein of Table 211.A.1, 211.A.2, 211.B.1, 211.B.2, 211.B.3, 211.B.4, 211.C.1, 211.C.2, or 211.C.3.

84. The method of any one of claims 1-78, wherein, in (II), the one or more proteins selected from Table 2011 is every protein of Table 2011.A.1, 2011.A.2, 2011.B.1, 2011.B.2, 2011.B.3, 2011.C.1, 2011.C.2, or 2011.C.3.

85. The method of any one of claims 1-84, wherein in (II), the one or more proteins selected from Table 212 are selected from Table 212.A.1, 212.A.2, 212.B.1, 212.B.2, 212.B.3, or 212.B.4.

86. The method of any one of claims 1-84, wherein in (II), the one or more proteins selected from Table 2012 are selected from Table 2012.A.1, 2012.A.2, or 2012.A.3.

87. The method of claim 85 or 86, wherein the subject is female.

88. The method of any one of claims 1-84, wherein, in (II), the one or more proteins selected from Table 212 is every protein of Table 212.A.1, 212.A.2, 212.B.1, 212.B.2, 212.B.3, or 212.B.4.

89. The method of any one of claims 1-84, wherein, in (II), the one or more proteins selected from Table 2012 is every protein of Table 2012.A.1, 2012.A.2, or 2012.A.3.

90. The method of any one of claims 1-89, wherein in (II), the one or more proteins selected from Table 213 are selected from Table 213.A.1, 213.A.2, 213.B.1, 213.B.2, 213.B.3, 213.C.1, 213.C.2, or 213.C.3.

91. The method of any one of claims 1-89, wherein in (II), the one or more proteins selected from Table 2013 are selected from Table 2013.A.1, 2013.A.2, 2013.A.3, 2013.B.1, 2013.B.2, 2013.B.3, 2013.C.1, 2013.C.2, or 2013.C.3.

92. The method of claim 90 or 91, wherein the subject is female.

93. The method of claim 90 or 91, wherein the subject is male.

94. The method of any one of claims 1-89, wherein, in (II), the one or more proteins selected from Table 213 is every protein of Table 213.A.1, 213.A.2, 213.B.1, 213.B.2, 213.B.3, 213.C.1, 213.C.2, or 213.C.3.

95. The method of any one of claims 1-89, wherein, in (II), the one or more proteins selected from Table 2013 is every protein of Table 2013.A.1, 2013.A.2, 2013.A.3, 2013.B.1, 2013.B.2, 2013.B.3, 2013.C.1, 2013.C.2, or 2013.C.3.

96. The method of any one of claims 1-95, wherein in (II), the one or more proteins selected from Table 214 are selected from Table 214.A.1, 214.A.2, 214.B.1, 214.B.2, 214.B.3, or 214.B.4.

97. The method of any one of claims 1-95, wherein in (II), the one or more proteins selected from Table 2014 are selected from Table 2014.A.1, 2014.A.2, or 2014.A.3.

98. The method of claim 96 or 97, wherein the subject is male.

99. The method of any one of claims 1-95, wherein, in (II), the one or more proteins selected from Table 214 is every protein of Table 214.A.1, 214.A.2, 214.B.1, 214.B.2, 214.B.3, or 214.B.4.

100. The method of any one of claims 1-95, wherein, in (II), the one or more proteins selected from Table 2014 is every protein of Table 2014.A.1, 2014.A.2, or 2014.A.3.

101. The method of any one of claims 1-100, wherein in (II), the one or more proteins selected from Table 215 are selected from Table 215.A.1, 215.A.2, 215.B.1, 215.B.2, 215.B.3, 215.B.4, 215.C.1, 215.C.2, 215.C.3, or 215.C.4.

102. The method of any one of claims 1-100, wherein in (II), the one or more proteins selected from Table 2015 are selected from Table 2015.A.1, 2015.A.2, 2015.A.3, 2015.B.1, 2015.B.2, 2015.B.3, 2015.C.1, 2015.C.2, or 2015.C.3.

103. The method of claim 101 or 102, wherein the subject is female.

104. The method of claim 100 or 102, wherein the subject is male.

105. The method of any one of claims 1-100, wherein, in (II), the one or more proteins selected from Table 215 is every protein of Table 215.A.1, 215.A.2, 215.B.1, 215.B.2, 215.B.3, 215.B.4, 215.C.1, 215.C.2, 215.C.3, or 215.C.4.

106. The method of any one of claims 1-100, wherein, in (II), the one or more proteins selected from Table 2015 is every protein of Table 2015.A.1, 2015.A.2, 2015.A.3, 2015.B.1, 2015.B.2, 2015.B.3, 2015.C.1, 2015.C.2, or 2015.C.3.

107. The method of any one of claims 1-106, wherein in (II), the one or more proteins selected from Table 216 are selected from Table 216.A.1, 216.A.2, 216.B.1, 216.B.2, 216.B.3, or 216.B.4.

108. The method of any one of claims 1-106, wherein in (II), the one or more proteins selected from Table 2016 are selected from Table 2016.A.1, 2016.A.2, or 2016.A.3.

109. The method of claim 107 or 108, wherein the subject is male.

110. The method of any one of claims 1-106, wherein, in (II), the one or more proteins selected from Table 216 is every protein of Table 216.A.1, 216.A.2, 216.B.1, 216.B.2, 216.B.3, or 216.B.4.

111. The method of any one of claims 1-106, wherein, in (II), the one or more proteins selected from Table 2016 is every protein of Table 2016.A.1, 2016.A.2, or 2016.A.3.

112. The method of any one of claims 1-111, wherein in (II), the one or more proteins selected from Table 217 are selected from Table 217.A.1, 217.A.2, 217.B.1, 217.B.2, 217.B.3, 217.B.4, 217.C.1, 217.C.2, 217.C.3, or 217.C.4.

113. The method of any one of claims 1-111, wherein in (II), the one or more proteins selected from Table 2017 are selected from Table 2017.A.1, 2017.A.2, 2017.A.3, 2017.B.1, 2017.B.2, 2017.B.3, 2017.C.1, 2017.C.2, or 2017.C.3.

114. The method of claim 112 or 113, wherein the subject is female.

115. The method of claim 112 or 113, wherein the subject is male.

116. The method of any one of claims 1-111, wherein, in (II), the one or more proteins selected from Table 217 is every protein of Table 217.A.1, 217.A.2, 217.B.1, 217.B.2, 217.B.3, 217.B.4, 217.C.1, 217.C.2, 217.C.3, or 217.C.4.

117. The method of any one of claims 1-111, wherein, in (II), the one or more proteins selected from Table 2017 is every protein of Table 2017.A.1, 2017.A.2, 2017.A.3, 2017.B.1, 2017.B.2, 2017.B.3, 2017.C.1, 2017.C.2, or 2017.C.3.

118. The method of any one of claims 1-117, wherein in (II), the one or more proteins selected from Table 218 are selected from Table 218.A.1, 218.A.2, 218.B.1, 218.B.2, 218.B.3, or 218.B.4.

119. The method of any one of claims 1-117, wherein in (II), the one or more proteins selected from Table 2018 are selected from Table 2018.A.1, 2018.A.2, or 2018.A.3.

120. The method of claim 118 or 119, wherein the subject is female.

121. The method of any one of claims 1-117, wherein, in (II), the one or more proteins selected from Table 218 is every protein of Table 218.A.1, 218.A.2, 218.B.1, 218.B.2, 218.B.3, or 218.B.4.

122. The method of any one of claims 1-117, wherein, in (II), the one or more proteins selected from Table 2018 is every protein of Table 2018.A.1, 2018.A.2, or 2018.A.3.

123. The method of any one of claims 1-20, wherein in (II), the concentration of each protein is from Table 251, 252, 253, or 254.

124. The method of claim 123, wherein the concentration of each protein is from Table 251.

125. The method of claim 124, wherein the subject is female.

126. The method of claim 123, wherein the concentration of each protein is from Table 252.

127. The method of claim 126, wherein the subject is female.

128. The method of claim 123, wherein the concentration of each protein is from Table 253.

129. The method of claim 128, wherein the subject is male.

130. The method of claim 123, wherein the concentration of each protein is from Table 254.

131. The method of claim 130, wherein the subject is male.

132. The method of any one of claims 123-131, wherein the cancer is early-stage.

133. A method of evaluating a subject for a cancer associated with a tissue selected from bladder, bone, brain, breast, cervix, colon, esophagus, kidney, liver, lung—small cell (SC), lung—non-small cell (NSC), ovary, pancreas, prostate, stomach, testis, thyroid, and uterus, the method comprising:
determining in a biological sample from the subject a concentration of one or more proteins selected from Tables 301-318 that respectively correspond to said tissues;
thereby evaluating the subject for cancer.

134. The method of claim 133, further comprising applying a classifier to the concentration of the one or more proteins that identifies whether the concentration of the one or more proteins is indicative of the subject having cancer associated with the tissue.

135. The method of claim 133 or 134, further comprising administering a treatment to the subject.

136. The method of any one of claims 133-135, wherein the subject is evaluated for a cancer selected from breast, colon, SC lung, NSC lung, ovary, or pancreas, and the one or more proteins are selected from Tables 304, 306, 310, 311, 312, or 313, respectively.

137. A method of treating cancer in a subject, comprising
(a) acquiring results from the method of any one of claims 133-136; and
(b) administering a treatment to the subject.

138. The method of claim 137, wherein the treatment is responsive to the results acquired in (a).

139. The method of claim 137 or 138, wherein (a) comprises:
(i) determining in a biological sample from the subject a concentration of one or more proteins selected from Tables 301-318 that respectively correspond to said tissues; and
(ii) applying a classifier to the concentration to identify with which tissue the cancer is associated.

140. A method of treating cancer in a subject, wherein the cancer is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising:
(a) acquiring results from an evaluation of the subject that determined the subject has cancer associated with the tissue;
(b) administering a treatment to the subject, wherein the evaluation comprises:
(i) determining in a biological sample from the subject a concentration of one or more proteins selected from Tables 301-318 that respectively correspond to said tissues; and
(ii) applying a classifier to the concentration to identify with which tissue the cancer is associated.

141. The method of any one of claims 137-140, wherein the results in (a) are acquired from a third party.

142. The method of any one of claims 133-141, wherein the cancer is associated with the bladder or is bladder cancer.

143. The method of claim 142, wherein the one or more proteins are selected from Table 301.A.1, 301.A2, 301.B.1, 301.B.2, 301.C.1, or 301.C.2.

144. The method of claim 143, wherein the subject is female.

145. The method of claim 143, wherein the subject is male.

146. The method of any one of claims 133-141, wherein the cancer is associated with bone or is bone cancer.

147. The method of claim 146, wherein the one or more proteins are selected from Table 302.A.1, 302.A2, 302.B.1, 302.B.2, 302.C.1, or 302.C.2.

148. The method of claim 147, wherein the subject is female.

149. The method of claim 147, wherein the subject is male.

150. The method of any one of claims 146-149, wherein the cancer is osteosarcoma.

151. The method of any one of claims 133-141, wherein the cancer is associated with the brain or is brain cancer.

152. The method of claim 482, wherein the one or more proteins are selected from Table 303.A.1, 303.A2, 303.B.1, 303.B.2, 303.C.1, or 303.C.2.

153. The method of claim 152, wherein the subject is female.

154. The method of claim 152, wherein the subject is male.

155. The method of any one of claims 151-154, wherein the cancer is astrocytoma.

156. The method of any one of claims 133-141, wherein the cancer is associated with the breast or is breast cancer.

157. The method of claim 503, wherein the one or more proteins are selected from Table 304.A.1, 304.A2, 304.B.1, or 304.B.2.

158. The method of claim 157, wherein the subject is female.

159. The method of any one of claims 133-141, wherein the cancer is associated with the cervix or is cervical cancer.

160. The method of claim 159, wherein the one or more proteins are selected from Table 305.A.1, 305.A2, 305.B.1, or 305.B.2.

161. The method of claim 160, wherein the subject is female.

162. The method of any one of claims 133-141, wherein the cancer is associated with the colon or is colorectal cancer.

163. The method of claim 535, wherein the one or more proteins are selected from Table 306.A.1, 306.A2, 306.B.1, 306.B.2, 306.C.1, or 306.C.2.

164. The method of claim 163, wherein the subject is female.

165. The method of claim 163, wherein the subject is male.

166. The method of any one of claims 133-141, wherein the cancer is associated with the esophagus or is esophageal cancer.

167. The method of claim 166, wherein the one or more proteins are selected from Table 307.A.1, 307.A2, 307.B.1, 307.B.2, 307.C.1, or 307.C.2.

168. The method of claim 167, wherein the subject is female.

169. The method of claim 167, wherein the subject is male.

170. The method of any one of claims 133-141, wherein the cancer is associated with the kidney or is kidney cancer.

171. The method of claim 170, wherein the one or more proteins are selected from Table 308.A.1, 308.A2, 308.B.1, 308.B.2, 308.C.1, or 308.C.2.

172. The method of claim 171, wherein the subject is female.

173. The method of claim 171, wherein the subject is male.

174. The method of any one of claims 133-141, wherein the cancer is associated with the liver or is liver cancer.

175. The method of claim 174, wherein the one or more proteins are selected from Table 309.A.1, 309.A2, 309.B.1, 309.B.2, 309.C.1, or 309.C.2.

176. The method of claim 175, wherein the subject is female.

177. The method of claim 175, wherein the subject is male.

178. The method of any one of claims 174-177, wherein the cancer is hepatocellular carcinoma.

179. The method of any one of claims 133-141, wherein the cancer is associated with the lung—small cell (SC) or is SC lung cancer.

180. The method of claim 179, wherein the one or more proteins are selected from Table 310.A.1, 310.A2, 310.B.1, 310.B.2, 310.C.1, or 310.C.2.

181. The method of claim 180, wherein the subject is female.

182. The method of claim 180, wherein the subject is male.

183. The method of any one of claims 133-141, wherein the cancer is associated with the lung—non-small cell (NSC) or is NSC lung cancer.

184. The method of claim 183, wherein the one or more proteins are selected from Table 311.A.1, 311.A2, 311.B.1, 311.B.2, 311.C.1, or 311.C.2.

185. The method of claim 184, wherein the subject is female.

186. The method of claim 184, wherein the subject is male.

187. The method of any one of claims 133-141, wherein the cancer is associated with the ovary or is ovarian cancer.

188. The method of claim 187, wherein the one or more proteins are selected from Table 312.A.1, 312.A2, 312.B.1, or 312.B.2.

189. The method of claim 188, wherein the subject is female.

190. The method of any one of claims 133-141, wherein the cancer is associated with the pancreas.

191. The method of claim 671, wherein the one or more proteins are selected from Table 313.A.1, 310.A2, 310.B.1, 310.B.2, 310.C.1, or 310.C.2.

192. The method of claim 191, wherein the subject is female.

193. The method of claim 191, wherein the subject is male.

194. The method of any one of claims 133-141, wherein the cancer is associated with the prostate.

195. The method of claim 691, wherein the one or more proteins are selected from Table 314.A.1, 314.A2, 314.B.1, or 314.B.2.

196. The method of claim 195, wherein the subject is male.

197. The method of any one of claims 133-141, wherein the cancer is associated with the stomach.

198. The method of claim 197, wherein the one or more proteins are selected from Table 315.A.1, 315.A2, 315.B.1, 315.B.2, 315.C.1, or 315.C.2.

199. The method of claim 198, wherein the subject is female.

200. The method of claim 198, wherein the subject is male.

201. The method of any one of claims 133-141, wherein the cancer is associated with the testis.

202. The method of claim 727, wherein the one or more proteins are selected from Table 316.A.1, 316.A2, 316.B.1, or 316.B.2.

203. The method of claim 202, wherein the subject is male.

204. The method of any one of claims 133-141, wherein the cancer is associated with the thyroid.

205. The method of claim 204, wherein the one or more proteins are selected from Table 317.A.1, 317.A2, 317.B.1, 317.B.2, 317.C.1, or 317.C.2.

206. The method of claim 205, wherein the subject is female.

207. The method of claim 205, wherein the subject is male.

208. The method of any one of claims 133-141, wherein the cancer is associated with the uterus.

209. The method of claim 208, wherein the one or more proteins are selected from Table 318.A.1, 318.A2, 318.B.1, or 318.B.2.

210. The method of claim 209, wherein the subject is female.

211. The method of any one of claims 133-210, wherein the cancer is early-stage.

212. The method of any one of claims 1-211, wherein the subject is asymptomatic for cancer.

213. The method of claim 212, wherein the subject is asymptomatic for a cancer selected from a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus.

214. The method of claim 213, wherein the subject is asymptomatic of a cancer comprising bladder cancer, bone cancer, brain cancer, breast cancer, cervical cancer, colon cancer, esophageal cancer, kidney cancer, liver cancer, lung cancer—small cell, lung cancer—non-small cell, ovarian cancer, pancreatic cancer, prostate cancer, stomach cancer, testicular cancer, thyroid cancer, or uterine cancer.

215. A method of evaluating a subject who is suspected of having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising:
determining in a biological sample from the subject a concentration of one or more proteins selected from Tables 201-218 or selected from Tables 2001-2018 that respectively correspond to said tissues; or determining in a biological sample from the subject a concentration of each protein from Table 251, 252, 253, or 254;
thereby evaluating the subject for cancer.

216. The method of claim 215, further comprising applying a classifier to the concentration of the one or more proteins that identifies whether the concentration of the one or more proteins is indicative of the subject having cancer associated with the tissue: or applying a classifier to the concentration of each protein that identifies whether the concentration of the proteins of Table 251, 252, 253, or 254 is indicative of the subject having cancer associated with the tissue.

217. The method of claim 215 or 216, further comprising administering a treatment to the subject.

218. A method of treating cancer in a subject, comprising
(a) acquiring results from the method of any one of claims 217-217; and
(b) administering a treatment to the subject.

219. The method of claim 218, wherein the treatment is responsive to the results acquired in (a).

220. The method of claim 218 or 219, wherein (a) comprises:
determining in a biological sample from the subject a concentration of one or more proteins selected from Tables 201-218 that respectively correspond to said tissues, and (applying a classifier to the concentration of the one or more proteins to identify with which tissue the cancer is associated; or
determining in a biological sample from the subject a concentration of each protein from Table 251, 252, 253, or 254, and applying a classifier to the concentration of each protein to identify with which tissue the cancer is associated.

221. A method of treating cancer in a subject who is suspected of having a cancer that is associated with a tissue selected from bladder, bone, brain, breast, cervix, colon, esophagus, kidney, liver, lung—small cell, lung—non-small cell, ovary, pancreas, prostate, stomach, testis, thyroid, and uterus, the method comprising:
(a) acquiring results from an evaluation of the subject that determined the subject has cancer associated with the tissue;
(b) administering a treatment to the subject,
wherein the evaluation comprises:
(i) determining in a biological sample from the subject a concentration of one or more proteins selected from Tables 201-218 or selected from Tables 2001-2018 that respectively correspond to said tissues, and (ii) applying a classifier to the concentration of the one or more proteins to identify with which tissue the cancer is associated' or
(i) determining in a biological sample from the subject a concentration of each protein from Table 251, 252, 253, or 254, and applying a classifier to the concentration of each protein to identify with which tissue the cancer is associated.

222. The method of any one of claims 218-221, wherein the results in (a) are acquired from a third party.

223. The method of any one of claims 215-222, wherein the subject is suspected of having a cancer associated with the bladder or of having bladder cancer.

224. The method of claim 223, wherein the one or more proteins are selected from Table 201 or Table 2001.

225. The method of claim 224, wherein the one or more proteins are selected from Table 201.A.1, 201.A.2, 2001.A.1, 2001.A.2, 2001.A.3, 201.B.1, 201.B.2, 201.B.3, 201.B.4, 2001.B.1, 2001.B.2, 2001.B.3, 201.C.1, 201.C.2, 201.C.3, 201.C.4, 2001.C.1, 2001.C.2, or 2001.C.3.

226. The method of claim 225, wherein the subject is female.

227. The method of claim 225, wherein the subject is male.

228. The method of claim 224, wherein the one or more proteins is every protein of Table 201.A.1, 201.A.2, 2001.A.1, 2001.A.2, 2001.A.3, 201.B.1, 201.B.2, 201.B.3, 201.B.4, 2001.B.1, 2001.B.2, 2001.B.3, 201.C.1, 201.C.2, 201.C.3, 201.C.4, 2001.C.1, 2001.C.2, or 2001.C.3.

229. The method of any one of claims 223-228, wherein the cancer is early-stage.

230. The method of any one of claims 215-222, wherein the subject is suspected of having a cancer associated with bone or of having one cancer.

231. The method of claim 230, wherein the subject is suspected of having an osteosarcoma.

232. The method of claim 230 or 231, wherein the one or more proteins are selected from Table 202 or Table 2002.

233. The method of claim 232, wherein the one or more proteins are selected from Table 202.A.1, 202.A.2, 2002.A.1, 2002.A.2, 2002.A.3, 202.B.1, 202.B.2, 202.B.3, 202.B.4, 2002.B.1, 2002.B.2, 2002.B.3, 202.C.1, 202.C.2, 202.C.3, 2002.C.1, 2002.C.2, or 2002.C.3.

234. The method of claim 233, wherein the subject is female.

235. The method of claim 233, wherein the subject is male.

236. The method of claim 232, wherein the one or more proteins is every protein of Table 202.A.1, 202.A.2, 2002.A.1, 2002.A.2, 2002.A.3, 202.B.1, 202.B.2, 202.B.3, 202.B.4, 2002.B.1, 2002.B.2, 2002.B.3, 202.C.1, 202.C.2, 202.C.3, 2002.C.1, 2002.C.2, or 2002.C.3.

237. The method of any one of claims 230-236, wherein the cancer is early-stage.

238. The method of any one of claims 215-222, wherein the subject is suspected of having a cancer associated with the brain or of having brain cancer.

239. The method of claim 238, wherein the subject is suspected of having an astrocytoma.

240. The method of claim 238 or 239, wherein the one or more proteins are selected from Table 203 or Table 2003.

241. The method of claim 240, wherein the one or more proteins are selected from Table 203.A.1, 203.A.2, 2003.A.1, 2003.A.2, 2003.A.3, 203.B.1, 203.B.2, 203.B.3, 203.B.4, 2003.B.1, 2003.B.2, 2003.B.3, 203.C.1, 203.C.2, 203.C.3, 203.C.4, 2003.C.1, 2003.C.2, or 2003.C.3.

242. The method of claim 241, wherein the subject is female.

243. The method of claim 241, wherein the subject is male.

244. The method of claim 240, wherein the one or more proteins is every protein of Table 203.A.1, 203.A.2, 2003.A.1, 2003.A.2, 2003.A.3, 203.B.1, 203.B.2, 203.B.3, 203.B.4, 2003.B.1, 2003.B.2, 2003.B.3, 203.C.1, 203.C.2, 203.C.3, 203.C.4, 2003.C.1, 2003.C.2, or 2003.C.3.

245. The method of any one of claims 238-244, wherein the cancer is early-stage.

246. The method of any one of claims 215-222, wherein the subject is suspected of having a cancer associated with the breast or of having breast cancer.

247. The method of claim 246, wherein the one or more proteins are selected from Table 204 or Table 2004.

248. The method of claim 860, wherein the one or more proteins are selected from Table 204.A.1, 204.A.2, 2004.A.1, 2004.A.2, 2004.A.3, 204.B.1, 204.B.2, 204.B.3, or 204.B.4.

249. The method of claim 248, wherein the subject is female.

250. The method of claim 247, wherein the one or more proteins is every protein of Table 204.A.1, 204.A.2, 2004.A.1, 2004.A.2, 2004.A.3, 204.B.1, 204.B.2, 204.B.3, or 204.B.4.

251. The method of any one of claims 246-250, wherein the cancer is early-stage.

252. The method of any one of claims 215-222, wherein the subject is suspected of having a cancer associated with the cervix or of having cervical cancer.

253. The method of claim 252, wherein the one or more proteins are selected from Table 205 or Table 2005.

254. The method of claim 253, wherein the one or more proteins are selected from Table 205.A.1, 205.A.2, 2005.A.1, 2005.A.2, 2005.A.3, 205.B.1, 205.B.2, 205.B.3, or 205.B.4.

255. The method of claim 254, wherein the subject is female.

256. The method of claim 253, wherein the one or more proteins is every protein of Table 205.A.1, 205.A.2, 2005.A.1, 2005.A.2, 2005.A.3, 205.B.1, 205.B.2, 205.B.3, or 205.B.4.

257. The method of any one of claims 215-222, wherein the subject is suspected of having a cancer associated with the colon or of having colorectal cancer.

258. The method of claim 257, wherein the one or more proteins are selected from Table 206 or Table 2006.

259. The method of claim 258, wherein the one or more proteins are selected from Table 206.A.1, 206.A.2, 2006.A.1, 2006.A.2, 2006.A.3, 206.B.1, 206.B.2, 206.B.3, 206.B.4, 2006.B.1, 2006.B.2, 2006.B.3, 206.C.1, 206.C.2, 206.C.3, 2006.C.1, 2006.C.2, or 2006.C.3.

260. The method of claim 259, wherein the subject is female.

261. The method of claim 259, wherein the subject is male.

262. The method of claim 258, wherein the one or more proteins is every protein of Table 206.A.1, 206.A.2, 2006.A.1, 2006.A.2, 2006.A.3, 206.B.1, 206.B.2, 206.B.3, 206.B.4, 2006.B.1, 2006.B.2, 2006.B.3, 206.C.1, 206.C.2, 206.C.3, 2006.C.1, 2006.C.2, or 2006.C.3.

263. The method of any one of claims 257-262, wherein the cancer is early-stage.

264. The method of any one of claims 215-222, wherein the subject is suspected of having a cancer associated with the esophagus or of esophageal cancer.

265. The method of claim 264, wherein the one or more proteins are selected from Table 207 or Table 2007.

266. The method of claim 265, wherein the one or more proteins are selected from Table 207.A.1, 207.A.2, 2007.A.1, 2007.A.2, 2007.A.3, 207.B.1, 207.B.2, 207.B.3, 207.B.4, 2007.B.1, 2007.B.2, 2007.B.3, 207.C.1, 207.C.2, 207.C.3, 2007.C.1, 2007.C.2, or 2007.C.3.

267. The method of claim 266, wherein the subject is female.

268. The method of claim 266, wherein the subject is male.

269. The method of claim 265, wherein the one or more proteins is every protein of Table 207.A.1, 207.A.2, 2007.A.1, 2007.A.2, 2007.A.3, 207.B.1, 207.B.2, 207.B.3, 207.B.4, 2007.B.1, 2007.B.2, 2007.B.3, 207.C.1, 207.C.2, 207.C.3, 2007.C.1, 2007.C.2, or 2007.C.3.

270. The method of any one of claims 264-269, wherein the cancer is early-stage.

271. The method of any one of claims 215-232, wherein the subject is suspected of having a cancer associated with the kidney or of having kidney cancer.

272. The method of claim 271, wherein the one or more proteins are selected from Table 208 or Table 2008.

273. The method of claim 272, wherein the one or more proteins are selected from Table 208.A.1, 208.A.2, 2008.A.1, 2008.A.2, 2008.A.3, 208.B.1, 208.B.2, 208.B.3, 208.B.4, 2008.B.1, 2008.B.2, 2008.B.3, 208.C.1, 208.C.2, 208.C.3, 208.C.4, 2008.C.1, 2008.C.2, or 2008.C.3.

274. The method of claim 273, wherein the subject is female.

275. The method of claim 273, wherein the subject is male.

276. The method of claim 272, wherein the one or more proteins is every protein of Table 208.A.1, 208.A.2, 2008.A.1, 2008.A.2, 2008.A.3, 208.B.1, 208.B.2, 208.B.3, 208.B.4, 2008.B.1, 2008.B.2, 2008.B.3, 208.C.1, 208.C.2, 208.C.3, 208.C.4, 2008.C.1, 2008.C.2, or 2008.C.3.

277. The method of any one of claims 271-276, wherein the cancer is early-stage.

278. The method of any one of claims 215-222, wherein the subject is suspected of having a cancer associated with the liver or of having liver cancer.

279. The method of claim 278, wherein the subject is suspected of having a hepatocellular carcinoma.

280. The method of claim 279, wherein the one or more proteins are selected from Table 209 or Table 2009.

281. The method of claim 280, wherein the one or more proteins are selected from Table 209.A.1, 209.A.2, 2009.A.1, 2009.A.2, 2009.A.3, 209.B.1, 209.B.2, 209.B.3, 209.B.4, 2009.B.1, 2009.B.2, 2009.B.3, 209.C.1, 209.C.2, 209.C.3, 209.C.4, 2009.C.1, 2009.C.2, or 2009.C.3.

282. The method of claim 281, wherein the subject is female.

283. The method of claim 281, wherein the subject is male.

284. The method of claim 280, wherein the one or more proteins is every protein of Table 209.A.1, 209.A.2, 2009.A.1, 2009.A.2, 2009.A.3, 209.B.1, 209.B.2, 209.B.3, 209.B.4, 2009.B.1, 2009.B.2, 2009.B.3, 209.C.1, 209.C.2, 209.C.3, 209.C.4, 2009.C.1, 2009.C.2, or 2009.C.3.

285. The method of any one of claims 278-284, wherein the cancer is early-stage.

286. The method of any one of claims 215-222, wherein the subject is suspected of having a cancer associated with the lung—small cell or of having small cell lung cancer.

287. The method of claim 286, wherein the one or more proteins are selected from Table 210 or Table 2010.

288. The method of claim 287, wherein the one or more proteins are selected from Table 210.A.1, 210.A.2, 2010.A.1, 2010.A.2, 2010.A.3, 210.B.1, 210.B.2, 210.B.3, 210.B.4, 2010.B.1, 2010.B.2, 2010.B.3, 210.C.1, 210.C.2, 210.C.3, 210.C.4, 2010.C.1, 2010.C.2, or 2010.C.3.

289. The method of claim 288, wherein the subject is female.

290. The method of claim 288, wherein the subject is male.

291. The method of claim 287, wherein the one or more proteins is every protein of Table 210.A.1, 210.A.2, 2010.A.1, 2010.A.2, 2010.A.3, 210.B.1, 210.B.2, 210.B.3, 210.B.4, 2010.B.1, 2010.B.2, 2010.B.3, 210.C.1, 210.C.2, 210.C.3, 210.C.4, 2010.C.1, 2010.C.2, or 2010.C.3.

292. The method of any one of claims 286-291, wherein the cancer is early-stage.

293. The method of any one of claims 215-222, wherein the subject is suspected of having a cancer associated with the lung—non-small cell or of having non-small cell lung cancer.

294. The method of claim 293, wherein the one or more proteins are selected from Table 211 or Table 2011.

295. The method of claim 294, wherein the one or more proteins are selected from Table 211.A.1, 211.A.2, 2011.A.1, 2011.A.2, 211.B.1, 211.B.2, 211.B.3, 211.B.4, 2011.B.1, 2011.B.2, 2011.B.3, 211.C.1, 211.C.2, 211.C.3, 2011.C.1, 2011.C.2, or 2011.C.3.

296. The method of claim 295, wherein the subject is female.

297. The method of claim 295, wherein the subject is male.

298. The method of claim 294, wherein the one or more proteins is every protein of Table 211.A.1, 211.A.2, 2011.A.1, 2011.A.2, 211.B.1, 211.B.2, 211.B.3, 211.B.4, 2011.B.1, 2011.B.2, 2011.B.3, 211.C.1, 211.C.2, 211.C.3, 2011.C.1, 2011.C.2, or 2011.C.3.

299. The method of any one of claims 293-298, wherein the cancer is early-stage.

300. The method of any one of claims 215-222, wherein the subject is suspected of having a cancer associated with the ovary or of having ovarian cancer.

301. The method of claim 300, wherein the one or more proteins are selected from Table 212 or Table 2012.

302. The method of claim 301, wherein the one or more proteins are selected from Table 212.A.1, 212.A.2, 2012.A.1, 2012.A.2, 2012.A.3, 212.B.1, 212.B.2, 212.B.3, or 212.B.4.

303. The method of claim 302, wherein the subject is female.

304. The method of claim 301, wherein the one or more proteins is every protein of Table 212.A.1, 212.A.2, 2012.A.1, 2012.A.2, 2012.A.3, 212.B.1, 212.B.2, 212.B.3, or 212.B.4.

305. The method of any one of claims 300-304, wherein the cancer is early-stage.

306. The method of any one of claims 215-222, wherein the subject is suspected of having a cancer associated with the pancreas or of having pancreatic cancer.

307. The method of claim 306, wherein the one or more proteins are selected from Table 213 or Table 2013.

308. The method of claim 307, wherein the one or more proteins are selected from Table 213.A.1, 213.A.2, 2013.A.1, 2013.A.2, 2013.A.3, 213.B.1, 213.B.2, 213.B.3, 2013.B.1, 2013.B.2, 2013.B.3, 213.C.1, 213.C.2, 213.C.3, 2013.C.1, 2013.C.2, or 2013.C.3.

309. The method of claim 308, wherein the subject is female.

310. The method of claim 308, wherein the subject is male.

311. The method of claim 307, wherein the one or more proteins is every protein of Table 213.A.1, 213.A.2, 2013.A.1, 2013.A.2, 2013.A.3, 213.B.1, 213.B.2, 213.B.3, 2013.B.1, 2013.B.2, 2013.B.3, 213.C.1, 213.C.2, 213.C.3, 2013.C.1, 2013.C.2, or 2013.C.3.

312. The method of any one of claims 306-311, wherein the cancer is early-stage.

313. The method of any one of claims 215-222, wherein the subject is suspected of having a cancer associated with the prostate or of having prostate cancer.

314. The method of claim 313, wherein the one or more proteins are selected from Table 214 or Table 2014.

315. The method of claim 314, wherein the one or more proteins are selected from Table 214.A.1, 214.A.2, 2014.A.1, 2014.A.2, 2014.A.3, 214.B.1, 214.B.2, 214.B.3, or 214.B.4.

316. The method of claim 315, wherein the subject is male.

317. The method of claim 314, wherein the one or more proteins is every protein of Table 214.A.1, 214.A.2, 2014.A.1, 2014.A.2, 2014.A.3, 214.B.1, 214.B.2, 214.B.3, or 214.B.4.

318. The method of any one of claims 313-317, wherein the cancer is early-stage.

319. The method of any one of claims 215-222, wherein the subject is suspected of having a cancer associated with the stomach or of having stomach cancer.

320. The method of claim 319, wherein the one or more proteins are selected from Table 215 or Table 2015.

321. The method of claim 320, wherein the one or more proteins are selected from Table 215.A.1, 215.A.2, 2015.A.1, 2015.A.2, 2015.A.3, 215.B.1, 215.B.2, 215.B.3, 215.B.4, 2015.B.1, 2015.B.2, 2015.B.3, 215.C.1, 215.C.2, 215.C.3, 215.C.4, 2015.C.1, 2015.C.2, or 2015.C.3.

322. The method of claim 321, wherein the subject is female.

323. The method of claim 321, wherein the subject is male.

324. The method of claim 320, wherein the one or more proteins is every protein of Table 215.A.1, 215.A.2, 2015.A.1, 2015.A.2, 2015.A.3, 215.B.1, 215.B.2, 215.B.3, 215.B.4, 2015.B.1, 2015.B.2, 2015.B.3, 215.C.1, 215.C.2, 215.C.3, 215.C.4, 2015.C.1, 2015.C.2, or 2015.C.3.

325. The method of any one of claims 319-324, wherein the cancer is early-stage.

326. The method of any one of claims 215-222, wherein the subject is suspected of having a cancer associated with the testis or of having testicular cancer.

327. The method of claim 326, wherein the one or more proteins are selected from Table 216 or Table 2016.

328. The method of claim 327, wherein the one or more proteins are selected from Table 216.A.1, 216.A.2, 2016.A.1, 2016.A.2, 2016.A.3, 216.B.1, 216.B.2, 216.B.3, or 216.B.4.

329. The method of claim 328, wherein the subject is male.

330. The method of claim 327, wherein the one or more proteins is every protein of Table 216.A.1, 216.A.2, 2016.A.1, 2016.A.2, 2016.A.3, 216.B.1, 216.B.2, 216.B.3, or 216.B.4.

331. The method of any one of claims 326-330, wherein the cancer is early-stage.

332. The method of any one of claims 215-222, wherein the subject is suspected of having a cancer associated with the thyroid.

333. The method of claim 332, wherein the one or more proteins are selected from Table 217 or Table 2017.

334. The method of claim 333, wherein the one or more proteins are selected from Table 217.A.1, 217.A.2, 2017.A.1, 2017.A.2, 2017.A.3, 217.B.1, 217.B.2, 217.B.3, 217.B.4, 2017.B.1, 2017.B.2, 2017.B.3, 217.C.1, 217.C.2, 217.C.3, 217.C.4, 2017.C.1, 2017.C.2, or 2017.C.3.

335. The method of claim 334, wherein the subject is female.

336. The method of claim 334, wherein the subject is male.

337. The method of claim 333, wherein the one or more proteins is every protein of Table 217.A.1, 217.A.2, 2017.A.1, 2017.A.2, 2017.A.3, 217.B.1, 217.B.2, 217.B.3, 217.B.4, 2017.B.1, 2017.B.2, 2017.B.3, 217.C.1, 217.C.2, 217.C.3, 217.C.4, 2017.C.1, 2017.C.2, or 2017.C.3.

338. The method of any one of claims 332-337, wherein the cancer is early-stage.

339. The method of any one of claims 215-222, wherein the subject is suspected of having a cancer associated with the uterus or of having uterine cancer.

340. The method of claim 339, wherein the one or more proteins are selected from Table 218 or Table 2018.

341. The method of claim 340, wherein the one or more proteins are selected from Table 218.A.1, 218.A.2, 2018.A.1, 2018.A.2, or 2018.A.3, 218.B.1, 218.B.2, 218.B.3, or 218.B.4.

342. The method of claim 341, wherein the subject is female.

343. The method of claim 340, wherein the one or more proteins is every protein of Table 218.A.1, 218.A.2, 2018.A.1, 2018.A.2, or 2018.A.3, 218.B.1, 218.B.2, 218.B.3, or 218.B.4.

344. The method of any one of claims 339-343, wherein the cancer is early-stage.

345. The method of any one of claims 215-222, comprising determining in a biological sample from the subject a concentration of each protein from Table 251, 252, 253, or 254.

346. The method of claim 345, wherein the subject is female.

347. The method of claim 345, wherein the subject is male.

348. The method of any one of claims 345-347, wherein the cancer is early-stage.

349. The method of any one of claims 215-348, wherein the subject is symptomatic of a cancer.

350. The method of any one of claims 1-349, wherein the sample is selected from a plasma sample, urine sample, saliva sample, cerebrospinal fluid (CSF) sample, sweat sample, and tear sample.

351. The method of any one of claims 1-349, wherein the number of proteins for which the concentration is determined is sufficient to achieve an area-under-the-curve (AUC) of a ROC curve of at least about 0.6.

352. The method of claim 351, wherein the number of proteins for which the concentration is determined is sufficient to achieve an AUC of a ROC curve of at least about 0.7.

353. The method of claim 352, wherein the number of proteins for which the concentration is determined is sufficient to achieve an AUC of a ROC curve of at least about 0.8.

354. The method of claim 353, wherein the number of proteins for which the concentration is determined is sufficient to achieve an AUC of a ROC curve of at least about 0.9.

355. The method of any one of claims 1-354, wherein the concentration of the two or more proteins is determined by one or more assays.

356. A method of measuring amounts of proteins in a subject, the method comprising determining individual amounts of one or more proteins selected from Table 101 or selected from Table 1001.

357. A method of measuring amounts of proteins in a subject, the method comprising determining individual amounts of one or more proteins selected from any of Tables 201-218, 2001-2018, 251, 252, 253, or 254.

358. A method of measuring amounts of proteins in a subject, the method comprising determining individual amounts of each protein of Table 251, 252, 253, or 254.

359. A method of measuring amounts of proteins in a subject, the method comprising determining individual amounts of one or more proteins selected from any of Tables 301-318.

360. A method of evaluating a treatment for cancer in a subject, the method comprising:
(a) administering a treatment for cancer to the subject, and
(b) determining in a biological sample from the subject a concentration of one or more proteins, wherein the one or more proteins are selected from any of Tables 101, 1001, 201-218, 2001-2018, or 301-318; or determining in a biological sample from the subject a concentration of each protein of Table 251, 252, 253, or 254;
thereby evaluating the subject for cancer.

361. A method of evaluating the efficacy of a treatment, comprising
(a) administering a treatment for cancer to the subject, and
(b) determining in a biological sample from the subject a concentration of one or more proteins, wherein the one or more proteins are selected from any of Tables 101, 1001, 201-218, 2001-2018, or 301-318; or determining in a biological sample from the subject a concentration of each protein of Table 251, 252, 253, or 254;
thereby evaluating the subject for cancer.

362. A method of treating cancer in a subject, comprising
(a) administering a treatment for cancer, and
(b) determining in a biological sample from the subject a concentration of one or more proteins to evaluate the efficacy of the treatment, wherein the one or more proteins are selected from any of Tables 101, 1001, 201-218, 2001-2018, or 301-318: or determining in a biological sample from the subject a concentration of each protein of Table 251, 252, 253, or 254.

363. A method of adjusting a treatment for cancer, comprising
(a) administering a treatment for cancer, and
(b) determining in a biological sample from the subject a concentration of one or more proteins, wherein the one or more proteins are selected from any of Tables 101, 1001, 201-218, 2001-2018, or 301-318: or determining in a biological sample from the subject a concentration of each protein of Table 251, 252, 253, or 254.

364. A method of treating cancer in a subject, comprising
(a) administering a treatment for cancer, and
(b) determining in a biological sample from the subject a concentration of one or more proteins to evaluate whether the treatment requires adjustment, wherein the one or more proteins are selected from any of Tables 101, 1001, 201-218, 2001-2018, or 301-318; or determining in a biological sample from the subject a concentration of each protein of Table 251, 252, 253, or 254.

365. A method of monitoring for cancer recurrence in a subject, comprising
(a) administering a treatment for cancer, and
(b) determining in a biological sample from the subject a concentration of one or more proteins to evaluate whether the treatment requires adjustment, wherein the one or more proteins are selected from any of Tables 101, 1001, 201-218, 2001-2018, or 301-318; or determining in a biological sample from the subject a concentration of each protein of Table 251, 252, 253, or 254.

366. The method of claim 1191, further comprising administering an adjusted treatment when it is determined that treatment requires adjustment.

367. A method of treating cancer in a subject, comprising
(a) administering a treatment for cancer, and
(b) determining in a biological sample from the subject a concentration of one or more proteins to evaluate whether cancer is recurring, wherein the one or more proteins are selected from any of Tables 101, 1001, 201-218, 2001-2018, or 301-318; or determining in a biological sample from the subject a concentration of each protein of Table 251, 252, 253, or 254.

368. The method of claim 367, further comprising administering a second treatment when it is determined that the cancer is recurring.

369. The method of any one of claims 360-368, wherein the one or more proteins are selected from Table 101.

370. The method of claim 369, further comprising applying a classifier to the concentration of the one or more proteins that identifies whether the concentration of the one or more proteins is indicative of the subject having any cancer.

371. The method of any one of claims 360-368, wherein the one or more proteins are selected from Table 1001.

372. The method of claim 371, further comprising applying a classifier to the concentration of the one or more proteins that identifies whether the concentration of the one or more proteins is indicative of the subject having any cancer.

373. The method of claim 360-368, wherein the one or more proteins are selected from Tables 201-218.

374. The method of claim 373, further comprising applying a classifier to the concentration of the one or more proteins selected from Tables 201-218 that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

375. The method of claim 360-368, wherein the one or more proteins are selected from Tables 2001-2018.

376. The method of claim 375, further comprising applying a classifier to the concentration of the one or more proteins selected from Tables 2001-2018 that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

377. The method of claim 360-368, wherein the one or more proteins are selected from Tables 301-318.

378. The method of claim 377, further comprising applying a classifier to the concentration of the one or more proteins selected from Tables 301-318 that identifies whether the concentration of the one or more proteins is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

379. The method of claim 360-368, wherein the concentration is of each protein from Table 251, 252, 253, or 254.

380. The method of claim 379, further comprising applying a classifier to the concentration of each protein from Table 251, 252, 253, or 254 that identifies whether the concentration is indicative of the subject having a cancer associated with a tissue selected from bladder, bone, brain, breast, cervix, colon, esophagus, kidney, liver, lung—small cell, lung—non-small cell, ovary, pancreas, prostate, stomach, testis, thyroid, and uterus.

381. The method of any one of claims 360-380, wherein the administration of the treatment in (a) is performed by a third party.

382. The method of any one of claims 360-380, wherein the determination in a biological sample from the subject a concentration of one or more proteins in (b) is performed by a third party.
